(19) United States
(12) Patent Application Publication
(10) Pub. No.: US 2026/0364193 A1
(43) Pub. Date: Nov. 19, 2026

(54) ANTI-VISTA ANTIBODY RNA OR NUCLEIC ACID CONJUGATES (ARCS OR ANCS), COMPOSITIONS CONTAINING, AND THERAPEUTIC USE THEREOF

(71) Applicant: LIFORDI IMMUNOTHERAPEUTICS, INC., Lebanon, NH (US)

(72) Inventors: Jay ROTHSTEIN, Lebanon, NH (US); Sergey SEREGIN, Lebanon, NH (US); Anna KUTA, Lebanon, NH (US); Xin HUANG, Lebanon, NH (US); Kierstin BELL, Lebanon, NH (US); Nicholas SCHWERTNER, Lebanon, NH (US); Catherine CARRIERE, Lebanon, NH (US); Maria DAY, Lebanon, NH (US); Roman UZHACHENKO, Lebanon, NH (US); Maheshwerreddy CHILAMARI, Lebanon, NH (US)

(21) Appl. No.: 19/490,406

(22) PCT Filed: Jun. 5, 2024
(86) PCT No.: PCT/US2024/032660

§ 371 Date: Dec. 5, 2025

Related U.S. Application Data
(60) Provisional application No. 63/506,177, filed on Jun. 5, 2023.
(60) Provisional application No. 63/611,302, filed on Dec. 18, 2023.

(51) Int. Cl. A61K 47/68; A61K 31/7105; A61P 37/06; C07K 16/28; C12N 9/22; C12N 15/11; C12N 15/113

(52) U.S. Cl. CPC A61K 47/6807; A61K 31/7105; A61K 47/6849; A61K 47/6889; A61P 37/06; C07K 16/2827; C12N 9/222; C12N 15/111; C12N 15/1137; C12N 2310/14; C12N 2310/20; C12Y 207/01153

(57) ABSTRACT

The present invention relates to anti-VISTA antibody RNA or nucleic acid conjugates (ARCs or ANCs) which specifically deliver at least one nucleic acid, e.g., RNAs into immune cells and the use of such ARCs or ANCs as therapeutics, e.g., for treating autoimmune, allergic and inflammatory conditions, or for treating cancer and/or for treating inflammatory symptoms associated therewith elicited by specific immune cell types which express VISTA.

### RELATED APPLICATIONS

The present PCT application claims benefit of priority to U.S. Provisional Application No. 63/506,177, filed on Jun. 5, 2023 and U.S. Provisional Application No. 63/611,302, filed on Dec. 18, 2023, the contents of both of which are incorporated by reference in their entireties.

### SEQUENCE LISTING DISCLOSURE

The contents of the electronic sequence listing (1143260_008613_SL.xml; Size: 707,682 bytes; and Date of Creation: Jun. 3, 2024) is herein incorporated by reference in its entirety.

### FIELD OF THE INVENTION

The present invention disclosed herein relates to anti-VISTA antibody RNA or nucleic acid conjugates (ARCs or ANCs) which specifically deliver at least one nucleic acid, e.g., RNAs or DNAs or nucleic acid/protein complexes into immune cells and the use of such ARCs or ANCs as therapeutics, e.g., for treating autoimmune and inflammatory conditions, or for treating cancer and/or symptoms associated therewith elicited by specific immune cell types.

### BACKGROUND OF THE INVENTION

Modulation of RNA function is a developing area of therapeutic interest. Drugs that affect mRNA stability like antisense oligonucleotides and short interfering RNAs are one way to modulate RNA function. Another group of oligonucleotides can modulate RNA function by altering the processing of pre-mRNA to include or exclude specific regions of pre-mRNAs from the ultimate gene product: the encoded protein. Accordingly, oligonucleotide therapeutics represent a means of modulating protein expression in disease states and as such have utility as therapeutics.

Additionally, the delivery of RNAs to target cells by incorporation into ANCs or ARCs is known. However, existing ANCs or ARCs do not target immune cell types.

### SUMMARY OF THE INVENTION

In one aspect, the present invention relates to anti-VISTA antibody RNA conjugates (ARCs) which may be used to specifically deliver nucleic acids, e.g., RNAs or DNAs or nucleic acid/protein complexes into immune cells.

In another aspect the present invention relates to the use of such ARCs as therapeutics, e.g., for treating autoimmune, inflammatory and cancer conditions.

In specific aspects the present invention provides an antibody-RNA or antibody-nucleic acid conjugate (“ARC” or “ANC”) which comprises (i) an antibody or antibody fragment which binds to an antigen specifically or predominantly expressed by one or more immune cell types; and to which is directly or indirectly conjugated (ii) one or more nucleic acids, preferably RNA or DNA oligonucleotides (“payload” or “payloads”) comprised of wild-type or modified nucleotides, which oligonucleotides specifically bind to a target gene expressed by an immune cell, optionally an immunomodulatory gene or to an RNA encoded thereby; and optionally (iii) a cleavable or non-cleavable linker or adaptor, e.g., a peptide intervening said (i) antibody or antibody fragment and said (ii) one or more nucleic acids; wherein such ARC or ANC when in contact with an immune cell which expresses the antigen bound by the (i) an antibody or antibody fragment is internalized by the immune cell and provides for the release of said (ii) one or more nucleic acids into the immune cell; thereby optionally modulating the expression and/or function of the targeted immune modulator.

In some specific aspects the ARC or ANC of any of the foregoing comprises one or more payloads which comprise one or more modified nucleotides, optionally at least one phosphonate and/or ribose modified nucleotide which facilitates direct or indirect attachment of the one or more payloads to the antibody or antibody fragment, optionally via a peptide linker, further optionally a cleavable or non-cleavable linker or adaptor, e.g., a peptide intervening said (i) antibody or antibody fragment and (ii) a payload.

In some specific aspects the ARC or ANC of any of the foregoing comprises a payload which is directly or indirectly conjugated to the antibody or antibody fragment via a reactive amine, optionally comprised on a lysine residue on the antibody or antibody fragment and/or on a peptide linking the (i) antibody or antibody fragment to (ii) one or more payloads.

In some specific aspects the ARC or ANC of any of the foregoing comprises an antibody or antibody fragment which binds to VISTA, preferably human VISTA.

In some specific aspects the ARC or ANC of any of the foregoing comprises an antibody or antibody fragment that binds to VISTA, preferably human VISTA, and comprises the same VH and VL CDRs as any one of anti-human VISTA antibodies comprising the sequences in FIG. 14 or in Appendix 1 or Appendix 3.

In some specific aspects in any of the foregoing the antibody or antibody fragment in the ARC or ANC binds to VISTA, preferably human VISTA, and comprises the same VH and/or VL regions and CDRs as any one of anti-human VISTA antibodies comprising the VH and/or VL sequences in FIG. 14 or an antibody or antibody fragment comprising VH and/or VL regions possessing a least 90, 95 or 99% sequence identity to the VH and/or VL regions as any one of anti-human VISTA antibodies comprising the VH and/or VL sequences in FIG. 14; or an antibody or antibody fragment which comprises the VH and/or VL sequences in Appendix 1 or Appendix 3; and which antibody or antibody fragment optionally comprises IgG1, IgG2, IgG3 or IgG4 constant domain polypeptides, further optionally IgG1 constant domain polypeptides, still further optionally IgG1 constant domain polypeptides having the sequences contained in Appendix 1 or 3.

In some specific aspects the antibody or antibody fragment on the ANC or ARC comprises a human Fc region, optionally human IgG1, IgG2, IgG3 or IgG4, further optionally modified to impair complement and/or FcR binding and/or to enhance FcRn binding.

In some specific aspects the ARC or ANC of any of the foregoing comprises one or more of a short interfering RNA (siRNA), antisense oligonucleotide (ASO), short hairpin RNA (shRNA), microRNA (miRNA), double-stranded RNA (dsRNA), transfer RNA (tRNA), ribosomal RNA (rRNA), or heterogeneous nuclear RNA (hnRNA).

In some specific aspects the ARC or ANC of any of the foregoing comprises a polynucleic acid molecule which is from about 10 to about 1000, 10 to about 500, 10 to about 400, 10 to about 300, 10 to about 200, 10 to about 150, 10 to about 100, 10 to about 50, about 10 to about 30, about 15 to about 30, about 18 to about 25, about 18 to about 24, about 19 to about 23, or about 20 to about 22 nucleotides in length; or which comprises a polynucleic acid molecule of about 50 nucleotides, about 45 nucleotides, about 40 nucleotides, about 35 nucleotides, about 30 nucleotides, about 25 nucleotides, about 20 nucleotides, about 19 nucleotides, about 18 nucleotides, about 17 nucleotides, about 16 nucleotides, about 15 nucleotides, about 14 nucleotides, about 13 nucleotides, about 12 nucleotides, about 11 nucleotides, or about 10 nucleotides in length.

In some specific aspects the ARC or ANC of any of the foregoing comprises a first polynucleotide and a second polynucleotide, optionally wherein the first polynucleotide is a sense strand or passenger strand and/or the second polynucleotide is an antisense strand or guide strand.

In some specific aspects the ARC or ANC of any of the foregoing comprises an siRNA, ASO, tRNA, rRNA, or a mRNA.

In some specific aspects the ARC or ANC of any of the foregoing comprises, or is encapsulated in or conjugated to a lipid nanoparticle.

In some specific aspects the ARC or ANC of any of the foregoing comprises at least one payload targets an immunomodulator selected from a cytokine, chemokine, interleukin, interferon, tumor necrosis factor, or receptor of any of the foregoing.

In some specific aspects the ARC or ANC of any of the foregoing comprises a payload which targets an RNA or DNA sequence encoding an immunomodulator selected from is IL-2, IL-3, IL-4, IL-5, IL-6, IL-7, IL-8, IL-10, IL-11, IL-12, 11-15, IL-17, IL-18, IL-22, IL-37, IL-1β, TGF-β, IFNα, IFNβ, IFNγ, TNF-α, TNF-β, GM-CSF, phosphatidylinositol-4,5-bisphosphate 3-kinase catalytic subunit alpha (PIK3CA), RAR related orphan receptor C (RORC) or any of the molecules having the sequences identified in FIG. 1 or FIG. 2.

In some specific aspects the ARC or ANC of any of the foregoing comprises an siRNA payload which targets an RNA or DNA encoding GLUT3 or PIK3CA, optionally a sequence in Appendix 2 or 4.

In some specific aspects the ARC or ANC of any of the foregoing comprises an antibody or antibody fragment which binds to at least one immune cell selected from PMBCs, a T cell, a T cell progenitor cell, a CD4+ T cell, a helper T cell, a regulatory T cell, a CD8+ T cell, a naïve T cell, an effector T cell, a memory T cell, a stem cell memory T (TSCM) cell, a central memory T (TCM) cell, an effector memory T (TEM) cell, a terminally differentiated effector memory T cell, a tumor-infiltrating lymphocyte (TIL), an immature T cell, a mature T cell, a cytotoxic T cell, a mucosa-associated invariant T (MAIT) cell, a TH1 cell, a TH2 cell, a TH3 cell, a TH17 cell, a TH9 cell, a TH22 cell, a follicular helper T cells, and a/b T cell, a g/d T cell, a Natural Killer T (NKT) cell, a cytokine-induced killer (CIK) cell, a lymphokine-activated killer (LAK) cell, a perforin-deficient cell, a granzyme-deficient cell, a B cell, a myeloid cell, a monocyte, a macrophage, an eosinophil, a neutrophil, and a dendritic cell.

In some specific aspects the ARC or ANC of any of the foregoing comprises an antibody or antibody fragment that binds to a myeloid cell and/or a T cell.

In some specific aspects the ARC or ANC of any of the foregoing comprises an antibody or antibody fragment that binds to a T cell or T cell progenitor cell or NK cell.

In some specific aspects the ARC or ANC of any of the foregoing comprises at least one nucleic acid payload, optionally an RNA or DNA, further optionally an siRNA or antisense RNA, that binds to a gene or nucleic acid, optionally an RNA or DNA, encoding an antigen selected from the group consisting of: (1) 17-IA, 4-1B, 4Dc, 6-keto-PGFIa, 8-iso-PGF2a, 8-oxo-dG, AI Adenosine Receptor, A33, ACE, ACE-2, Activin, Activin A, Activin AB, Activin B, Activin C, Activin RIA, Activin RIA ALK-2, Activin RIB ALK-4, Activin RIIA, Activin RUB, ADAM, ADAM10, ADAM12, ADAM 15, ADAM 17/T ACE, ADAM8, ADAM9, ADAMTS, ADAMTS4, ADAMTS5, Addressins, aFGF, ALCAM, ALK, ALK-1, ALK-7, alpha-l-antitrypsin, alpha-V/beta-1 antagonist, ANG, Ang, APAF-1, APE, APJ, APP, APRIL, AR, ARC, ART, Artemin, anti-Id, ASPARTIC, Atrial natriuretic factor, av/b3 integrin, Axl, b2M, B7-1, B7-2, B7-H, B-lymphocyte Stimulator (BlyS), BACE, BACE-1, Bad, BAFF, BAFF-R, Bag-1, BAK, Bax, BCA-1, BCAM, Bel, BCMA, BDNF, b-ECGF, bFGF, BID, Bik, BFM, BLC, BL-CAM, BLK, BMP, BMP-2 BMP-2a, BMP-3 Osteogenin, BMP-4 BMP-2b, BMP-5, BMP-6 Vgr-1, BMP-7 (OP-1), BMP-8 (BMP-8a, OP-2), BMPR, BMPR-IA (ALK-3), BMPR-IB (ALK-6), BRK-2, RPK-1, BMPR-II (BRK-3), BMPs, b-NGF, BOK, Bombesin, Bone-derived neurotrophic factor, BPDE, BPDE-DNA, BTC, complement factor 3 (C3), C3a, C4, C5, C5a, CIO, CA125, CAD-8, Calcitonin, cAMP, carcinoembryonic antigen (CEA), carcinoma-associated antigen, Cathepsin A, Cathepsin B, Cathepsin C/DPPI, Cathepsin D, Cathepsin E, Cathepsin H, Cathepsin L, Cathepsin O, Cathepsin S, Cathepsin V, Cathepsin X/Z/P, CBL, CCI, CCK2, CCL, CCL1, CCL11, CCL12, CCL13, CCL 14, CCL15, CCL16, CCLI 7, CCL18, CCL19, CCL2, CCL20, CCL21, CCL22, CCL23, CCL24, CCL25, CCL26, CCL27, CCL28, CCL3, CCL4, CCL5, CCL6, CCL7, CCL8, CCL9/10, CCR, CCR1, CCR10, CCR10, CCR2, CCR3, CCR4, CCR5, CCR6, CCR7, CCR8, CCR9, CD1, CD2, CD4, CD5, CD6, CD7, CD8, CD10, CDIIa, CD11b, CD11c, CD13, CD14, CD15, CD16, CD18, CD19, CD20, CD21, CD22, CD23, CD25, CD27L, CD28, CD29, CD30, CD30L, CD32, CD33 (p67 proteins), CD34, CD38, CD40, CD40L, CD44, CD45, CD46, CD49a, CD52, CD54, CD55, CD56, CD61, CD64, CD66e, CD74, CD80 (B7-1), CD89, CD95, CD123, CD137, CD138, CD140a, CD146, CD147, CD148, CD152, CD164, CEACAM5, CFTR, cGMP, CINC, Clostridium botulinum toxin, Clostridium perfringens toxin, CKb8-I, CLC, CMV, CMV UL, CNTF, CNTN-1, COX, C-Ret, CR6-2, CT-1, CTACK, CTGF, CTLA-4, CX3CL1, CX3CR1, CXCL, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCR, CXCR1, CXCR2, CXCR3, CXCR4, CXCR5, CXCR6, cytokeratin tumor-associated antigen, DAN, DCC, DcR3, DC-SIGN, Decay accelerating factor, des(I-3)-IGF-I (brain IGF-1), Dhh, digoxin, DNAM-1, Dnase, Dpp, DPPIV/CD26, Dtk, ECAD, EDA, EDA-A1, EDA-A2, EDAR, EGF, EGFR (ErbB-1), EMA, EMMPRIN, EN A, endothelin receptor, Enkephalinase, eNOS, Eot, eotaxinl, EpCAM, Ephrin B2/EphB4, EPO, ERCC, E-selectin, ET-1, Factor Ila, Factor VII, Factor VIIIc, Factor IX, fibroblast activation protein (FAP), Fas, FcRI, FEN-1, Ferritin, FGF, FGF-19, FGF-2, FGF3, FGF-8, FGFR, FGFR-3, Fibrin, FL, FLIP, Flt-3, Flt-4, Follicle stimulating hormone, Fractalkine, FZD1, FZD2, FZD3, FZD4, FZD5, FZD6, FZD7, FZD8, FZD9, FZD10, G250, Gas 6, GCP-2, GCSF, GD2, GD3, GDF, GDF-1, GDF-3 (Vgr-2), GDF-5 (BMP-14, CDMP-1), GDF-6 (BMP-13, CDMP-2), GDF-7 (BMP-12, CDMP-3), GDF-8 (Myostatin), GDF-9, GDF-15 (MIC-1), GDNF, GFAP, GFRa-1, GFR-alpha1, GFR-alpha2, GFR-alpha3, GITR, Glucagon, Glut 4, glycoprotein Ib/IIIa (GP IIb/IIIa), GM-CSF, gpl30, gp72, GRO, Growth hormone releasing factor, Hapten (NP-cap or NIP-cap), HB-EGF, HCC, HCMV gB envelope glycoprotein, HCMV) gH envelope glycoprotein, HCMV UL, Hemopoietic growth factor (HGF), Hep B gpl20, heparanase, Her2, Her2/neu (ErbB-2), Her3 (ErbB-3), Her4 (ErbB-4), herpes simplex virus (HSV) gB glycoprotein, HSV gD glycoprotein, HGFA, High molecular weight melanoma-associated antigen (HMW-MAA), HIV gpl20, HIV IIB gp 120 V3 loop, HLA, HLA-DR, HM1.24, HMFG PEM, HRG, Hrk, human cardiac myosin, human cytomegalovirus (HCMV), human growth hormone (HGH), HVEM, 1-309, IAP, ICAM, ICAM-1, ICAM-3, ICE, ICOS, IFNg, Ig, IgA receptor, IgE, IGF, IGF binding proteins, IGF-1R, IGFBP, IGF-I, IGF-II, IL, IL-1, IL-1R, IL-2, IL-2R, IL-4, IL-4R, IL-5, IL-5R, IL-6, IL-6R, IL-8, IL-9, IL-10, IL-12, IL-13, IL-15, IL-18, IL-18R, IL-23, interferon (INF)-alpha, INF-beta, INF- gamma, Inhibin, iNOS, Insulin A-chain, Insulin B-chain, Insulin-like growth factor 1, integrin alpha2, integrin alpha3, integrin alpha4, integrin alpha4/beta1, integrin, alpha4/beta7, integrin alpha5 (alphaV), integrin alpha5/beta1, integrin alpha5/beta3, integrin alpha6, integrin beta1, integrin beta2, interferon gamma, IP-10, 1-TAC, JE, Kallikrein 2, Kallikrein 5, Kallikrein 6, Kallikrein 11, Kallikrein 12, Kallikrein 14, Kallikrein 15, Kallikrein LI, Kallikrein L2, Kallikrein L3, Kallikrein L4, KC, KDR, Keratinocyte Growth Factor (KGF), laminin 5, LAMP, LAP, LAP (TGF-1), Latent TGF-1, Latent TGF-1 bpl, LBP, LDGF, LECT2, Lefty, Lewis-Y antigen, Lewis-Y related antigen, LFA-1, LFA-3, Lfo, LIF, LIGHT, lipoproteins, LIX, LKN, Lptn, L-Selectin, LT-a, LT-b, LTB4, LTBP-1, Lung surfactant, Luteinizing hormone, Lymphotoxin Beta Receptor, Mac-1, MAdCAM, MAG, MAP2, MARC, MCAM, MCAM, MCK-2, MCP, M-CSF, MDC, Mer, a metalloprotease, MGDF receptor, MGMT, MHC (HLA-DR), MIF, MIG, MIP, MIP-1-alpha, MK, MMAC1, MMP, MMP-1, MMP-10, MMP-11, MMP-12, MMP-13, MMP-14, MMP-15, MMP-2, MMP-24, MMP-3, MMP-7, MMP-8, MMP-9, MPIF, Mpo, MSK, MSP, mucin (Mucl), MUC18, Muellerian- inhibiting substance, Mug, MuSK, NAIP, NAP, NCAD, N-Cadherin, NCA 90, NCAM, Neprilysin, Neurotrophin-3, -4, or -6, Neurturin, Neuronal growth factor (NGF), NGFR, NGF-beta, nNOS, NO, NOS, Npn, NRG-3, NT, NTN, OB, OGG1, OPG, OPN, OSM, OX40L, OX40R, p150, p95, PADPr, Parathyroid hormone, PARC, PARP, PBR, PBSF, PCAD, P-Cadherin, PCNA, PDGF, PDGF, PDK-1, PECAM, PEM, PF4, PGE, PGF, PG12, PGJ2, PIN, PLA2, placental alkaline phosphatase (PLAP), PIGF, PLP, PP14, Proinsulin, Prorelaxin, Protein C, PS, PSA, PSCA, prostate specific membrane antigen (PSMA), PTEN, PTHrp, Ptk, PTN, R51, RANK, RANKL, RANTES, Relaxin A-chain, Relaxin B-chain, renin, respiratory syncytial virus (RSV) F, RSV Fgp, Ret, Rheumatoid factors, RLIP76, RPA2, RSK, S100, SCF/KL, SDF-1, SERINE, Serum albumin, sFRP-3, Shh, SIGIRR, SK-1, SLAM, SLPI, SMAC, SMDF, SMOH, SOD, SPARC, Stat, STEAP, STEAP-II, TACE, TACI, TAG-72 (tumor- associated glycoprotein-72), TARC, TCA-3, T-cell receptors (e.g., T-cell receptor alpha/beta), TdT, TECK, TEM1, TEM5, TEM7, TEM8, TERT, testicular PLAP-like alkaline phosphatase, TfR, TGF, TGF-alpha, TGF-beta, TGF-beta Pan Specific, TGF-beta RI (ALK-5), TGF-beta RII, TGF-beta RIIb, TGF-beta RIII, TGF-beta1, TGF-beta2, TGF-beta3, TGF-beta4, TGF-beta5, Thrombin, Thymus Ck-1, Thyroid stimulating hormone, Tie, TIMP, TIQ, TMEFF2, Tmpo, TMPRSS2, TNF, TNF-alpha, TNF-alpha beta, TNF-beta2, TNFc, TNF-RI, TNF-RII, TNFRSF10A (TRAIL RI Apo-2, DR4), TNFRSFIOB (TRAIL R2 DR5, KILLER, TRICK-2A, TRICK-B), TNFRSF10C (TRAIL R3 DcRI, LIT, TRID), TNFRSF10D (TRAIL R4 DcR2, TRUNDD), TNFRSFIIA (RANK ODF R, TRANCE R), TNFRSFIIB (OPG OCIF, TR1), TNFRSF12 (TWEAK R FN14), TNFRSF13B (TACI), TNFRSF13C (BAFF R), TNFRSF14 (HVEM ATAR, HveA, LIGHT R, TR2), TNFRSF16 (NGFR p75NTR), TNFRSF17 (BCMA), TNFRSF 18 (GITR AITR), TNFRSF19 (TROY TAJ, TRADE), TNFRSF19L (RELT), TNFRSFIA (TNF RI CD120a, p55-60), TNFRSFIB (TNF RII CD120b, p75-80), TNFRSF26 (TNFRH3), TNFRSF3 (LTbR TNF Rill, TNFC R), TNFRSF4 (OX40 ACT35, TXGP1 R), TNFRSF 5 (CD40 p50), TNFRSF6 (Fas Apo-1, APT1, CD95), TNFRSF6B (DcR3 M68, TR6), TNFRSF7 (CD27), TNFRSF8 (CD30), TNFRSF9 (4-1BB CD137, ILA), TNFRSF21 (DR6), TNFRSF22 (DcTRAIL R2 TNFRH2), TNFRST23 (DcTRAIL RI TNFRH1), TNFRSF25 (DR3 Apo-3, LARD, TR-3, TRAMP, WSL-1), TNFSF10 (TRAIL Apo-2 Ligand, TL2), TNFSF11 (TRANCE/RANK Ligand ODF, OPG Ligand), TNFSF12 (TWEAK Apo-3 Ligand, DR3 Ligand), TNFSF13 (APRIL TALL2), TNFSF13B (BAFF BLYS, TALL1, THANK, TNFSF20), TNFSF14 (LIGHT HVEM Ligand, LTg), TNFSF15 (TLIA/VEGI), TNFSF18 (GITR Ligand AITR Ligand, TL6), TNFSFIA (TNF-a Conectin, DIF, TNFSF2), TNFSF1B (TNF-b LTa, TNFSF1), TNFSF3 (LTb TNFC, p33), TNFSF4 (OX40 Ligand gp34, TXGP1), TNFSF5 (CD40 Ligand CD154, gp39, HIGM1, IMD3, TRAP), TNFSF6 (Fas Ligand Apo-1 Ligand, APT1 Ligand), TNFSF7 (CD27 Ligand CD70), TNFSFS8 (CD30 Ligand CD153), TNFSF9 (4-1BB Ligand CD137 Ligand), TP-1, t-PA, Tpo, TRAIL, TRAIL R, TRAIL-RI, TRAIL-R2, TRANCE, transferring receptor, TRF, Trk, TROP-2, TSG, TSLP, tumor-associated antigen CA 125, tumor-associated antigen expressing Lewis Y related carbohydrate, TWEAK, TXB2, Ung, uPAR, uPAR-1, Urokinase, VCAM, VCAM-1, VECAD, VE-Cadherin, VE-cadherin-2, VEFGR-1 (fit-1), VEGF, VEGFR, VEGFR-3 (flt-4), VEGI, VFM, Viral antigens, VLA, VLA-1, VLA-4, VNR integrin, von Willebrand's factor, WIF-1, WNT1, WNT2, WNT2B/13, WNT3, WNT3A, WNT4, WNT5A, WNT5B, WNT6, WNT7A, WNT7B, WNT8A, WNT8B, WNT9A, WNT9A, WNT9B, WNT10A, WNT10B, WNT11, WNT16, XCL1, XCL2, XCR1, XCR1, XEDAR, XIAP, XPD, CTLA4 (cytotoxic T lymphocyte antigen-4), PD1 (programmed cell death protein 1), PD-L1 (programmed cell death ligand 1), LAG-3 (lymphocyte activation gene-3), TIM-3 (T cell immunoglobulin and mucin protein-3), and hormone receptors; or (2) an antigen selected from the group consisting of: BCMA, CTLA4 (cytotoxic T lymphocyte antigen-4), PD1 (programmed cell death protein 1), PD-L1 (programmed cell death ligand 1), LAG-3 (lymphocyte activation gene-3), TIM-3, CD20, CD2, CD19, Her2, EGFR, EpCAM, FcyRIIIa (CD16), FcyRIIa (CD32a), FcyRIIb (CD32b), FcyRI (CD64), Toll-like receptors (TLRs), TLR4, TLR9, cytokines, IL-2, IL-5, IL-13, IL-6, IL-17, IL-12, IL-23, TNFa, TGFβ, cytokine receptors, IL-2R, chemokines, chemokine receptors, growth factors, VEGF, and HGF; or (3) an antigen selected from CD1a,b,c,d; CD2, CD3, CD4, Cd5,CD6, CD7, CD8, CD9, CD10, CD11a, b, c, d; CDw12, CD13, CD14, oCD15, CD15s, CD15u, CD16, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42a,b,c,d; CD43, CD44, CD45, CD45RO. CD45RA, CD45RB, CD46, CD47, CD48, CD49a, CD49b, CD49c, CD49d, CD49e, CD49f, CD5O, CD51, CD52, CD53, CD54, CD55m CD56, CD57, CD58, CD59, CD60a, CD60b, CD61, CD61E, CD62L, CD62P, CD63, CD64, CD65, CD66a, CD66b, CD66c, CD66d,CD66e, CD68, CD69, CD70, CD71, CD72, CD73, CD74, CD75, CD75s, CD77, CD78, CD79α,β, CD80, CD81, CD82, CD83, CDw84, CD85, CD86, CD87, CD88, CD89, CD90, CD91, CD92, Cd92, CD93, CD94, CD95, CD96, CD97, CD98, CD99, CD100, CD101, CD102, CD103, CD104, CD105, CD106, CD107a, CD108, CD109, CD110, CD111, CD112, CD114, CD115, CD116, CD117, CD118, CD119, CD120a, CD120b, CD121a, CDw121b, CD122, CD123, CD124, CD125, CD126, CD127, CDw128, CD129, CD130, CDw131, CD132, CD133, CD134, CD135, CDw136, CDw137, CD138, CD139, CD140a,b, CD141, CD142, CD143, CD144, CD145, CD146, CD147, CD148, CD149, CD150, CD151, CD152, CD153, CD154, CD155, CD156b, CD157, CD158, CD158a, CD159a, CD160, CD161, CD162, CD162R, CD163, CD164, CD165, CD166, CD167a, CD168, CD169, CD170, CD171, CD172a, CD173, CD174, CD175, CD175s, CD176, CD177, CD178, CD179a, CD179b, CD180, CD183, CD184, CD195, CDw197, CD200, CD201, CD202b, CD203c, CD204, CD205, CD206, CD207, CD208, CD209, CDw210, CD212, CD213a1, CD213a2, CDw217, CD220, CD221, CD222, CD223,CD224, CD225, CD226, CD227, CD228, CD229. CD230, CD231, CD232, CD233, CD234, CD235a, CD235b, CD236, CD236R, CD238, CD239, CD240CE, CD240D, CD241, CD242, CD243, CD244, CD245, CD246, CD247 and the like; or (4) any of IL4ra, TNFa, BTK, RORgt, PIK3CA, JAK1, JAK3, TYK2 Glut1, Glut3, TAP1, CIITA, cGAS, IRF5, STAT3, STAT6, TAK1(MAP3K7), HPK1; or any of SOCS1, CD39, Cbl, or PTPN22; or (5) any of Glut1, PI3K, BTK, TNF, or RORC; or (6) any of PD-1, PD-L1, PD-L2, CTLA-4, B7-1, B7-2, LAG-3, HHLA2, TNFRSF12A, HLA-G, NECTIN2, TNFRSF25, TNFSF14, LAIR1, TNFSF15, TNFSF4, KIR2DL4, PDCD1, LGALS9, and PVR.

In some specific aspects the ARC or ANC of any of the foregoing comprises a nucleic acid payload, optionally an RNA, further optionally an siRNA or antisense RNA, having a payload comprising a sequence selected from those recited in Appendix 2 or Appendix 4; or which comprises an INX-201 ARC selected from those recited in Appendix 2 or which comprises an INX-201 ARC selected from those comprising the amino acid sequences and payload sequences recited Appendix 4.

In some specific aspects the ARC or ANC of any of the foregoing comprises at least 2 different RNA payloads which target the same or different immunomodulatory genes or mRNAs, optionally an immune target as disclosed above.

In some specific aspects in the ARC or ANC of any of the foregoing, the nucleic acid, optionally RNA payload, is linked to the antibody or antibody fragment via a cleavable or non-cleavable linker.

In some specific aspects the ARC or ANC of any of the foregoing is used to deliver one or more gene editing nucleic acids (e.g., CRISPR guide RNA (gRNA or sgRNA)) and optionally an CRISPR-associated endonuclease or a nucleic acid which encodes for a CRISPR-associated endonuclease.

In some specific aspects the ARC or ANC of any of the foregoing comprises a PD of at least 1 day, 2 days, 3 days, 4 days, 5 days, a week, 2 weeks, 3 weeks, 4 weeks, 5 weeks, or 6 weeks or longer.

In some specific aspects the ARC or ANC of any of the foregoing does not elicit any appreciable toxicity to non-target cells.

In some specific aspects the invention provides a composition comprising an ARC or ANC of any of the foregoing, and a pharmaceutically acceptable carrier or excipient, wherein said ARCs or ANCs are optionally comprised in or on a lipid nanoparticle.

In some specific aspects the invention provides a method of therapy or prophylaxis, which comprises the administration of an ARC or ANC of any of the foregoing, or a composition containing, to a subject in need thereof.

In some aspects the invention relates to the administration of an ARC or ANC of any of the foregoing, or a composition containing, to a subject in need thereof for treatment or prevention of a neoplastic, proliferative, neurodegenerative, neuroinflammatory, infectious, autoimmune, allergic, or inflammatory condition or a pathologic symptom associated with any of said conditions.

In some aspects the invention relates to the administration of an ARC or ANC of any of the foregoing, or a composition containing, to a subject in need thereof for treatment or prevention of an autoimmune disease, e.g., one involving myeloid or T cells.

In some aspects the invention relates to the administration of an ARC or ANC of any of the foregoing, or a composition containing, to a subject in need thereof for treatment or prevention of a neoplastic, proliferative, neurodegenerative, neuroinflammatory, infectious, autoimmune or inflammatory disease and/or to prevent or inhibit at least one pathologic symptom associated therewith.

In some aspects the invention relates to the administration of an ARC or ANC of any of the foregoing, or a composition containing, to a subject in need thereof for treatment or prevention of an autoimmune disease selected from one or more of Acromegaly, Acquired aplastic anemia, Acquired hemophilia, Agammaglobulinemia, primary, Alopecia areata, Ankylosing spondylitis (AS), Anti-NMDA receptor encephalitis, Antiphospholipid syndrome (APS)|catastrophic antiphospholipid syndrome (CAPS)/Asherson's syndrome, Arteriosclerosis, Autoimmune Addison's disease (AAD), Autoimmune autonomic ganglionopathy (AAG)/autoimmune dysautonomia|autoimmune gastrointestinal dysmotility (AGID), Autoimmune encephalitis|acute disseminated encephalomyelitis (ADEM), Autoimmune gastritis, Autoimmune hemolytic anemia (AIHA), Autoimmune hepatitis (AIH), Autoimmune hyperlipidemia, Autoimmune hypophysitis, Autoimmune inner ear disease (AIED), Autoimmune lymphoproliferative syndrome (ALPS), Autoimmune myelofibrosis, Autoimmune myocarditis, Autoimmune oophoritis, Autoimmune pancreatitis (AIP), Autoimmune polyglandular syndromes, types I, II, & III (APS type 1, APS type 2, APS type 3, APECED), Autoimmune progesterone dermatitis, Autoimmune retinopathy (AIR), Autoimmune sudden sensorineural hearing loss (SNHL), Balo disease, Beh et's disease, Birdshot chorioretinopathy/birdshot uveitis, Bullous pemphigoid, Castleman disease, Celiac disease, Chagas disease, Chronic inflammatory demyelinating polyneuropathy (CIDP), Chronic urticaria (CU), Churg-Strauss syndrome/eosinophilic granulomatosis with polyangiitis (EGPA), Cogan's syndrome, Cold agglutinin disease, CREST syndrome|limited cutaneous systemic sclerosis, Crohn's disease (CD), Cronkhite-Canada syndrome (CSS), Cryptogenic organizing pneumonia (COP), Dermatitis herpetiformis, Dermatomyositis, Diabetes, type 1, Discoid lupus, Dressier's syndrome/postmyocardial infarction/postpericardiotomy syndrome, Eczema/Atopic Dermatitis, Endometriosis, Eosinophilic esophagitis, Eosinophilic fasciitis, Erythema nodosum, Essential mixed cryoglobulinemia, Evans syndrome, Fibrosing alveolitis/Idiopathic pulmonary fibrosis (IPF), Giant cell arteritis/temporal arteritis/Horton's disease, Giant Cell Myocarditis, Glomerulonephritis, Goodpasture's syndrome/anti-GBM/anti-TBM disease, Granulomatosis with polyangiitis (GPA)/Wegener's granulomatosis, Grave's disease/thyroid eye disease, Guillain-Barre syndrome (GBS), Hashimoto's thyroiditis/chronic lymphocytic thyroiditis/autoimmune thyroiditis, Henoch-Schönlein purpura/IgA vasculitis, Hidradenitis suppurativa, Hurst's disease/acute hemorrhagic leukoencephalitis (AHLE), Hypogammaglobulinemia, IgA nephropathy/Berger's disease, Immune-mediated necrotizing myopathy (IMNM), Immune thrombocytopenia (ITP)/autoimmune thrombocytopenic purpura/autoimmune thrombocytopenia, Inclusion body myositis, IgG4-related sclerosing disease (ISD), Interstitial cystitis, Juvenile idiopathic arthritis/Adult-onset Still's disease. Juvenile polymyositis|Juvenile dermatomyositis|juvenile myositis, Kawasaki disease, Lambert-Eaton myasthenic syndrome (LEMS), Leukocytoclastic vasculitis, Lichen planus, Lichen sclerosus, Ligneous conjunctivitis, Linear IgA disease (LAD)|linear IgA bullous dermatosis (LABD), Lupus nephritis, Lyme disease/chronic Lyme disease/post-treatment Lyme disease syndrome (PTLDS), Lymphocytic colitis/microscopic colitis, Lymphocytic hypophystitis/autoimmune hypophystitis, Meniere's disease, Microscopic polyangiitis (MPA)/ANCA-associated vasculitis, Mixed connective tissue disease (MCTD), Mooren's ulcer, Mucha-Habermann disease, Multifocal motor neuropathy, Multiple sclerosis (MS), Myalgic encephalomyelitis (ME)/Chronic fatigue syndrome (CFS), Myasthenia gravis (MG), Narcolepsy, Neuromyelitis Optica/Devic's disease, Ocular cicatricial pemphigoid, Opsoclonus-myoclonus syndrome (OMS), Palindromic rheumatism, Paraneoplastic cerebellar degeneration, Paraneoplastic pemphigus, Parry-Romberg syndrome (PRS)/Hemifacial atrophy (HFA)/Progressive facial hemiatrophy, Paroxysmal nocturnal hemoglobinuria (PNH), Peripheral uveitis/pars planitis, PANS/PANDAS, Parsonage-Turner syndrome, Pemphigus gestationis/herpes gestationis, Pemphigus foliaceus, Pemphigus vulgaris, Pernicious anemia, POEMS syndrome, Polyarteritis nodosa, Polymyalgia rheumatica, Polymyositis, Postural orthostatic tachycardia syndrome (POTS), Primary biliary cirrhosis (PBC)/primary biliary cholangitis, Primary sclerosing cholangitis (PSC), Psoriasis, Palmoplantar Pustulosis, Psoriatic arthritis, Pulmonary fibrosis, idiopathic (IPF), Pure red cell aplasia (PRCA), Pyoderma gangrenosum, Rasmussen's encephalitis, Raynaud's syndrome/phenomenon, Reactive arthritis/Reiter's syndrome, Reflex sympathetic dystrophy syndrome (RSD)/Complex regional pain syndrome (CRPS), Relapsing polychondritis, Restless leg syndrome (RLS)/Willis-Ekbom disease, Rheumatic fever, Rheumatoid arthritis, Sarcoidosis, Schmidt syndrome/autoimmune polyendocrine syndrome type II, Scleritis, Scleroderma, Sclerosing Mesenteritis/Mesenteric Panniculitis, Serpiginous choroidopathy, Sjögren's syndrome, Stiff person syndrome (SPS), Small fiber sensory neuropathy, Systemic lupus erythematosus (SLE), Subacute bacterial endocarditis (SBE), Subacute cutaneous lupus, Susac syndrome, Sydenham's chorea, Sympathetic ophthalmia, Takayasu's arteritis (vasculitis), Testicular autoimmunity (vasculitis, orchitis), Tolosa-Hunt syndrome, Transverse myelitis (TM), Tubulointerstitial nephritis uveitis syndrome (TINU), Ulcerative colitis (UC), Undifferentiated connective tissue disease (UCTD), Uveitis|anterior/intermediate/posterior, Vasculitis, VEXAS Syndrome, Vitiligo, and Vogt-Koyanagi-Harada syndrome (VKH), and/or to prevent or inhibit at least one pathologic symptom associated therewith.

In some aspects the invention relates to the administration of an ARC or ANC of any of the foregoing, or a composition containing, to a subject in need thereof for treatment or prevention of Addison disease, arthritis, celiac disease, lupus, Grave's disease, myasthenia gravis, multiple sclerosis, ITP, rheumatoid arthritis, colitis, inflammatory bowel disease, pernicious anemia, Hashimoto's thyroiditis Sjogren's disease, asthma, type 2 diabetes, and autoimmune type I diabetes and/or to prevent or inhibit at least one pathologic symptom associated therewith.

In some aspects the invention relates to the administration of an ARC or ANC of any of the foregoing, or a composition containing, to a subject in need thereof for treatment or prevention of an inflammatory disease selected from the group consisting of Fatty liver disease, Endometriosis, Type 2 diabetes, mellitus, Type 1 diabetes mellitus, Inflammatory bowel disease (IBD), Asthma, Rheumatoid arthritis, asthma, Obesity, Fibromyalgia, Lupus SLE, osteoarthritis, Rheumatoid Arthritis, Shingles Herpes Zoster, and Vasculitis and/or to prevent or inhibit at least one pathologic symptom associated therewith.

In some aspects the invention relates to the administration of an ARC or ANC of any of the foregoing, or a composition containing, to a subject in need thereof for treatment or prevention of a neurodegenerative or neuroinflammatory disease, e.g., Alzheimer's disease, Amyotrophic lateral sclerosis, Friedreich ataxia, Huntington's disease, Lewy body disease, aphasia, Parkinson's disease or Spinal muscular atrophy and/or to prevent or inhibit at least one pathologic symptom associated therewith.

In some aspects the invention relates to the administration of an ARC or ANC of any of the foregoing, or a composition containing, to a subject in need thereof for treatment or prevention of cancer or to prevent cancer reoccurrence and/or to inhibit least one pathologic symptom associated with specific immune cell types.

In some aspects the invention relates to the administration of an ARC or ANC of any of the foregoing, or a composition containing, to a subject in need thereof for treatment or prevention of a solid tumor and/or to prevent or inhibit at least one pathologic symptom associated therewith.

In some aspects the invention relates to the administration of an ARC or ANC of any of the foregoing, or a composition containing, to a subject in need thereof for treatment or prevention of a hematologic malignancy and/or to prevent or inhibit at least one pathologic symptom associated therewith.

In some aspects the invention relates to the administration of an ARC or ANC of any of the foregoing, or a composition containing, to a subject in need thereof for treatment or prevention of a relapsed or refractory cancer, or a metastatic cancer, optionally a relapsed or refractory solid tumor, or a metastatic solid tumor, a relapsed or refractory hematologic malignancy, or a metastatic hematologic malignancy.

In some aspects the invention relates to the administration of an ARC or ANC of any of the foregoing, or a composition containing, to a subject in need thereof for treatment or prevention of a solid tumor selected from anal cancer, appendix cancer, bile duct cancer (i.e., cholangiocarcinoma), bladder cancer, brain tumor, breast cancer, cervical cancer, colon cancer, cancer of Unknown Primary (CUP), esophageal cancer, eye cancer, fallopian tube cancer, gastroenterological cancer, kidney cancer, liver cancer, lung cancer, medulloblastoma, melanoma, oral cancer, ovarian cancer, pancreatic cancer, parathyroid disease, penile cancer, pituitary tumor, prostate cancer, rectal cancer, skin cancer, stomach cancer, testicular cancer, throat cancer, thyroid cancer, uterine cancer, vaginal cancer, or vulvar cancer and/or to prevent or inhibit at least one pathologic symptom associated therewith.

In some aspects the invention relates to the administration of an ARC or ANC of any of the foregoing, or a composition containing, to a subject in need thereof for treatment or prevention of a hematologic malignancy, optionally a leukemia, a lymphoma, a myeloma, a non-Hodgkin's lymphoma, or a Hodgkin's lymphoma. In some instances, the hematologic malignancy comprises chronic lymphocytic leukemia (CLL), small lymphocytic lymphoma (SLL), high risk CLL, a non-CLL/SLL lymphoma, prolymphocytic leukemia (PLL), follicular lymphoma (FL), diffuse large B-cell lymphoma (DLBCL), mantle cell lymphoma (MCL), Waldenström's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis and/or to prevent or inhibit at least one pathologic symptom associated therewith.

In some aspects the invention relates to the administration of an ARC or ANC of any of the foregoing, or a composition containing, to a subject in need thereof for treatment or prevention of a hematologic malignancy selected from chronic lymphocytic leukemia (CLL), small lymphocytic lymphoma (SLL), high risk CLL, a non-CLL/SLL lymphoma, prolymphocytic leukemia (PLL), follicular lymphoma (FL), diffuse large B-cell lymphoma (DLBCL), mantle cell lymphoma (MCL), Waldenström's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some cases, the hematologic malignancy is a relapsed or refractory hematologic malignancy, or a metastatic hematologic malignancy and/or to prevent or inhibit at least one pathologic symptom associated therewith.

In some aspects the invention relates to the administration of an ARC or ANC of any of the foregoing, or a composition containing, to a subject in need thereof for treatment or prevention of an autoimmune disease selected from the group consisting of Addison disease, arthritis, celiac disease, lupus, Grave's disease, myasthenia gravis, multiple sclerosis, ITP, rheumatoid arthritis, colitis, inflammatory bowel disease, pernicious anemia, Hashimoto's thyroiditis Sjogren's disease, asthma, type 2 diabetes, and autoimmune type I diabetes and/or to prevent or inhibit at least one pathologic symptom associated therewith.

In some aspects the invention relates to the administration of an ARC or ANC of any of the foregoing, or a composition containing, to a subject in need thereof for treatment of cancer, and the ARC or ANC comprises a nucleic acid, optionally an ASO or siRNA which modulates or blocks the expression of any of PD-1, PD-L1, PD-L2, CTLA-4, B7-1, B7-2, LAG-3, HHLA2, TNFRSF12A, HLA-G, NECTIN2, TNFRSF25, TNFSF14, LAIR1, TNFSF15, TNFSF4, KIR2DL4, PDCD1, LGALS9, VSIr (VISTA) and PVR.

In some aspects the invention relates to the administration of an ARC or ANC of any of the foregoing, or a composition containing, to a subject in need thereof for inhibiting or treating immune senescence associated with disease or aging, and the ARC or ANC optionally comprises a nucleic acid, further optionally an ASO or siRNA which modulates or blocks the expression of any of PD-1, PD-L1, PD-L2, CTLA-4, B7-1, B7-2, LAG-3, HHLA2, TNFRSF12A, HLA-G, NECTIN2, TNFRSF25, TNFSF14, LAIR1, TNFSF15, TNFSF4, KIR2DL4, PDCD1, LGALS9, VSIr (VISTA) and PVR.

### BRIEF DESCRIPTION OF THE DRAWINGS

FIG. 1 contains a list of immunologically relevant siRNAs and ASO payloads which are embraced by the invention and which have been tested. In the proof-of-concept experiments K562-VISTA cells were transfected with 200 nM siRNA (or ASO) alone, and target knockdown was analyzed by qRTPCR using the ddct method and reported as fold over control. Scrambled siRNA or ASO controls were also ordered from IDT. Payloads marked with (*) were selected for conjugation to a delivery vehicle (anti-VISTA Mab INX201).

FIG. 2 contains a list of other immunologically relevant siRNAs and ASO payloads which have been identified and tested. K562-VISTA cells were transfected with 200 nM siRNA (or ASO) alone, and target knockdown was analyzed by qRTPCR using the ddct method and reported as fold over control. Scrambled siRNA or ASO controls were also ordered from IDT. Payloads marked with (*) were selected for conjugation to a delivery vehicle (anti-VISTA Mab INX201). Only CD39 ARC is described in this report from the second round of payloads; other ARC's conjugation is planned/ongoing.

FIG. 3 contains SDS-PAGE results for exemplary ARCs according to the invention (INX201 ARCs) confirming effective RNA conjugation. INX201 ARCs were resolved on reducing SDS-PAGE followed by silver stain. Lane labels: 201—free INX201 anti-VISTA Mab; CD45—ARC with CD45 siRNA; SOCS1—ARC with SOCS1 ASO; a discrete pattern of multiple conjugates is observed, confirming efficient conjugation. The HC or LC shift with siRNA is more profound than ASO, based on the molecular weight of the payload (~17 vs. ~6 kDa).

FIG. 4A-B shows that an exemplary ARC according to the invention efficiently binds to and provides for effective internalization from the surface and intracellular retention of siRNA inside K562-VISTA cells. Free INX201 (line with circles) or eGFP ARC (line with squares) were used at 200 nM. A) Time course of antibody binding and internalization. B) RNA accumulation assay, measured by Cy5. MFI—mean fluorescent intensity; representative of two independent experiments shown.

FIG. 5A-C shows that an exemplary ARCs according to the invention (INX201 ARC's) knockdown protein expression is equivalent to transfection in vitro. K562-VISTA WT cells or eGFP+ cell pools were used for this study. A) cells were treated for 28 hours with no drug (left bar) or 200 nM eGFP ARC (right bar). The maximal levels of eGFP protein knockdown were established by transfecting eGFP siRNA into the same cells (~50%, measured at 24 hours). ARC-mediated knockdown was similar to the maximal possible knockdown levels (based on siRNA sequence). B) Dash line represents the maximal level of CD45 protein knockdown expected, based on the payload (siRNA) potency, which is 50%, as measured from free transfected CD45 siRNA at 48 hours (right bar); cells treated for 72 hours with 200 nM eGFP ARC (middle bar) or with no drug (left bar). C)CD45 levels measured in a repeat experiment. Cells were treated for 72 hours with no drug (left bar), free siRNA, no transfection (middle bar), and 200 nM ARC (right bar).

FIG. 6A-C shows that exemplary ARCs according to the invention (INX201 ARCs) inhibit TNFa from PBMC. Human PBMCs were activated with (A) 10 ng/ml LPS or (B) anti-CD3/CD28 beads, bead to T cell ratio 1:2, and treated with TNFa ARC (0-200 nM) or free RNA (200-1000 nM) for 48 hours (LPS) or 72 hours (beads). There was an efficient dose-dependent reduction in TNFa levels with ARC but not free siRNA. C) qRTPCR performed from ARC-treated versus untreated PBMCs, collected at 72 hours, confirmed efficient target knockdown; no ARC was visualized as 0.1 nM (due to log scale).

FIG. 7A-B shows that an exemplary ARC according to the invention (INX201 ARC) slow down the proliferation of T cells. Human PBMCs were activated with anti-CD3/CD28 beads, bead to T cell ratio 1:2, and treated with (A) TNFa ARC (0-200 nM) or free RNA (200-1000 nM) or (B) PI3K ARC (0-200 nM) or free RNA (200-1000 nM) for 72 hours. There was an efficient dose-dependent reduction in newly proliferating T cells with ARC but not free siRNA treatments. Proliferation was analyzed by cell trace violet dilution, visualized by flow cytometry; no ARC was visualized as 0.1 nM (due to log scale). The curves were generated from inhibitor vs. response three-parameter non-linear regression analysis (GraphPad Prism 9). A single technical replicate per concentration point was used.

FIG. 8A-B shows that an exemplary ARC according to the invention reduced PBMC activation (INX201-BTK ARC). Human PBMCs were activated with (A) 10 ng/ml LPS or (B) anti-CD3/CD28 beads, bead to T cell ratio 1:2, and treated with BTK ARC (0-200 nM, triangles) or free RNA (200-1000 nM, stars), or free INX201 (square) for 48 hours (LPS) or 72 hours (beads). The curves were generated from inhibitor vs. response three-parameter non-linear regression analysis (GraphPad Prism 9). CD69 levels were measured in A; % newly proliferating cells in B; A single technical replicate per concentration point was used; no ARC was visualized as 0.1 nM (due to log scale); unstim—unstimulated cells; MFI—mean fluorescent intensity.

FIG. 9A-B shows that an exemplary ARC according to the invention reduced cytokine production (INX201-Glut1 ARC). Purified human T cells were activated with anti-CD3/CD28 beads, bead to a T cell ratio 1:2, and treated with Glut1 ARC (0-200 nM) for 72 hours. A single technical replicate per concentration point was used. Two human donors were tested: donor 1—solid line, donor 2—dashed line; no ARC was visualized as 0.1 nM (due to log scale). A) IFNg and B) IL17A were measured by Luminex.

FIG. 10A-C also shows that an exemplary ARC according to the invention reduced cytokine production (INX201-Glut1 ARC). Purified human T cells were activated with anti-CD3/CD28 beads, bead to a T cell ratio 1:2, and treated with RORC ARC (0-200 nM) for 72 hours. A single technical replicate per concentration point was used. Two human donors were tested: donor1—solid line, donor 2—dashed line; no ARC was visualized as 0.1 nM (due to log scale). A) IFNg, B) IL-6, and C) IL12p40 were measured by Luminex.

FIG. 11 shows that an exemplary ARC according to the invention (INX201-CD39 ASO) successfully targets human PBMC and enhances immune responses in human PBMC. Human PBMCs were activated with anti-CD3/CD28 beads and treated with CD39 ARC (0-200 nM, triangles) or free INX201 (0-200 nM, circles) for 72 hours. The curves were generated from inhibitor vs. response three-parameter non-linear regression analysis (GraphPad Prism 9). A single technical replicate per concentration point was used; no ARC was visualized as 0.1 nM (due to the log scale).

FIG. 12A-B schematically shows how the inventive ARC platform may be optimized by using anti-VISTA Fab instead of a Mab. A) Comparison of binding to human VISTA ECD between INX201 Mab (squares) and INX201 Fab (circles) by ELISA. B) K562-VISTA cell-based competition assay, where the pre-bound increasing concentrations of INX201 Mab (squares) or INX201 Fab (circles) block available VISTA on the cell surface (hence reduced VISTA MFI measurement by INX201-AF488; the curves were generated from inhibitor vs. response three-parameter non-linear regression analysis (GraphPad Prism 9). n=2 technical replicate per concentration point was used; no Ab was visualized as 0.0001 nM (due to log scale).

FIG. 13 shows that exemplary ARCs according to the invention do not impact T cell viability. Human PBMCs were activated with anti-CD3/CD28 beads and treated with PI3K ARC (0-200 nM, triangles) or free INX201 (0-200 nM, squares) for 72 hours. The curves were generated from inhibitor vs. response three-parameter non-linear regression analysis (GraphPad Prism 9). A single technical replicate per concentration point was used; no ARC was visualized as 0.1 nM (due to the log scale); unstim—unstimulated cells.

FIG. 14 shows the CDR and variable sequences at exemplary anti-human VISTA antibodies which may be used in ARCs or ANCs according to the invention.

FIG. 15 shows that INX201 (anti-VISTA) PI3K ARC, but not isotype control IgG1 PI3K ARC specifically knockdown PI3K expression. K562-VISTA cells were transfected with 40-200 nM were treated with 40-200 nM INX201-PI3K ARC or isotype control IgG1-PI3K ARC, and target knockdown was analyzed by qRTPCR using the ddct method and reported as % target suppression, where 0% suppression were INX201 alone samples; PI3K ARC: anti-human VISTA mAb conjugated to PI3K siRNA; PI3K isotype ARC: IgG control conjugated to PI3K siRNA; INX201, mAb unconjugated: naked anti-human VISTA mAb. A single technical replicate per concentration point was used.

FIG. 16A-B shows that human Pl3K and GLUT3 ARCs are functional in vitro. Human PBMCs were activated with anti-CD3/CD28 beads for 72 hours, bead to T cell ratio 1:2, and treated with (A) PI3K ARC or free INX201 (0-200 nM), or (B) GLUT3 ARC or free INX201 (0-200 nM). The curves were generated from inhibitor vs. response three-parameter non-linear regression analysis (GraphPad Prism 10). IL5/IL13/TNFa/IL17F cytokine levels were measured by Luminex. CD69 levels were measured in live CD45+/HLA-DR−/CD56−/CD3+/CD4+/CD45RA−/CD27− T effector memory cells; CD25 levels are measured in live CD45+/HLA-DR−/CD56−/CD3 cells. A single technical replicate per concentration point was used in A; n=2 technical replicate per concentration point was used in B; no ARC was visualized as 0.1 nM (due to log scale); MFI—mean fluorescent intensity. Data represented as mean±SEM, where appropriate.

FIG. 17A-C shows that PI3K ARC reduces inflammatory cytokine responses in xeno-GvHD. NSG mice were injected i.v. with PI3K ARC (triangles, n=6) or INX201 (circles, n=6) at 5 mg/Kg together with human PBMC transfer. 18 hours after injection mice were stimulated with 0.5 mg/kg LPS, intraperitoneally. A) xeno-GvHD LPS stimulation model. Schematic of the experiment is shown. B) Changes in plasma human cytokine levels on day 7 (IFNg) or 4 hours (IL6, TNFa). Statistical analysis was performed by Student's t-test. Data represented as mean±SEM, *—p<0.05; **—p<0.01. C) Heat map based on Z-score is shown for cytokine levels at 4 hours post LPS stimulation (n=6 per group).

FIG. 18 shows that PI3K ARC diminishes LPS induced T cell proliferation in vivo. NSG mice were injected i.v. with PI3K ARC (triangles, n=6) or INX201 (circles, n=6) at 5 mg/Kg together with human PBMC transfer. 18 hours after injection mice were stimulated with 0.5 mg/kg LPS, intraperitoneally. Changes in blood T cell numbers at day 14, (n=6 mice per group) are shown for both CD4 and CD8. Statistical analysis was performed by Student's t-test. Data represented as mean±SEM, *—p<0.05.

FIG. 19 shows that PI3K ARC does not reduce percent of regulatory T cells in vivo. NSG mice were injected i.v. with PI3K ARC (triangles, n=10) or INX201 (circles, n=8) at 5 mg/Kg together with human PBMC transfer (right graph). In separate groups of mice, 18 hours after PBMC injection mice were stimulated with 0.5 mg/kg LPS, intraperitoneally (n=6 for both INX201 and PI3K ARC groups, left graph). Blood samples were processed on day 28. Percent changes in Treg are shown. Statistical analysis was performed by Student's t-test. Data represented as mean±SEM ns—not significant.

FIG. 20 shows that GLUT3 ARC reduces inflammatory cytokine responses in xeno-GvHD. NSG mice were injected i.v. with PI3K ARC (triangles, n=10) or INX201 (circles, n=8) at 5 mg/Kg together with human PBMC transfer. Changes in plasma human cytokine levels on day 7 are shown. Statistical analysis was performed by Student's t-test. Data represented as mean±SEM, **—p<0.01; ****—p<0.0001.

### DETAILED DESCRIPTION OF THE INVENTION

The present invention disclosed herein relates to anti-VISTA antibody oligonucleotide conjugates (ARCs or ANCs) which specifically deliver RNAs to immune cells and use thereof as therapeutics, e.g., for treating autoimmune and inflammatory conditions.

### DEFINITIONS

Listed below are definitions of various terms used to describe this disclosure. These definitions apply to the terms as they are used throughout this specification and claims, unless otherwise limited in specific instances, either individually or as part of a larger group.

The term “about” will be understood by persons of ordinary skill in the art and will vary to some extent on the context in which it is used. As used herein when referring to a measurable value such as an amount, a temporal duration, and the like, the term “about” is meant to encompass variations of ±20% or ±10%, including ±5%, ±1%, and +0.1% from the specified value, as such variations are appropriate to perform the disclosed methods.

The term “antibody RNA conjugate” or “antibody nucleic acid conjugate” or “ARC’ or “ANC” herein generally refers to a conjugate comprising (i) an antibody or antibody fragment (such as a Fab) which specifically binds specifically or preferentially to one or more targeted immune cell types, to which is directly or indirectly attached (e.g., via a cleavable or non-cleavable peptide linker or other cleavable or non-cleavable linker) to (ii) one or more nucleic acids, typically oligonucleotides, e.g., DNAs or RNAs, which oligonucleotides may be comprised of wild-type or modified nucleotides, and which oligonucleotides specifically target immunomodulator genes or RNAs encoded thereby and which modulate the expression and/or activity of the immunomodulator protein encoded thereby. In preferred embodiments the antibody or antibody fragment is an internalizing antibody, i.e., when it binds to the target antigen on target immune cells it internalizes the immune cell and delivers the oligonucleotide cargo comprised on the ARC or ANC into the immune cell. In exemplary embodiments the antibody or antibody fragment is an internalizing antibody or antibody fragment which specifically binds to VISTA, preferably human VISTA.

The term “alkyl” refers to saturated, straight- or branched-chain hydrocarbon moieties containing, in certain embodiments, between one and six, or one and eight carbon atoms, respectively. Examples of Ci-6-alkyl moieties include, but are not limited to, methyl, ethyl, propyl, isopropyl, «-butyl, tert-butyl, neopentyl, n-hexyl moieties; and examples of Ci-s-alkyl moieties include, but are not limited to, methyl, ethyl, propyl, isopropyl, «-butyl, tert-butyl, neopentyl, n-hexyl, heptyl, and octyl moieties.

The number of carbon atoms in an alkyl substituent can be indicated by the prefix “C_{x-y},” where x is the minimum and y is the maximum number of carbon atoms in the substituent. Likewise, a Cₓ chain means an alkyl chain containing x carbon atoms.

The term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—H—CH₃, —CH₂—S—CH₂—CH₃, and —CH₂—CH₂—S(═O)—CH₃. Up to two heteroatoms may be consecutive, such as, for example, —CH₂—H—OCH, or —CH₂—CH₂—S—S—CH.

The term “aryl,” employed alone or in combination with other terms, means, unless otherwise stated, a carbocyclic aromatic system containing one or more rings (typically one, two, or three rings), wherein such rings may be attached together in a pendent manner, such as a biphenyl, or may be fused, such as naphthalene. Examples of aryl groups include phenyl, anthracyl, and naphthyl. In various embodiments, examples of an aryl group may include phenyl (e.g., C₆-aryl) and biphenyl (e.g., Ci₂-aryl). In some embodiments, aryl groups have from six to sixteen carbon atoms. In some embodiments, aryl groups have from six to twelve carbon atoms (e.g., C₆-i₂-aryl). In some embodiments, aryl groups have six carbon atoms (e.g., Ce-aryl).

As used herein, the term “heteroaryl” or “heteroaromatic” refers to a heterocycle having aromatic character. Heteroaryl substituents may be defined by the number of carbon atoms, e.g., Ci-9-heteroaryl indicates the number of carbon atoms contained in the heteroaryl group without including the number of heteroatoms. For example, a Ci-9-heteroaryl will include an additional one to four heteroatoms. A polycyclic heteroaryl may include one or more rings that are partially saturated. Non-limiting examples of heteroaryls include pyridyl, pyrazinyl, pyrimidinyl (including, e.g., 2- and 4-pyrimidinyl), pyridazinyl, thienyl, furyl, pyrrolyl (including, e.g., 2-pyrrolyl), imidazolyl, thiazolyl, oxazolyl, pyrazolyl (including, e.g., 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Non-limiting examples of polycyclic heterocycles and heteroaryls include indolyl (including, e.g., 3-, 4-, 5-, 6- and 7-indolyl), indolinyl, quinolyl, tetrahydroquinolyl, isoquinolyl (including, e.g., 1- and 5-isoquinolyl), 1,2,3,4-tetrahydroisoquinolyl, cinnolinyl, quinoxalinyl (including, e.g., 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (including, e.g., 3-, 4-, 5-, 6- and 7-benzofuryl), 2,3-dihydrobenzofuryl, 1,2-benzisoxazolyl, benzothienyl (including, e.g., 3-, 4-, 5-, 6-, and 7-benzothienyl), benzoxazolyl, benzothiazolyl (including, e.g., 2-benzothiazolyl and 5-benzothiazolyl), purinyl, benzimidazolyl (including, e.g., 2-enzimidazolyl), benzotriazolyl, thioxanthinyl, carbazolyl, carbolinyl, acridinyl, pyrrolizidinyl, and quinolizidinyl.

The term “protecting group” or “chemical protecting group” refers to chemical moieties that block some or all reactive moieties of a compound and prevent such moieties from participating in chemical reactions until the protective group is removed, for example, those moieties listed and described in T.W. Greene, P.G.M. Wuts, Protective Groups in Organic Synthesis, 3rd ed. John Wiley & Sons (1999). It may be advantageous, where different protecting groups are employed, that each (different) protective group be removable by a different means. Protective groups that are cleaved under totally disparate reaction conditions allow differential removal of such protecting groups. For example, protective groups can be removed by acid, base, and hydrogenolysis. Groups such as trityl, monomethoxytrityl, dimethoxytrityl, acetal and tert-butyldimethylsilyl are acid labile and may be used to protect carboxy and hydroxy reactive moieties in the presence of amino groups protected with Cbz groups, which are removable by hydrogenolysis, and Fmoc groups, which are base labile. Carboxylic acid moieties may be blocked with base labile groups such as, without limitation, methyl, or ethyl, and hydroxy reactive moieties may be blocked with base labile groups such as acetyl in the presence of amines blocked with acid labile groups such as tert-butyl carbamate or with carbamates that are both acid and base stable but hydrolytically removable.

Carboxylic acid and hydroxyl reactive moieties may also be blocked with hydrolytically removable protective groups such as the benzyl group, while amine groups may be blocked with base labile groups such as Fmoc. A particularly useful amine protecting group is trifluoroacetamide. Carboxylic acid reactive moieties may be blocked with oxidatively-removable protective groups such as 2,4-dimethoxybenzyl, while coexisting amino groups may be blocked with fluoride labile silyl carbamates.

Allyl blocking groups are useful in the presence of acid- and base-protecting groups since the former are stable and can be subsequently removed by metal or pi-acid catalysts. For example, an allyl-blocked carboxylic acid can be deprotected with a palladium(O)-catalyzed reaction in the presence of acid labile t-butyl carbamate or base-labile acetate amine protecting groups. Yet another form of protecting group is a resin to which a compound or intermediate may be attached. As long as the residue is attached to the resin, that functional group is blocked and cannot react. Once released from the resin, the functional group is available to react.

The term “nucleobase,” “base pairing moiety,” “nucleobase-pairing moiety,” or “base” refers to the heterocyclic ring portion of a nucleoside, nucleotide, and/or morpholino subunit. Nucleobases may be naturally occurring, or may be modified or analogs of these naturally occurring nucleobases, e.g., one or more nitrogen atoms of the nucleobase may be independently at each occurrence replaced by carbon. Exemplary analogs include hypoxanthine (the base component of the nucleoside inosine); 2, 6-diaminopurine; 5-methyl cytosine; C5-propynyl-modified pyrimidines; 10-(9-(aminoethoxy)phenoxazinyl) (G-clamp) and the like.

Further examples of base pairing moieties include, but are not limited to, uracil, thymine, adenine, cytosine, guanine and hypoxanthine having their respective amino groups protected by acyl protecting groups, 2-fluorouracil, 2-fluorocytosine, 5-bromouracil, 5-iodouracil, 2, 6-diaminopurine, azacytosine, pyrimidine analogs such as pseudoisocytosine and pseudouracil and other modified nucleobases such as 8-substituted purines, xanthine, or hypoxanthine (the latter two being the natural degradation products). The modified nucleobases disclosed in Chiu and Rana, R N A, 2003, 9, 1034-1048, Limbach et al. Nucleic Acids Research, 1994, 22, 2183-2196 and Revankar and Rao, Comprehensive Natural Products Chemistry, vol. 7, 313, are also contemplated, the contents of which are incorporated herein by reference.

Further examples of base pairing moieties include, but are not limited to, expanded-size nucleobases in which one or more benzene rings has been added. Nucleic base replacements described in the Glen Research catalog (www.glenresearch.com); Krueger A T et al, Acc. Chem. Res., 2007, 40, 141-150; Kool, E T, Acc. Chem. Res., 2002, 35, 936-943; Benner S. A., et al., Nat. Rev. Genet., 2005, 6, 553-543; Romesberg, F. E., et al, Curr. Opin. Chem. Biol, 2003, 7, 723-733; Hirao, L, Curr. Opin. Chem. Biol, 2006, 10, 622-627, the contents of which are incorporated herein by reference, are contemplated as useful for the synthesis of the oligomers described herein.

The terms “oligonucleotide” or “oligomer” refer to a compound comprising a plurality of linked nucleosides, nucleotides, or a combination of both nucleosides and nucleotides. In specific embodiments provided herein, an oligonucleotide is a morpholino oligonucleotide.

The phrase “morpholino oligonucleotide” or “PMO” refers to a modified oligonucleotide having morpholino subunits linked together by phosphoramidate or phosphorodiamidate linkages, joining the morpholino nitrogen of one subunit to the 5′-exocyclic carbon of an adjacent subunit. Each morpholino subunit comprises a nucleobase-pairing moiety effective to bind, by nucleobase-specific hydrogen bonding, to a nucleobase in a target.

The terms “antisense oligomer,” “antisense compound” and “antisense oligonucleotide” or “ASO” are used interchangeably and refer to a sequence of subunits, each bearing a base-pairing moiety, linked by intersubunit linkages that allow the base-pairing moieties to hybridize to a target sequence in a nucleic acid (typically an RNA) by Watson-Crick base pairing, to form a nucleic acid:oligomer heteroduplex within the target sequence. The oligomer may have exact (perfect) or near (sufficient) sequence complementarity to the target sequence; variations in sequence near the termini of an oligomer are generally preferable to variations in the interior.

Such an antisense oligomer can be designed to block or inhibit translation of mRNA or to inhibit/alter natural or abnormal pre-mRNA splice processing, and may be said to be “directed to” or “targeted against” a target sequence with which it hybridizes. The target sequence is typically a region including an AUG start codon of an mRNA, a Translation Suppressing Oligomer, or splice site of a pre-processed mRNA, a Splice Suppressing Oligomer (SSO). The target sequence for a splice site may include an mRNA sequence having its 5′ end 1 to about 25 base pairs downstream of a normal splice acceptor junction in a preprocessed mRNA. In various embodiments, a target sequence may be any region of a preprocessed mRNA that includes a splice site or is contained entirely within an exon coding sequence or spans a splice acceptor or donor site. An oligomer is more generally said to be “targeted against” a biologically relevant target, such as a protein, virus, or bacteria, when it is targeted against the nucleic acid of the target as described above.

The antisense oligonucleotide and the target RNA are complementary to each other when a sufficient number of corresponding positions in each molecule are occupied by nucleotides which can hydrogen bond with each other, such that stable and specific binding occurs between the oligonucleotide and the target. Thus, “specifically hybridizable” and “complementary” are terms which are used to indicate a sufficient degree of complementarity or precise pairing such that stable and specific binding occurs between the oligonucleotide and the target. It is understood in the art that the sequence of an oligonucleotide need not be 100% complementary to that of its target sequence to be specifically hybridizable. An oligonucleotide is specifically hybridizable when binding of the oligonucleotide to the target molecule interferes with the normal function of the target RNA, and there is a sufficient degree of complementarity to avoid non-specific binding of the antisense oligonucleotide to non-target sequences under conditions in which specific binding is desired, i.e., under physiological conditions in the case of in vivo assays or therapeutic treatment, and in the case of in vitro assays, under conditions in which the assays are performed.

Oligonucleotides may also include nucleobase (often referred to in the art simply as “base”) modifications or substitutions. Oligonucleotides containing a modified or substituted base include oligonucleotides in which one or more purine or pyrimidine bases most commonly found in nucleic acids are replaced with less common or non-natural bases. In some embodiments, the nucleobase is covalently linked at the N9 atom of the purine base, or at the Ni atom of the pyrimidine base, to the morpholine ring of a nucleotide or nucleoside.

Purine bases comprise a pyrimidine ring fused to an imidazole ring. Adenine and guanine are the two purine nucleobases most commonly found in nucleic acids. These may be substituted with other naturally-occurring purines, including but not limited to N6-methyladenine, N2-methylguanine, hypoxanthine, and 7-methylguanine.

Pyrimidine bases comprise a six-membered pyrimidine ring. Cytosine, uracil, and thymine are the pyrimidine bases most commonly found in nucleic acids. These may be substituted with other naturally-occurring pyrimidines, including but not limited to 5-methylcytosine, 5-hydroxymethylcytosine, pseudouracil, and 4-thiouracil. In one embodiment, the oligonucleotides described herein contain thymine bases in place of uracil.

Other modified or substituted bases include, but are not limited to, 2,6-diaminopurine, orotic acid, agmatidine, lysidine, 2-thiopyrimidine (e.g. 2-thiouracil, 2-thiothymine), G-clamp and its derivatives, 5-substituted pyrimidine (e.g. 5-halouracil, 5-propynyluracil, 5-propynylcytosine, 5-aminomethyluracil, 5-hydroxymethyluracil, 5-aminomethylcytosine, 5-hydroxymethylcytosine, Super T), 7-deazaguanine, 7-deazaadenine, 7-aza-2,6-diaminopurine, 8-aza-7-deazaguanine, 8-aza-7-deazaadenine, 8-aza-7-deaza-2,6-diaminopurine, Super G, Super A, and N4-ethylcytosine, or derivatives thereof; N2-cyclopentylguanine (cPent-G), N2-cyclopentyl-2-aminopurine (cPent-AP), and N2-propyl-2-aminopurine (Pr-AP), pseudouracil or derivatives thereof; and degenerate or universal bases, like 2,6-difluorotoluene or absent bases like abasic sites (e.g. 1-deoxyribose, 1,2-dideoxyribose, I-deoxy-2-O-methylribose; or pyrrolidine derivatives in which the ring oxygen has been replaced with nitrogen (azaribose)). Pseudouracil is a naturally occurring isomerized version of uracil, with a C-glycoside rather than the regular N-glycoside as in uridine.

Certain modified or substituted nucleobases reportedly are particularly useful for increasing the binding affinity of the antisense oligonucleotides. These include 5-substituted pyrimidines, 6-azapyrimidines and N-2, N-6 and 0-6 substituted purines, including 2-aminopropyladenine, 5-propynyluracil and 5-propynylcytosine. In various embodiments, nucleobases may include 5-methylcytosine substitutions, which have been shown to increase nucleic acid duplex stability by 0.6-1.2 °C.

Modified or substituted nucleobases are also useful for facilitating purification of antisense oligonucleotides. For example, antisense oligonucleotides may contain three or more (e.g., 3, 4, 5, 6 or more) consecutive guanine bases. In certain antisense oligonucleotides, a string of three or more consecutive guanine bases can result in aggregation of the oligonucleotides, complicating purification. In such antisense oligonucleotides, one or more of the consecutive guanines can be substituted with hypoxanthine. The substitution of hypoxanthine for one or more guanines in a string of three or more consecutive guanine bases can reduce aggregation of the antisense oligonucleotide, thereby facilitating purification.

The oligonucleotides disclosed herein are synthesized and do not include antisense compositions of biological origin. The molecules of the disclosure may also be mixed, encapsulated, conjugated or otherwise associated with other molecules, molecule structures or mixtures of compounds, as for example, liposomes, receptor targeted molecules, oral, rectal, topical or other formulations, for assisting in uptake, distribution, or absorption, or a combination thereof.

The terms “complementary” and “complementarity” refer to oligonucleotides (i.e., a sequence of nucleotides) related by base-pairing rules. For example, the sequence “T-G-A (5-3′),” is complementary to the sequence “T-C-A (5′-3′).” Complementarity may be “partial,” in which only some of the nucleic acids' bases are matched according to base pairing rules. Or, there may be “complete,” “total,” or “perfect” (100%) complementarity between the nucleic acids. The degree of complementarity between nucleic acid strands has significant effects on the efficiency and strength of hybridization between nucleic acid strands. While perfect complementarity is often desired, some embodiments can include one or more but preferably 6, 5, 4, 3, 2, or 1 mismatches with respect to the target RNA. Such hybridization may occur with “near” or “substantial” complementarity of the antisense oligomer to the target sequence, as well as with exact complementarity. In some embodiments, an oligomer may hybridize to a target sequence at about 50%, 55%, 60%, 65%, 70%, 75%, 80%, 85%, 90%, 95%, 99% or 100% complementarity. Variations at any location within the oligomer are included. In certain embodiments, variations in sequence near the termini of an oligomer are generally preferable to variations in the interior, and if present are typically within about 6, 5, 4, 3, 2, or 1 nucleotides of the 5-terminus, 3-terminus, or both termini.

The term “peptide” refers to a compound comprising a plurality of linked amino acids, e.g., which may be used to link a desired moiety, e.g., an oligonucleotide to an antibody or antibody fragment, typically an antibody or antibody fragment which specifically binds to one or more immune cell types, e.g., immune cells which are involved in an autoimmune or inflammatory disease condition.

The term “internalizing antibody or antibody fragment” in the present invention generally refers to an antibody which internalizes immune cells when it binds to its target antigen and thereupon delivers one or more payloads which are attached thereto, e.g., an oligonucleotide (RNA or DNA comprised of wild-type or modified nucleotides), into targeted immune cells. In exemplary embodiments the internalizing antibody or antibody fragment is an antibody which binds to VISTA, preferably human VISTA.

The terms “cell penetrating peptide” and “CPP” are used interchangeably and refer to cationic cell penetrating peptides, also called transport peptides, carrier peptides, or peptide transduction domains. Such peptides have the capability of inducing or enhancing cell penetration of a given cell culture population.

The term “treatment” refers to the application of one or more specific procedures used for the amelioration of a disease. In certain embodiments, the specific procedure is the administration of one or more pharmaceutical agents. “Treatment” of an individual (e.g. a mammal, such as a human) or a cell is any type of intervention used in an attempt to alter the natural course of the individual or cell. Treatment includes, but is not limited to, administration of a pharmaceutical composition, and may be performed either prophylactically or subsequent to the initiation of a pathologic event or contact with an etiologic agent. Treatment includes any desirable effect on the symptoms or pathology of a disease or condition, and may include, for example, minimal changes or improvements in one or more measurable markers of the disease or condition being treated. Also included are “prophylactic” treatments, which can be directed to reducing the rate of progression of the disease or condition being treated, delaying the onset of that disease or condition, or reducing the severity of its onset. An “effective amount” or “therapeutically effective amount” refers to an amount of therapeutic compound, such as an antisense oligomer, administered to a mammalian subject, either as a single dose or as part of a series of doses, which is effective to produce a desired therapeutic effect.

The term “amelioration” means a lessening of severity of at least one indicator of a condition or disease. In certain embodiments, amelioration includes a delay or slowing in the progression of one or more indicators of a condition or disease. The severity of indicators may be determined by subjective or objective measures which are known to those skilled in the art.

As used herein, “pharmaceutically acceptable salts” refers to derivatives of the disclosed oligonucleotides wherein the parent oligonucleotide is modified by converting an existing acid or base moiety to its salt form. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

### Oligonucleotides Comprised in Inventive ARCs or ANCs

An oligonucleotides herein generally refers to a polynucleic acid molecule which when delivered into target immune cells modulates the expression and/or the activity of a protein which modulates immunity, which gene is expressed by an immune cell. In some embodiments, a polynucleic acid molecule described herein modulates the expression of an immunomodulatory gene which may be wild type or may comprise one or more mutations, e.g., those which corelate to a disease condition associated with expression of the gene. In some instances, the polynucleic acid molecule hybridizes to a target region of wild type DNA or RNA encoding the immunomodulatory gene or a fragment thereof. In some instances, the polynucleic acid molecule is a polynucleic acid molecule that hybridizes to a target region of DNA or RNA encoded thereby comprising a mutation (e.g., a substitution, a deletion, or an addition).

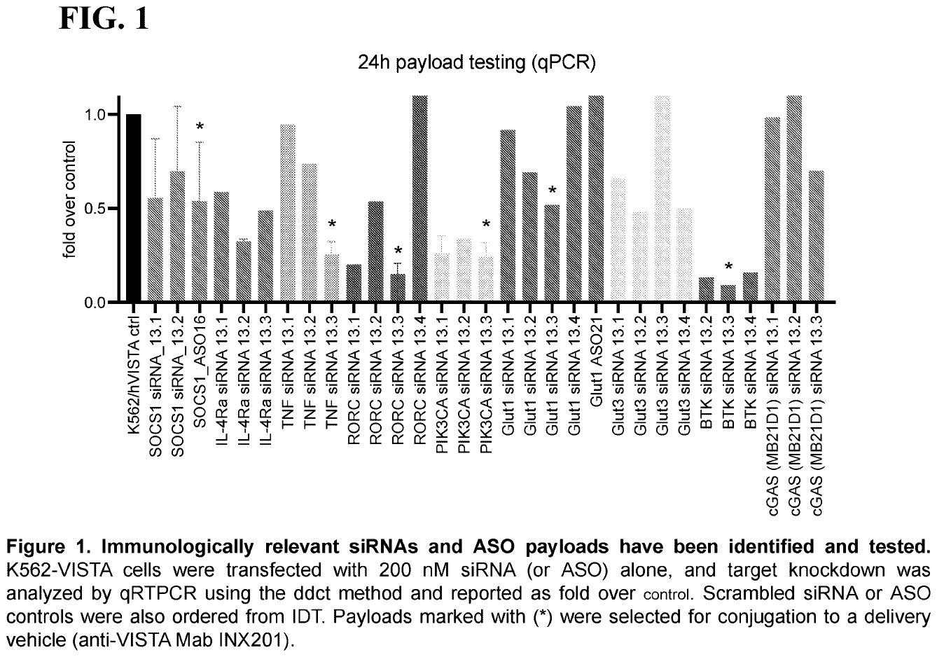

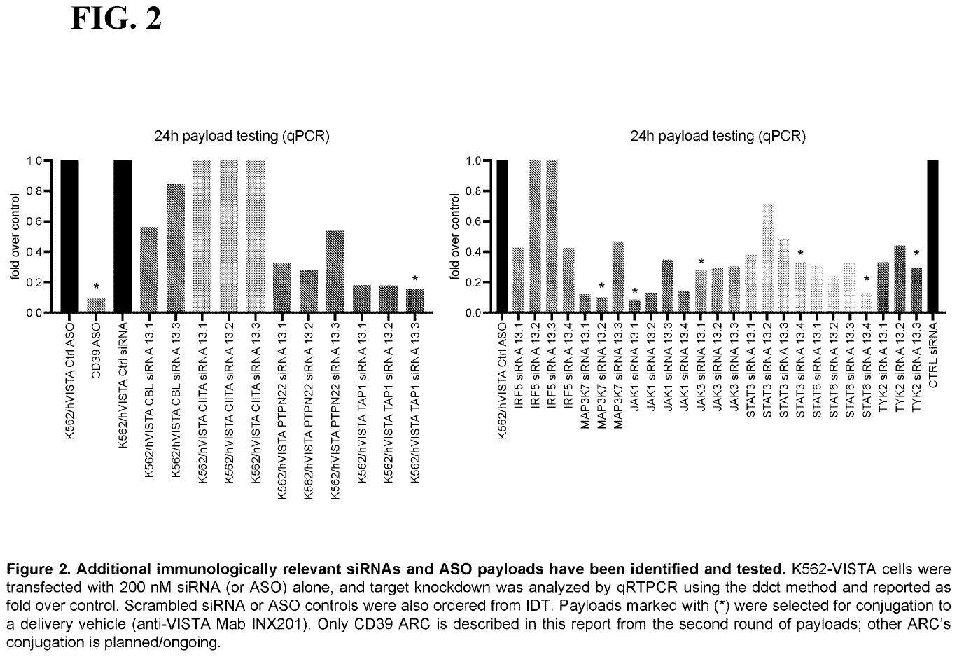

In some embodiments, the immunomodulatory gene and the RNA is selected from those identified in FIG. 1 or FIG. 2, or in Appendix 2 and Appendix 4.

In some embodiments, a polynucleic acid molecule hybridizes to a target region of a DNA or RNA encoding the targeted immunomodulatory protein which comprises one or more mutations.

In some embodiments, a polynucleic acid molecule comprises a sequence having at least 50%, 55%, 60%, 65%, 70%, 75%, 80%, 85%, 90%, 95%, 96%, 97%, 98%, 99%, or 100% sequence identity to a targeted gene sequence listed in FIG. 1 or FIG. 2. In In some embodiments, a polynucleic acid molecule described herein comprises RNA or DNA. In some cases, the polynucleic acid molecule comprises RNA.

In some instances, the RNA comprises short interfering RNA (siRNA), short hairpin RNA (shRNA), microRNA (miRNA), double-stranded RNA (dsRNA), transfer RNA (tRNA), ribosomal RNA (rRNA), or heterogeneous nuclear RNA (hnRNA). In some instances, RNA comprises shRNA. In some instances, RNA comprises miRNA. In some instances, RNA comprises dsRNA. In some instances, RNA comprises tRNA. In some instances, RNA comprises rRNA. In some instances, RNA comprises hnRNA. In some instances, the RNA comprises siRNA. In some instances, the polynucleic acid molecule comprises siRNA.

In some embodiments, a polynucleic acid molecule is from about 10 to about 50 nucleotides in length. In some instances, the polynucleic acid molecule is from about 10 to about 30, from about 15 to about 30, from about 18 to about 25, from about 18 to about 24, from about 19 to about 23, or from about 20 to about 22 nucleotides in length.

In some embodiments, a polynucleic acid molecule is about 50 nucleotides in length. In some instances, the polynucleic acid molecule is about 45 nucleotides in length. In some instances, the polynucleic acid molecule is about 40 nucleotides in length. In some instances, the polynucleic acid molecule is about 35 nucleotides in length. In some instances, the polynucleic acid molecule is about 30 nucleotides in length. In some instances, the polynucleic acid molecule is about 25 nucleotides in length. In some instances, the polynucleic acid molecule is about 20 nucleotides in length. In some instances, the polynucleic acid molecule is about 19 nucleotides in length. In some instances, the polynucleic acid molecule is about 18 nucleotides in length. In some instances, the polynucleic acid molecule is about 17 nucleotides in length. In some instances, the polynucleic acid molecule is about 16 nucleotides in length. In some instances, the polynucleic acid molecule is about 15 nucleotides in length. In some instances, the polynucleic acid molecule is about 14 nucleotides in length. In some instances, the polynucleic acid molecule is about 13 nucleotides in length. In some instances, the polynucleic acid molecule is about 12 nucleotides in length. In some instances, the polynucleic acid molecule is about 11 nucleotides in length. In some instances, the polynucleic acid molecule is about 10 nucleotides in length. In some instances, the polynucleic acid molecule is from about 10 to about 50 nucleotides in length. In some instances, the polynucleic acid molecule is from about 10 to about 45 nucleotides in length. In some instances, the polynucleic acid molecule is from about 10 to about 40 nucleotides in length. In some instances, the polynucleic acid molecule is from about 10 to about 35 nucleotides in length. In some instances, the polynucleic acid molecule is from about 10 to about 30 nucleotides in length. In some instances, the polynucleic acid molecule is from about 10 to about 25 nucleotides in length. In some instances, the polynucleic acid molecule is from about 10 to about 20 nucleotides in length. In some instances, the polynucleic acid molecule is from about 15 to about 25 nucleotides in length. In some instances, the polynucleic acid molecule is from about 15 to about 30 nucleotides in length. In some instances, the polynucleic acid molecule is from about 12 to about 30 nucleotides in length.

In some embodiments, a polynucleic acid molecule comprises a first polynucleotide. In some instances, the polynucleic acid molecule comprises a second polynucleotide. In some instances, the polynucleic acid molecule comprises a first polynucleotide and a second polynucleotide. In some instances, the first polynucleotide is a sense strand or passenger strand. In some instances, the second polynucleotide is an antisense strand or guide strand.

In some embodiments, a polynucleic acid molecule is a first polynucleotide. In some embodiments, the first polynucleotide is from about 10 to about 50 nucleotides in length. In some instances, the first polynucleotide is from about 10 to about 30, from about 15 to about 30, from about 18 to about 25, from about 18 to about 24, from about 19 to about 23, or from about 20 to about 22 nucleotides in length.

In some instances, a first polynucleotide is about 50 nucleotides in length. In some instances, the first polynucleotide is about 45 nucleotides in length. In some instances, the first polynucleotide is about 40 nucleotides in length. In some instances, the first polynucleotide is about 35 nucleotides in length. In some instances, the first polynucleotide is about 30 nucleotides in length. In some instances, the first polynucleotide is about 25 nucleotides in length. In some instances, the first polynucleotide is about 20 nucleotides in length. In some instances, the first polynucleotide is about 19 nucleotides in length. In some instances, the first polynucleotide is about 18 nucleotides in length. In some instances, the first polynucleotide is about 17 nucleotides in length. In some instances, the first polynucleotide is about 16 nucleotides in length. In some instances, the first polynucleotide is about 15 nucleotides in length. In some instances, the first polynucleotide is about 14 nucleotides in length. In some instances, the first polynucleotide is about 13 nucleotides in length. In some instances, the first polynucleotide is about 12 nucleotides in length. In some instances, the first polynucleotide is about 11 nucleotides in length. In some instances, the first polynucleotide is about 10 nucleotides in length. In some instances, the first polynucleotide is from about 10 to about 50 nucleotides in length. In some instances, the first polynucleotide is from about 10 to about 45 nucleotides in length. In some instances, the first polynucleotide is from about 10 to about 40 nucleotides in length. In some instances, the first polynucleotide is from about 10 to about 35 nucleotides in length. In some instances, the first polynucleotide is from about 10 to about 30 nucleotides in length. In some instances, the first polynucleotide is from about 10 to about 25 nucleotides in length. In some instances, the first polynucleotide is from about 10 to about 20 nucleotides in length. In some instances, the first polynucleotide is from about 15 to about 25 nucleotides in length. In some instances, the first polynucleotide is from about 15 to about 30 nucleotides in length. In some instances, the first polynucleotide is from about 12 to about 30 nucleotides in length.

In some embodiments, a polynucleic acid molecule is a second polynucleotide. In some embodiments, the second polynucleotide is from about 10 to about 50 nucleotides in length. In some instances, the second polynucleotide is from about 10 to about 30, from about 15 to about 30, from about 18 to about 25, from about 18 to about 24, from about 19 to about 23, or from about 20 to about 22 nucleotides in length.

In some instances, a second polynucleotide is about 50 nucleotides in length. In some instances, the second polynucleotide is about 45 nucleotides in length. In some instances, the second polynucleotide is about 40 nucleotides in length. In some instances, the second polynucleotide is about 35 nucleotides in length. In some instances, the second polynucleotide is about 30 nucleotides in length. In some instances, the second polynucleotide is about 25 nucleotides in length. In some instances, the second polynucleotide is about 20 nucleotides in length. In some instances, the second polynucleotide is about 19 nucleotides in length. In some instances, the second polynucleotide is about 18 nucleotides in length. In some instances, the second polynucleotide is about 17 nucleotides in length. In some instances, the second polynucleotide is about 16 nucleotides in length. In some instances, the second polynucleotide is about 15 nucleotides in length. In some instances, the second polynucleotide is about 14 nucleotides in length. In some instances, the second polynucleotide is about 13 nucleotides in length. In some instances, the second polynucleotide is about 12 nucleotides in length. In some instances, the second polynucleotide is about 11 nucleotides in length. In some instances, the second polynucleotide is about 10 nucleotides in length. In some instances, the second polynucleotide is from about 10 to about 50 nucleotides in length. In some instances, the second polynucleotide is from about 10 to about 45 nucleotides in length. In some instances, the second polynucleotide is from about 10 to about 40 nucleotides in length. In some instances, the second polynucleotide is from about 10 to about 35 nucleotides in length. In some instances, the second polynucleotide is from about 10 to about 30 nucleotides in length. In some instances, the second polynucleotide is from about 10 to about 25 nucleotides in length. In some instances, the second polynucleotide is from about 10 to about 20 nucleotides in length. In some instances, the second polynucleotide is from about 15 to about 25 nucleotides in length. In some instances, the second polynucleotide is from about 15 to about 30 nucleotides in length. In some instances, the second polynucleotide is from about 12 to about 30 nucleotides in length.

In some embodiments, a polynucleic acid molecule comprises a first polynucleotide and a second polynucleotide. In some instances, the polynucleic acid molecule further comprises a blunt terminus, an overhang, or a combination thereof. In some instances, the blunt terminus is a 5′ blunt terminus, a 3′ blunt terminus, or both. In some cases, the overhang is a 5′ overhang, 3′ overhang, or both. In some cases, the overhang comprises 1, 2, 3, 4, 5, 6, 7, 8, 9, or 10 non-base pairing nucleotides. In some cases, the overhang comprises 1, 2, 3, 4, 5, or 6 non-base pairing nucleotides. In some cases, the overhang comprises 1, 2, 3, or 4 non-base pairing nucleotides. In some cases, the overhang comprises 1 non-base pairing nucleotide. In some cases, the overhang comprises 2 non-base pairing nucleotides. In some cases, the overhang comprises 3 non-base pairing nucleotides. In some cases, the overhang comprises 4 non-base pairing nucleotides.

In some embodiments, the sequence of a polynucleic acid molecule is at least 40%, 50%, 55%, 60%, 65%, 70%, 75%, 80%, 85%, 90%, 95%, 98%, 99%, or 99.5% complementary to a target sequence. In some embodiments, the sequence of the polynucleic acid molecule is at least 50% complementary to a target sequence. In some embodiments, the sequence of the polynucleic acid molecule is at least 60% complementary to a target sequence. In some embodiments, the sequence of the polynucleic acid molecule is at least 70% complementary to a target sequence. In some embodiments, the sequence of the polynucleic acid molecule is at least 80% complementary to a target sequence. In some embodiments, the sequence of the polynucleic acid molecule is at least 90% complementary to a target sequence. In some embodiments, the sequence of the polynucleic acid molecule is at least 95% complementary to a target sequence. In some embodiments, the sequence of the polynucleic acid molecule is at least 99% complementary to a target sequence. In some instances, the sequence of the polynucleic acid molecule is 100% complementary to a target sequence.

In some embodiments, the sequence of a polynucleic acid molecule has 5 or less mismatches to a target sequence. In some embodiments, the sequence of the polynucleic acid molecule has 4 or less mismatches to a target sequence. In some instances, the sequence of the polynucleic acid molecule has 3 or less mismatches to a target sequence. In some cases, the sequence of the polynucleic acid molecule has 2 or less mismatches to a target sequence. In some cases, the sequence of the polynucleic acid molecule has 1 or less mismatches to a target sequence.

In some embodiments, the specificity of a polynucleic acid molecule that hybridizes to a target sequence described herein is a 95%, 98%, 99%, 99.5% or 100% sequence complementarity of the polynucleic acid molecule to a target sequence. In some instances, the hybridization is a high stringent hybridization condition.

In some embodiments, a polynucleic acid molecule has reduced off-target effect. In some instances, “off-target” or “off-target effects” refer to any instance in which a polynucleic acid polymer directed against a given target causes an unintended effect by interacting either directly or indirectly with another mRNA sequence, a DNA sequence or a cellular protein or other moiety. In some instances, an “off-target effect” occurs when there is a simultaneous degradation of other transcripts due to partial homology or complementarity between that other transcript and the sense and/or antisense strand of the polynucleic acid molecule.

In some embodiments, a polynucleic acid molecule comprises natural, synthetic, or artificial nucleotide analogues or bases. In some cases, the polynucleic acid molecule comprises combinations of DNA, RNA and/or nucleotide analogues. In some instances, the synthetic or artificial nucleotide analogues or bases comprise modifications at one or more of ribose moiety, phosphate moiety, nucleoside moiety, or a combination thereof.

In some embodiments, nucleotide analogues or artificial nucleotide base comprise a nucleic acid with a modification at a 2′ hydroxyl group of the ribose moiety. In some instances, the modification includes an H, OR, R, halo, SH, SR, NH2, NHR, NR2, or CN, wherein R is an alkyl moiety. Exemplary alkyl moiety includes, but is not limited to, halogens, sulfurs, thiols, thioethers, thioesters, amines (primary, secondary, or tertiary), amides, ethers, esters, alcohols and oxygen. In some instances, the alkyl moiety further comprises a modification. In some instances, the modification comprises an azo group, a keto group, an aldehyde group, a carboxyl group, a nitro group, a nitroso, group, a nitrile group, a heterocycle (e.g., imidazole, hydrazino or hydroxylamino) group, an isocyanate or cyanate group, or a sulfur containing group (e.g., sulfoxide, sulfone, sulfide, or disulfide). In some instances, the alkyl moiety further comprises a hetero substitution. In some instances, the carbon of the heterocyclic group is substituted by a nitrogen, oxygen, or sulfur. In some instances, the heterocyclic substitution includes but is not limited to, morpholino, imidazole, and pyrrolidino.

In some instances, the modification at the 2′ hydroxyl group is a 2′-O-methyl modification or a 2′-O-methoxyethyl (2′-O-MOE) modification. In some cases, the 2′-O-methyl modification adds a methyl group to the 2′ hydroxyl group of the ribose moiety whereas the 2′O-methoxyethyl modification adds a methoxyethyl group to the 2′ hydroxyl group of the ribose moiety.

In some instances, the modification at the 2′ hydroxyl group is a 2′-O-aminopropyl modification in which an extended amine group comprising a propyl linker binds the amine group to the 2′ oxygen. In some instances, this modification neutralizes the phosphate derived overall negative charge of the oligonucleotide molecule by introducing one positive charge from the amine group per sugar and thereby improves cellular uptake properties due to its zwitterionic properties.

In some instances, the modification at the 2′ hydroxyl group is a locked or bridged ribose modification (e.g., locked nucleic acid or LNA) in which the oxygen molecule bound at the 2′ carbon is linked to the 4′ carbon by a methylene group, thus forming a 2′-C,4′-C-oxy-methylene-linked bicyclic ribonucleotide monomer. Exemplary representations of the chemical structure of LNA are known in the art.

In some instances, the modification at the 2′ hydroxyl group comprises ethylene nucleic acids (ENA) such as for example 2′-4′-ethylene-bridged nucleic acid, which locks the sugar conformation into a C₃′-endo sugar puckering conformation. ENA are part of the bridged nucleic acids class of modified nucleic acids that also comprises LNA.

In some embodiments, additional modifications at the 2′ hydroxyl group include 2′-deoxy, T-deoxy-2′-fluoro, 2′-O-aminopropyl (2′-O-AP), 2′-O-dimethylaminoethyl (2′-O-DMAOE), 2′-O- dimethylaminopropyl (2′-O-DMAP), T-O- dimethylaminoethyloxyethyl (2′-O-DMAEOE), or 2′-O- N-methylacetamido (2′-O-NMA).

In some embodiments, nucleotide analogues comprise modified bases such as, but not limited to, 5-propynyluridine, 5-propynylcytidine, 6-methyladenine, 6-methylguanine, N, N,-dimethyladenine, 2-propyladenine, 2-propylguanine, 2-aminoadenine, 1-methylinosine, 3-methyluridine, 5-methylcytidine, 5-methyluridine and other nucleotides having a modification at the 5 position, 5-(2—amino) propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2, 2-dimethylguanosine, 5-methylaminoethyluridine, 5-methyloxyuridine, deazanucleotides (such as 7-deaza-adenosine, 6-azouridine, 6-azocytidine, or 6-azothymidine), 5-methyl-2-thiouridine, other thio bases (such as 2-thiouridine and 4-thiouridine and 2-thiocytidine), dihydrouridine, pseudouridine, queuosine, archaeosine, naphthyl and substituted naphthyl groups, any O- and N-alkylated purines and pyrimidines (such as N6-methyladenosine, 5-methylcarbonylmethyluridine, uridine 5-oxyacetic acid, pyridine-4-one, or pyridine-2-one), phenyl and modified phenyl groups such as aminophenol or 2,4, 6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyl nucleotides, and alkylcarbonylalkylated nucleotides. Modified nucleotides also include those nucleotides that are modified with respect to the sugar moiety, as well as nucleotides having sugars or analogs thereof that are not ribosyl. For example, the sugar moieties, in some cases are, or are based on, mannoses, arabinoses, glucopyranoses, galactopyranoses, 4′-thioribose, and other sugars, heterocycles, or carbocycles. The term nucleotide also includes what are known in the art as universal bases. By way of example, universal bases include but are not limited to 3-nitropyrrole, 5-nitroindole, or nebularine.

In some embodiments, nucleotide analogues further comprise morpholinos, peptide nucleic acids (PNAs), methylphosphonate nucleotides, thiolphosphonate nucleotides, 2′-fluoro N3-P5′-phosphoramidites, 1′, 5′-anhydrohexitol nucleic acids (HNAs), or a combination thereof. Morpholino or phosphorodiamidate morpholino oligo (PMO) comprises synthetic molecules whose structure mimics natural nucleic acid structure but deviates from the normal sugar and phosphate structures. In some instances, the five member ribose ring is substituted with a six member morpholino ring containing four carbons, one nitrogen and one oxygen. In some cases, the ribose monomers are linked by a phosphordiamidate group instead of a phosphate group. In such cases, the backbone alterations remove all positive and negative charges making morpholinos neutral molecules capable of crossing cellular membranes without the aid of cellular delivery agents such as those used by charged oligonucleotides.

In some embodiments, peptide nucleic acid (PNA) does not contain sugar ring or phosphate linkage and the bases are attached and appropriately spaced by oligoglycine-like molecules, therefore, eliminating a backbone charge.

In some embodiments, one or more modifications optionally occur at the internucleotide linkage. In some instances, modified internucleotide linkage includes, but is not limited to, phosphorothioates; phosphorodithioates; methylphosphonates; 5′-alkylenephosphonates; 5′-methylphosphonate; 3′-alkylene phosphonates; borontrifluoridates; borano phosphate esters and selenophosphates of 3′-5′linkage or 2′-5′linkage; phosphotriesters; thionoalkylphosphotriesters; hydrogen phosphonate linkages; alkyl phosphonates; alkylphosphonothioates; arylphosphonothioates; phosphoroselenoates; phosphorodiselenoates; phosphinates; phosphoramidates; 3′-alkylphosphoramidates; aminoalkylphosphoramidates; thionophosphoramidates; phosphoropiperazidates; phosphoroanilothioates; phosphoroanilidates; ketones; sulfones; sulfonamides; carbonates; carbamates; methylenehydrazos; methylenedimethylhydrazos; formacetals; thioformacetals; oximes; methyleneiminos; methylenemethyliminos; thioamidates; linkages with riboacetyl groups; aminoethyl glycine; silyl or siloxane linkages; alkyl or cycloalkyl linkages with or without heteroatoms of, for example, 1 to 10 carbons that are saturated or unsaturated and/or substituted and/or contain heteroatoms; linkages with morpholino structures, amides, or polyamides wherein the bases are attached to the aza nitrogens of the backbone directly or indirectly; and combinations thereof.

In some instances, the modification is a methyl or thiol modification such as methylphosphonate or thiolphosphonate modification. Exemplary thiolphosphonate nucleotides and methylphosphonate nucleotides are known on the art and include 2′-fluoro N3-P5′-phosphoramidites. In some instances, a modified nucleotide includes, but is not limited to, hexitol nucleic acid (or 1′, 5′-anhydrohexitol nucleic acids (HNA)).

In some embodiments, one or more modifications further optionally include modifications of the ribose moiety, phosphate backbone and the nucleoside, or modifications of the nucleotide analogues at the 3′ or the 5′ terminus. For example, the 3′ terminus optionally includes a 3′ cationic group, or by inverting the nucleoside at the 3′-terminus with a 3′-3′ linkage. In another alternative, the 3′-terminus is optionally conjugated with an aminoalkyl group, e.g., a 3′ C5-aminoalkyl dT. In an additional alternative, the 3′-terminus is optionally conjugated with an abasic site, e.g., with an apurinic or apyrimidinic site. In some instances, the 5′-terminus is conjugated with an aminoalkyl group, e.g., a 5′-O-alkylamino substituent. In some cases, the 5′-terminus is conjugated with an abasic site, e.g., with an apurinic or apyrimidinic site.

In some embodiments, a polynucleic acid molecule comprises one or more artificial nucleotide analogues described herein. In some instances, the polynucleic acid molecule comprises 1, 2, 3, 4, 5, 6, 7, 8, 9, 10, 11, 12, 13, 14, 15, 16, 17, 18, 20, 25, or more artificial nucleotide analogues described herein. In some embodiments, the artificial nucleotide analogues include 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O- dimethylaminoethyloxyethyl (2′-O-DMAEOE), or 2′-O-N-methylacetamido (2′-O-NMA) modified, LNA, ENA, PNA, HNA, morpholino, methylphosphonate nucleotides, thiolphosphonate nucleotides, 2′-fluoro N3-P5′-phosphoramidites, or a combination thereof. In some instances, the polynucleic acid molecule comprises 1, 2, 3, 4, 5, 6, 7, 8, 9, 10, 11, 12, 13, 14, 15, 16, 17, 18, 20, 25, or more of the artificial nucleotide analogues selected from 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O-N-methylacetamido (2′-O-NMA) modified, LNA, ENA, PNA, HNA, morpholino, methylphosphonate nucleotides, thiolphosphonate nucleotides, 2′-fluoro N3-P5′-phosphoramidites, or a combination thereof. In some instances, the polynucleic acid molecule comprises 1, 2, 3, 4, 5, 6, 7, 8, 9, 10, 11, 12, 13, 14, 15, 16, 17, 18, 20, 25, or more of 2′-O-methyl modified nucleotides. In some instances, the polynucleic acid molecule comprises 1, 2, 3, 4, 5, 6, 7, 8, 9, 10, 11, 12, 13, 14, 15, 16, 17, 18, 20, 25, or more of 2′-O-methoxy ethyl (2′-O-MOE) modified nucleotides. In some instances, the polynucleic acid molecule comprises 1, 2, 3, 4, 5, 6, 7, 8, 9, 10, 11, 12, 13, 14, 15, 16, 17, 18, 20, 25, or more of thiolphosphonate nucleotides.

In some instances, a polynucleic acid molecule comprises at least one of: from about 5% to about 100% modification, from about 10% to about 100% modification, from about 20% to about 100%) modification, from about 30% to about 100% modification, from about 40% to about 100%) modification, from about 50% to about 100% modification, from about 60% to about 100% modification, from about 70% to about 100% modification, from about 80% to about 100% modification, and from about 90% to about 100% modification.

In some cases, a polynucleic acid molecule comprises at least one of: from about 10% to about 90%) modification, from about 20% to about 90% modification, from about 30% to about 90%) modification, from about 40% to about 90% modification, from about 50% to about 90% modification, from about 60% to about 90% modification, from about 70% to about 90% modification, and from about 80% to about 100% modification.

In some cases, a polynucleic acid molecule comprises at least one of: from about 10% to about 80%) modification, from about 20% to about 80% modification, from about 30% to about 80%) modification, from about 40% to about 80% modification, from about 50% to about 80% modification, from about 60% to about 80% modification, and from about 70% to about 80% modification.

In some instances, a polynucleic acid molecule comprises at least one of: from about 10% to about 70%) modification, from about 20% to about 70% modification, from about 30% to about 70%) modification, from about 40% to about 70% modification, from about 50% to about 70% modification, and from about 60% to about 70% modification.

In some instances, a polynucleic acid molecule comprises at least one of: from about 10% to about 60%) modification, from about 20% to about 60% modification, from about 30% to about 60%) modification, from about 40% to about 60% modification, and from about 50% to about 60% modification.

In some cases, a polynucleic acid molecule comprises at least one of: from about 10% to about 50%) modification, from about 20% to about 50% modification, from about 30% to about 50% modification, and from about 40% to about 50% modification.

In some cases, a polynucleic acid molecule comprises at least one of: from about 10% to about 40%) modification, from about 20% to about 40% modification, and from about 30% to about 40%) modification.

In some cases, a polynucleic acid molecule comprises at least one of: from about 10% to about 30%) modification, and from about 20% to about 30% modification.

In some cases, a polynucleic acid molecule comprises from about 10% to about 20% modification. In some cases, a polynucleic acid molecule comprises from about 15% to about 90%, from about 20% to about 80%, from about 30% to about 70%, or from about 40% to about 60% modifications.

In additional cases, a polynucleic acid molecule comprises at least about 15%, 20%, 30%, 40%, 50%, 60%, 70%, 80%, 90%, 95%, or 99% modification.

In some embodiments, a polynucleic acid molecule comprises at least about 1, about 2, about 3, about 4, about 5, about 6, about 7, about 8, about 9, about 10, about 11, about 12, about 13, about 14, about 15, about 16, about 17, about 18, about 19, about 20, about 21, about 22, or more modifications.

In some instances, a polynucleic acid molecule comprises at least about 1, about 2, about 3, about 4, about 5, about 6, about 7, about 8, about 9, about 10, about 11, about 12, about 13, about 14, about 15, about 16, about 17, about 18, about 19, about 20, about 21, about 22 or more modified nucleotides.

In some instances, about 5 to about 100% of a polynucleic acid molecule comprise an artificial nucleotide analogue described herein. In some instances, about 5%, 10%, 15%, 20%, 25%, 30%, 35%, 40%, 45%, 50%, 55%, 60%, 65%, 70%, 75%, 80%, 85%, 90%, 95%, or 100% of a polynucleic acid molecule comprise an artificial nucleotide analogue. In some instances, about 5%, 10%, 15%, 20%, 25%, 30%, 35%, 40%, 45%, 50%, 55%, 60%, 65%, 70%, 75%, 80%, 85%, 90%, 95%, or 100% of a polynucleic acid molecule comprise an artificial nucleotide analogue. In some instances, about 5% of a polynucleic acid molecule comprise an artificial nucleotide analogue. In some instances, about 10% of a polynucleic acid molecule comprise an artificial nucleotide analogue.

In some embodiments, a polynucleic acid molecule is assembled from two separate polynucleotides wherein one polynucleotide comprises the sense strand and the second polynucleotide comprises the antisense strand of the polynucleic acid molecule. In other embodiments, the sense strand is connected to the antisense strand via a linker molecule, which in some instances is a polynucleotide linker or a non-nucleotide linker.

In some embodiments, a polynucleic acid molecule comprises a sense strand and antisense strand, wherein pyrimidine nucleotides in the sense strand comprise 2′-O-methylpyrimidine nucleotides and purine nucleotides in the sense strand comprise 2′-deoxy purine nucleotides. In some embodiments, a polynucleic acid molecule comprises a sense strand and antisense strand, wherein pyrimidine nucleotides present in the sense strand comprise 2′-deoxy-2′-fluoro pyrimidine nucleotides and wherein purine nucleotides present in the sense strand comprise 2′-deoxy purine nucleotides.

In some embodiments, a polynucleic acid molecule comprises a sense strand and antisense strand, wherein the pyrimidine nucleotides when present in said antisense strand are 2′-deoxy-2′-fluoro pyrimidine nucleotides and the purine nucleotides when present in said antisense strand are 2′-O-methyl purine nucleotides.

In some embodiments, a polynucleic acid molecule comprises a sense strand and antisense strand, wherein the pyrimidine nucleotides when present in said antisense strand are 2′-deoxy-2′-fluoro pyrimidine nucleotides and wherein the purine nucleotides when present in said antisense strand comprise 2′-deoxy-purine nucleotides.

In some embodiments, a polynucleic acid molecule comprises a sense strand and antisense strand, wherein the sense strand includes a terminal cap moiety at the 5′-end, the 3′-end, or both of the 5′ and 3′ ends of the sense strand. In other embodiments, the terminal cap moiety is an inverted deoxy abasic moiety.

In some embodiments, a polynucleic acid molecule comprises a sense strand and an antisense strand, wherein the antisense strand comprises a phosphate backbone modification at the 3′ end of the antisense strand. In some instances, the phosphate backbone modification is a phosphorothioate.

In some embodiments, a polynucleic acid molecule comprises a sense strand and an antisense strand, wherein the antisense strand comprises a glyceryl modification at the 3′ end of the antisense strand.

In some embodiments, a polynucleic acid molecule comprises a sense strand and an antisense strand, in which the sense strand comprises one or more, for example, about 1, 2, 3, 4, 5, 6, 7, 8, 9, 10, 11, 12, 13, 14, 15, 16, 17, 18, 19, 20, or more phosphorothioate internucleotide linkages, and/or one or more (e.g., about 1, 2, 3, 4, 5, 6, 7, 8, 9, 10 or more) 2′-deoxy, 2′-O-methyl, 2′-deoxy-2′-fluoro, and/or about one or more (e.g., about 1, 2, 3, 4, 5, 6, 7, 8, 9, 10 or more) universal base modified nucleotides, and optionally a terminal cap molecule at the 3′-end, the 5′-end, or both of the 3′- and 5′-ends of the sense strand; and in which the antisense strand comprises about 1 to about 10 or more, specifically about 1, 2, 3, 4, 5, 6, 7, 8, 9, 10, 11, 12, 13, 14, 15, 16, 17, 18, 19, 20, or more phosphorothioate internucleotide linkages, and/or one or more (e.g., about 1, 2, 3, 4, 5, 6, 7, 8, 9, 10 or more) 2′-deoxy, 2′-O-methyl, 2′-deoxy-2′-fluoro, and/or one or more (e.g., about 1, 2, 3, 4, 5, 6, 7, 8, 9, 10 or more) universal base modified nucleotides, and optionally a terminal cap molecule at the 3′-end, the 5′-end, or both of the 3′- and 5′-ends of the antisense strand. In other embodiments, one or more, for example about 1, 2, 3, 4, 5, 6, 7, 8, 9, 10, or more, pyrimidine nucleotides of the sense and/or antisense strand are chemically-modified with 2′-deoxy, 2′-O-methyl and/or 2′-deoxy-2′-fluoro nucleotides, with or without one or more, for example about 1, 2, 3, 4, 5, 6, 7, 8, 9, 10, or more, phosphorothioate internucleotide linkages and/or a terminal cap molecule at the 3-end, the 5′-end, or both of the 3′- and 5′-ends, being present in the same or different strand.

In some embodiments, a polynucleic acid molecule comprises a sense strand and an antisense strand, in which the sense strand comprises about 1 to about 25, for example, about 1, 2, 3, 4, 5, 6, 7, 8, 9, 10, 11, 12, 13, 14, 15, 16, 17, 18, 19, 20, or more phosphorothioate internucleotide linkages, and/or one or more (e.g., about 1, 2, 3, 4, 5, 6, 7, 8, 9, 10, or more) 2′-deoxy, 2′-O-methyl, 2′-deoxy-2′-fluoro, and/or one or more (e.g., about 1, 2, 3, 4, 5, 6, 7, 8, 9, 10, or more) universal base modified nucleotides, and optionally a terminal cap molecule at the 3-end, the 5′-end, or both of the 3′- and 5′-ends of the sense strand; and in which the antisense strand comprises about 1 to about 25 or more, for example about 1, 2, 3, 4, 5, 6, 7, 8, 9, 10, 11, 12, 13, 14, 15, 16, 17, 18, 19, 20, or more phosphorothioate internucleotide linkages, and/or one or more (e.g., about 1, 2, 3, 4, 5, 6, 7, 8, 9, 10 or more) 2′-deoxy, 2′-O-methyl, 2′-deoxy-2′-fluoro, and/or one or more (e.g., about 1, 2, 3, 4, 5, 6, 7, 8, 9, 10 or more) universal base modified nucleotides, and optionally a terminal cap molecule at the 3′-end, the 5-end, or both of the 3′- and 5′-ends of the antisense strand. In other embodiments, one or more, for example about 1, 2, 3, 4, 5, 6, 7, 8, 9, 10, or more, pyrimidine nucleotides of the sense and/or antisense strand are chemically-modified with 2′-deoxy, 2′-O-methyl and/or 2′-deoxy-2′-fluoro nucleotides, with or without about 1 to about 25 or more, for example about 1, 2, 3, 4, 5, 6, 7, 8, 9, 10, 11, 12, 13, 14, 15, 16, 17, 18, 19, 20, or more phosphorothioate internucleotide linkages and/or a terminal cap molecule at the 3′-end, the 5′-end, or both of the 3′- and 5′-ends, being present in the same or different strand.

In some embodiments, a polynucleic acid molecule comprises a sense strand and an antisense strand, in which the antisense strand comprises one or more, for example, about 1, 2, 3, 4, 5, 6, 7, 8, 9, 10, 11, 12, 13, 14, 15, 16, 17, 18, 19, 20, or more phosphorothioate internucleotide linkages, and/or about one or more (e.g., about 1, 2, 3, 4, 5, 6, 7, 8, 9, 10 or more) 2′-deoxy, 2′-O-methyl, 2′-deoxy-2′-fluoro, and/or one or more (e.g., about 1, 2, 3, 4, 5, 6, 7, 8, 9, 10 or more) universal base modified nucleotides, and optionally a terminal cap molecule at the 3′-end, the 5′-end, or both of the 3′- and 5′-ends of the sense strand; and wherein the antisense strand comprises about 1 to about 10 or more, specifically about 1, 2, 3, 4, 5, 6, 7, 8, 9, 10 or more phosphorothioate internucleotide linkages, and/or one or more (e.g., about 1, 2, 3, 4, 5, 6, 7, 8, 9, 10 or more) 2′-deoxy, 2′-O-methyl, 2′-deoxy-2′-fluoro, and/or one or more (e.g., about 1, 2, 3, 4, 5, 6, 7, 8, 9, 10 or more) universal base modified nucleotides, and optionally a terminal cap molecule at the 3′-end, the 5′-end, or both of the 3′- and 5′-ends of the antisense strand. In other embodiments, one or more, for example about 1, 2, 3, 4, 5, 6, 7, 8, 9, 10, 11, 12, 13, 14, 15, 16, 17, 18, 19, 20, or more pyrimidine nucleotides of the sense and/or antisense strand are chemically-modified with 2′-deoxy, 2′-O-methyl and/or 2′-deoxy-2′-fluoro nucleotides, with or without one or more, for example, about 1, 2, 3, 4, 5, 6, 7, 8, 9, 10 or more phosphorothioate internucleotide linkages and/or a terminal cap molecule at the 3′-end, the 5′-end, or both of the 3′ and 5′-ends, being present in the same or different strand.

In some embodiments, a polynucleic acid molecule comprises a sense strand and an antisense strand, in which the antisense strand comprises about 1 to about 25 or more, for example, about 1, 2, 3, 4, 5, 6, 7, 8, 9, 10, 11, 12, 13, 14, 15, 16, 17, 18, 19, 20, or more phosphorothioate internucleotide linkages, and/or one or more (e.g., about 1, 2, 3, 4, 5, 6, 7, 8, 9, 10 or more) 2′-deoxy, 2′-O-methyl, 2′-deoxy-2′-fluoro, and/or one or more (e.g., about 1, 2, 3, 4, 5, 6, 7, 8, 9, 10 or more) universal base modified nucleotides, and optionally a terminal cap molecule at the 3′-end, the 5-end, or both of the 3′- and 5′-ends of the sense strand; and wherein the antisense strand comprises about 1 to about 25 or more, for example about 1, 2, 3, 4, 5, 6, 7, 8, 9, 10, 11, 12, 13, 14, 15, 16, 17, 18, 19, 20, or more phosphorothioate internucleotide linkages, and/or one or more (e.g., about 1, 2, 3, 4, 5, 6, 7, 8, 9, 10 or more) 2′-deoxy, 2′-O-methyl, 2′-deoxy-2′-fluoro, and/or one or more (e.g., about 1, 2, 3, 4, 5, 6, 7, 8, 9, 10 or more) universal base modified nucleotides, and optionally a terminal cap molecule at the 3′-end, the 5′-end, or both of the 3′- and 5′-ends of the antisense strand. In other embodiments, one or more, for example about 1, 2, 3, 4, 5, 6, 7, 8, 9, 10 or more pyrimidine nucleotides of the sense and/or antisense strand are chemically-modified with 2′-deoxy, 2′-O-methyl and/or 2′-deoxy-2′-fluoro nucleotides, with or without about 1 to about 5, for example about 1, 2, 3, 4, 5 or more phosphorothioate internucleotide linkages and/or a terminal cap molecule at the 3′-end, the 5′-end, or both of the 3′- and 5′-ends, being present in the same or different strand.

In some embodiments, a polynucleic acid molecule described herein is a chemically-modified short interfering nucleic acid molecule having about 1 to about 25, for example, about 1, 2, 3, 4, 5, 6, 7, 8, 9, 10, 11, 12, 13, 14, 15, 16, 17, 18, 19, 20 or more phosphorothioate internucleotide linkages in each strand of the polynucleic acid molecule.

In another embodiment, a polynucleic acid molecule described herein comprises 2′-5′ internucleotide linkages. In some instances, the 2′-5′ internucleotide linkage(s) is at the 3′-end, the 5′-end, or both of the 3′- and 5′-ends of one or both sequence strands. In additional instances, the 2′-5′ internucleotide linkage(s) is present at various other positions within one or both sequence strands, for example, about 1, 2, 3, 4, 5, 6, 7, 8, 9, 10, or more including every internucleotide linkage of a pyrimidine nucleotide in one or both strands of the polynucleic acid molecule comprise a 2′-5′ internucleotide linkage, or about 1, 2, 3, 4, 5, 6, 7, 8, 9, 10, or more including every internucleotide linkage of a purine nucleotide in one or both strands of the polynucleic acid molecule comprise a 2′-5′ internucleotide linkage.

In some embodiments, a polynucleic acid molecule is a single stranded polynucleic acid molecule that mediates RNAi activity in a cell or reconstituted in vitro system, wherein the polynucleic acid molecule comprises a single stranded polynucleotide having complementarity to a target nucleic acid sequence, and wherein one or more pyrimidine nucleotides present in the polynucleic acid are 2′-deoxy-2′-fluoro pyrimidine nucleotides (e.g., wherein all pyrimidine nucleotides are 2′-deoxy-2′-fluoro pyrimidine nucleotides or alternately a plurality of pyrimidine nucleotides are 2′-deoxy-2′-fluoro pyrimidine nucleotides), and wherein one or more purine nucleotides present in the polynucleic acid are 2′-deoxy purine nucleotides (e.g., wherein all purine nucleotides are 2′-deoxy purine nucleotides or alternately a plurality of purine nucleotides are 2′-deoxy purine nucleotides), and a terminal cap modification, that is optionally present at the 3′-end, the 5′-end, or both of the 3′ and 5′-ends of the antisense sequence, the polynucleic acid molecule optionally further comprising about 1 to about 4 (e.g., about 1, 2, 3, or 4) terminal 2′-deoxy nucleotides at the 3′-end of the polynucleic acid molecule, wherein the terminal nucleotides further comprise one or more (e.g., 1, 2, 3, or 4) phosphorothioate internucleotide linkages, and wherein the polynucleic acid molecule optionally further comprises a terminal phosphate group, such as a 5′-terminal phosphate group.

In some cases, one or more artificial nucleotide analogues are resistant toward nucleases such as for example ribonuclease such as RNase H, deoxyribunuclease such as DNase, or exonuclease such as 5′-3′ exonuclease and 3′-5′ exonuclease when compared to natural polynucleic acid molecules. In some instances, artificial nucleotide analogues comprising 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O- dimethylaminoethyloxyethyl (2′-O-DMAEOE), or 2′-O-N-methylacetamido (2′-O-NMA) modified, LNA, ENA, PNA, HNA, morpholino, methylphosphonate nucleotides, thiolphosphonate nucleotides, 2′-fluoro N3-P5′-phosphoramidites, or combinations thereof are resistant toward nucleases such as for example ribonuclease such as RNase H, deoxyribunuclease such as DNase, or exonuclease such as 5′-3′ exonuclease and 3′-5′ exonuclease. In some instances, 2′-O-methyl modified polynucleic acid molecule is nuclease resistant (e.g., RNase H, DNase, 5′-3′ exonuclease or 3′-5′ exonuclease resistant). In some instances, 2′O-methoxyethyl (2′-O-MOE) modified polynucleic acid molecule is nuclease resistant (e.g., RNase H, DNase, 5′-3′ exonuclease or 3′-5′ exonuclease resistant). In some instances, 2′-O-aminopropyl modified polynucleic acid molecule is nuclease resistant (e.g., RNase H, DNase, 5′-3′ exonuclease or 3-5′ exonuclease resistant). In some instances, 2′-deoxy modified polynucleic acid molecule is nuclease resistant (e.g., RNase H, DNase, 5′-3′ exonuclease or 3′-5′ exonuclease resistant). In some instances, T-deoxy-2′-fluoro modified polynucleic acid molecule is nuclease resistant (e.g., RNase H, DNase, 5′-3′ exonuclease or 3′-5′ exonuclease resistant). In some instances, 2′-O-aminopropyl (2′-O-AP) modified polynucleic acid molecule is nuclease resistant (e.g., RNase H, DNase, 5′-3′ exonuclease or 3′-5′ exonuclease resistant). In some instances, 2′-O-dimethylaminoethyl (2-O-DMAOE) modified polynucleic acid molecule is nuclease resistant (e.g., RNase H, DNase, 5′-3′ exonuclease or 3-5′ exonuclease resistant). In some instances, 2′-O-dimethylaminopropyl (2′-O-DMAP) modified polynucleic acid molecule is nuclease resistant (e.g., RNase H, DNase, 5′-3′ exonuclease or 3′-5′ exonuclease resistant). In some instances, T-O- dimethylaminoethyloxyethyl (2′-O-DMAEOE) modified polynucleic acid molecule is nuclease resistant (e.g., RNase H, DNase, 5′-3′ exonuclease or 3′-5′ exonuclease resistant). In some instances, 2′-O-N-methylacetamido (2′-O-NMA) modified polynucleic acid molecule is nuclease resistant (e.g., RNase H, DNase, 5′-3′ exonuclease or 3-5′ exonuclease resistant). In some instances, LNA modified polynucleic acid molecule is nuclease resistant (e.g., RNase H, DNase, 5′-3′ exonuclease or 3′-5′ exonuclease resistant). In some instances, ENA modified polynucleic acid molecule is nuclease resistant (e.g., RNase H, DNase, 5′-3′ exonuclease or 3′-5′ exonuclease resistant). In some instances, HNA modified polynucleic acid molecule is nuclease resistant (e.g., RNase H, DNase, 5-3′ exonuclease or 3′-5′ exonuclease resistant). In some instances, morpholinos are nuclease resistant (e.g., RNase H, DNase, 5′-3′ exonuclease or 3′-5′ exonuclease resistant). In some instances, PNA modified polynucleic acid molecule is resistant to nucleases (e.g., RNase H, DNase, 5′-3′ exonuclease or 3′-5′ exonuclease resistant). In some instances, methylphosphonate nucleotides modified polynucleic acid molecule is nuclease resistant (e.g., RNase H, DNase, 5′-3′ exonuclease or 3′-5′ exonuclease resistant). In some instances, thiolphosphonate nucleotides modified polynucleic acid molecule is nuclease resistant (e.g., RNase H, DNase, 5′-3′ exonuclease or 3′-5′ exonuclease resistant). In some instances, polynucleic acid molecule comprising 2′-fluoro N3-P5′-phosphoramidites is nuclease resistant (e.g., RNase H, DNase, 5′-3′ exonuclease or 3′-5′ exonuclease resistant). In some instances, the 5′ conjugates described herein inhibit 5′-3′ exonucleolytic cleavage. In some instances, the 3′ conjugates described herein inhibit 3′-5′ exonucleolytic cleavage.

In some embodiments, one or more artificial nucleotide analogues have increased binding affinity toward their mRNA target relative to an equivalent natural polynucleic acid molecule. The one or more of the artificial nucleotide analogues comprising 2′-O-methyl, 2′-0-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O- dimethylaminoethyloxyethyl (2′-O-DMAEOE), or 2′-O-N-methylacetamido (2′-O-NMA) modified, LNA, ENA, PNA, HNA, morpholino, methylphosphonate nucleotides, thiolphosphonate nucleotides, or 2′-fluoro N3-P5′-phosphoramidites have increased binding affinity toward their mRNA target relative to an equivalent natural polynucleic acid molecule. In some instances, 2′-O-methyl modified polynucleic acid molecule has increased binding affinity toward their mRNA target relative to an equivalent natural polynucleic acid molecule. In some instances, 2′-O-methoxy ethyl (2′-O-MOE)-modified polynucleic acid molecule has increased binding affinity toward their mRNA target relative to an equivalent natural polynucleic acid molecule. In some instances, 2′-O-aminopropyl modified polynucleic acid molecule has increased binding affinity toward their mRNA target relative to an equivalent natural polynucleic acid molecule. In some instances, 2′-deoxy modified polynucleic acid molecule has increased binding affinity toward their mRNA target relative to an equivalent natural polynucleic acid molecule. In some instances, T-deoxy-2′-fluoro modified polynucleic acid molecule has increased binding affinity toward their mRNA target relative to an equivalent natural polynucleic acid molecule. In some instances, 2′-O-aminopropyl (2′-O-AP)-modified polynucleic acid molecule has increased binding affinity toward their mRNA target relative to an equivalent natural polynucleic acid molecule. In some instances, 2′-O-dimethylaminoethyl (2′-O-DMAOE)-modified polynucleic acid molecule has increased binding affinity toward their mRNA target relative to an equivalent natural polynucleic acid molecule. In some instances, 2′-O-dimethylaminopropyl (2′-O-DMAP)-modified polynucleic acid molecule has increased binding affinity toward their mRNA target relative to an equivalent natural polynucleic acid molecule. In some instances, T-O-dimethylaminoethyloxyethyl (2′-O-DMAEOE)-modified polynucleic acid molecule has increased binding affinity toward their mRNA target relative to an equivalent natural polynucleic acid molecule. In some instances, 2′-O-N-methylacetamido (2′-O-NMA)-modified polynucleic acid molecule has increased binding affinity toward their mRNA target relative to an equivalent natural polynucleic acid molecule. In some instances, LNA-modified polynucleic acid molecule has increased binding affinity toward their mRNA target relative to an equivalent natural polynucleic acid molecule. In some instances, ENA-modified polynucleic acid molecule has increased binding affinity toward their mRNA target relative to an equivalent natural polynucleic acid molecule. In some instances, PNA-modified polynucleic acid molecule has increased binding affinity toward their mRNA target relative to an equivalent natural polynucleic acid molecule. In some instances, HNA-modified polynucleic acid molecule has increased binding affinity toward their mRNA target relative to an equivalent natural polynucleic acid molecule. In some instances, morpholino-modified polynucleic acid molecule has increased binding affinity toward their mRNA target relative to an equivalent natural polynucleic acid molecule. In some instances, methylphosphonate nucleotide-modified polynucleic acid molecule has increased binding affinity toward their mRNA target relative to an equivalent natural polynucleic acid molecule. In some instances, thiolphosphonate nucleotide-modified polynucleic acid molecule has increased binding affinity toward their mRNA target relative to an equivalent natural polynucleic acid molecule. In some instances, polynucleic acid molecule comprising 2′-fluoro N3-P5′-phosphoramidites has increased binding affinity toward their mRNA target relative to an equivalent natural polynucleic acid molecule. In some cases, the increased affinity is illustrated with a lower Kd, a higher melt temperature (Tm), or a combination thereof.

In some embodiments, a polynucleic acid molecule is a chirally pure (or stereo pure) polynucleic acid molecule, or a polynucleic acid molecule comprising a single enantiomer. In some instances, the polynucleic acid molecule comprises L-nucleotide. In some instances, the polynucleic acid molecule comprises D-nucleotides. In some instance, a polynucleic acid molecule composition comprises less than 30%, 25%, 20%, 15%, 10%, 5%, 4%, 3%, 2%, 1%, or less of its mirror enantiomer. In some cases, a polynucleic acid molecule composition comprises less than 30%, 25%, 20%, 15%, 10%, 5%, 4%, 3%, 2%, 1%, or less of a racemic mixture.

In some embodiments, a polynucleic acid molecule may be further modified to include an aptamer-conjugating moiety. In some instances, the aptamer conjugating moiety is a DNA aptamer-conjugating moiety. In some instances, the aptamer conjugating moiety is Alphamer (Centauri Therapeutics), which comprises an aptamer portion that recognizes a specific cell-surface target and a portion that presents a specific epitopes for attaching to circulating antibodies. In some instance, a polynucleic acid molecule described herein is further modified to include an aptamer conjugating moiety as described in: U.S. Pat. Nos. 8,604,184, 8,591,910, and 7,850,975.

In some embodiments, a polynucleic acid molecule described herein is modified to increase its stability. In some embodiments, the polynucleic acid molecule is RNA (e.g., siRNA), and the polynucleic acid molecule is modified to increase its stability. In some instances, the polynucleic acid molecule is modified by one or more of the modifications described above to increase its stability. In some cases, the polynucleic acid molecule is modified at the 2′ hydroxyl position, such as by 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O-N-methylacetamido (2′-O-NMA) modification or by a locked or bridged ribose conformation (e.g., LNA or ENA). In some cases, the polynucleic acid molecule is modified by 2′-O-methyl and/or 2′-O-methoxyethyl ribose. In some cases, the polynucleic acid molecule also includes morpholinos, PNAs, HNA, methylphosphonate nucleotides, thiolphosphonate nucleotides, and/or 2′-fluoro N3-P5′-phosphoramidites to increase its stability. In some instances, the polynucleic acid molecule is a chirally pure (or stereo pure) polynucleic acid molecule. In some instances, the chirally pure (or stereo pure) polynucleic acid molecule is modified to increase its stability. Suitable modifications to the RNA to increase stability for delivery will be apparent to the skilled person.

In some embodiments, a polynucleic acid molecule in an ARC or ANC according to the invention has RNAi activity that modulates expression of RNA encoded by the target immunomodulator gene. In some instances, a polynucleic acid molecule described herein is a double-stranded siRNA molecule that down-regulates expression of the target immunomodulatory protein, wherein one of the strands of the double-stranded siRNA molecule comprises a nucleotide sequence that is complementary to a nucleotide sequence of the immunomodulator gene or RNA encoded by the immunomodulator gene or a portion thereof, and wherein the second strand of the double-stranded siRNA molecule comprises a nucleotide sequence substantially similar to the nucleotide sequence of the immunomodulator gene or RNA encoded by the immunomodulator gene or a portion thereof. In some cases, a polynucleic acid molecule described herein is a double-stranded siRNA molecule that down-regulates expression of the immunomodulator gene, wherein each strand of the siRNA molecule comprises about 15 to 25, 18 to 24, or 19 to about 23 nucleotides, and wherein each strand comprises at least about 14, 17, or 19 nucleotides that are complementary to the nucleotides of the other strand. In some cases, a polynucleic acid molecule described herein is a double-stranded siRNA molecule that down-regulates expression of the immunomodulator gene, wherein each strand of the siRNA molecule comprises about 19 to about 23 nucleotides, and wherein each strand comprises at least about 19 nucleotides that are complementary to the nucleotides of the other strand. In some instances, the RNAi activity occurs within a cell. In other instances, the RNAi activity occurs in a reconstituted in vitro system.

In some instances, a polynucleic acid molecule in an ARC or ANC according to the invention is a double-stranded polynucleotide molecule comprising self-complementary sense and antisense regions, wherein the antisense region comprises a nucleotide sequence that is complementary to a nucleotide sequence in a target nucleic acid molecule or a portion thereof and the sense region has a nucleotide sequence corresponding to the target nucleic acid sequence or a portion thereof. In some instances, the polynucleic acid molecule is assembled from two separate polynucleotides, where one strand is the sense strand and the other is the antisense strand, wherein the antisense and sense strands are self-complementary (e.g., each strand comprises a nucleotide sequence that is complementary to a nucleotide sequence in the other strand; such as where the antisense strand and sense strand form a duplex or double stranded structure, for example wherein the double stranded region is about 19, 20, 21, 22, 23, or more base pairs); the antisense strand comprises nucleotide sequence that is complementary to a nucleotide sequence in a target nucleic acid molecule or a portion thereof and the sense strand comprises a nucleotide sequence corresponding to the target nucleic acid sequence or a portion thereof Alternatively, the polynucleic acid molecule is assembled from a single oligonucleotide, where the self-complementary sense and antisense regions of the polynucleic acid molecule are linked by means of a nucleic acid based or non-nucleic acid-based linker(s).

In some cases, a polynucleic acid molecule in an ARC or ANC according to the invention is a polynucleotide with a duplex, asymmetric duplex, hairpin or asymmetric hairpin secondary structure, having self-complementary sense and antisense regions, wherein the antisense region comprises a nucleotide sequence that is complementary to a nucleotide sequence in a separate target nucleic acid molecule or a portion thereof and the sense region has a nucleotide sequence corresponding to the target nucleic acid sequence or a portion thereof. In other cases, the polynucleic acid molecule is a circular single-stranded polynucleotide having two or more loop structures and a stem comprising self-complementary sense and antisense regions, wherein the antisense region comprises nucleotide sequence that is complementary to a nucleotide sequence in a target nucleic acid molecule or a portion thereof and the sense region has a nucleotide sequence corresponding to the target nucleic acid sequence or a portion thereof, and wherein the circular polynucleotide is processed either in vivo or in vitro to generate an active polynucleic acid molecule capable of mediating RNAi. In additional cases, the polynucleic acid molecule also comprises a single stranded polynucleotide having a nucleotide sequence complementary to a nucleotide sequence in a target nucleic acid molecule or a portion thereof (for example, where such polynucleic acid molecule does not require the presence within the polynucleic acid molecule of a nucleotide sequence corresponding to the target nucleic acid sequence or a portion thereof), wherein the single stranded polynucleotide further comprises a terminal phosphate group, such as a 5′-phosphate (see for example Martinez et al., 2002, Cell, 110, 563-574 and Schwarz et al., 2002, Molecular Cell, 10, 537-568), or 5′,3′-diphosphate.

In some instances, an asymmetric duplex is a linear polynucleic acid molecule comprising an antisense region, a loop portion that comprises nucleotides or non-nucleotides, and a sense region that comprises fewer nucleotides than the antisense region to the extent that the sense region has enough complimentary nucleotides to base pair with the antisense region and form a duplex with loop. For example, an asymmetric hairpin polynucleic acid molecule comprises an antisense region having length sufficient to mediate RNAi in a cell or in vitro system (e.g. about 19 to about 22 nucleotides) and a loop region comprising about 4 to about 8 nucleotides, and a sense region having about 3 to about 18 nucleotides that are complementary to the antisense region. In some cases, the asymmetric hairpin polynucleic acid molecule also comprises a 5′-terminal phosphate group that is chemically modified. In additional cases, the loop portion of the asymmetric hairpin polynucleic acid molecule comprises nucleotides, non-nucleotides, linker molecules, or conjugate molecules.

In some embodiments, an asymmetric duplex is a polynucleic acid molecule having two separate strands comprising a sense region and an antisense region, wherein the sense region comprises fewer nucleotides than the antisense region to the extent that the sense region has enough complimentary nucleotides to base pair with the antisense region and form a duplex. For example, an asymmetric duplex polynucleic acid molecule comprises an antisense region having length sufficient to mediate RNAi in a cell or in vitro system (e.g. about 19 to about 22 nucleotides) and a sense region having about 3 to about 18 nucleotides that are complementary to the antisense region.

In some cases, a universal base refers to nucleotide base analogs that form base pairs with each of the natural DNA/RNA. bases with little discrimination between them. Non-limiting examples of universal bases include C-phenyl, C-naphthyl and other aromatic derivatives, inosine, azole carboxamides, and nitroazole derivatives such as 3-mtropyrrole, 4-nitroindoie, 5-nitroindole, and 6-nitroindole as known in the art (see for example Loakes, 2001, Nucleic Acids Research, 29, 2437-2447).

### Synthesis of Polynucleic Acid Molecules for Use in Inventive ARCs or ANCs

The subject ARCs or ANCs comprise one or more polynucleic acid molecules, typically RNAs, which polynucleic acid molecules may be synthesized as disclosed herein or using other known methods. In some embodiments, a polynucleic acid molecule described herein is constructed using chemical synthesis and/or enzymatic ligation reactions using procedures known in the art. For example, a polynucleic acid molecule is chemically synthesized using naturally occurring nucleotides or variously modified nucleotides designed to increase the biological stability of the molecules or to increase the physical stability of the duplex formed between the polynucleic acid molecule and target nucleic acids. Exemplary methods include those described in: U.S. Pat. Nos. 5,142,047; 5,185,444; 5,889,136; 6,008,400; and 6,111,086; PCT Publication No. WO2009099942; or European Publication No. 1579015. Additional exemplary methods include those described in: Griffey et al., “2′-O-aminopropyl ribonucleotides: a zwitterionic modification that enhances the exonuclease resistance and biological activity of antisense oligonucleotides,” J. Med. Chem. 39(26):5100-5109 (1997)); Obika, et al. “Synthesis of 2′-O,4′-C-methyleneuridine and -cytidine. Novel bicyclic nucleosides having a fixed C3, -endo sugar puckering”. Tetrahedron Letters 38 (50): 8735 (1997); Koizumi, M. “ENA oligonucleotides as therapeutics”. Current opinion in molecular therapeutics 8 (2): 144-149 (2006); and Abramova et al., “Novel oligonucleotide analogues based on morpholino nucleoside subunits-antisense technologies: new chemical possibilities,” Indian Journal of Chemistry 48B: 1721-1726 (2009). Alternatively, the polynucleic acid molecule is produced biologically using an expression vector into which a polynucleic acid molecule has been subcloned in an antisense orientation (i.e., RNA transcribed from the inserted polynucleic acid molecule will be of an antisense orientation to a target polynucleic acid molecule of interest).

In some embodiments, a polynucleic acid molecule is synthesized via a tandem synthesis methodology, wherein both strands are synthesized as a single contiguous oligonucleotide fragment or strand separated by a cleavable linker which is subsequently cleaved to provide separate fragments or strands that hybridize and permit purification of the duplex.

In some instances, a polynucleic acid molecule is also assembled from two distinct nucleic acid strands or fragments wherein one fragment includes the sense region and the second fragment includes the antisense region of the molecule.

Additional modification methods for incorporating, for example, sugar, base and phosphate modifications include: Eckstein et al., International Publication PCT No. WO 92/07065; Perrault et al. Nature, 1990, 344, 565-568; Picken et al. Science, 1991, 253, 314-317; Usman and Cedergren, Trends in Biochem. Sci, 1992, 17, 334-339; Usman et al. International Publication PCT No. WO 93/15187; Sproat, U.S. Pat. No. 5,334,711 and Beigelman et al., 1995, J. Biol. Chem., 270, 25702; Beigelman et al., International PCT publication No. WO 97/26270; Beigelman et al., U.S. Pat. No. 5,716,824; Usman et al., U.S. Pat. No. 5,627,053; Woolf et al., International PCT Publication No. WO 98/13526; Thompson et al., U.S. Ser. No. 60/082,404 which was filed on Apr. 20, 1998; Karpeisky et al., 1998, Tetrahedron Lett., 39, 1131; Earnshaw and Gait, 1998, Biopolymers (Nucleic Acid Sciences), 48, 39-55; Verma and Eckstein, 1998, Annu. Rev. Biochem., 67, 99-134; and Burlina et al, 1997, Bioorg. Med. Chem., 5, 1999-2010. Such publications describe general methods and strategies to determine the location of incorporation of sugar, base, and/or phosphate modifications and the like into nucleic acid molecules without modulating catalysis.

In some instances, while chemical modification of the polynucleic acid molecule internucleotide linkages with phosphorothioate, phosphorodithioate, and/or 5′-methylphosphonate linkages improves stability, excessive modifications sometimes cause toxicity or decreased activity. Therefore, when designing nucleic acid molecules, the amount of these internucleotide linkages in some cases is minimized. In such cases, the reduction in the concentration of these linkages lowers toxicity, and increases efficacy and specificity of these molecules.

### Diseases Treatable Using Inventive ARCs or ANCs

In some embodiments, an ARC or ANC according to the invention or a pharmaceutical composition containing as described herein is used for the treatment of a disease or disorder, typically an autoimmune or inflammatory disorder, cancer or a symptom associated therewith elicited by specific immune cell types.

In some instances the ARC or ANC or a composition containing is used for the treatment of an autoimmune disease, e.g., one involving myeloid or T cells.

In some instances the ARC or ANC or a composition containing is used for the treatment of a neoplastic, proliferative, neurodegenerative, neuroinflammatory, infectious, autoimmune or inflammatory disease or symptom thereof.

In some instances the ARC or ANC or a composition containing is used to treat an autoimmune disease is selected from one or more of Acromegaly, Acquired aplastic anemia, Acquired hemophilia, Agammaglobulinemia, primary, Alopecia areata, Ankylosing spondylitis (AS), Anti-NMDA receptor encephalitis, Antiphospholipid syndrome (APS)|catastrophic antiphospholipid syndrome (CAPS)/Asherson's syndrome, Arteriosclerosis, Autoimmune Addison's disease (AAD), Autoimmune autonomic ganglionopathy (AAG)/autoimmune dysautonomia|autoimmune gastrointestinal dysmotility (AGID), Autoimmune encephalitis|acute disseminated encephalomyelitis (ADEM), Autoimmune gastritis, Autoimmune hemolytic anemia (AIHA), Autoimmune hepatitis (AIH), Autoimmune hyperlipidemia, Autoimmune hypophysitis, Autoimmune inner ear disease (AIED), Autoimmune lymphoproliferative syndrome (ALPS), Autoimmune myelofibrosis, Autoimmune myocarditis, Autoimmune oophoritis, Autoimmune pancreatitis (AIP), Autoimmune polyglandular syndromes, types I, II, & III (APS type 1, APS type 2, APS type 3, APECED), Autoimmune progesterone dermatitis, Autoimmune retinopathy (AIR), Autoimmune sudden sensorineural hearing loss (SNHL), Balo disease, Behçet's disease, Birdshot chorioretinopathy/birdshot uveitis, Bullous pemphigoid, Castleman disease, Celiac disease, Chagas disease, Chronic inflammatory demyelinating polyneuropathy (CIDP), Chronic urticaria (CU), Churg-Strauss syndrome/eosinophilic granulomatosis with polyangiitis (EGPA),Cogan's syndrome, Cold agglutinin disease, CREST syndrome|limited cutaneous systemic sclerosis, Crohn's disease (CD), Cronkhite-Canada syndrome (CSS), Cryptogenic organizing pneumonia (COP), Dermatitis herpetiformis, Dermatomyositis, Diabetes, type 1, Discoid lupus, Dressler's syndrome/postmyocardial infarction/postpericardiotomy syndrome, Eczema/Atopic Dermatitis, Endometriosis, Eosinophilic esophagitis, Eosinophilic fasciitis, Erythema nodosum, Essential mixed cryoglobulinemia, Evans syndrome, Fibrosing alveolitis/Idiopathic pulmonary fibrosis (IPF), Giant cell arteritis/temporal arteritis/Horton's disease, Giant Cell Myocarditis, Glomerulonephritis, Goodpasture's syndrome/anti-GBM/anti-TBM disease, Granulomatosis with polyangiitis (GPA)/Wegener's granulomatosis, Grave's disease/thyroid eye disease, Guillain-Barre syndrome (GBS), Hashimoto's thyroiditis/chronic lymphocytic thyroiditis/autoimmune thyroiditis, Henoch-Schönlein purpura/IgA vasculitis, Hidradenitis suppurativa, Hurst's disease/acute hemorrhagic leukoencephalitis (AHLE), Hypogammaglobulinemia, IgA nephropathy/Berger's disease, Immune-mediated necrotizing myopathy (IMNM), Immune thrombocytopenia (ITP)/autoimmune thrombocytopenic purpura/autoimmune thrombocytopenia, Inclusion body myositis, IgG4-related sclerosing disease (ISD), Interstitial cystitis, Juvenile idiopathic arthritis/Adult-onset Still's disease. Juvenile polymyositis|Juvenile dermatomyositis|juvenile myositis, Kawasaki disease, Lambert-Eaton myasthenic syndrome (LEMS), Leukocytoclastic vasculitis, Lichen planus, Lichen sclerosus, Ligneous conjunctivitis, Linear IgA disease (LAD)|linear IgA bullous dermatosis (LABD), Lupus nephritis, Lyme disease/chronic Lyme disease/post-treatment Lyme disease syndrome (PTLDS), Lymphocytic colitis/microscopic colitis, Lymphocytic hypophystitis/autoimmune hypophystitis, Meniere's disease, Microscopic polyangiitis (MPA)/ANCA-associated vasculitis, Mixed connective tissue disease (MCTD), Mooren's ulcer, Mucha-Habermann disease, Multifocal motor neuropathy, Multiple sclerosis (MS), Myalgic encephalomyelitis (ME)/Chronic fatigue syndrome (CFS), Myasthenia gravis (MG), Narcolepsy, Neuromyelitis Optica/Devic's disease, Ocular cicatricial pemphigoid, Opsoclonus-myoclonus syndrome (OMS), Palindromic rheumatism, Paraneoplastic cerebellar degeneration, Paraneoplastic pemphigus, Parry-Romberg syndrome (PRS)/Hemifacial atrophy (HFA)/Progressive facial hemiatrophy, Paroxysmal nocturnal hemoglobinuria (PNH), Peripheral uveitis/pars planitis, PANS/PANDAS, Parsonage-Turner syndrome, Pemphigus gestationis/herpes gestationis, Pemphigus foliaceus, Pemphigus vulgaris, Pernicious anemia, POEMS syndrome, Polyarteritis nodosa, Polymyalgia rheumatica, Polymyositis, Postural orthostatic tachycardia syndrome (POTS), Primary biliary cirrhosis (PBC)/primary biliary cholangitis, Primary sclerosing cholangitis (PSC), Psoriasis, Palmoplantar Pustulosis, Psoriatic arthritis, Pulmonary fibrosis, idiopathic (IPF), Pure red cell aplasia (PRCA), Pyoderma gangrenosum, Rasmussen's encephalitis, Raynaud's syndrome/phenomenon, Reactive arthritis/Reiter's syndrome, Reflex sympathetic dystrophy syndrome (RSD)/Complex regional pain syndrome (CRPS), Relapsing polychondritis, Restless leg syndrome (RLS)/Willis-Ekbom disease, Rheumatic fever, Rheumatoid arthritis, Sarcoidosis, Schmidt syndrome/autoimmune polyendocrine syndrome type II, Scleritis, Scleroderma, Sclerosing Mesenteritis/Mesenteric Panniculitis, Serpiginous choroidopathy, Sjogren's syndrome, Stiff person syndrome (SPS), Small fiber sensory neuropathy, Systemic lupus erythematosus (SLE), Subacute bacterial endocarditis (SBE), Subacute cutaneous lupus, Susac syndrome, Sydenham's chorea, Sympathetic ophthalmia, Takayasu's arteritis (vasculitis), Testicular autoimmunity (vasculitis, orchitis), Tolosa-Hunt syndrome, Transverse myelitis (TM), Tubulointerstitial nephritis uveitis syndrome (TINU), Ulcerative colitis (UC), Undifferentiated connective tissue disease (UCTD), Uveitis|anterior/intermediate/posterior, Vasculitis, VEXAS Syndrome, Vitiligo, and Vogt-Koyanagi-Harada syndrome (VKH),

In some instances the ARC or ANC is used for treatment of an autoimmune disease selected from the group consisting of Addison disease, arthritis, celiac disease, lupus, Grave's disease, myasthenia gravis, multiple sclerosis, ITP, rheumatoid arthritis, colitis, inflammatory bowel disease, pernicious anemia, Hashimoto's thyroiditis Sjogren's disease, asthma, type 2 diabetes, and autoimmune type I diabetes.

In some instances the ARC or ANC or a composition containing is used for treatment of an inflammatory disease selected from the group consisting of Fatty liver disease, Endometriosis, Type 2 diabetes, mellitus, Type 1 diabetes mellitus, Inflammatory bowel disease (IBD), Asthma, Rheumatoid arthritis, asthma, Obesity, Fibromyalgia, Lupus SLE, osteoarthritis, Rheumatoid Arthritis, Shingles Herpes Zoster, and Vasculitis.

In some instances the ANC or ARC or a composition containing is used to treat a neurodegenerative or neuroinflammatory disease, e.g., Alzheimer's disease, Amyotrophic lateral sclerosis, Friedreich ataxia, Huntington's disease, Lewy body disease, aphasia, Parkinson's disease or Spinal muscular atrophy.

In some instances the ARC or ANC or a composition containing is used for treatment of cancer or a symptom thereof associated with specific immune cell types.

In some embodiments, an ARC or ANC comprising a polynucleic acid molecule or a pharmaceutical composition described herein is used for the treatment of cancer. In some instances, the cancer is a solid tumor. In some instances, the cancer is a hematologic malignancy. In some instances, the cancer is a relapsed or refractory cancer, or a metastatic cancer. In some instances, the solid tumor is a relapsed or refractory solid tumor, or a metastatic solid tumor. In some cases, the hematologic malignancy is a relapsed or refractory hematologic malignancy, or a metastatic hematologic malignancy.

In some embodiments, the cancer is a solid tumor. Exemplary solid tumor includes, but is not limited to, anal cancer, appendix cancer, bile duct cancer (i.e., cholangiocarcinoma), bladder cancer, brain tumor, breast cancer, cervical cancer, colon cancer, cancer of Unknown Primary (CUP), esophageal cancer, eye cancer, fallopian tube cancer, gastroenterological cancer, kidney cancer, liver cancer, lung cancer, medulloblastoma, melanoma, oral cancer, ovarian cancer, pancreatic cancer, parathyroid disease, penile cancer, pituitary tumor, prostate cancer, rectal cancer, skin cancer, stomach cancer, testicular cancer, throat cancer, thyroid cancer, uterine cancer, vaginal cancer, or vulvar cancer.

In some instances, a polynucleic acid molecule or a pharmaceutical composition described herein is used for the treatment of a solid tumor. In some instances, a polynucleic acid molecule or a pharmaceutical composition described herein is used for the treatment of anal cancer, appendix cancer, bile duct cancer (i.e., cholangiocarcinoma), bladder cancer, brain tumor, breast cancer, cervical cancer, colon cancer, cancer of Unknown Primary (CUP), esophageal cancer, eye cancer, fallopian tube cancer, gastroenterological cancer, kidney cancer, liver cancer, lung cancer, medulloblastoma, melanoma, oral cancer, ovarian cancer, pancreatic cancer, parathyroid disease, penile cancer, pituitary tumor, prostate cancer, rectal cancer, skin cancer, stomach cancer, testicular cancer, throat cancer, thyroid cancer, uterine cancer, vaginal cancer, or vulvar cancer. In some instances, the solid tumor is a relapsed or refractory solid tumor, or a metastatic solid tumor.

In some instances, the cancer is a hematologic malignancy. In some instances, the hematologic malignancy is a leukemia, a lymphoma, a myeloma, a non-Hodgkin's lymphoma, or a Hodgkin's lymphoma. In some instances, the hematologic malignancy comprises chronic lymphocytic leukemia (CLL), small lymphocytic lymphoma (SLL), high risk CLL, a non-CLL/SLL lymphoma, prolymphocytic leukemia (PLL), follicular lymphoma (FL), diffuse large B-cell lymphoma (DLBCL), mantle cell lymphoma (MCL), Waldenström's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

In some instances, a polynucleic acid molecule or a pharmaceutical composition described herein is used for the treatment of a hematologic malignancy. In some instances, a polynucleic acid molecule or a pharmaceutical composition described herein is used for the treatment of a leukemia, a lymphoma, a myeloma, a non-Hodgkin's lymphoma, or a Hodgkin's lymphoma. In some instances, the hematologic malignancy comprises chronic lymphocytic leukemia (CLL), small lymphocytic lymphoma (SLL), high risk CLL, a non-CLL/SLL lymphoma, prolymphocytic leukemia (PLL), follicular lymphoma (FL), diffuse large B-cell lymphoma (DLBCL), mantle cell lymphoma (MCL), Waldenström's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some cases, the hematologic malignancy is a relapsed or refractory hematologic malignancy, or a metastatic hematologic malignancy.

### Pharmaceutical Formulation

In some embodiments, the pharmaceutical formulations comprising an ARC or ANC according to the invention are administered to a subject by multiple administration routes including, but not limited to, parenteral (e.g., intravenous, subcutaneous, intramuscular), oral, intranasal, buccal, rectal, or transdermal administration routes. In some instances, the pharmaceutical composition describe herein is formulated for parenteral (e.g., intravenous, subcutaneous, intramuscular) administration. In other instances, the pharmaceutical composition describe herein is formulated for oral administration. In still other instances, the pharmaceutical composition describe herein is formulated for intranasal administration.

In some embodiments, the pharmaceutical formulations include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

In some instances, the pharmaceutical formulation includes multiparticulate formulations. In some instances, the pharmaceutical formulation includes nanoparticle formulations. In some instances, nanoparticles comprise cMAP, cyclodextrin, or lipids. In some cases, nanoparticles comprise solid lipid nanoparticles, polymeric nanoparticles, self-emulsifying nanoparticles, liposomes, microemulsions, or micellar solutions. Additional exemplary nanoparticles include, but are not limited to, paramagnetic nanoparticles, superparamagnetic nanoparticles, metal nanoparticles, fullerene-like materials, inorganic nanotubes, dendrimers (such as with covalently attached metal chelates), nanofibers, nanohorns, nano-onions, nanorods, nanoropes, and quantum dots. In some instances, a nanoparticle is a metal nanoparticle, e.g., a nanoparticle of scandium, titanium, vanadium, chromium, manganese, iron, cobalt, nickel, copper, zinc, yttrium, zirconium, niobium, molybdenum, ruthenium, rhodium, palladium, silver, cadmium, hafnium, tantalum, tungsten, rhenium, osmium, iridium, platinum, gold, gadolinium, aluminum, gallium, indium, tin, thallium, lead, bismuth, magnesium, calcium, strontium, barium, lithium, sodium, potassium, boron, silicon, phosphorus, germanium, arsenic, antimony, and combinations, alloys, or oxides thereof.

In some instances, a nanoparticle includes a core or a core and a shell, as in a core-shell nanoparticle.

In some instances, a nanoparticle is further coated with molecules for attachment of functional elements (e.g., with one or more of a polynucleic acid molecule or binding moiety described herein). In some instances, a coating comprises chondroitin sulfate, dextran sulfate, carboxymethyl dextran, alginic acid, pectin, carrageenan, fucoidan, agaropectin, porphyran, karaya gum, gellan gum, xanthan gum, hyaluronic acids, glucosamine, galactosamine, chitin (or chitosan), polyglutamic acid, polyaspartic acid, lysozyme, cytochrome C, ribonuclease, trypsinogen, chymotrypsinogen, a-chymotrypsin, polylysine, polyarginine, histone, protamine, ovalbumin, dextrin, or cyclodextrin. In some instances, a nanoparticle comprises a graphene-coated nanoparticle.

In some cases, a nanoparticle has at least one dimension of less than about 500 nm, 400 nm, 300 nm, 200 nm, or 100 nm.

In some instances, the nanoparticle formulation comprises paramagnetic nanoparticles, superparamagnetic nanoparticles, metal nanoparticles, fullerene-like materials, inorganic nanotubes, dendrimers (such as with covalently attached metal chelates), nanofibers, nanohorns, nano-onions, nanorods, nanoropes or quantum dots. In some instances, a polynucleic acid molecule or a binding moiety described herein is conjugated either directly or indirectly to the nanoparticle. In some instances, at least 1, 5, 10, 15, 20, 30, 40, 50, 60, 70, 80, 90, 100, or more polynucleic acid molecules or binding moieties described herein are conjugated either directly or indirectly to a nanoparticle.

In some embodiments, the pharmaceutical formulation comprise a delivery vector, e.g., a recombinant vector, for the delivery of the polynucleic acid molecule into cells. In some instances, the recombinant vector is DNA plasmid. In other instances, the recombinant vector is a viral vector. Exemplary viral vectors include vectors derived from adeno-associated virus, retrovirus, adenovirus, or alphavirus. In some instances, the recombinant vectors capable of expressing the polynucleic acid molecules provide stable expression in target cells. In additional instances, viral vectors are used that provide for transient expression of polynucleic acid molecules.

In some embodiments, the pharmaceutical formulations include a carrier or carrier materials selected on the basis of compatibility with the composition disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. Pharmaceutically compatible carrier materials include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

In some instances, the pharmaceutical formulations further include pH adjusting agents or buffering agents, which include acids such as acetic, boric, citric, lactic, phosphoric, and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate, and ammonium chloride. Such acids, bases and buffers are included in an amount required to maintain pH of the composition in an acceptable range.

In some instances, the pharmaceutical formulation includes one or more salts in an amount required to bring osmolality of the composition into an acceptable range. Such salts include those having sodium, potassium, or ammonium cations and chloride, citrate, ascorbate, borate, phosphate, bicarbonate, sulfate, thiosulfate, or bisulfite anions; suitable salts include sodium chloride, potassium chloride, sodium thiosulfate, sodium bisulfite, and ammonium sulfate.

In some instances, the pharmaceutical formulations further include diluent which are used to stabilize compounds because they provide a more stable environment. Salts dissolved in buffered solutions (which also provide pH control or maintenance) are utilized as diluents in the art, including, but not limited to a phosphate-buffered saline solution. In certain instances, diluents increase bulk of the composition to facilitate compression or create sufficient bulk for homogenous blend for capsule filling. Such compounds include e.g., lactose, starch, mannitol, sorbitol, dextrose, microcrystalline cellulose such as Avicel*; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

In some cases, the pharmaceutical formulations include disintegration agents or disintegrants to facilitate the breakup or disintegration of a substance. The term “disintegrate” includes both the dissolution and dispersion of the dosage form when contacted with gastrointestinal fluid. Examples of disintegration agents include a starch, e.g., a natural starch such as corn starch or potato starch, a pregelatinized starch such as National 1551 or Amijel®, or sodium starch glycolate such as Promogel® or Explotab®; a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose; a cross-linked starch such as sodium starch glycolate, a cross-linked polymer such as crospovidone; a cross-linked polyvinylpyrrolidone, alginate such as alginic acid or a salt of alginic acid such as sodium alginate, a clay such as Veegum® HV (magnesium aluminum silicate); a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

In some instances, the pharmaceutical formulations include filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

Lubricants and glidants are also optionally included in the pharmaceutical formulations described herein for preventing, reducing, or inhibiting adhesion or friction of materials. Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and the like.

Plasticizers include compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. Plasticizers also function as dispersing agents or wetting agents.

Solubilizers include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, dimethyl isosorbide and the like.

Stabilizers include compounds such as any antioxidation agents, buffers, acids, preservatives and the like.

Suspending agents include compounds such as polyvinylpyrrolidone (e.g., polyvinylpyrrolidone K12, polyvinylpyrrolidone K17, polyvinylpyrrolidone K25, or polyvinylpyrrolidone K30), vinyl pyrrolidone/vinyl acetate copolymer (S630), polyethylene glycol (e.g., the polyethylene glycol has a molecular weight of about 300 to about 6000, or about 3350 to about 4000, or about 7000 to about 5400), sodium carboxymethylcellulose, methylcellulose, hydroxypropylmethylcellulose, hydroxymethylcellulose acetate stearate, polysorbate-80, hydroxyethylcellulose, sodium alginate, gums (such as, e.g., gum tragacanth and gum acacia, guar gum, xanthans, including xanthan gum), sugars, cellulosics (such as, e.g., sodium carboxymethylcellulose, methylcellulose, sodium carboxymethylcellulose, hydroxypropylmethylcellulose, hydroxyethylcellulose), polysorbate-80, sodium alginate, polyethoxylated sorbitan monolaurate, polyethoxylated sorbitan monolaurate, povidone, and the like.

Surfactants include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Additional surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. Sometimes, surfactants are included to enhance physical stability or for other purposes.

Viscosity enhancing agents include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans, and combinations thereof.

Wetting agents include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts, and the like.

### Therapeutic Regimens

In some embodiments, the pharmaceutical compositions comprising an ARC or ANC according to the invention are administered for therapeutic applications. In some embodiments, the pharmaceutical composition is administered once per day, twice per day, three times per day or more. The pharmaceutical composition is administered daily, every day, every alternate day, five days a week, once a week, every other week, two weeks per month, three weeks per month, once a month, twice a month, three times per month, or more. The pharmaceutical composition is administered for at least 1 month, 2 months, 3 months, 4 months, 5 months, 6 months, 7 months, 8 months, 9 months, 10 months, 11 months, 12 months, 18 months, 2 years, 3 years, or more.

In some embodiments, one or more pharmaceutical compositions in an ARC or ANC according to the invention are administered simultaneously, sequentially, or at an interval period of time. In some embodiments, one or more pharmaceutical compositions are administered simultaneously. In some cases, one or more pharmaceutical compositions are administered sequentially. In additional cases, one or more pharmaceutical compositions are administered at an interval period of time (e.g., the first administration of a first pharmaceutical composition is on day one followed by an interval of at least 1, 2, 3, 4, 5, or more days prior to the administration of at least a second pharmaceutical composition).

In some embodiments, two or more different pharmaceutical compositions are co-administered. In some instances, the two or more different pharmaceutical compositions are co-administered simultaneously. In some cases, the two or more different pharmaceutical compositions are co-administered sequentially without a gap of time between administrations. In other cases, the two or more different pharmaceutical compositions are co-administered sequentially with a gap of about 0.5 hour, 1 hour, 2 hour, 3 hour, 12 hours, 1 day, 2 days, or more between administrations.

In the case wherein the patient's status does improve, upon the doctor's discretion the administration of the composition is given continuously; alternatively, the dose of the composition being administered is temporarily reduced or temporarily suspended for a certain length of time (i.e., a “drug holiday”). In some instances, the length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday is from 10%-100%, including, by way of example only, 10%, 15%, 20%, 25%, 30%, 35%, 40%, 45%, 50%, 55%,60%,65%,70%,75%,80%,85%,90%,95%, or 100%.

Once improvement of the patient's conditions has occurred, a maintenance dose is administered if necessary. Subsequently, the dosage or the frequency of administration, or both, are optionally reduced, as a function of the symptoms, to a level at which the improved disease, disorder or condition is retained.

In some embodiments, the amount of a given agent that corresponds to such an amount varies depending upon factors such as the particular compound, the severity of the disease, the identity (e.g., weight) of the subject or host in need of treatment, but nevertheless is routinely determined in a manner known in the art according to the particular circumstances surrounding the case, including, e.g., the specific agent being administered, the route of administration, and the subject or host being treated. In some instances, the desired dose is conveniently presented in a single dose or as divided doses administered simultaneously (or over a short period of time) or at appropriate intervals, for example as two, three, four or more sub-doses per day.

The foregoing ranges are merely suggestive, as the number of variables in regard to an individual treatment regime is large, and considerable excursions from these recommended values are not uncommon. Such dosages are altered depending on a number of variables, not limited to the activity of the compound used, the disease or condition to be treated, the mode of administration, the requirements of the individual subject, the severity of the disease or condition being treated, and the judgment of the practitioner.

In some embodiments, toxicity and therapeutic efficacy of such therapeutic regimens are determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it is expressed as the ratio between LD50 and ED50. Compounds exhibiting high therapeutic indices are preferred. The data obtained from cell culture assays and animal studies are used in formulating a range of dosage for use in human. The dosage of such compounds lies preferably within a range of circulating concentrations that include the ED50 with minimal toxicity. The dosage varies within this range depending upon the dosage form employed and the route of administration utilized.

### Kits/Article of Manufacture

In certain embodiments, are kits and articles of manufacture for use with one or more of the compositions and methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment. For example, the container(s) include an nucleic acid molecule described herein which specifically binds to an immunomodulator gene or RNA encoded thereby. Such kits optionally include an identifying description or label or instructions relating to its use in the methods described herein.

A kit typically includes labels listing contents and/or instructions for use and package inserts with instructions for use. A set of instructions will also typically be included.

In one embodiment, a label is on or associated with the container. In one embodiment, a label is on a container when letters, numbers, or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in a form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert.

### EXAMPLES

The examples set forth below is for the purpose of illustration and to describe certain specific embodiments of the disclosure. However, the scope of the claims is not to be in any way limited by the examples set forth herein. Various changes and modifications to the disclosed embodiments will be apparent to those skilled in the art and such changes and modifications including, without limitation, those relating to the chemical structures, substituents, derivatives, formulations or methods of the disclosure may be made without departing from the spirit of the disclosure and the scope of the appended claims.

### Example 1: Targeted Delivery of RNA to Immune Cells for the Treatment of Autoimmune Disease

In this example we provide proof of concept that a direct antibody-RNA conjugate (ARC) platform may be used to deliver siRNAs or antisense oligonucleotides (ASO) into immune cells, e.g., myeloid and lymphoid cells. These RNA-ARCs may be used to specifically inhibit mRNAs for key immune pathways, including PIK3CA and TNF as well as other known and novel pathways involved in immune regulation, tolerance, and metabolism. siRNAs inhibiting important immunological targets (Glut1, PI3K, BTK, TNF, RORC), delivered via ARCs, retain their functionality and inhibit activation of the respective immune pathways, making them attractive targets in autoimmune disease treatment space. Conversely, ASO-mediated inhibition of CD39, an ectonucleotidase involved in generating extracellular adenosine through ATP hydrolysis, results in an increased activation and cytokine production by T cells [PMID: 30871609]. ARC-delivered CD39 retained its activity and should be considered a promising approach in cancer treatment.

INX201, a humanized anti-human VISTA antibody on a human IgG1/kappa backbone with L234A/L235A/E269R/K322A silencing mutations in the Fc region, was used as a payload delivery vehicle. In this example, we show that:
1) we can conjugate siRNA or ASO to an anti-VISTA Mab and efficiently deliver RNAs into the target cells via VISTA binding and internalization;
2) ARC has similar binding and internalization properties as compared to free INX201 Mab;
3) known publicly described siRNA payloads, specific to immunologically relevant targets, (https://www.idtdna.com/pages/products/functional-genomics/dsirnas-and-trifecta-rnai-kits), which knock-down genes in regular transfection setting (inhibit target mRNA levels to <30%) retain their function when conjugated to a Mab and delivered into the target cells;
4) siRNA conjugated to an anti-VISTA Mab INX201 facilitated siRNA accumulation and function inside K562-VISTA cells;
5) siRNA conjugated to an anti-VISTA Mab INX201 had function in human PBMC and Tcells, manifested in reduced proliferation and cytokine production; and
6) CD39 ASO [PMID: 30871609]conjugated to an anti-VISTA Mab INX201 had function in human PBMC, manifested in increased cytokine production.

### ABBREVIATIONS USED IN THIS EXAMPLE

PBS phosphate-buffered saline
Fc heavy chain constant region (hinge/CH2/CH3) of an antibody
Mab monoclonal antibody
Fab fragment antigen-binding region of the antibody
min minute
hr hour
RPMI culture medium (developed at Roswell Park Memorial Institute)
PSG Penicillin-Streptomycin-Glutamine
FBS fetal bovine serum
KO knockout (lacking specific gene)
WT wild type
ml milliliter
L microliter
MFI mean fluorescent intensity (signal used in flow cytometry)
EC50 half maximal effective concentration
nM nanomolar
rpm revolutions per minute
RT room temperature
PBMC peripheral blood mononuclear cells
xeno-GvHD xenogeneic graft versus host disease (autoimmune disease animal model)
VISTA V-domain Ig suppressor of T cell activation
ASO antisense oligonucleotides
siRNA small interfering RNA (dsRNA of 20-24 nucleotides)
ARC antibody RNA conjugate
qRTPCR quantitative reverse transcription, polymerase chain reaction
LPS lipopolysaccharide
DAR drug antibody ratio
QC quality control

### INTRODUCTION

Applicant has previously developed an IgG1 anti-VISTA Mab, INX201. This Mab is a humanized anti-human VISTA antibody on a human IgG1/kappa backbone with L234A/L235A/E269R/K322A silencing mutations in the Fc region, was used as a payload delivery vehicle.

In this example we describe relevant experiments for establishing conjugation and functional proof of concept studies using INX201 delivery vehicle and publicly available siRNA or ASO payloads (see Table 1-2). Exemplary Ab sequences are provided in Appendix 1; exemplary payload sequences used for conjugation are identified in Appendix 2.

TABLE 1
anti-VISTA Mab and Fab for payload delivery.

| antibody | lot # | mg/ml |
|---|---|---|
| INX201 Mab | BP-3009-019-6.2 | 5.63 |
| INX201 Fab | 134705.1.a | 5.24 |

TABLE 2
Exemplary Targets. Payloads for targets in bold were validated
as ARCs in this example with confirmed functional activities.

| Validated pathways | | |
|---|---|---|
| with tox concerns | Novel Al | Cancer |
| IL4ra | Glut1, Glut3 | SOCS1 |
| TNFa | TAP1 | CD39 |
| BTK | CIITA | Cbl |
| RORgt | cGAS | PTPN22 |
| PIK3CA | IRF5 | |
| JAK1, JAK3 | STAT3 | |
| TYK2 | STAT6 | |
| | TAK1(MAP3K7) | |
| | HPK1 | |

### Materials and Methods

K562-VISTA cell line assays: binding, internalization, transfections, qPCR

### Materials

U-bottom 96-well plates (Falcon, #353077)
K562-VISTA (VISTA expressing cells, made in-house from WT K562, ATCC CCL-243)
IMDM media (ATCC, 30-2005)+10% FBS (ATCC, 30-2020)+P/S (Gibco, 15140-122) add G418 at 1:100 (selection for VISTA+expression)
Lonza Nucleofector 2b
MACS Quant
Cell Line Nucleofector Kit V (Lonza, VCA-1003)
DNA: pRP[Exp]-CAG>3×NLS/EGFP, Vector ID: VB900137-6122yyc (VectorBuilder, MaxH VB900137-6122yyc), for generation of GFP+ cell pool; the vector contains PuroR puromycin resistance gene
Puromycin
Anti-human IgG Fc secondary Ab (Biolegend, B172272)
Test articles: free INX201 or INX201 ARCs, for example (INX201-eGFP_siRNA_Cy5)
All payloads were synthesized at IDT and contained 3′-sense strand Cy5 label

### K562-VISTA Suspension Cells

1. Passage cells after reaching 0.5-1×10⁶ cells/mL. Maintain culture between 0.5-1×10⁶ viable cells/mL. Seed out 1-2×10⁶ cells/mL. Subculture 2 days before Nucleofection. Optimal density of Nucleofection: 2-5×10⁵.
2. The base medium for this cell line is ATCC-formulated Iscove's Modified Dulbecco's Medium, Catalog No. 30-2005. To make the complete growth medium, add the following components to the base medium: FBS to a final concentration of 10%
3. Cultures can be maintained by the addition or replacement of fresh medium. Start new cultures at 1×10⁵ viable cells/mL. Subculture at 1×10⁶ cells/ml

### Transfections

### Day 0

1. Add a supplement to the Nucleofector solution.
   a. The ratio of Nucleofector solution to supplement is 4.5:1.
   b. For a single reaction, use 82 μl of Nucleofector solution plus 18 μl of supplement to make 100 μl of the total reaction volume.
2. Prepare 12-well plates by filling wells with 1.5 mL of media and equilibrating media in plates in the incubator (1×10⁶ cells/sample).
3. Centrifuge the required number of cells (1×10⁶ cells/sample) at 200×g for 10 min at RT°C in 15 mL conical tubes. Remove the supernatant completely.
4. Resuspend the cell pellet in 100 μL RT°C Nucleofector solution per sample,
   a. Avoid leaving the cells in Nucleofector solution for extended periods of time (15 min).
5. Combine 100 μl of cell suspension with 2 g DNA (eGFP plasmid) or 20-200 nM siRNA or ASO payload.
6. Transfer cell/DNA or cell/RNA suspension into a certified cuvette (included in a kit).
7. Select program: K562: T-016 (or T-003).
8. Add 0.5 mL pre-equilibrated culture medium to the cuvette and gently transfer the sample into the pre-equilibrated 12-well plates (final volume 2 ml media per well).

### Day 1 (24h)

1. Proceed to RNA isolation and qRTPCR protocol for payload testing

### Day 2 (Stable GFP+Cell Pool Generation)

1. Begin antibiotic selection at 48 hours post transfection, Puromycin
   a. K562: 0.5 μg/mL for 7-21 days
2. Resuspend cells in 1% BSA/PBS at 5×10⁶/mL and test periodically by flow cytometry (eGFP signal)
   Resuspending ASO and siRNA from IDT
1) Store arrived items at −20 C (dry)
2) Centrifuge tubes, 500 g, 5 min
3) Resuspend ASO in TE buffer (30 min), 100 4M stock, if any precipitate remains, try heating the oligo at 55 °C for 1-5 minutes, then vortex thoroughly
4) Resuspend siRNA (duplex) in nuclease-free duplex buffer (or in nuclease-free water), 100 μM stock
5) Vortex briefly, followed by heating 94 C for 2 min, remove the tube from the heat source, and allow to cool to room temperature, the product is now resuspended duplex
6) Resuspended ASO and siRNA should be stored at −20 C

### VISTA Binding and Internalization Assay

1. Plate K562-VISTA cells in a U-bottom 96-well plate (see table below for volumes and cell #)

TABLE 3
VISTA binding and internalization assay details.

| | INX201-eGFP-siRNA-Cy5 |
|---|---|
| Time points | 5 time points: 0 h, 0.5 h, 1 h, 2 h, 4 h, 24 h |
| Cells/well | 5 × 10⁵ cells in 150 μL |
| Ab/well | 9.0 μg in 150 μL - top point of the titration |
| Final volume/well | 300 μL = 50 μL × [5 time points + 1] |

2. Prepare INX201-RNA dilution series (5× titration starting from 60 g/mL [2×])
   a. 4 points+0 Ab
3. Add the Ab titration to the cells
   a. INX201-eGFP-siRNA-Cy5—150 μL/well
   b. INX201 free Mab
4. Incubate on ice for 30 min
5. Collect 50 μL aliquot at 0 h and transfer to a 96-well V bottom plate kept on ice, add 200 μl of ice-cold PBS and centrifuge for 2 min at 500 g
   a. perform a-hIgG staining on the collected cells (Step 8)
6. Transfer the plates with the rest of the cells to the 37 °C
7. Collect 50 μL aliquots at 30 min, one h, two h, and four h and transfer to a 96-well V bottom plate kept on ice, add 200 p1 of ice-cold PBS and centrifuge for 2 min at 500 g
8. For each time point (including 0 h), perform a-hIgG staining:
   a. Add 50 μL of the secondary Ab mix (1:150 2× mix, 1:300 final)
   b. Stain at RT for 20 min
   c. Wash with 200 4 L of PBS
   d. Spin down at 500 g, 4 °C, 5 min
   e. Resuspend in 100 μL of PBS
9. Run and analyze via flow cytometry
   a. INX201-siRNA-Cy5*—MACS Quant

TABLE 4
VISTA binding and internalization flow cytometry panel.

| Channel | Ab | Cat# | DILUTION (make 2x) |
|---|---|---|---|
| BV421 | a-huIgG | Biolegend, | 1:300 |
| | | B172272 | |
| FITC | GFP | N/A | N/A |
| Cy5 | INX201-RNA | N/A | N/A |

10. Analyze binding as MFI of secondary Ab for INX201 or ARC from 0 h aliquot (30 min staining on ice)
11. Analyze internalization via the time course of the MFI signal.

### Quantitative RT-PCR Protocol

### Materials

RNeasy Plus Mini kit (Qiagen 74136) or NucleoSpin® RNA Plus (Macherey-Nagel 740984)
TaqMan reverse transcription reagents (ThermoFisher N8080234)
Taqman master mix 2× kit (ThermoFisher 4369016)
Taqman probes for Human GAPDH: Hs01922876_u1 (ThermoFisher 4331182)
Taqman probes for Human targets (ThermoFisher 4331182); see Table 5 below

The same protocol is used for K562 or immune cells (PBMC)

TABLE 5
Probes for qRTPCR

| cat # | probe | species |
|---|---|---|
| 4331182 | TaqMan (FAM); Assay ID: Hs01076112_m1 (human RORC) | human |
| 4331182 | TaqMan Gene Expression Assay (FAM); Assay ID: Hs04189703_g1, human CD45/PTPRC | human |
| 4331182 | TaqMan Gene Expression Assay (FAM); Assay ID: Hs00864158_g1, human SOCS1 | human |
| 4331182 | TaqMan Gene Expression Assay (FAM); Assay ID: Mr04329676_mr, eGFP probe | human |
| 4331182 | TaqMan Gene Expression Assay (FAM); Assay ID: Hs00892681_m1, human SLC2A1 (GLUT1) | human |
| 4331182 | TaqMan Gene Expression Assay (FAM); Assay ID: Hs00359840_m1, human SLC2A3 (GLUT3) | human |
| 4331182 | TagMan Gene Expression Assay (FAM); Assay ID: Hs00403553_m1, human Mb21d1 (cGAS) | human |
| 4331182 | TagMan Gene Expression Assay (FAM); Assay ID: Hs00174128_m1, human TNF | human |
| 4331182 | TagMan Gene Expression Assay (FAM); Assay ID: Hs01587526_g1, human Ptpn22 | human |
| 4331182 | TagMan Gene Expression Assay (FAM); Assay ID: Hs00969556_m1, human CD39/Entpd1 | human |
| 4331182 | TaqMan Gene Expression Assay (FAM); Assay ID: Hs00965056_m1, human IL4R | human |
| 4331182 | TaqMan Gene Expression Assay (FAM); Assay ID: Hs00975865_m1, human BTK | human |
| 4331182 | TaqMan Gene Expression Assay (FAM); Assay ID: Hs00907957_m1, human PI3CA | human |
| 4331182 | TaqMan Gene Expression Assay (FAM); Assay ID: Hs01011446_m1, human cbl | human |
| 4331182 | TaqMan Gene Expression Assay (FAM); Assay ID: Hs00388675_m1, human TAP1 | human |
| 4331182 | TaqMan Gene Expression Assay (FAM); Assay ID: Hs00172106_m1, human CIITA | human |
| 4331182 | Hs00374280_m1 (STAT3) | human |
| 4331182 | Hs00598625_m1 (STAT6) | human |
| 4331182 | Hs00158114_m1 (IRF5) | human |
| 4331182 | Hs01026983_m1 (JAK1) | human |
| 4331182 | Hs00354555_m1 (JAK3) | human |
| 4331182 | Hs01105947_g1 (TYK2) | human |
| 4331182 | Hs00177373_m1 (MAP3K7, TAK1) | human |
| 4331182 | Mm00441480_m1 (Slc2A1) | mouse |
| 4331182 | Mm01160477_m1 (STAT6) | mouse |
| 4331182 | Mm00443258_m1 (TNFa) | mouse |
| 4331182 | Mm00435673_m1 (PIK3CA) | mouse |

### RNA Isolation

### RNeasy Plus Mini Kit (Qiagen 74136)

With the RNeasy Plus procedure, all RNA molecules longer than 200 nucleotides are isolated. The procedure provides enrichment for mRNA.
It is essential to use the correct amount of starting material (<2e6 large cells) to obtain optimal RNA yield and purity.
Perform all steps of the procedure at room temperature.
1. Harvest cells.
   Resuspend cell in 375 pl RLT buffer.
      Buffer RLT Plus may form a precipitate during storage. If necessary, redissolve by warming, and then place at room temperature.
2. Transfer 370 μl of the cell lysate to a gDNA Eliminator spin column placed in a 2 ml collection tube (supplied). Centrifuge for 30 s at ≥8,000×g. Discard the column, and save the flow-through.
Make sure that no liquid remains on the column membrane after centrifugation. If necessary, repeat the centrifugation.
3. Add 350 μl of 70% ethanol to the flow-through, and mix well by pipetting.
   If some lysate was lost during homogenization and DNA removal, adjust the volume of ethanol accordingly. When purifying RNA from certain cell lines, precipitates may be visible after adding ethanol. This does affect the procedure.
4. Transfer up to 700 μl of the sample, including any precipitate that may have formed, to an RNeasy spin column placed in a 2 ml collection tube (supplied). Close the lid gently, and centrifuge for 15 s at ≥8,000×g. Discard the flow-through.
5. Add 700 pl Buffer RW1 to the RNeasy spin column. Close the lid gently, and centrifuge for 15 s at ≥8,000×g to wash the spin column membrane. Discard the flow-through.
6. Add 500 μl Buffer RPE to the RNeasy spin column. Close the lid gently, and centrifuge for 15 s at 8,000×g to wash the spin column membrane. Discard the flow-through.
   Buffer RPE is supplied as a concentrate. Before using it for the first time, add four volumes of ethanol (96-100%), as indicated on the bottle.
7. Add 500 μl Buffer RPE to the RNeasy spin column. Close the lid gently, and centrifuge for 2 min at ≥8,000×g to wash the spin column membrane.
8. Place the RNeasy spin column in a new 2 ml collection tube (supplied), and discard the old collection tube with the flow-through. Centrifuge at full speed for 1 min.
9. Place the RNeasy spin column in a new 1.5 ml collection tube (supplied). Add 30~40 μl RNase-free water directly to the spin column membrane. Close the lid gently, and centrifuge for 1 min at ≥8,000×g to elute the RNA.
10. Evaluate concentration by nanodrop.

### Reverse Transcription

Try using random hexamers first, especially if the efficiency of downstream priming is low, for long reverse transcripts, or reverse transcripts containing hairpins.
The RNA segment to be transcribed and later amplified can be at least 3 kb long.
Do not increase reverse transcriptase. For the synthesis of longer RNA transcripts, incubation time may be increased to 60 minutes.
With high G+C content DNA, melting at 97 °C for the first few cycles helps produce a single-stranded template for PCR amplification.
1. The reaction volume is 20 μl.
2. Thaw all reagents and keep them on ice. Mix and briefly centrifuge the components. Keep RNase Inhibitor and MultiScribe RT in a freezer.
3. Aliquot 400 ng of RNA and make the final volume 8.6 μl by adding water.
4. Combine the following components, and mix well.

| 25 mM MgCl₂ | 1.4μ | |
|---|---|---|
| 10X RT buffer | 2 | μl |
| 10 mM Dntp mix | 4 | μl |
| 0.1M DTT | 1 | μl |
| RNase inhibitor | 1 | μl |
| MultiScribe RT | 1 | μl |
| Random hexamers | 1 | μl |
| | 11.4 | μl |

5. Add 11.4 μl of the reaction mix to RNA.
6. Set parameter: 25 °C, 10 min ® 37 °C, 30 min ® 99 °C, 5 min ® 4 °C.

### Alternative Means for Reverse Transcription

### GoScript Reverse Transcriptase (Promega)

1. The reaction volume is 20 μl.
2. Thaw all reagents and keep them on ice. Mix and briefly centrifuge the components. Keep RNase Inhibitor and MultiScribe RT in a freezer.
3. Aliquot 800 ng of RNA and make the final volume 10 μl by adding water.
4. Combine the following components, and mix well.

| | | |
|---|---|---|
| 25 mM MgCl₂ | 1.5 | μl |
| 5X RT buffer | 4 | μl |
| dNTP mix | 1 | μl |
| RNase inhibitor | 0.5 | μl |
| GoScript RT | 1 | μl |
| Random hexamers | 1 | μl |
| H2O | 1 | μl |
| | 10 | μl |

5. Add 10 μl of the reaction mix to RNA.
6. Set parameter: 25 °C, 5 min; 42 °C, 60 min; 95 °C, 5 min; 4C, forever.
   gPCR
1. The reaction volume is 20 μl.
2. Combine the following components.

| | | |
|---|---|---|
| Water | 7 | μl |
| Taqman master mix | 10 | μl |
| 20X Primer | 1 | μl |
| cDNA | 2 | μl |
| TOTAL VOLUME | 20 | μl |

Spin. Set parameter: 95 °C, 10 min ® 95 °C, 15 sec; 60 °C, 1 min, for 40 cycles.

### Isolation of Human PBMCs and T Cells

### Isolation of Human PBMCs

Human peripheral blood was obtained from apheresis cones provided by volunteer donors at the DHMC blood donor program. The cone blood was diluted at 1:4 in PBS and carefully deposited on top of 13 ml of Histopaque 1077. After 20 min centrifugation at 850 g (room temperature, no brake on deceleration), the mononuclear cells from the Histopaque/PBS interfaced were collected. After one wash in PBS, the PBMC were resuspended at 100×10⁶ cells/ml for freezing (90% DMSO, 10% FBS) or at 10×10⁶ cells/ml for experiments (complete RPMI media).

### Isolation of Human T Cells

1. The manufacturer's instructions for EasySep Direct Human T Cell Isolation Kit were followed to isolate T cells from human blood:
   a. Add 50 μL (50 μL/mL of the sample) of Isolation Cocktail from EasySep Direct Human T Cell Isolation Kit (STEMCELL Technologies, Cat #19661) into the blood. Mix well by inverting tubes several times.
   b. Vortex RapidSpheres 30 seconds. Add 50 μL of RapidSpheres into the blood and mix well.
   c. Incubate at room temperature for 5 minutes.
   d. Transfer the mix to a 14 mL polystyrene round bottom tube.
   e. Add D-PBS to bring volume to 14 mL and mix gently using a pipette up and down 2-3 times.
   f. Place the tube (without lid) into the magnet and incubate at room temperature (RT) for 5 minutes.
   g. Carefully pipette the enriched cell suspension into a new 14 mL tube.
   h. Add 50 AL/ml of blood of RapidSpheres into enriched cells and mix well. Incubate at RT for 5 minutes.
   i. Place the tube (without lid) into the magnet and incubate at room temperature (RT) for 5 minutes (second separation).
   j. Carefully pipette the enriched cell suspension into a new 14 mL tube.
   k. Place the new 14 mL tube containing the second separation cells into the magnet and incubate at RT for 5 minutes.
   l. Carefully pipette the enriched cell suspension into a new 50 mL tube. Top up to 50 ml with D-PBS.
   m. Spin down cells at 515 g at ten °C for 5 minutes and discard the supernatant.
   n. Resuspend cells in 5 mL of the D-PBS and count.
2. Count the T cells by Cell Counter using AOPI. Adjust the volume of the D-PBS to reach 1×10⁶ cells/mL.
   Labeling of PBMCs or T cells with Cell Trace Violet
1. Add 20 μl DMSO to a vial of Cell Trace™ Violet staining solution.
2. Add directly to the 10 ml of the cell suspension to the final concentration of 2 4M.
3. Incubate cells for 20 minutes in a 37 °C water bath.
4. Add 40 ml RPMI to the cells to absorb any unbound dye, incubate cells for 3 minutes, centrifuge cells for 5 minutes at 300×g and resuspend the cell pellet in pre-warmed complete RPMI.

### Human PBMC and T Cell Activation and Proliferation Assays

### Materials

RPMI 1640 Medium (Gibco, 11875-093)
10% FBS (ATCC, 30-2020)
100 U/ml penicillin/streptomycin (Gibco, 15140122)
U-bottom 96-well plates
INX201 and INX201 antibody RNA conjugates (ARCs) using the following payloads: RORC, PIK3CA, TNFa, BTK, Glut1, CD45 (siRNA); CD39 ASO

### Anti-CD3/CD28 Beads—Stimulation of Human PBMCs or T Cells

CD3/CD28 T-cell Activator Dynabeads were used to activate human T cells for 3 days.
a. Mix Dynabeads by vortexing and aliquot 2.5 μl/well of beads in a 2 ml Eppendorf tube.
b. Add media to 2 ml, vortex, and place the tube on the magnet.
c. When the beads settle, remove the media and resuspend beads in the volume equivalent to the initial volume taken from the vial.
d. Add beads directly to the cells and plate in a U bottom 96-well plate, and add 100 μl of media to the total volume of 200 p1.
e. Cell number: 0.5 million/well for PBMCs; 0.1 million/well for T cells. Bead to T cell ratio 1:2.

### LPS Stimulation of Human PBMC

1. PBMCs were stimulated with 10 ng/ml LPS (Ams bio)
2. At 48 hours, media was collected for cytokine analysis, cells for flow cytometry

### INX201 or ARCs Titration

1. Prepare INX201 or ARCs in culture media to 2× final concentration (200 nM as maximum concentration), and make serial dilutions. In these experiments DAR was not quantified and we therefore used Ab concentration in ARC titration experiments.

### Flow Cytometry-Based Proliferation Analysis

1. Following incubation, centrifuge plate for 4 min, 515 g, and collect supernatant for cytokine analysis (freeze at −80 C until use).
2. Wash the cell pellet with PBS, centrifuge, and remove the supernatant.
3. Stain cells in 50 μl of corresponding antibody mix (in PBS) for 30 min at RT with shaking (400 rpm). In viability experiments, cells were stained in annexin buffer (Biolegend, 422201) and washed in PBS. Cells were resuspended in 100 μl PBS and analyzed on a flow cytometer.

TABLE 6
PBMC panel (LPS stimulation)

| Ch | Antibodies | Cat # | Dilution |
|---|---|---|---|
| | Human Fc Block | Fisher, 50-112-9053 | 200 |
| V1 | CD16 | Biolegend, 302038 | 200 |
| V2 | Live-dead-Yellow | Thermo, L34968 | 2000 |
| B1 | CD25 | Biolegend, 302605 | 200 |
| B2 | CD69 | Biolegend, 310906 | 200 |
| B3 | CD3-PercpCy5.5 | BD Biosciences, 552852 | 200 |
| B4 | CD19 | Biolegend, 302216 | 200 |
| R1 | Cy5 label | Custom made | na |
| R2 | CD14, HCD14 | Biolegend, 325620 | 100 |

TABLE 7
T cell panel (CD3/CD28 stimulation)

| Ch | Antibodies | Cat # | Dilution |
|---|---|---|---|
| | Human Fc Block | Fisher, 50-112-9053 | 200 |
| V1 | CTV | Thermo, C34557 | na |
| V2 | CD25 | Biolegend, 356120 | 200 |
| B1 | CD3 | Biolegend, 317306 | 200 |
| B2 | CD69 | Biolegend, 310906 | 200 |
| B3 | CD4 | Biolegend, 300530 | 200 |
| B4 | CD14 | Biolegend, 301814 | 100 |
| R1 | Cy5 label | Custom made | na |
| R2 | Live-dead-NearIR | Thermo, L34975 | 2000 |

TABLE 8
Viability panel (CD3/CD28 stimulation)

| Ch | Antibodies | Cat # | Dilution |
|---|---|---|---|
| | Human Fc Block | Fisher, 50-112-9053 | 200 |
| V1 | CTV | Thermo, C34557 | na |
| V2 | CD4 | Biolegend, 300546 | 200 |
| B1 | Annexin V | Biolegend, 640906 | 200 |
| B2 | CD69 | Biolegend, 310906 | 200 |
| B3 | CD73 | Biolegend, 344014 | 200 |
| B4 | CD3 | Biolegend, 317334 | 200 |
| R1 | Cy5 label | Custom made | na |
| R2 | Live-dead-NearIR | Thermo, L34975 | 2000 |

### Results

siRNA and ASO Payload Selection

Applicant has selected a variety of immunologically relevant targets, including ones with (1) existing approved drugs against (TNFa); (2) validated pathways with on-target toxicity concerns (PI3K), as well as (3) novel targets (Glut1), see Table 2. The first class of targets is used for proof of concept studies (TNFa), whereas the other targets corroborate the intrinsic advantages of the subject ARCs/ANCs which preferentially target myeloid and lymphoid cells. These immune-targeted RNA-ARCs should minimize or eliminate toxicities associated with non-immune tissue targeting (PI3K) and/or broad expression of the target (Glut1) or the nature of the target, such as it being transcription factor (RORC), which toxicities heretofore have limited or precluded the successful development of biologics and/or small molecule inhibitors against these types of “difficult” targets (TNFa [PMID: 34301319, PMID: 29158574]; BTK [PMID: 27192942, PMID: 31431692, PMID: 33122850, PMID: 369036451; PI3K [PMID: 31928691, PMID: 34127844, PMID: 26093105]; RORC [PMID: 30010338, PMID: 34040108]; Glut1 [PMID: 35878663, PMID: 34018847]; CD39 [PMID: 30871609]).

TABLE 9
Exemplary Payloads

| | target | | | | |
|---|---|---|---|---|---|
| target name | gene | modality | pathway description | problem to address | REFs |
| Tumor Necrosis Factor α | TNFa | siRNA | TNF pathway, cytokine | proof of concept for | PMID: 34301319, PMID: 29158574 |
| | | ARC | responses | ImmuNext's targeted ARC | |
| Bruton's Tyrosine Kinase | BTK | siRNA | NFKB/ERK cytokine | on-target non-immune | PMID: 27192942, PMID: 31431692, |
| | | ARC | responses | toxicities | PMID: 33122850, PMID: 36903645 |
| phosphoinositide 3- | PIK3CA | siRNA | PI3K/AKT/mTOR pathway | on-target toxicities | PMID: 31928691, PMID: 34127844, |
| kinase, p110α | | ARC | | | PMID: 26093105 |
| Retinoic Acid Receptor- | RORC | siRNA | Th17 pathway, IL23/17 axis | need to improve precision | PMID: 30010338, PMID: 34040108 |
| Related Orphan | | ARC | | targeting (only T cells) | |
| Receptor-γt | | | | | |
| glucose transporter 1 | Glut1/ | siRNA | glucose transport, immune | broad expression of the | PMID: 35878663, PMID: 34018847 |
| | SLC2A1 | ARC | cell activation | target | |
| Ectonucleoside triphosphate | CD39 | ASO | hydrolyzes extracellular | need to improve precision | PMID: 30871609 |
| diphosphohydrolase-1 | | ARC | ATP, CD8 exhaustion marker | targeting (only T cells) | |

siRNA or ASO unconjugated payloads (1-4 per target) were ordered from IDT, (https://www.idtdna.com/pages/products/functional-genomics/dsirnas-and-trifecta-rnai-kits). K562-VISTA cells were transfected with 200 nM siRNA (or ASO) alone, and target knockdown was analyzed by qRTPCR using the ddct method and reported as fold over control. Scrambled siRNA or ASO controls were also ordered from IDT (FIGS. 1-2). Payloads marked with (*) were selected for conjugation to a delivery vehicle (anti-VISTA Mab INX201).

As shown in FIG. 1, immunologically relevant siRNAs and ASO payloads have been identified and tested. K562-VISTA cells were transfected with 200 nM siRNA (or ASO) alone, and target knockdown was analyzed by qRTPCR using the ddct method and reported as fold over control. Scrambled siRNA or ASO controls were also ordered from IDT. Payloads marked with (*) were selected for conjugation to a delivery vehicle (anti-VISTA Mab INX201).

As is further shown in FIG. 2, additional immunologically relevant siRNAs and ASO payloads have been tested in the inventive ARCs. In these experiments K562-VISTA cells were transfected with 200 nM siRNA (or ASO) alone, and target knockdown was analyzed by qRTPCR using the ddct method and reported as fold over control. Scrambled siRNA or ASO controls were also ordered from IDT. Payloads marked with (*) were selected for conjugation to a delivery vehicle (anti-VISTA Mab INX201). The results obtained with a CD39 ARC is described herein as well as other ARCs comprising payloads which target other genes.

### Antibody RNA Conjugation and QC.

Anti-VISTA Mab and payloads used for conjugations are described in Tables 1-2 and Appendices 1-2. Oligo Conjugation Kit (Abcam, #ab218260) was used for all conjugations. All RNA oligos were purified by HPLC and resuspended at 100 μM. Lysine-based conjugation via the amine-reactive group, a widely utilized non-specific conjugation strategy was used. All payloads were synthesized at IDT and contained a 3′-sense strand Cy5 label and 5′-amine on the antisense strand (see Appendix 2).

### Reducing SDS-PAGE and a Silver Stain.

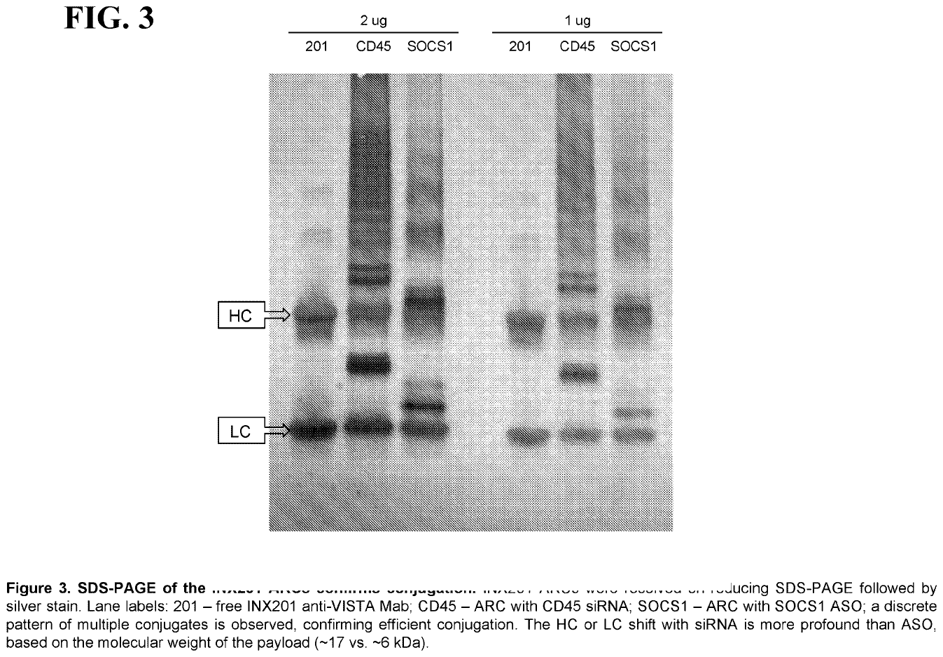

The level of ARC conjugation was analyzed by SDS-PAGE and subsequent silver stain to visualize protein. Briefly, ARCs were mixed with reducing 2× Laemmli Sample Buffer (Bio-Rad, #1610737) and incubated at 80 °C for 5 min. Reduced samples were resolved using 4-15% Mini-Protean TGX Precast Gel (Bio-Rad, #4561083) following the manufacturer's instructions. After the electrophoresis, the gel was rinsed with Mili-Q water and stained for protein using SilverQuest reagents (Thermo Fisher, #LC6070). CD45 siRNA ARC and SOCS1 ASO ARC were analyzed as representative ARCs for both siRNA and ASO payloads. Both showed expected conjugation patterns (FIG. 3). Nanodrop measurements performed for ASO ARC were: 37.2 μM for RNA and 8.0 μM for protein; therefore, the DAR was ~4.65.

The SDS-PAGE experimental results in FIG. 3 with INX201 ARCs confirms effective conjugation. In these experiments INX201 ARCs were resolved on reducing SDS-PAGE followed by silver stain. Lane labels: 201—free INX201 anti-VISTA Mab; CD45—ARC with CD45 siRNA; SOCS1—ARC with SOCS1 ASO; a discrete pattern of multiple conjugates is observed, confirming efficient conjugation. The HC or LC shift with siRNA is more profound than ASO, based on the molecular weight of the payload (~17 vs. ~6 kDa).

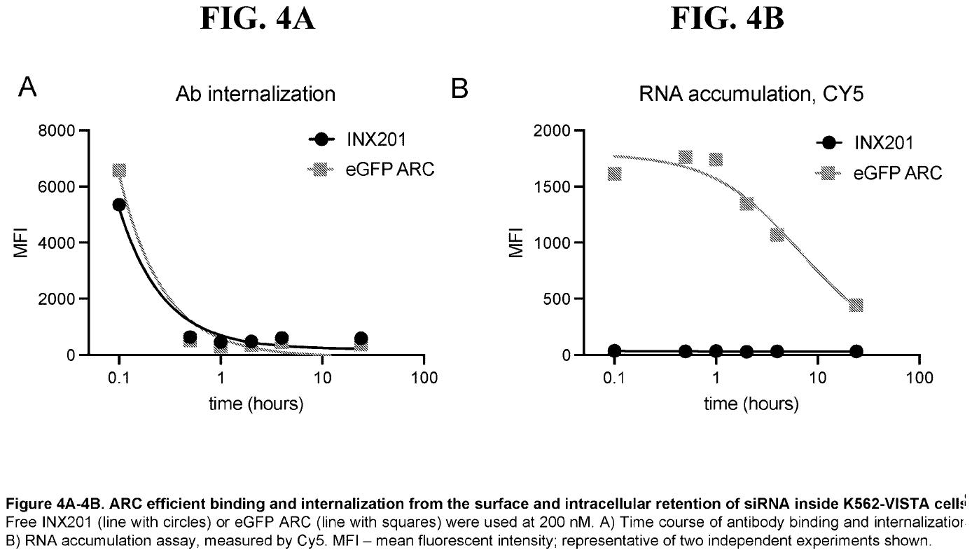

Binding (to VISTA target) and internalization were compared between free INX201 and INX201 eGFP siRNA ARC. K562-VISTA cells were incubated with 200 nM of free Mab or ARC. Binding and Internalization were measured in a time course. Binding to target was equivalent between free antibody and ARC as evident as a zero hour time point (visualized as 0.1 h, FIG. 4A). Internalization was also similar between free INX201 antibody and ARC and was rapid and efficient, as previously described by us. Specifically, within 30 minutes, over 90% of antibodies were internalized in both ARC and free Ab samples. It is evident by the lack of antibodies detected on the cell surface in the time course assay (FIG. 4A).

A Cy5 label, present on siRNA, was used to detect RNA inside the cells (given the lack of antibody detection on the cell surface due to internalization in the ARC samples, we assume that most of Cy5 detection is intracellular). As evidenced by a time course experiment, Cy5 was readily detected inside K562-VISTA cells and was preserved for the duration of the experiment (24 hours).

The experimental results in FIG. 4 show that the exemplary ARCs efficiently bind to and internalize from the surface and provide for efficient and prolonged intracellular retention of siRNA inside K562-VISTA cells. Free INX201 (line with circles) or eGFP ARC (line with squares) were used at 200 nM. A) Time course of antibody binding and internalization. B) RNA accumulation assay, measured by Cy5. MFI—mean fluorescent intensity; representative of two independent experiments shown.

### ARC Functional Testing in the K562-VISTA Cells

K562-VISTA cells were transfected with plasmid DNA expressing eGFP, and a GFP+ cell pool was used 14-28 days after transfection. The cell pool was treated with no drug or eGFP ARC. eGFP or CD45 protein levels were measured by flow cytometry (Table 10). Maximal inhibition of about 50% in eGFP protein level was achieved by ARC, which was similar as compared to eGFP mRNA knockdown levels, measured by qPCR in the same cell pool, treated with transfected eGFP free siRNA (Figure SA).

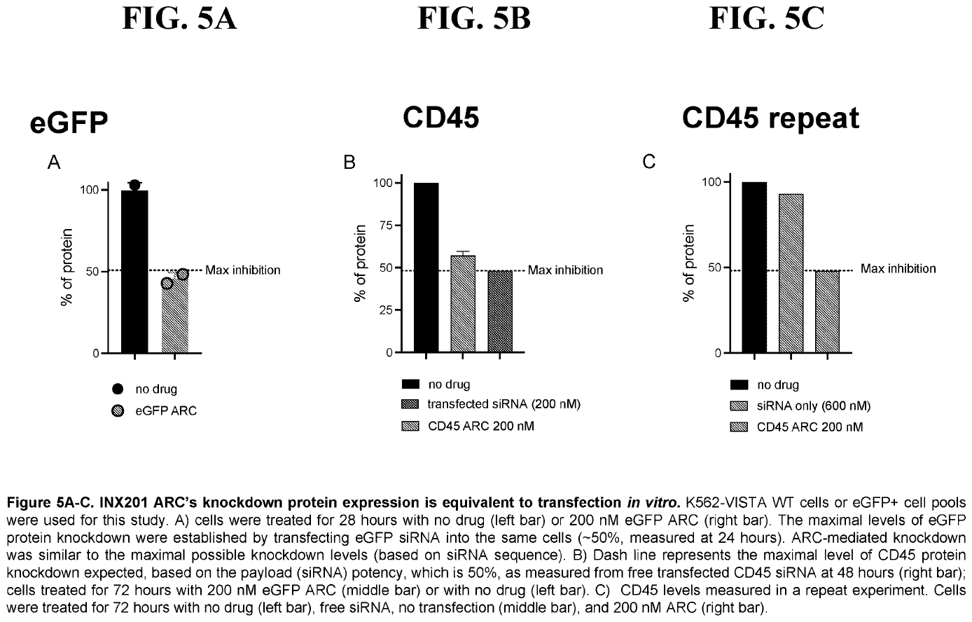

K562-VISTA cells are naturally CD45+. CD45 is expressed on the surface of the cells and therefore presents an attractive target for proof of concept studies in measuring the target (CD45) knockdown. Cells were untreated or treated with siRNA by transfection or CD45 ARC (200 nM). For transfection experiments, CD45 levels were measured by flow cytometry at 48 hours; for CD45 ARC experiments, CD45 levels were measured at 72 hours. Two independent experiments were performed with similar results (FIG. 5B-C). About 50% of CD45 protein knockdown from the surface was observed in ARC-treated cells but not in untreated cells. Transfected siRNA inhibited protein to similar levels (FIG. 5B), and siRNA freely added to cells without transfection was unable to inhibit CD45 levels (FIG. 5C). Free siRNA was added in excess to ensure that any knockdown observed by ARC was not due to any residual presence of free siRNA payload in ARC conjugate.

TABLE 10
Flow cytometry panel/channels used for ARC QC in K562 cells.

| Ch | Antibodies | Cat # | Dilution |
|---|---|---|---|
| | Human Fc Block | Fisher, 50-112-9053 | 200 |
| V2 | Live-dead-Yellow | Thermo, L34968 | 2000 |
| B1 | CD45-FITC, HI30 clone | Biolegend, 304054 | 750 |
| R1 | Cy5 label | Custom made | na |

As can be seen from the experimental results in Figure SA-C, INX201 ARC's knockdown protein expression is equivalent to transfection in vitro. K562-VISTA WT cells or eGFP+ cell pools were used for this experiment. In the experiments in FIG. 5A the cells were treated for 28 hours with no drug (left bar) or 200 nM eGFP ARC (right bar). The maximal levels of eGFP protein knockdown were established by transfecting eGFP siRNA into the same cells (~50%, measured at 24 hours). ARC-mediated knockdown was similar to the maximal possible knockdown levels (based on siRNA sequence). In the experiments in FIG. 5B, the dash line represents the maximal level of CD45 protein knockdown expected, based on the payload (siRNA) potency, which is 50%, as measured from free transfected CD45 siRNA at 48 hours (right bar); cells treated for 72 hours with 200 nM eGFP ARC (middle bar) or with no drug (left bar). In the experiments in FIG. 5C, CD45 levels measured in a repeat experiment. Cells were treated for 72 hours with no drug (left bar), free siRNA, no transfection (middle bar), and 200 nM ARC (right bar).

### ARC Functional Testing in Immune Cells

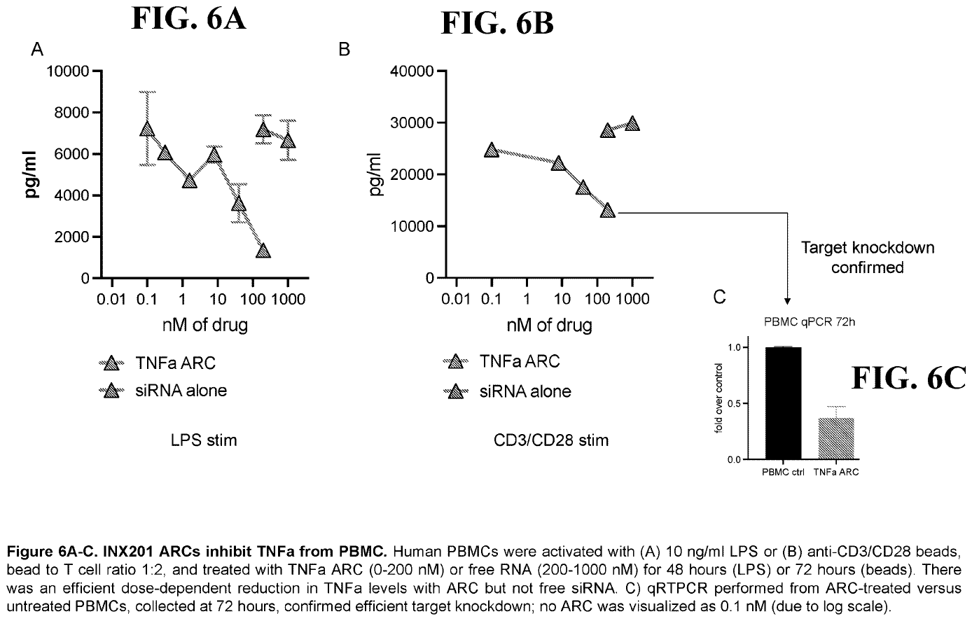

In additional experiments shown in FIG. 6A-C human PBMCs were stimulated with LPS or anti-CD3/CD28 dynabeads as described in the materials and methods. Media was collected for cytokine analysis by Luminex at 48 hours for LPS stimulation and at 72 hours for CD3/CD28 stimulation. TNFa ARC was supplied at the stimulation time at the concentration range from 200 nM to 0.32 nM. Free siRNA alone (no transfection) was used as a control and applied at 200-1000 nM. TNFa protein, measured by Luminex, was efficiently knocked down by TNFa ARC in a dose-dependent manner for both LPS-stimulated PBMCs (FIG. 6A) and for anti-CD3/CD28-stimulated PBMCs (FIG. 6B). The level of knockdown was >80% (LPS stimulation) and ~50% (anti-CD3/CD28 stimulation). For anti-CD3/CD28 stimulation, we used cells collected at 72 hours for qRTPCR to validate that the target TNFa was knocked down at the mRNA level. In this case, we saw about 60% target knockdown on RNA level in PBMCs, which correlated well with the 50% reduction in TNFa cytokine levels (FIG. 6C).

More specifically, the experiments shown in FIG. 6A-C show that INX201 ARCs inhibit TNFa from PBMC. Human PBMCs were activated with (A) 10 ng/ml LPS or (B) anti-CD3/CD28 beads, bead to T cell ratio 1:2, and treated with TNFa ARC (0-200 nM) or free RNA (200-1000 nM) for 48 hours (LPS) or 72 hours (beads). There was an efficient dose-dependent reduction in TNFa levels with ARC but not free siRNA. C) qRTPCR performed from ARC-treated versus untreated PBMCs, collected at 72 hours, confirmed efficient target knockdown; no ARC was visualized as 0.1 nM (due to log scale).

In vitro PBMC or T cells proliferation, assays show that blockade of immunologically relevant targets by siRNA, delivered via ARC (Glut1, PI3K, BTK, TNF, RORC), results in a reduction of cytokine production or T cell proliferation without impacting T cell viability (FIGS. 6-13). The reduction in proliferation is manifested predominantly in slowing the proliferation down (the last two peaks of newly proliferating cells are affected), indicating an important feature of siRNA-ARC therapy to control the proliferation of effector cells without acting as a global immunosuppressor.

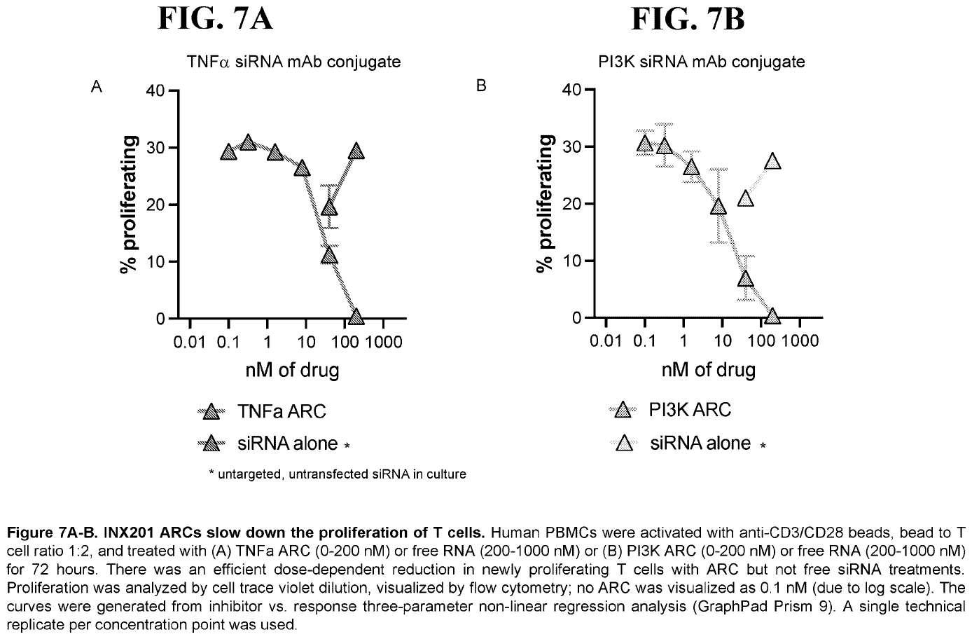

Specifically, TNFa ARC, but not free TNFa siRNA, slowed down the proliferation of T cells (FIG. 7A); PI3K ARC, but not free PI3K siRNA, also slowed down the proliferation of T cells in a dose-dependent manner (FIG. 7B).

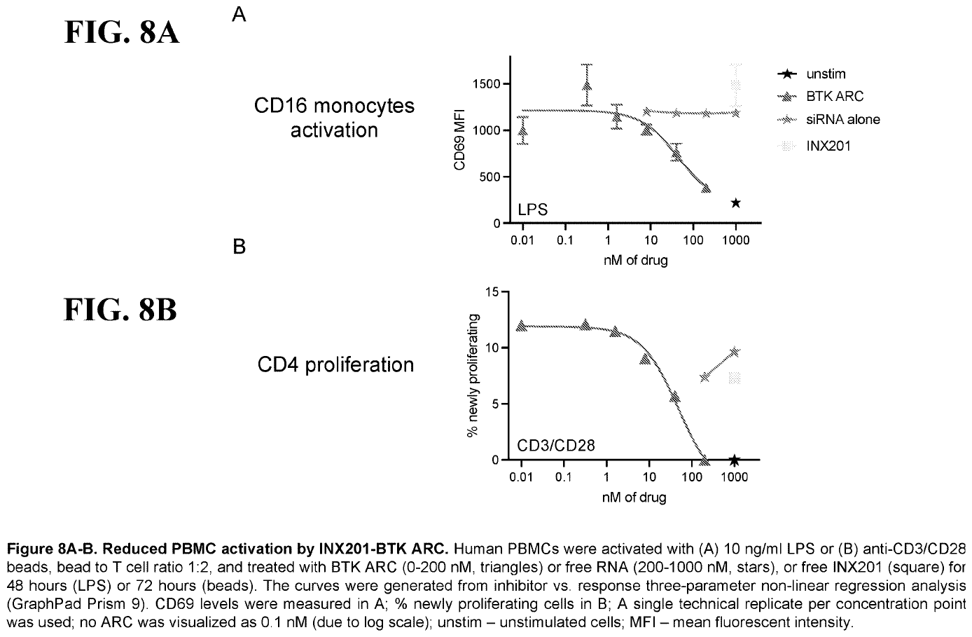

In additional experiments shown in FIG. 8, it was further demonstrated that INX201 ARCs slow down the proliferation of T cells. Human PBMCs were activated with anti-CD3/CD28 beads, bead to T cell ratio 1:2, and treated with (A) TNFa ARC (0-200 nM) or free RNA (200-1000 nM) or (B) PI3K ARC (0-200 nM) or free RNA (200-1000 nM) for 72 hours. As can be seen there was an efficient dose-dependent reduction in newly proliferating T cells with the exemplary ARC but not free siRNA treatments. Proliferation was analyzed by cell trace violet dilution, visualized by flow cytometry; no ARC was visualized as 0.1 nM (due to log scale). The curves were generated from inhibitor vs. response three-parameter non-linear regression analysis (GraphPad Prism 9). A single technical replicate per concentration point was used.

BTK ARC was tested in human PBMCs activated with LPS or anti-CD3/CD28 dynabeads as described above. In LPS stimulation experiments BTK ARC, but not free BTK siRNA, was able to reduce activation of CD16+ monocytes as measured by CD69 levels at 48 hours post drug addiction (FIG. 8A). CD69 levels were reduced to levels comparable to unstimulated cells. BTK ARC also slowed down the proliferation of T cells as measured at 72 hours post-stimulation (FIG. 8B). Free INX201 antibody or free untransfected siRNA payload had no impact on these assays.

More specifically, the experiments shown in FIG. 8A-B show reduced PBMC activation by INX201-BTK ARC. In these experiments Human PBMCs were activated with (A) 10 ng/ml LPS or (B) anti-CD3/CD28 beads, bead to T cell ratio 1:2, and treated with BTK ARC (0-200 nM, triangles) or free RNA (200-1000 nM, stars), or free INX201 (square) for 48 hours (LPS) or 72 hours (beads). The curves were generated from inhibitor vs. response three-parameter non-linear regression analysis (GraphPad Prism 9). CD69 levels were measured in A; % newly proliferating cells in B; A single technical replicate per concentration point was used; no ARC was visualized as 0.1 nM (due to log scale); unstim—unstimulated cells; MFI—mean fluorescent intensity.

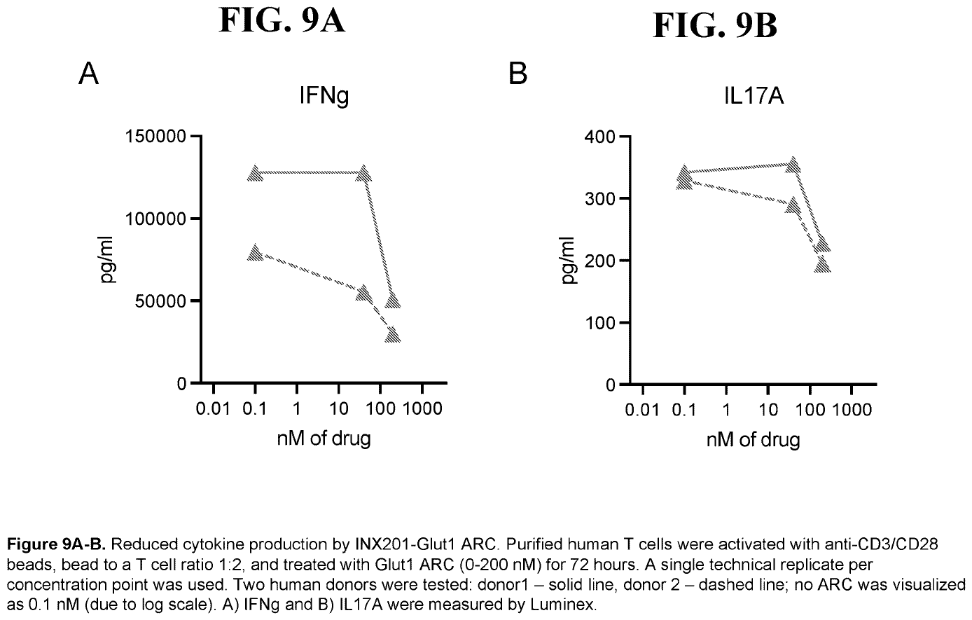

In the experiments in FIG. 9A-B a Glut1 ARC was tested on purified human T cells, stimulated with anti-CD3/CD28 dynabeads for 72 hours. Following incubation, media was collected, and cytokine levels were measured by Luminex for two human donors. IFNg and IL17A levels were reduced 2-3 fold in a dose-dependent manner when T cells were treated with Glut1 ARC (FIG. 9A-B).

More specifically, the experiments shown in FIG. 9A-B show reduced cytokine production by INX201-Glut1 ARC wherein purified human T cells were activated with anti-CD3/CD28 beads, bead to a T cell ratio 1:2, and treated with Glut1 ARC (0-200 nM) for 72 hours. A single technical replicate per concentration point was used. Two human donors were tested: donor 1—solid line, donor 2—dashed line; no ARC was visualized as 0.1 nM (due to log scale). A) IFNg and B) IL17A were measured by Luminex.

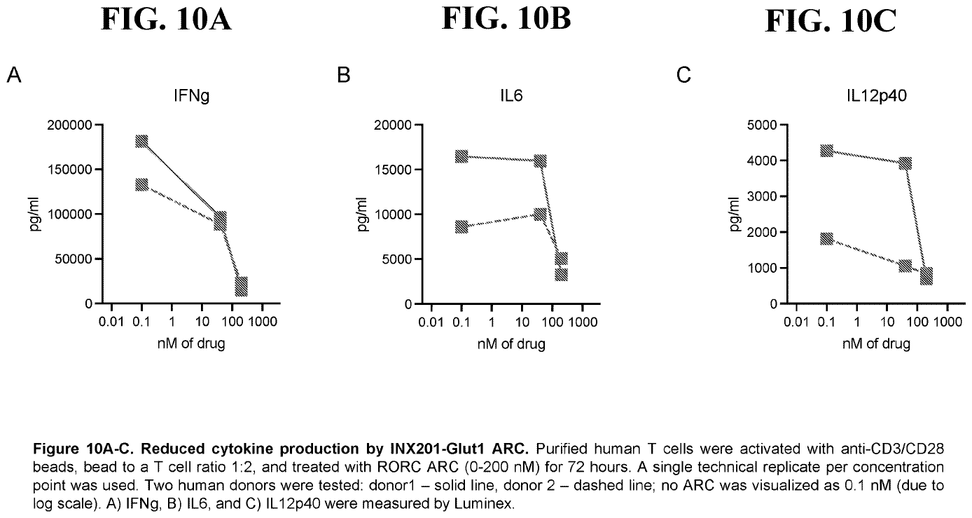

In the experiments shown in FIG. 10A-C, RORC ARC was also tested on purified human T cells, stimulated with anti-CD3/CD28 dynabeads for 72 hours. Following incubation, media was collected, and cytokine levels were measured by Luminex for two human donors. IFNg, IL6, and IL12p40 levels were reduced 2-4 fold in a dose-dependent manner when T cells were treated with RORC ARC (FIG. 10A-C).

More specifically, in the experiments shown in FIG. 10A-C reduced cytokine production by INX201-Glut1 ARC was demonstrated using purified human T cells activated with anti-CD3/CD28 beads, bead to a T cell ratio 1:2, and treated with RORC ARC (0-200 nM) for 72 hours. A single technical replicate per concentration point was used. Two human donors were tested: donor 1—solid line, donor 2—dashed line; no ARC was visualized as 0.1 nM (due to log scale). A) IFNg, B) IL6, and C) IL12p40 were measured by Luminex.

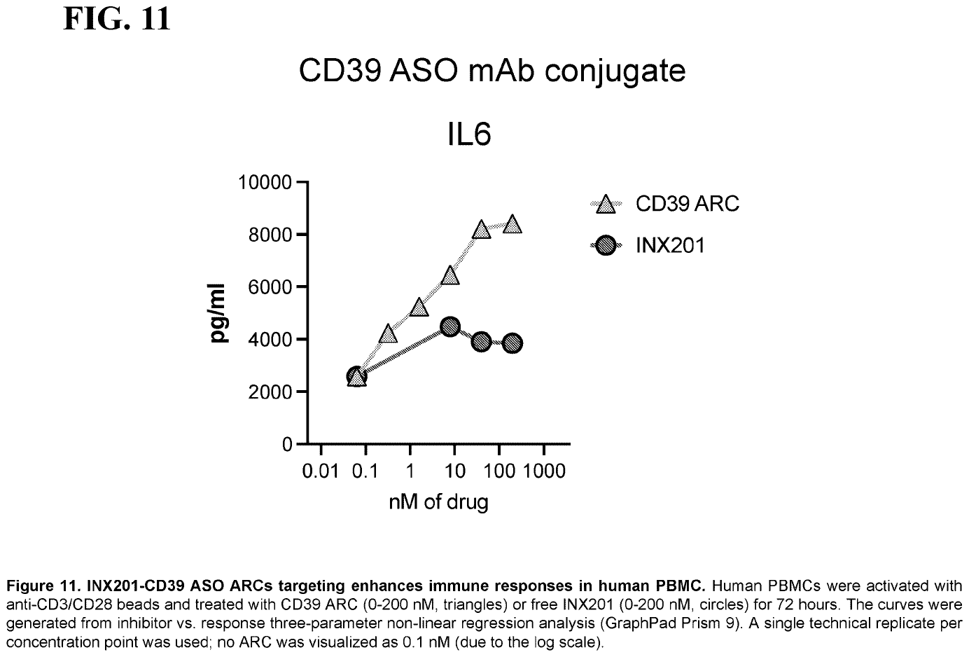

The experiments shown in FIG. 11 further revealed that in contrast to diminishing T cell responses, CD39 knockdown using ARC enhanced immune responses in stimulated PBMCs. Specifically, the experiment demonstrated that IL-6 levels increased in a dose-dependent manner after PBMCs were treated with CD39 ARC as compared to free antibody INX201 (FIG. 11).

More specifically, in the experiments shown in FIG. 11 showed that INX201-CD39 ASO ARCs targeting enhanced immune responses in human PBMCs which were activated with anti-CD3/CD28 beads and treated with CD39 ARC (0-200 nM, triangles) or free INX201 (0-200 nM, circles) for 72 hours. The curves were generated from inhibitor vs. response three-parameter non-linear regression analysis (GraphPad Prism 9). A single technical replicate per concentration point was used; no ARC was visualized as 0.1 nM (due to the log scale).

### Delivery Platform Optimization: Mab and Fab

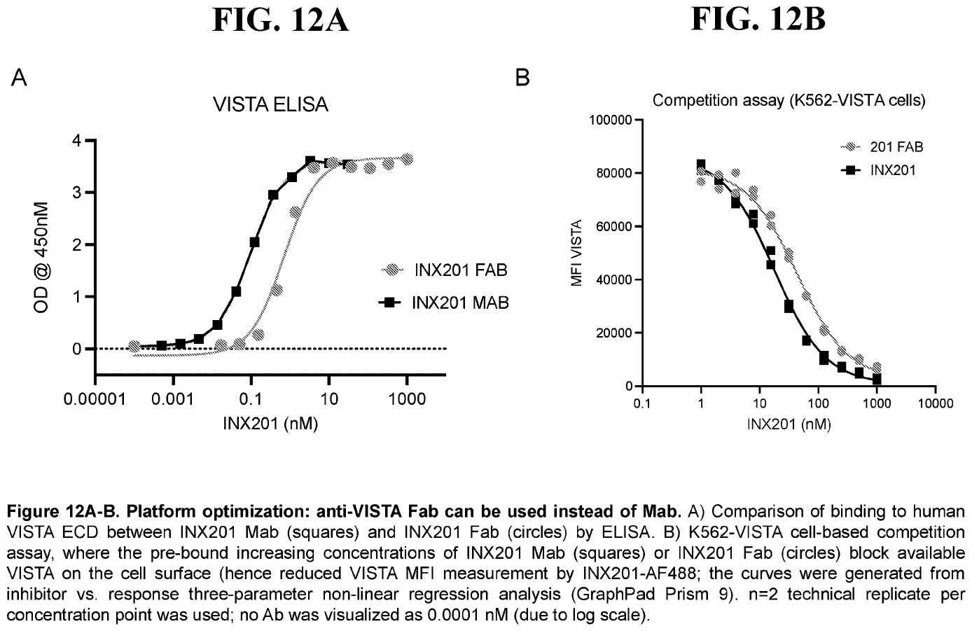

To optimize Applicant's Ab delivery platform, we have designed INX201 Fab, which will be used for conjugation to make a DAR2 antibody fragment with the respective payloads. To ensure INX201 Fab acts exactly as whole Mab, we have performed ELISA and competition cell-based assays directly comparing INX201 Mab and Fab (FIG. 12A-B). FIG. 12A shows that binding to the target (VISTA) is comparable between INX201 Mab and Fab (0.1 nM vs. 0.7 nM EC50, three-fold differences are expected due to stoichiometry). In the competition assay, which was performed on the K562-VISTA cell line, cells were pre-incubated with increasing concentrations of the unlabeled INX201 Mab or INX201 Fab to block the target on the surface of the cells, followed by detecting the available residual target on the surface using labeled INX201-AF488 antibody by flow cytometry. As is evident from the graph, both INX201 Mab and INX201 Fab were able to efficiently block VISTA targets on the cell surface in a dose-dependent manner (FIG. 12B), again confirming that INX201 Fab behaves functionally exactly as INX201 Mab.

### Vista ELISA Setup.

First, a 96-well flat-bottom plate (Thermo Scientific Nunc Immuno Maxisorp, cat #442404) was coated with hIX50 (human VISTA ECD, produced at Aragen Bioscience for ImmuNext) at 20 nM in PBS for one hour at room temperature (RT). The wells were washed three times with PT (PBS with 0.05% Tween 20) and then blocked with PTB (PBS with 0.05% Tween 20 and 1% BSA) for 30 minutes at RT. INX201 Fab was diluted from 1000 nM to 0.02 nM in PTB and then added to the wells for 1 hour at RT, or INX201 Mab was diluted from 20 nM to 0.002 nM in PTB and then added to the wells for 1 hour at RT. Following incubation, the wells were washed three times with PT, then mouse anti-human kappa coupled to HRP (SouthernBiotech, cat #9230-05) was used as a detection reagent at a dilution of 1/2000 and incubated for 1.5 hours at RT. Following three washes, the ELISA reaction was revealed using TMB (Thermo Scientific, cat #34028) as a colorimetric substrate. After a few minutes at RT, the reaction was stopped with 1M H2SO4. OD450 was read on Molecular devices Spectramax M3 plate Reader and analyzed with SoftMaxPro software.

More specifically, FIG. 12 shows platform optimization using an anti-VISTA Fab instead of Mab. The experiment in FIG. 12A compares binding to human VISTA ECD between INX201 Mab (squares) and INX201 Fab (circles) by ELISA. The experiment in FIG. 12B shows the results of a K562-VISTA cell-based competition assay, where the pre-bound increasing concentrations of INX201 Mab (squares) or INX201 Fab (circles) block available VISTA on the cell surface (hence reduced VISTA MFI measurement by INX201-AF488; the curves were generated from inhibitor vs. response three-parameter non-linear regression analysis (GraphPad Prism 9). n=2 technical replicate per concentration point was used; no Ab was visualized as 0.0001 nM (due to log scale).

### Testing the Impact of an Exemplary Inventive ARC on T Cell Viability

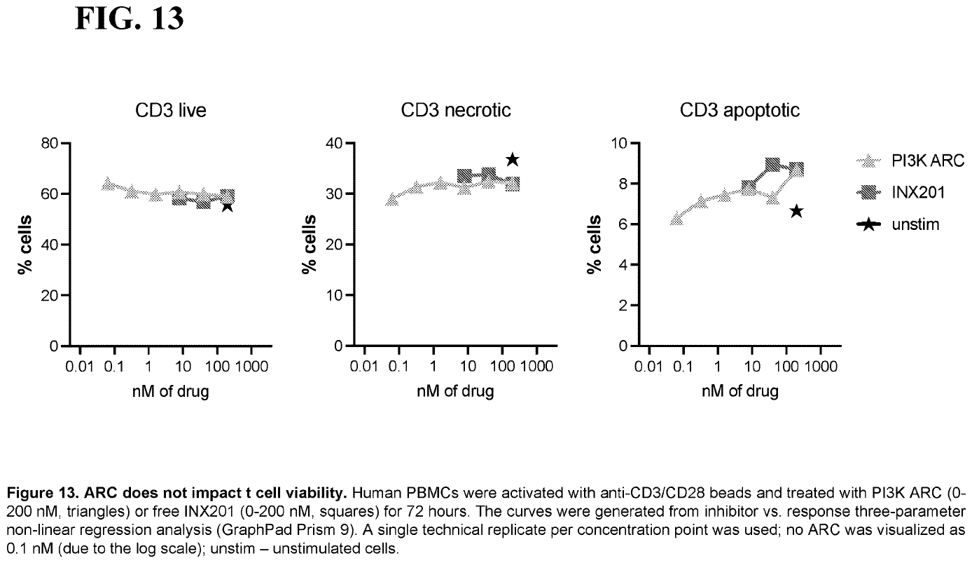

Human PBMCs were stimulated with anti-CD3/CD28 dynabeads as described in the materials and methods. T-cell viability was studied in a dedicated experiment where following 72-hour stimulation, cells were stained using an antibody panel, shown in Table 8. Live cells were defined as negative for Annexin V and live-dead; apoptotic cells were Annexin V and live-dead double-positive, and necrotic cells were Annexin V negative but live-dead positive. No difference in cell viability and percent of apoptotic or necrotic T cells was found between INX201 ARC and free INX201, as well as no Ab or ARC dose-dependent changes in viability were noted (FIG. 13).

More specifically, FIG. 13 shows that the exemplary ARC does not impact T cell viability in experiments wherein human PBMCs were activated with anti-CD3/CD28 beads and treated with PI3K ARC (0-200 nM, triangles) or free INX201 (0-200 nM, squares) for 72 hours. The curves were generated from inhibitor vs. response three-parameter non-linear regression analysis (GraphPad Prism 9). A single technical replicate per concentration point was used; no ARC was visualized as 0.1 nM (due to the log scale); unstim—unstimulated cells.

### Example 2: In Vivo Evaluation of Anti-VISTA Antibody RNA Conjugates (ARCs) in GVHD Model

In this example the efficacy of anti-VISTA antibody RNA conjugates (ARCs) was assessed in a GVHD model. These experiments again used INX201 as a payload delivery vehicle. These experiments provide further proof of concept that INX201 may be used as a delivery vehicle for delivery of siRNA payloads to immune cells. In these proof of concept experiments the siRNA payloads target PIK3CA and GLUT3 (IDT). The Ab sequences used in these experiments are in Appendix 3; and the payload sequences used for conjugation are contained in Appendix 4.

Table 11 below shows the anti-VISTA and isotype control MAbs, and ARCs used for delivery of payloads.

TABLE 11
Anti-VISTA and isotype control Mabs,
and ARCs for payload delivery.

| | | | | |
|---|---|---|---|---|
| 1 | INX201- PI3K ARC, lot SS1 | 2 | 167000 | 11976 |
| 1 | Isotype control IgG1-PI3K ARC, | 1 | 167000 | 5988 |
| | lot 102406.1.a | | | |

As has been afore-noted, the antisense strand of siRNA represents an active drug. Once delivered inside cells of interest via our VISTA delivery platform, it binds to and triggers degradation of its target mRNA, thereby reducing the level of target mRNA and protein.

### Graft Versus Host Disease (GvHD) Model

The GVHD model was used to assess the efficacy of anti-VISTA antibody RNA conjugates (ARCs) because it is a well accepted autoimmune and inflammatory model. Moreover, humanized mouse models of xenogeneic Graft versus Host Disease (GvHD) allow the study of immunomodulatory compounds specific to human drug targets in vivo and their effects on markers of inflammation such as inflammatory cytokines. These GVHD models are based on the transfer of human peripheral blood mononuclear cells (PBMCs) in immuno-deficient mouse strains.

The NSG (NOD-scid IL-2Rγnull) mice GVHD model combines the features of the NOD/ShiLtJ background, the severe combined immune deficiency mutation (scid), and IL2 receptor gamma chain deficiency. As a result, these mice lack mature T cells, B cells, or functional NK cells, and are deficient in cytokine signaling, leading to better engraftment of human hematopoietic stem cells and peripheral blood mononuclear cells than any other published mouse strain.

In the NSG model of xeno-GvHD, donor human T-cells expand robustly in recipient mice and induce anti-host cell reactivity leading to cutaneous tissue infiltration. Over time the mice lose weight and if left untreated will succumb to GvHD. One hallmark of the disease is the massive production of pro-inflammatory cytokines, which can be measured in plasma [3]. The timeframe of disease progression can range from 3 to 7 weeks. Both the GLUT3 and PI3K pathways have been described as important for human T cell activation and proliferation [4-8], therefore, xeno-GvHD is particularly an excellent model to study the impact of GLUT3 or PI3K knockdown by our technology.

The materials and methods used in these in vivo experiments are set forth below.

### Materials and Methods

### Materials and Reagents

1. LPS, Chondrex (cat #170031)
2. NOD.Cg-Prkdcscid Il2rgtm1Wjl/SzJ male 8 week-old (NSG mice), Jackson labs (cat #1557)
3. ACK lysing buffer, Gibco (cat #A10492-01)
4. FACS lysis buffer (BD Biosciences, cat #349202)
5. PBS, Corning (cat #21-040-CV)
6. MILLIPLEX® MAP Human Cytokine/Chemokine/Growth Factor Panel A 48 Plex Premixed Magnetic Bead Panel—Immunology Multiplex Assay, EMD Millipore (cat #3 HCYTA-60K-PX48)
7. eBioscience™ Foxp3/Transcription Factor Staining Buffer Set, ThermoFisher, 00-5523-00
8. RPMI 1640 Medium (Gibco, 11875-093)
9. 10% FBS (ATCC, 30-2020)
10. 100 U/ml penicillin/streptomycin (Gibco, 15140122)

### Isolation of Human T Cells

The same procedures disclosed in Example 1 were used to isolate human T cells.

Cell Labeling of PBMCs or T Cells with Cell Trace Violet

The same procedures disclosed in Example 1 were used to label PBMCs or T cells with Cell Trace Violet.

### Human PBMC and T Cell Activation and Proliferation Assays

The same procedures disclosed in Example 1 were used for the Human PBMC and T cell activation and proliferation assays.

### INX201 or ARCs Titration

The same procedures disclosed in Example 1 were used to titrate INX201 and the ARCS.

### Flow Cytometry-Based Cell Activation Analysis

The same procedures disclosed in Example 1 were used for the flow cytometry-based cell activation analysis. The In vitro flow cytometry panel used in the analysis is set forth in Table 12.

TABLE 12
In vitro flow cytometry panel.

| channel | Marker | Clone | Fluorescent Tag | Supplier | Catalog # | comp plan |
|---|---|---|---|---|---|---|
| V3 | CellTrace Violet (CTV) | — | — | Thermo Fisher Scientific | C34557 | cells |
| V8 | CD45 | HI30 | cFluor V547 | Cytek Biosciences | R7-20011 | cells |
| R8 | MHC Class II (HLA-DR) | L243 | cFluor R840 | Cytek Biosciences | R7-20293 | cells |
| vioR7 | Viability | | ViaDye Red | Cytek Biosciences | R7-60008 | cells |
| R3 | CD3 | SK7 | cFluor R685 | Cytek Biosciences | R7-20345 | cells |
| B8 | CD8 | SK1 | PerCP | Cytek Biosciences | 67-0087-T500 | cells |
| B3 | CD4 | SK3 | cFluor B548 | Cytek Biosciences | R7-20043 | cells |
| B2 | CD45RA | HI100 | FITC | Cytek Biosciences | 35-0458-T100 | cells |
| R1 | CD25 | BC96 | APO | Cytek Biosciences | 20-0259-T100 | beads |
| R7 | CD27 | O323 | APC-Cy7 | Cytek Biosciences | 25-0279-T100 | beads |
| V15 | CD127 | eBioRDR5 | Super Bright 780 | Thermo Fisher Scientific | 78-1278-42 | beads |
| V11 | CD69 | FN50 | BV650 | Thermo Fisher Scientific | 416-0699-42 | beads |
| B6 | CD70 | 113-16 | PE-Dazzle 594 | Biolegend | 355124 | beads |
| B13 | CD71 | OKT9 | PE-Cy7 | Biolegend | 334112 | beads |
| B10 | CD19 | HIB19 | cFluor BYG710 | Cytek Biosciences | R7-20009 | cells |
| V13 | CD11b | ICRF44 | Super Bright 702 | Thermo Fisher Scientific | 67-0118-42 | cells |
| R4 | CD56 | 5.1H11 | cFluor R720 | Cytek Biosciences | R7-20089 | cells |
| B4 | CD80 | 2D10.4 | PE | Cytek Biosciences | 50-0809-T100 | beads |
| V5 | CD86 | IT2.2 | BV480 | Thermo Fisher Scientific | 414-0869-42 | beads |
| V3* | CD14 | MEM-18 | cFluor V450 | Cytek Biosciences | R7-20395 | cells |
| V1* | CD197 (CCR7) | | BV421 | Biolegend | 353208 | cells |
| V10* | CD16 | eBioCB16 | Super Bright 600 | Thermo Fisher Scientific | 63-0168-42 | cells |

*last 3 Abs were not used in the experiment described.

### Xeno-GvHD Model

### Study Design

The recipient mice were 8-week-old male NSG purchased from Jackson Laboratory. Unless specified, 10 mice per group were enrolled.

On day 0 mice were injected intravenously (i.v.) with 10×10⁶ human PBMCs pre-mixed with the antibodies in 200 μl of PBS via tail vein injection. On day 14 (or other time points specified), mice were bled, plasma was collected for cytokine analysis and T cell numbers were evaluated using flow cytometry. Mice were weighed thrice a week to monitor disease progression and euthanized if their weight dropped below 75% of their initial weight.

### Human PBMC Isolation

Human peripheral blood was obtained from apheresis cones provided by volunteer donors at the DHMC blood donor program. The cone blood was diluted at 1:4 in PBS and carefully deposited on top of 13 ml of Histopaque 1077. After 20 min centrifugation at 850 g (room temperature, no brake on deceleration), the mononuclear cells from the Histopaque/PBS interfaced were collected. After 1 wash in PBS, the PBMC were resuspended in PBS at 100×10⁶ cells/ml for injection.

### Antibody Dosing

INX201 alone or INX201 antibody RNA conjugate (ARC) was injected at 5 mg/kg (mixed with hPBMC). A single dose of ARC (or Ab) was used in all of the experiments described in this example.

### LPS Early Stimulation Models

In some experiments, we evaluated early human PBMC cytokine responses in vivo following stimulation by LPS (0.5 mg/Kg, injected i.p.) 18 hours post PBMC+ARC injection. In these early stimulation models, mice were injected with 10 million PBMCs per mouse. Plasma was collected for cytokine measurements by Luminex MILLIPLEX at early time points (4 hours to 7 days post stimulation).

### Evaluation of “Absolute” Immune Cell Change in Blood

At day 14 (or other specified time points) after PBMC cell transfer, human T cell absolute numbers were evaluated by flow cytometry on retro-orbital bleed. To obtain a close to absolute cell count, 100 μl of whole blood was stained by directly adding the antibody cocktail (Table 3)+mouse and human Fc block. After 30 min, of RT incubation, 1 ml of BD FACS lysing solution was added to each sample. After 20 min incubation, samples were washed once in a large volume of PBS, then resuspended in 100 μl of PBS and ran on run on a MACSQuant or Northern Lights (Cytek) flow cytometer and analyzed with the FlowJo program.

The antibody cocktail to effect the cell count in these experiments are set forth in Table 13 below.

TABLE 13
Antibody cocktail for absolute cell count

| Antibodies | Cat # | Dilution |
|---|---|---|
| hCD25-FITC | Biolegend, 302604 | 20 |
| hTIGIT-PE | Biolegend, 372704 | 10 |
| mCD45-PerCP.Cy5.5 | Biolegend, 103132 | 20 |
| hCD45RO-PE-cy7 | Biolegend, 304230 | 20 |
| hCD4-APC-Cy7 | Biolegend, 300518 | 20 |
| hCD3-BV421 | Biolegend, 300434 | 20 |
| hCD45-BV510 | Biolegend, 304036 | 20 |
| Mouse Fc Block | Miltenyi Biotec, 130-092-575; | 20 |
| Human Fc Block | Fisher Scientific (eBioscience), | 20 |
| | 50-112-9053 | |

Evaluation of Treg cells in the blood (day 28 or as specified)

The procedures used in these experiments to evaluate Treg cells in the blood (day 28 or as specified) are as follows:
1. Aliquot 0.1 ml of blood into a deep well 1.3 ml plate.
2. Add 1 ml ACK, and incubate for 10 min.
3. Spin at 450 g for 3 min, decant supernatant and repeat lysis for 5 min if the pellet is still red.
4. Wash with PBS (1.0 ml), spin at 450 g for 3 min, decant supernatant, transfer to V-bottom plate in 200 μl PBS 5. Spin at 450 g for 3 min, add 50 ml of Ab mix (Table 4, except anti-FOXP3 and RORgt Abs), incubate at RT for 30 min, and wash once with PBS. Spin at 450 g for 3 min, decant supernatant.
6. The FoxP3 Fixation/Permeabilization was used according to manufacturer instructions for FOXP3 and RORgt staining.
7. After fixation and permeabilization, cells were resuspended in 50 μl of permeabilization buffer with anti-FOXP3 and RORgt Abs, both 1:50 dilution, for 30 min RT. Cells were washed once with permeabilization buffer and resuspended in 100 μl of PBS to run on a MACSQuant or Nothern Lights (Cytek) flow cytometer and analyzed with the FlowJo program.

The Regulatory T cell antibody panel used in these experiments are in as

TABLE 14
Regulatory T cell antibody panel

| Antibodies | Cat # | Dilution |
|---|---|---|
| hCD25-FITC | Biolegend, 302604 | 200 |
| hTIGIT-PE | Biolegend, 372704 | 100 |
| mCD45-PerCP.Cy5.5 | Biolegend, 103132 | 200 |
| hCD45RO-PE-cy7 | Biolegend, 304230 | 200 |
| ROR gamma (t) - APC | ThermoFisher, 17-6988-82 | 50 |
| hCD4-APC-cy7 | Biolegend, 300518 | 200 |
| hFOXP3-BV421 | Biolegend, 320124 | 50 |
| hCD45-BV510 | Biolegend, 304036 | 200 |
| Mouse Fc Block | Miltenyi Biotec, 130-092-575; | 200 |
| Human Fc Block | Fisher Scientific (eBioscience), | 200 |
| | 50-112-9053 | |

### Results

### PI3K and GLUT3 ARCs Conjugation and In Vitro QC

Anti-VISTA Mab, payloads used for conjugations and ARCs are described in Table 11 and Appendixes 1-2. Oligo Conjugation Kit (Abcam, #ab218260) was used for all conjugations. All RNA oligos were purified by HPLC, resuspended at 100 μM and annealed. Lysine-based conjugation via the amine-reactive group, one of the most widely utilized non-specific conjugation strategies, was used. All payloads were synthesized at IDT and contained a 3′-sense strand Cy5 label and 5′-amine on the antisense strand (see appendix 2). Drug-to-antibody ratio (DAR) was assessed by UV/Vis spectroscopy (Nanodrop) and was estimated to be 1.0 for both ARCs described.

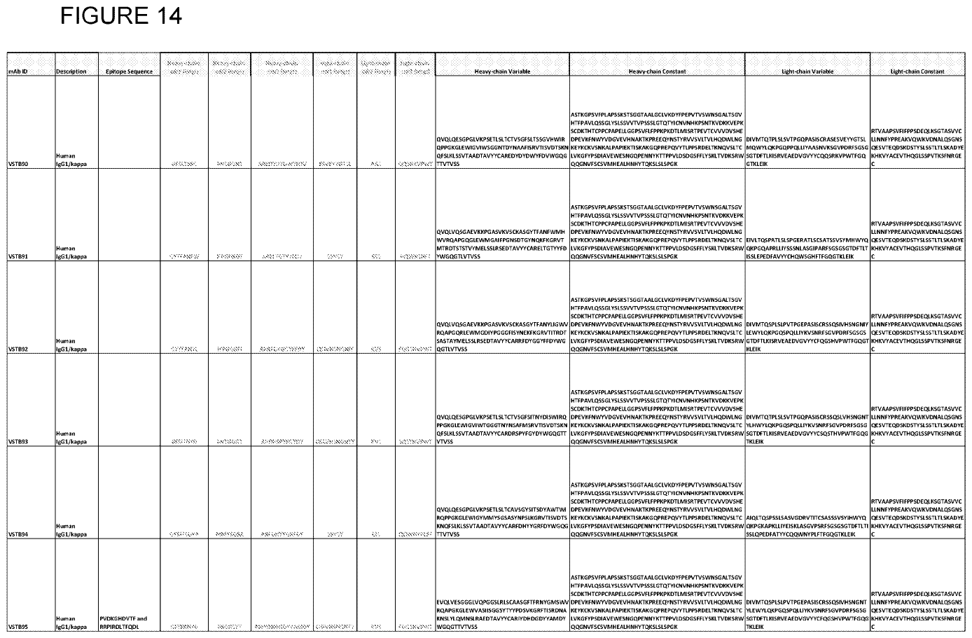

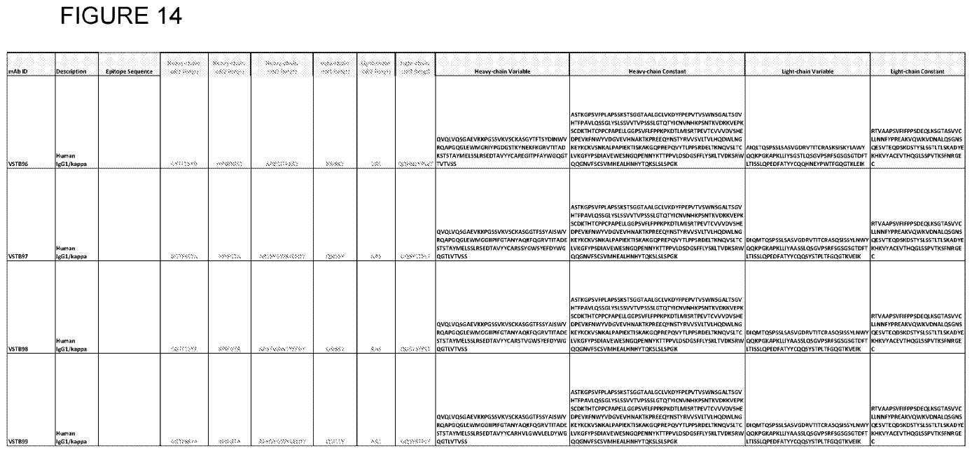

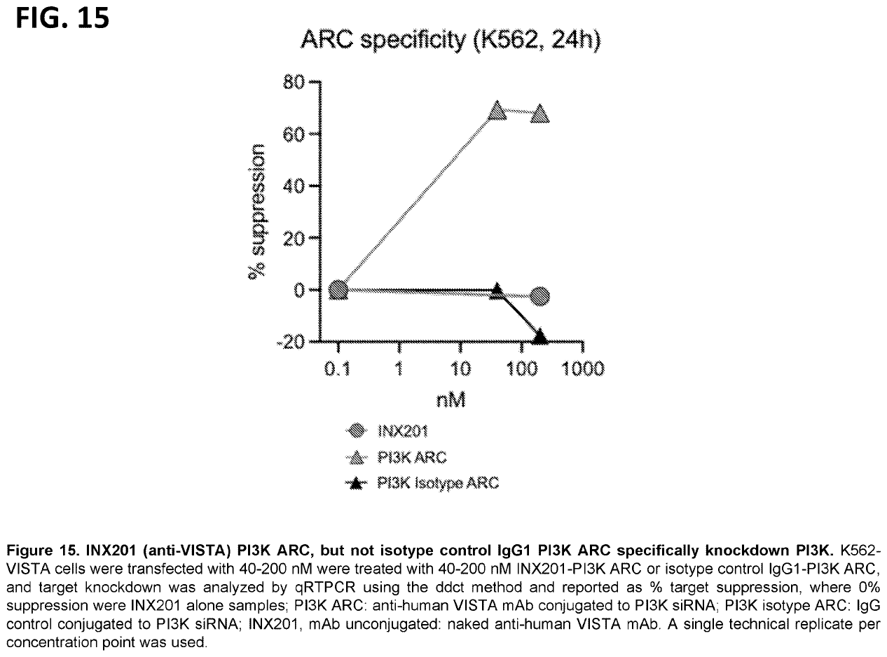

K562-VISTA cells (described in ARC_01) were treated with 40-200 nM INX201-PI3K ARC or isotype control IgG1-PI3K ARC (table 1). PI3K knockdown was assessed at 24 hours by qRTPCR. INX201-PI3K ARC achieved 70% target knockdown, while INX201 Ab alone or isotype control PI3K ARC showed no PI3K knockdown (FIG. 15) indicating specificity.

As is shown by the data in FIG. 15, INX201 (anti-VISTA) PI3K ARC, but not isotype control IgG1 PI3K ARC specifically knockdown PI3K. In these experiments K562-VISTA cells were transfected with 40-200 nM were treated with 40-200 nM INX201-PI3K ARC or isotype control IgG1-PI3K ARC, and target knockdown was analyzed by qRTPCR using the ddct method and reported as % target suppression, where 0% suppression were INX201 alone samples; PI3K ARC: anti-human VISTA mAb conjugated to PI3K siRNA; PI3K isotype ARC: IgG control conjugated to PI3K siRNA; INX201, mAb unconjugated: naked anti-human VISTA mAb. A single technical replicate per concentration point was used.

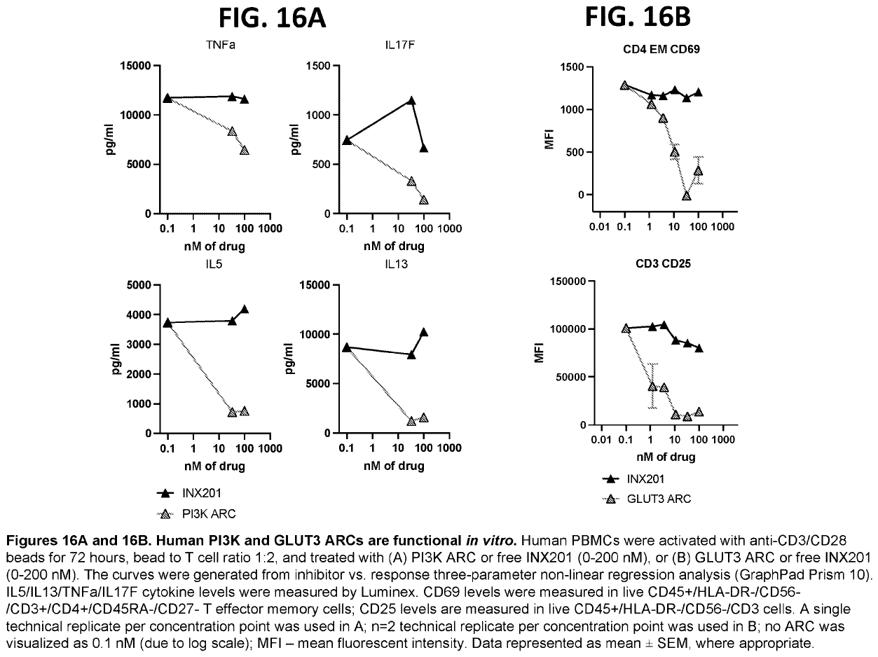

ARCs were tested in vitro using human PBMCs activated with anti-CD3/CD28 dynabeads for 72 hours as described above. Following incubation, media was collected, and cytokine levels were measured by Luminex. PI3K ARC, but not free INX201 antibody reduced pro-inflammatory cytokine production. Specifically, PI3K ARC reduced IL5 and IL13 levels 3-6 fold; TNFa and IL17F levels ~2 fold, all as compared to free INX201 levels (FIG. 16A).

As can be seen from FIG. 16A, GLUT3 ARC, but not free INX201 Ab reduced T cell activation in a dose dependent manner. Moreover, CD69 levels on effector memory T cells (TEM) and CD25 levels on CD3 T cells were reduced with GLUT3 ARC in a dose dependent manner with IC50 of 1 and 6 nM respectively (FIG. 16B). Note, with DAR of 1.0, the amount of “drug” (siRNA) is equivalent to amount (nM) of Ab delivery vehicle.

More particularly, in the experiments of FIG. 16A-B, human PI3K and GLUT3 ARCs are demonstrated to be functional in vitro. In the experiments human PBMCs were activated with anti-CD3/CD28 beads for 72 hours, bead to T cell ratio 1:2, and treated with (A) PI3K ARC or free INX201 (0-200 nM), or (B) GLUT3 ARC or free INX201 (0-200 nM). The curves were generated from inhibitor vs. response three-parameter non-linear regression analysis (GraphPad Prism 10). IL5/IL13/TNFa/IL17F cytokine levels were measured by Luminex. CD69 levels were measured in live CD45+/HLA-DR−/CD56−/CD3+/CD4+/CD45RA−/CD27-T effector memory cells; CD25 levels are measured in live CD45+/HLA-DR−/CD56−/CD3 cells. A single technical replicate per concentration point was used in A; n=2 technical replicate per concentration point was used in B; no ARC was visualized as 0.1 nM (due to log scale); MFI—mean fluorescent intensity. Data represented as mean t SEM, where appropriate.

PI3K ARC treatment decreases human cytokine responses and T cell proliferation in vivo

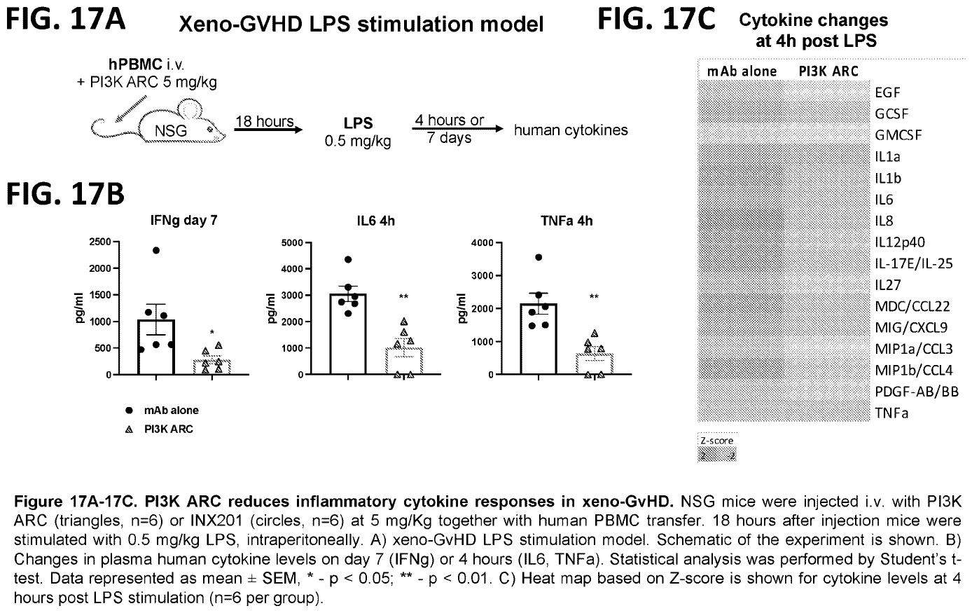

In the one experiment where survival was monitored, we observed no differences between PI3K ARC and INX201 alone in weight loss and median survival (data not shown) in both classical xeno-GvHD and with early LPS stimulation. Accordingly, as an alternative to the classical xeno-GvHD model, in which human T cells proliferate and human cytokine levels increase over time, we thought to induce early cytokine production by human PBMCs shortly (18 hours) after injection into the host (NSG mice) using stimulation such as LPS. 18 hours post hPBMC+Ab i.v. injections, the mice were injected i.p. LPS, and blood cytokine responses were measured at 4 hours and 7 days post LPS injection (FIG. 17A). As can be seen the peak levels for most cytokines were at 4 hours. PI3K ARC treatment diminished the production of IL6 and TNFa up to 3-fold, as compared to INX201 alone at 4 hours post LPS injection. PI3K ARC treatment also reduced delayed IFNg responses on day 7 (FIG. 17B). PI3K ARC overall had a global impact on cytokine production, generally reducing most of them at early time point (4h) post LPS stimulation as shown in FIG. 17C. Reductions in pro-inflammatory cytokines (FIG. 3C) did not reach statistically significant levels, due to variability in responses. PI3K ARC did not show any impact on human cytokine production in groups without LPS stimulation (data not shown).

The experiments in FIG. 17, clearly show that PI3K ARC reduces inflammatory cytokine responses in xeno-GvHD mice. In the experiments in the figure NSG mice were injected i.v. with PI3K ARC (triangles, n=6) or INX201 (circles, n=6) at 5 mg/Kg together with human PBMC transfer. 18 hours after injection mice were stimulated with 0.5 mg/kg LPS, intraperitoneally. FIG. 17A shows a schematic of the experiment in the xeno-GvHD LPS stimulation model. FIG. 17B shows changes in plasma human cytokine levels on day 7 (IFNg) or 4 hours (IL6, TNFa). Statistical analysis was performed by Student's t-test. Data represented as mean±SEM, *—p<0.05; **—p<0.01. FIG. 17C contains a heat map based on Z-score is shown for cytokine levels at 4 hours post LPS stimulation (n=6 per group).

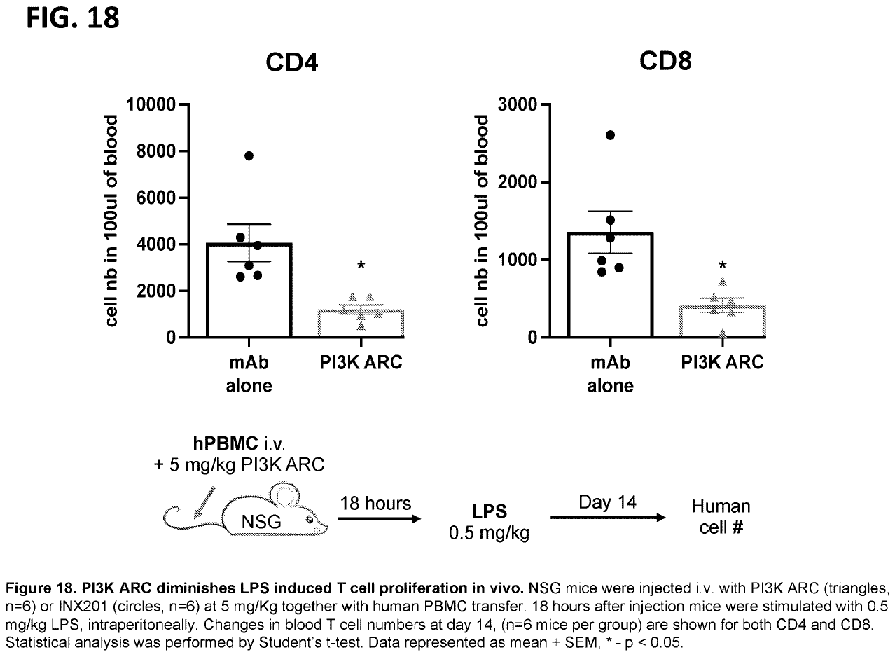

The results in the GVHD model further revealed that PI3K ARC treatment reduced T cell proliferation in response to LPS stimulation in xeno-GvHD, as evidenced by diminished (over 2-fold) CD4 and CD8 T cell numbers in PI3K ARC-treated groups on day 14 (FIG. 18). No differences in T cell numbers were found in classical xeno-GvHD without LPS stimulation (data not shown).

More specifically, the data in FIG. 18 showed that PI3K ARC diminishes LPS induced T cell proliferation in vivo. In the experiments in the figure NSG mice were injected i.v. with PI3K ARC (triangles, n=6) or INX201 (circles, n=6) at 5 mg/Kg together with human PBMC transfer. 18 hours after injection mice were stimulated with 0.5 mg/kg LPS, intraperitoneally. Changes in blood T cell numbers at day 14, (n=6 mice per group) are shown for both CD4 and CD8. Statistical analysis was performed by Student's t-test. Data represented as mean±SEM, *—p<0.05.

PI3K ARC does not Reduce Percent of Regulatory T Cells In Vivo

To test whether PI3K ARC has any impact on regulatory T cell (Treg) number in xeno-GvHD model we monitored Treg cell levels in the blood of NSG mice in the same xeno-GvHD model. Again, NSG mice were injected i.v. with 10 million hPBMC (mixed with human INX201 alone or PI3K ARC, at 5 mg/kg). In separate arms of the experiment some mice were stimulated with LPS 18 hours post PBMC+Ab or ARC injections. LPS was administered i.p.

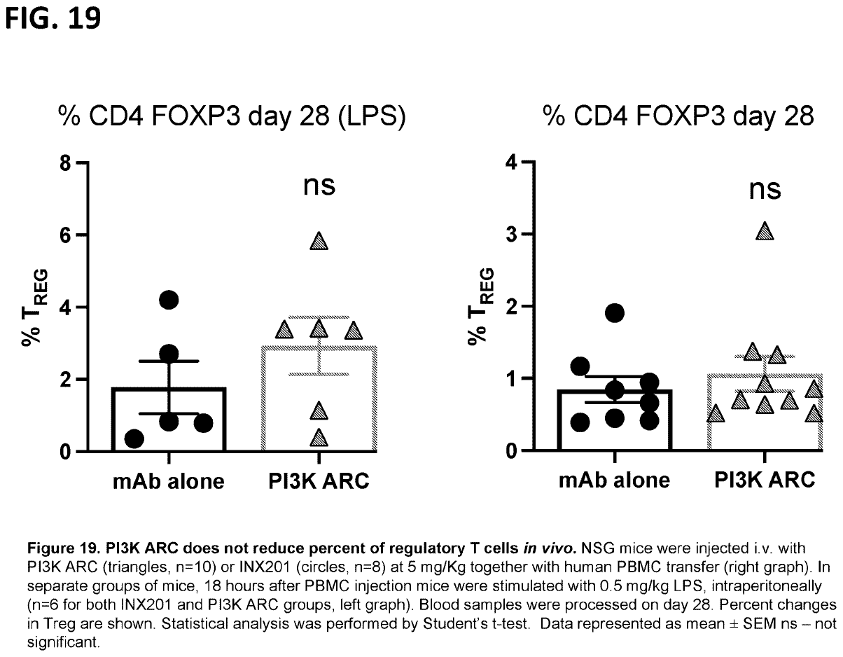

On day 28, human CD4⁺FOXP3⁺ (Treg) cell frequencies were measured in blood. We observed that PI3K ARC treatment did not reduce the frequency of Tregs, as compared to INX201 (FIG. 19). In the KLPS-stimulated groups there was a trend for increased frequency of Tregs, however the differences were not statistically significant. No substantial amounts of Th17 cells were detected in the blood (data not shown). Taken together, our data indicate that PI3K ARC does not reduce percent of regulatory T cells in vivo.

More specifically the experiments in FIG. 19 revealed that PI3K ARC does not reduce the percentage of regulatory T cells in vivo. In the experiments NSG mice were injected i.v. with PI3K ARC (triangles, n=10) or INX201 (circles, n=8) at 5 mg/Kg together with human PBMC transfer (right graph). In separate groups of mice, 18 hours after PBMC injection mice were stimulated with 0.5 mg/kg LPS, intraperitoneally (n=6 for both INX201 and PI3K ARC groups, left graph). Blood samples were processed on day 28. Percent changes in Tregs are shown. Statistical analysis was performed by Student's t-test. The data represented as mean±SEM ns—are not statistically significant.

### GLUT3 ARC Treatment Decreases Human Cytokine Responses In Vivo

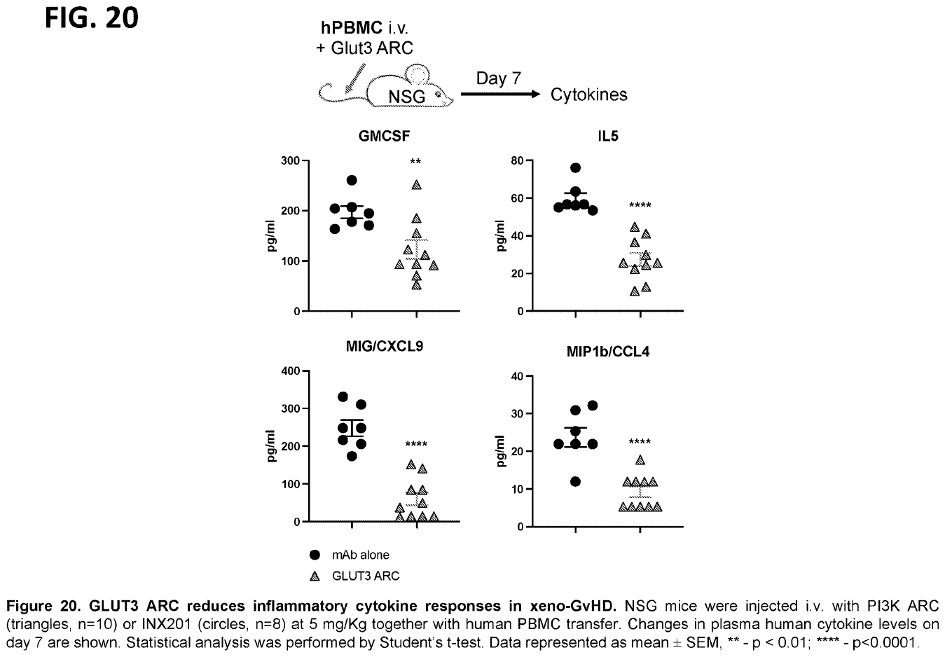

One classical hallmark of GVHD or xeno-GvHD disease progression is the massive production of pro-inflammatory cytokines, which can be measured in plasma. Based thereon we evaluated if GLUT3 ARC might impact blood cytokine responses on day 7. We found that GLUT3 ARC reduced the levels of GMCSF, CCL4 and IL5 about 2-fold and levels of CXCL9 3-fold, as compared to INX201 Ab alone (FIG. 20).

More specifically the experiments in FIG. 20 clearly show that GLUT3 ARC reduces inflammatory cytokine responses in xeno-GvHD. In these experiments NSG mice were injected i.v. with PI3K ARC (triangles, n=10) or INX201 (circles, n=8) at 5 mg/Kg together with human PBMC transfer. Changes in plasma human cytokine levels on day 7 are shown. Statistical analysis was performed by Student's t-test. Data represented as mean±SEM, **—p<0.01; ****—p<0.0001.

### CONCLUSIONS

Our experiments effected using the xeno-GvHD model further corroborate the ability of anti-VISTA antibody RNA conjugates (ARCs) according to the invention when used in vitro and in vivo to functionally deliver siRNAs into immune cells and to knock down expression of targeted mRNAs therein. In particular, the experimental data showed that:
PI3K and GLUT3 ARCs—INX201 antibody RNA conjugates, delivering GLUT3 or PIK3CA siRNA to VISTA+ cells are functional in vitro
   Reduce T cell cytokine responses
   Reduce T Cell CD69/CD25 activation profile
GLUT3 and PI3K ARCs are functional in vivo
   GLUT3 treatment decreases human cytokine responses in vivo (xeno-GvHD)
   PI3K ARC treatment decreases human cytokine responses in vivo
   PI3K ARC treatment decreases T cell proliferation in vivo
   PI3K ARC does not reduce percent of regulatory T cells in vivo

### Therapeutic Applications of Inventive ARCs or ANCs

To the best of the Applicant's knowledge ARCs or ANCs which specifically target immune cells and which deliver into immune cells one or more nucleic acids which modulate the expression and/or activity of specific immunomodulators and/or immunomodulatory pathways expressed by specific immune cells have not been described to date. Therefore, this invention provides a novel platform for treating diseases involving specific immune cell types without causing toxicity to non-targeted immune or other non-immune cell types.

Potential indications where the subject ARCs may be used as therapeutics or prophylactics include e.g., autoimmune diseases and inflammatory diseases, e.g., those with a prominent myeloid and/or T cell component (based on where VISTA is expressed and, therefore it is where the drug will be specifically delivered). Potential indications include diseases where limiting activation and growth of autoreactive effector T cells might be beneficial to control/decrease disease. Our targeting technology can help remove on-target toxicities associated with target expression in non-immune tissues; it can help drug previous undruggable targets due to either too broad an expression profile of the target or localization of the target (transcription factors). Specifically, rheumatoid arthritis, colitis, or systemic lupus erythematosus include autoimmune indications appropriate for ARC therapeutics.

Numerous cancer indications also potentially may be treated with the inventive ARCs, because, similar to the CD39 knockdown approach shown herein, an siRNA or ASO according to the invention can be administered in order to act on a target such as an Immune-checkpoint inhibitor protein (e.g., PD-1, PD-L1, CTLA-4, B7-1, B7-2, LAG-3, HHLA2, TNFRSF12A, HLA-G, NECTIN2, TNFRSF25, TNFSF14, LAIR1, TNFSF15, TNFSF4, KIR2DL4, PDCD1, LGALS9, and PVR) which blocks or suppresses anti-tumor responses from being elicited in the host. Removing or inhibiting this blockade will enable the host to activate the immune system and innate antitumor immunity more efficiently.

### Conclusions

In the subject patent application, we have shown that:
1) we can conjugate siRNA or ASO to an anti-VISTA Mab and efficiently deliver RNAs (e.g., siRNAs and ASOs) into the target cells via VISTA binding and internalization;
2) ARC has similar binding and internalization properties as compared to free INX201 Mab;
3) exemplary siRNA payloads, specific to immunologically relevant targets, retain their function when conjugated to an anti-VISTA Mab and delivered into the target cells;
4) siRNA conjugated to an anti-VISTA Mab INX201 facilitated siRNA accumulation and function inside K562-VISTA cells;
5) siRNA conjugated to an anti-VISTA Mab INX201 had function in human PBMCs and T cells, and effectively reduced proliferation and cytokine production (siRNA for five targets are described in the subject example);
6) CD39 ASO conjugated to an anti-VISTA Mab INX201 functioned in human PBMCs, i.e., it provided for increased cytokine production, showing that the inventive ARCs are promising for cancer treatment;
7) exemplary PI3K and GLUT3 ARCs—INX201 antibody RNA conjugates, delivering GLUT3 or PIK3CA siRNA to VISTA+ cells are functional in vitro, i.e., reduce T cell cytokine responses, reduce T Cell CD69/CD25 activation profile; and
8) exemplary GLUT3 and PI3K ARCs are functional in vivo, e.g., GLUT3 treatment decreases human cytokine responses in vivo (xeno-GvHD), PI3K ARC treatment decreases human cytokine responses in vivo, PI3K ARC treatment decreases T cell proliferation in vivo, and PI3K ARC does not reduce percent of regulatory T cells in vivo.

### Additional Proof of Concept Studies

While Applicant has demonstrated the suitability of ARCs or ANCs comprising an exemplary anti-VISTA antibody and siRNA payloads which target GLUT3 or PIK3CA as therapeutics in vitro and in vivo in the GVHD model, the inventive ARCs or ANCs have much broader applicability. This can be corroborated in additional experiments such as the following:
Optimize payload sequences, test additional targets
Perform ARC conjugation to Cys via maleimide or bromoacetimide reactive groups
Generate functional data using other in vivo models

### Addendum to Application

### Sequences not Entered in ST.26 Xml File Due to Sequence Length

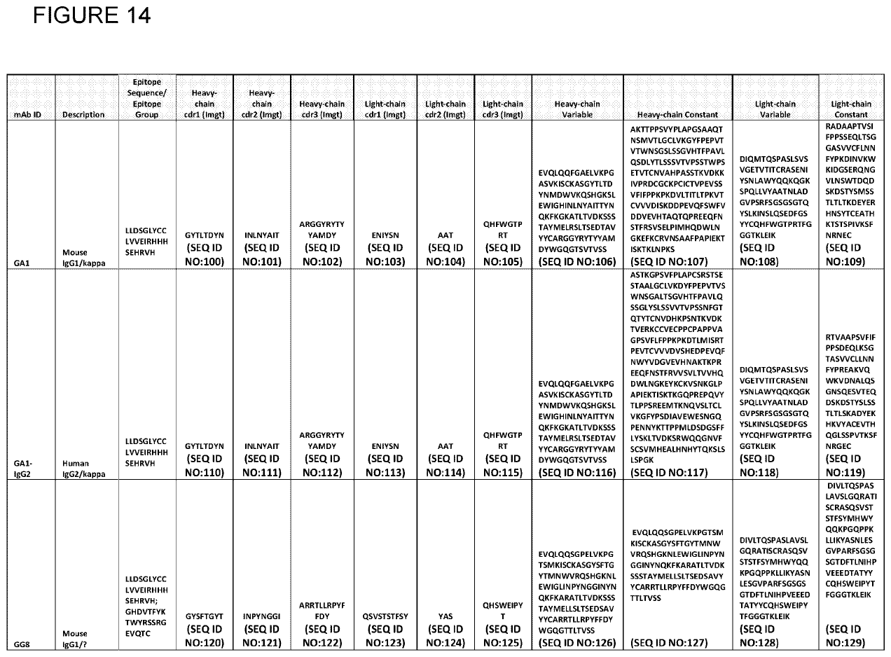

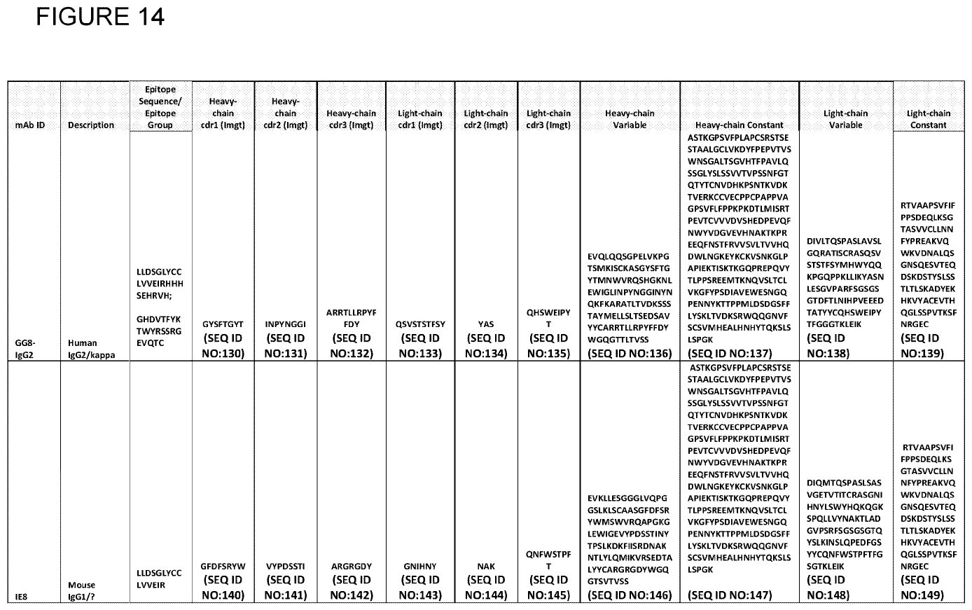

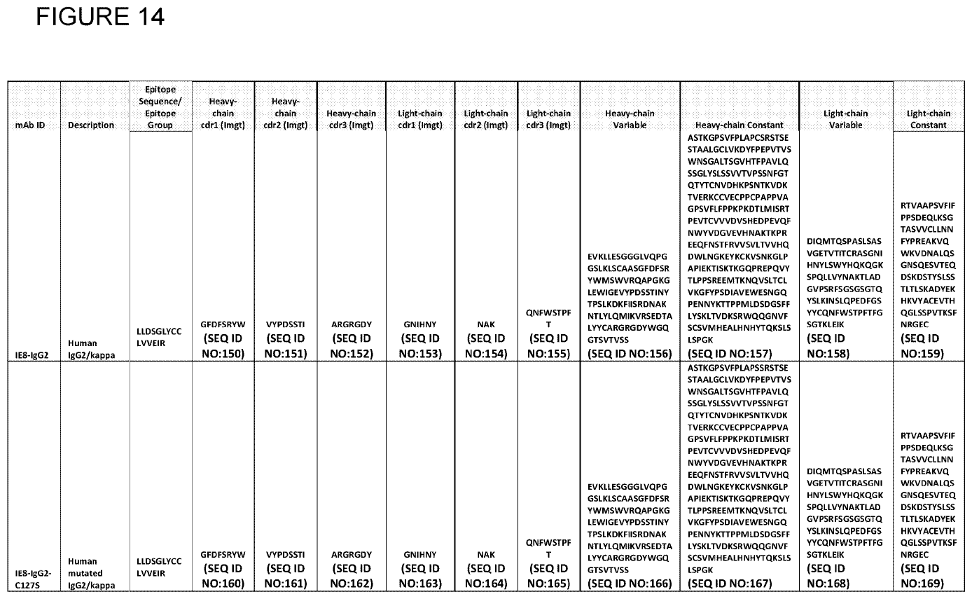

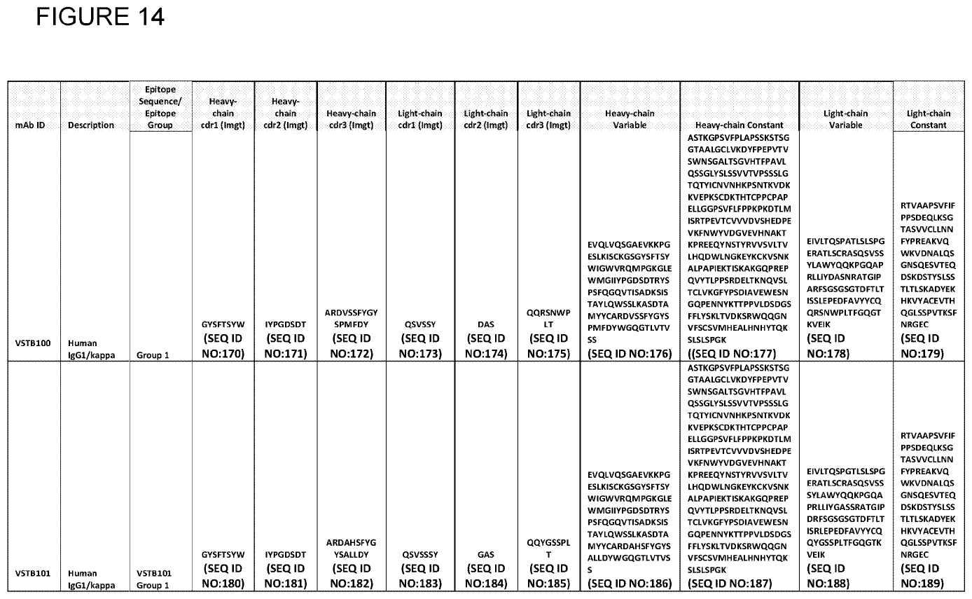

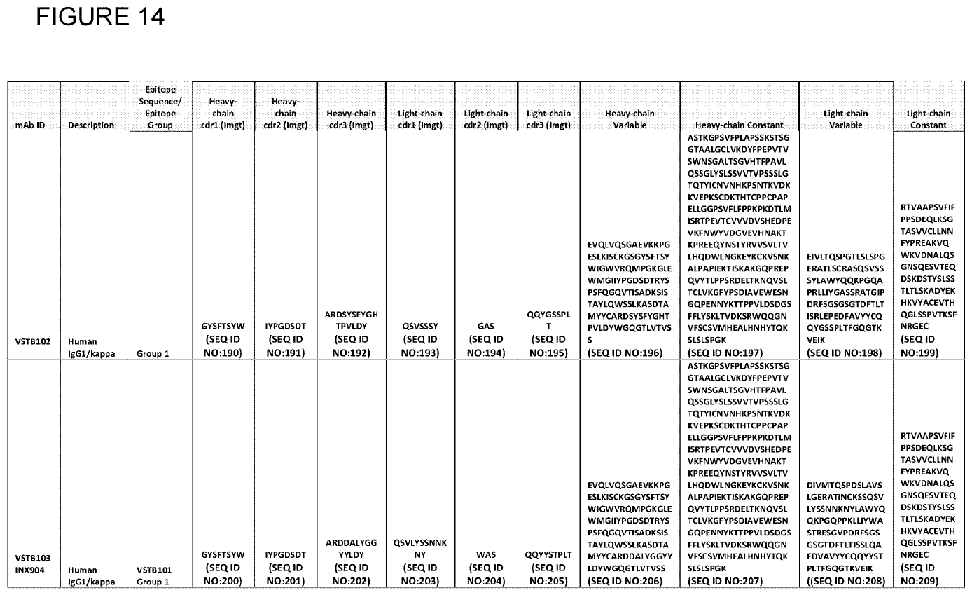

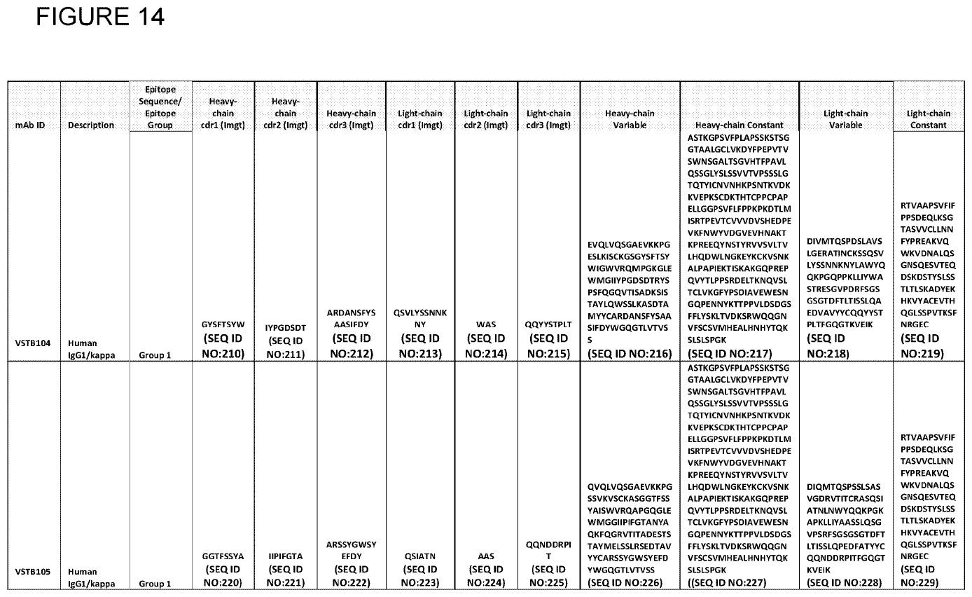

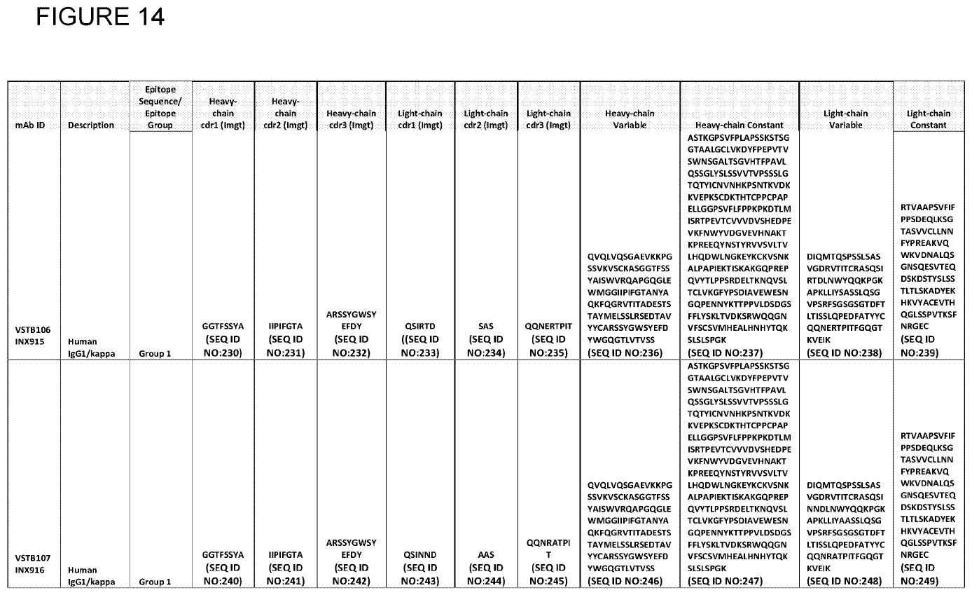

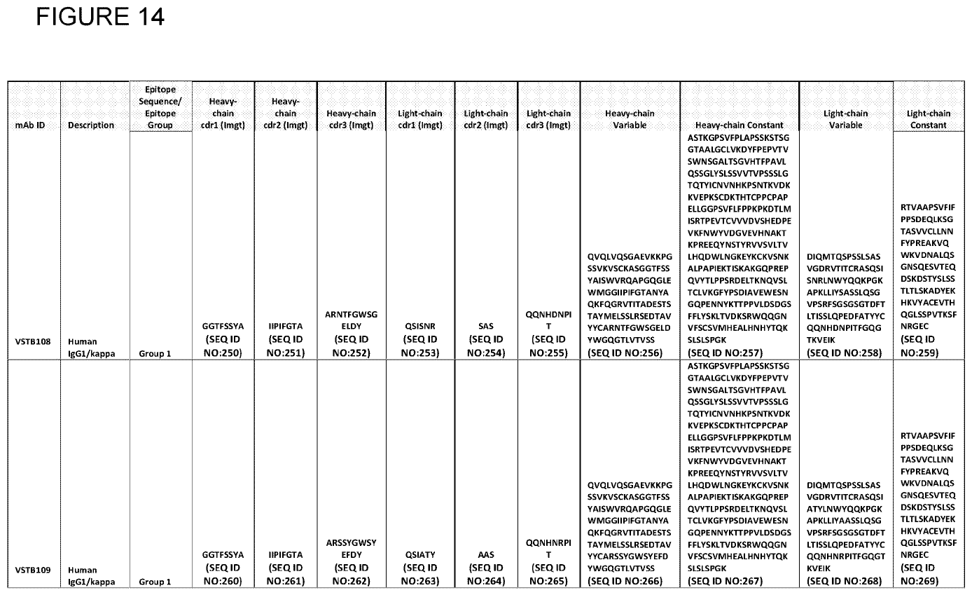

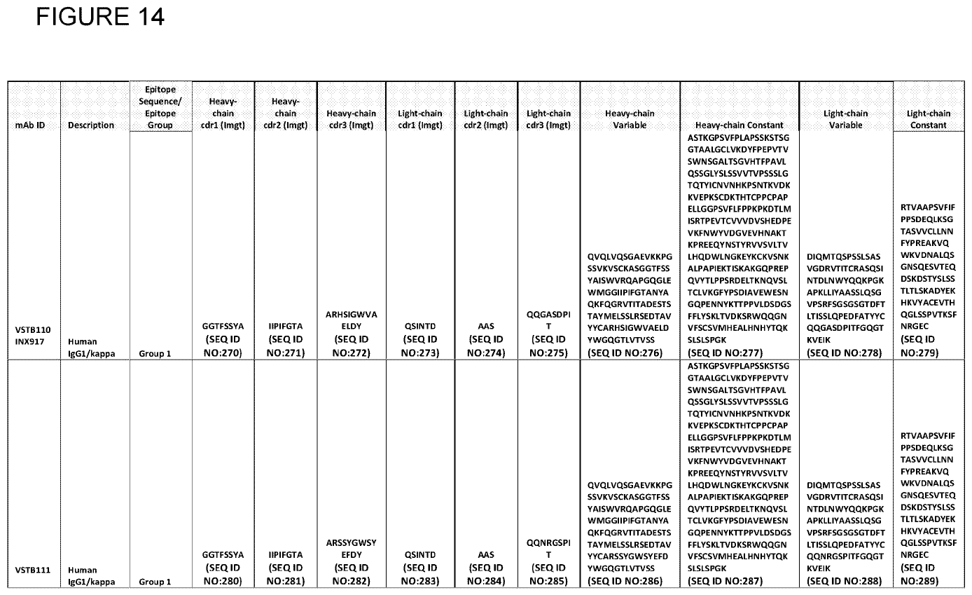

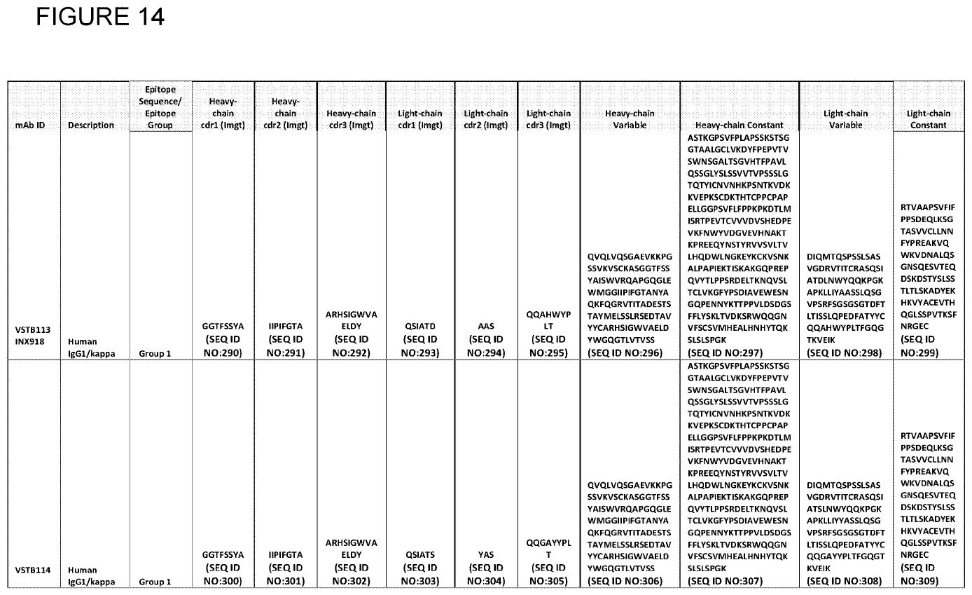

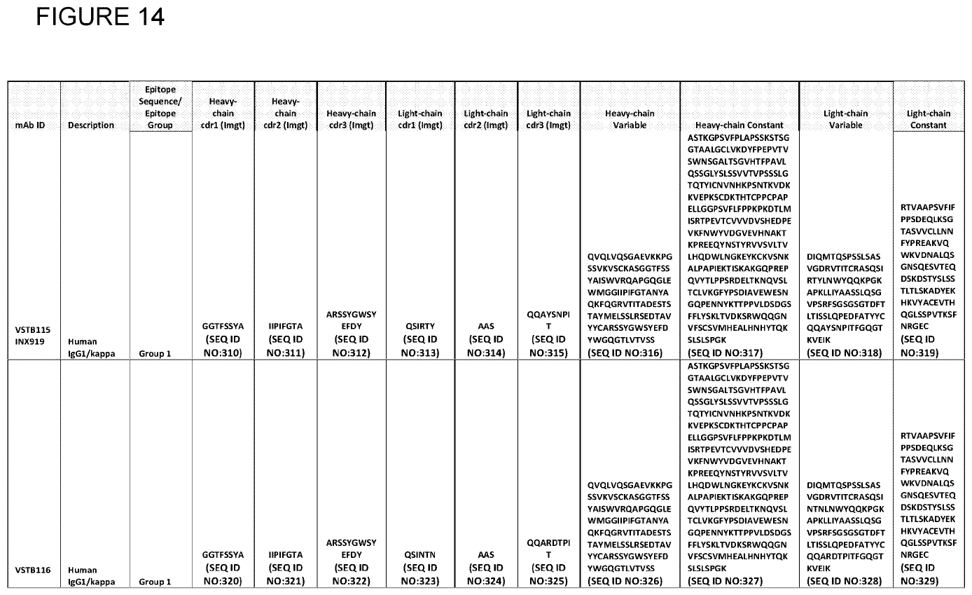

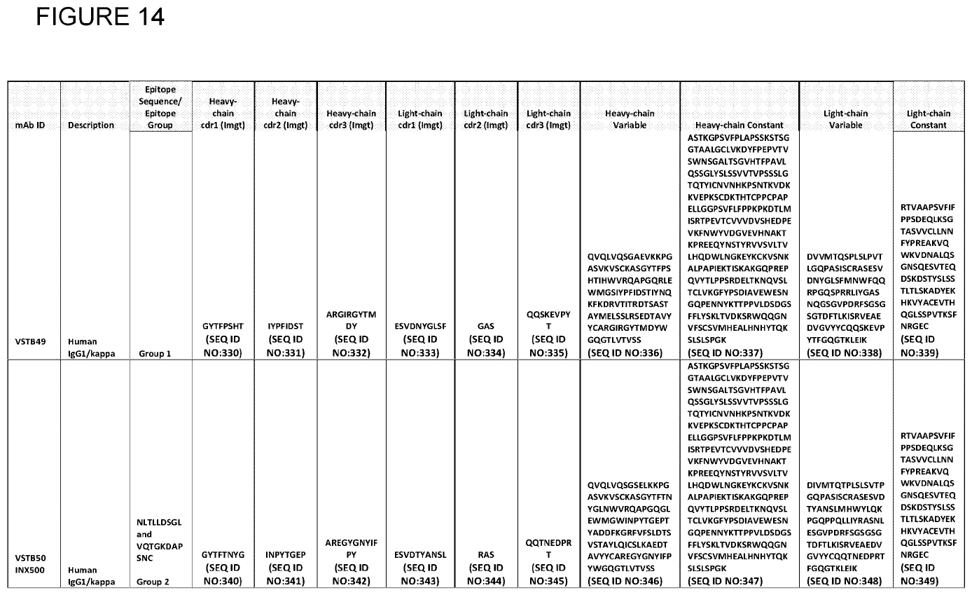

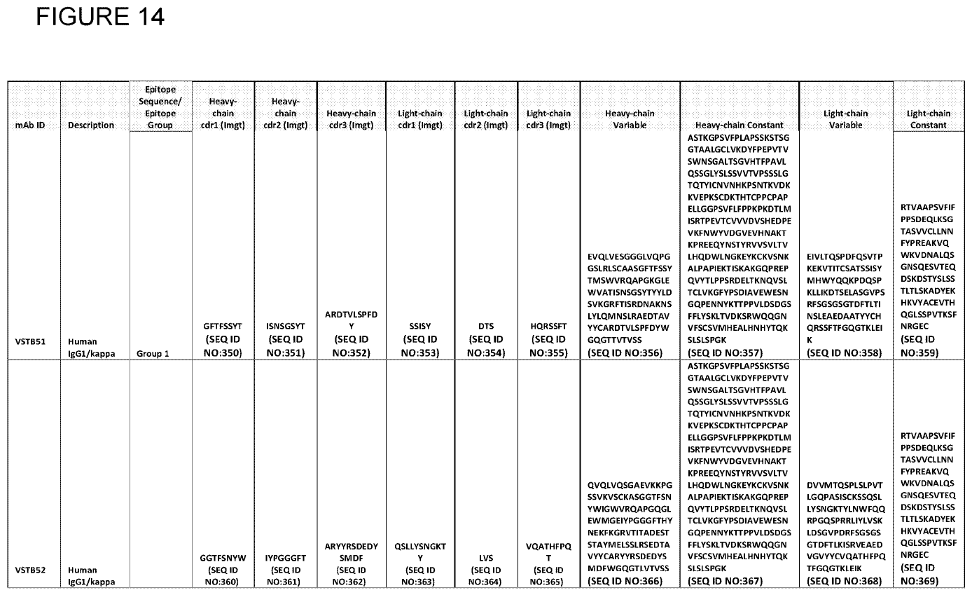

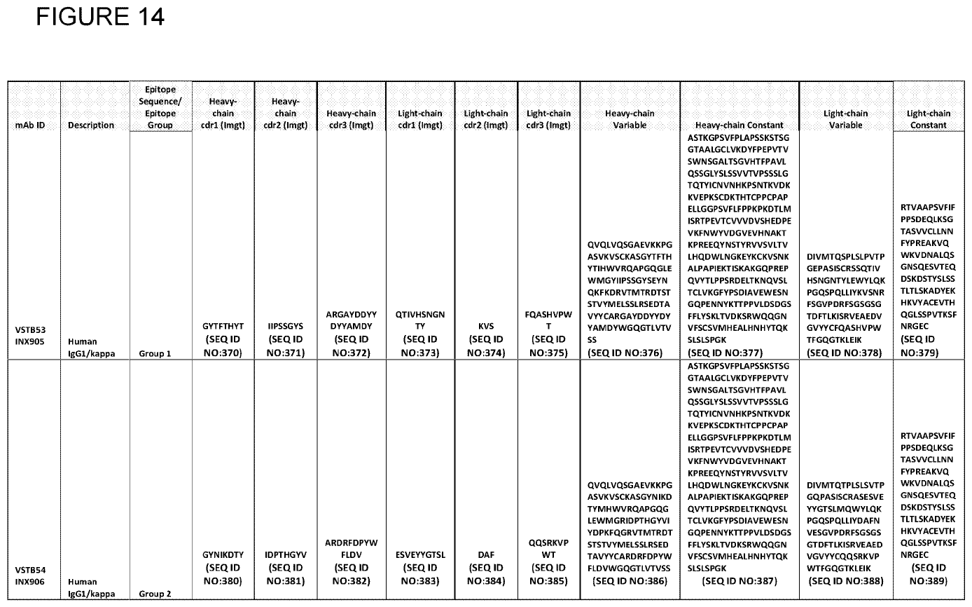

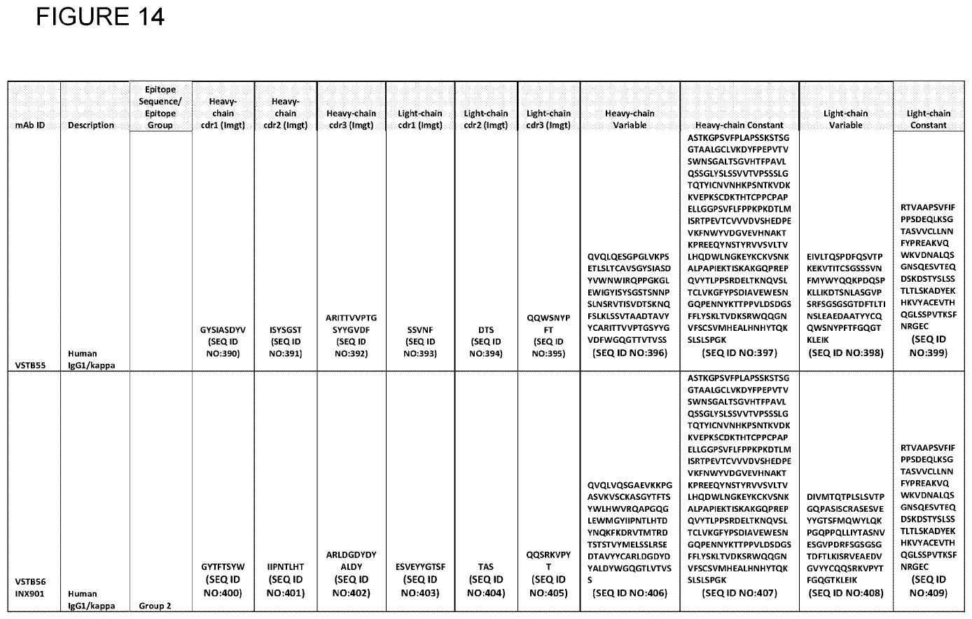

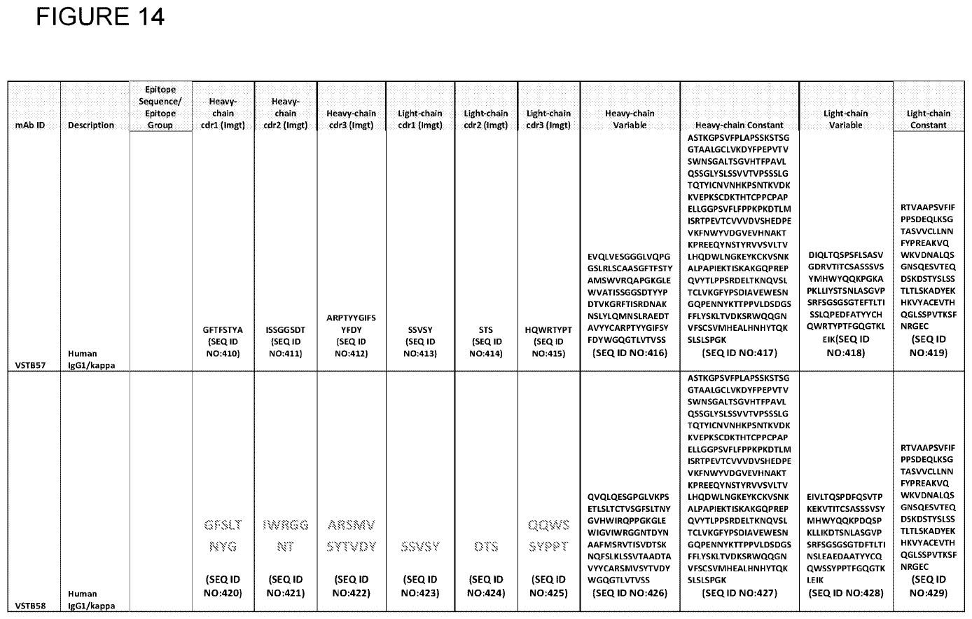

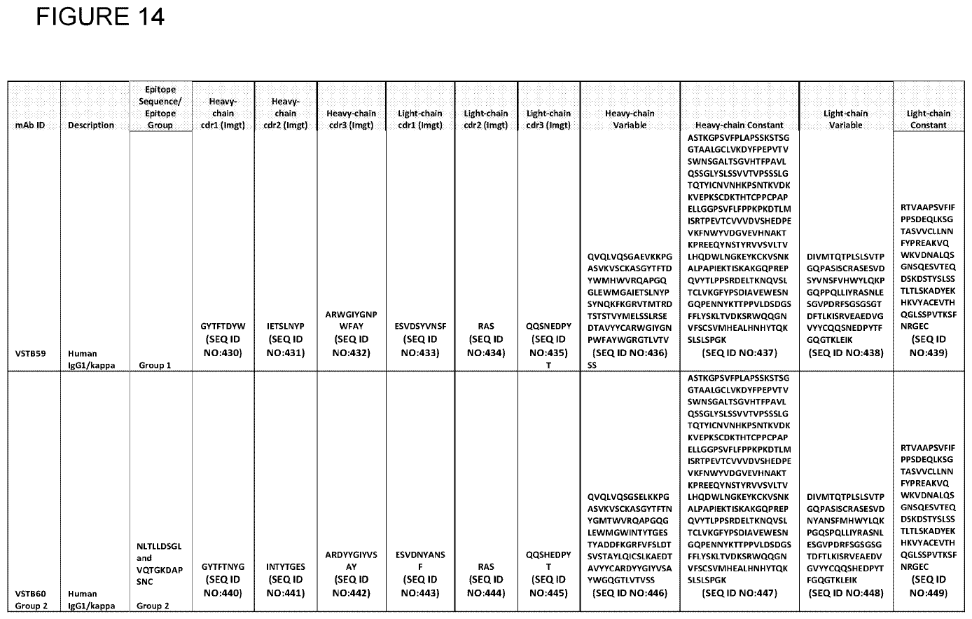

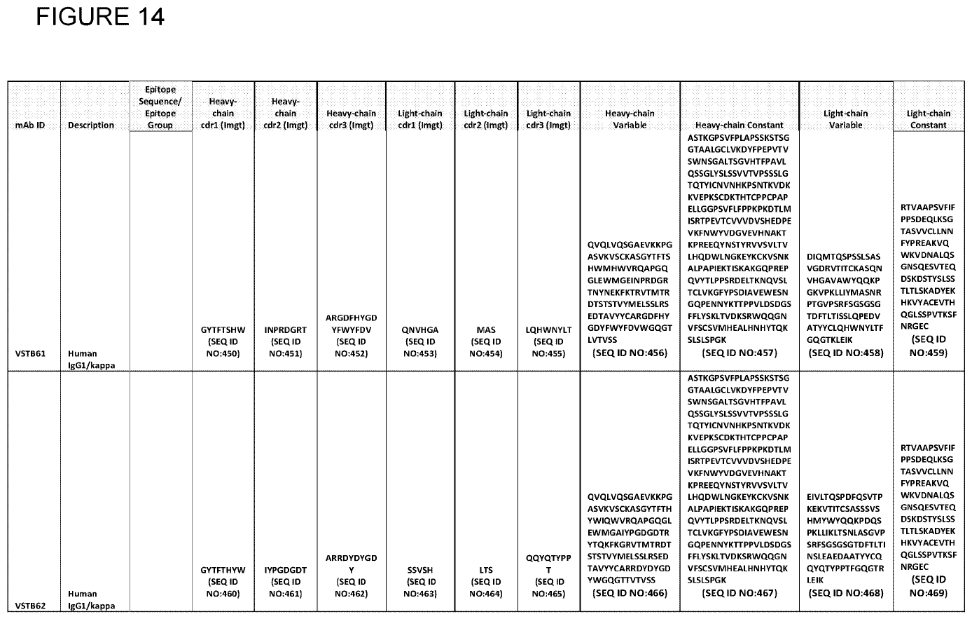

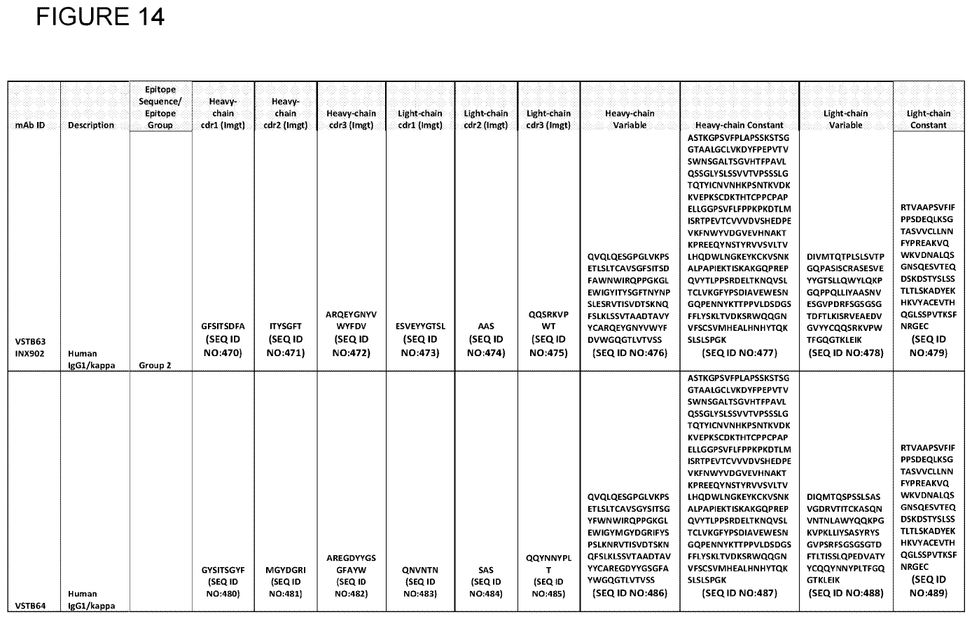

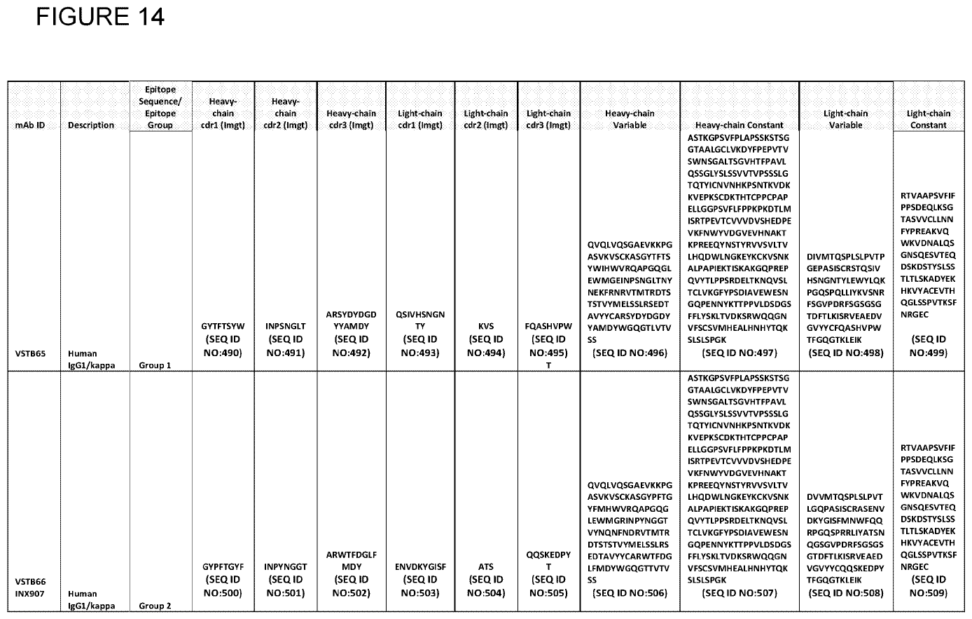

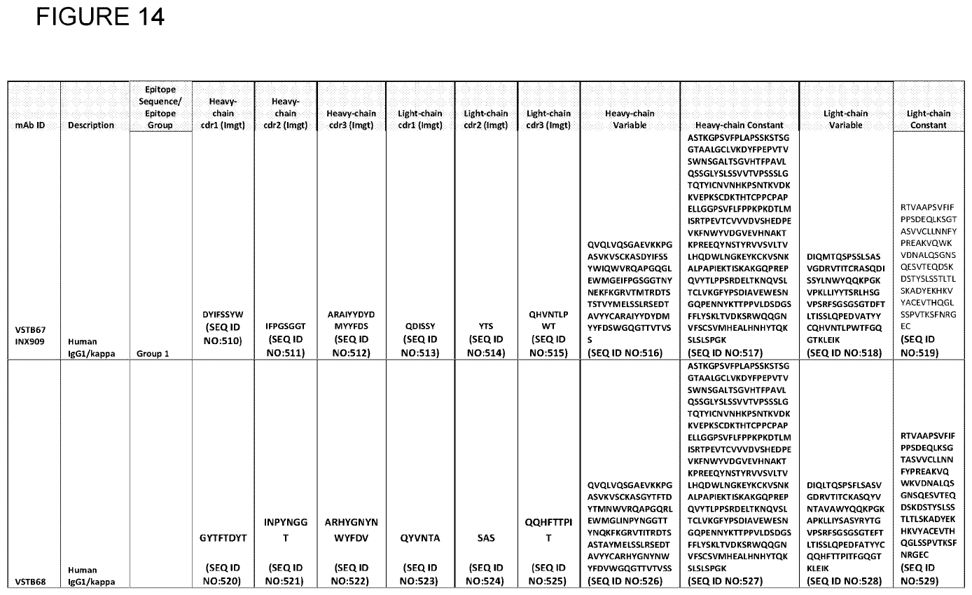

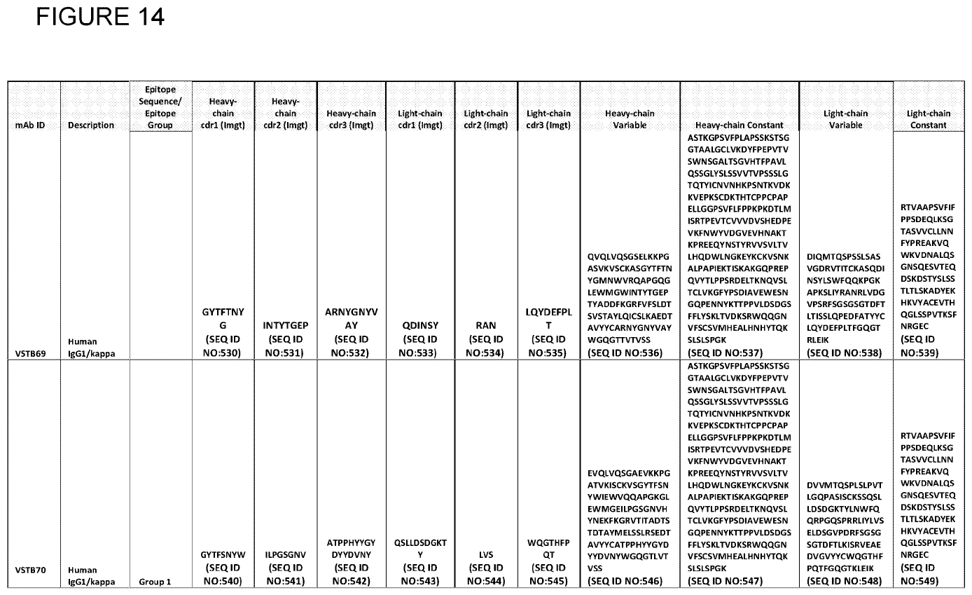

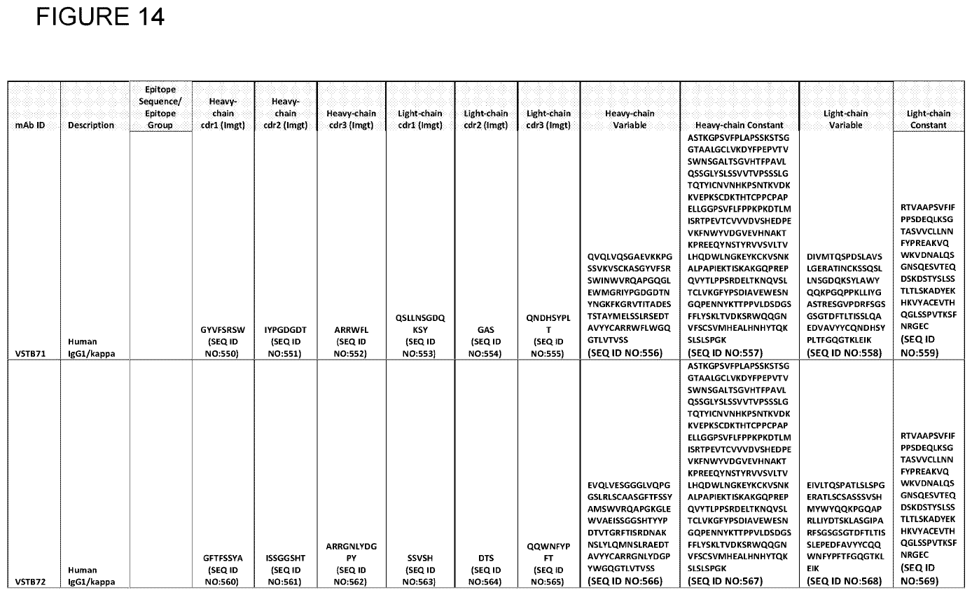

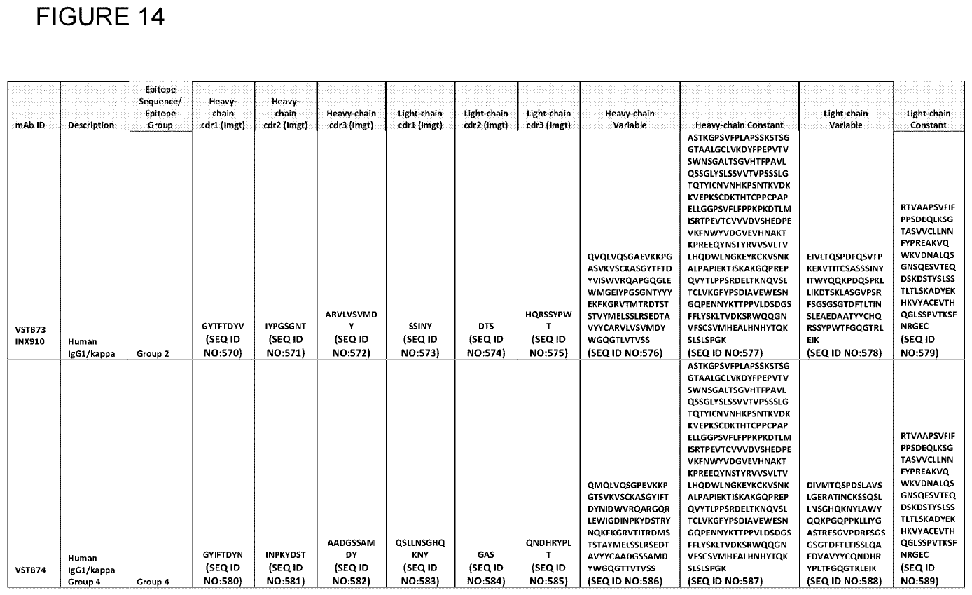

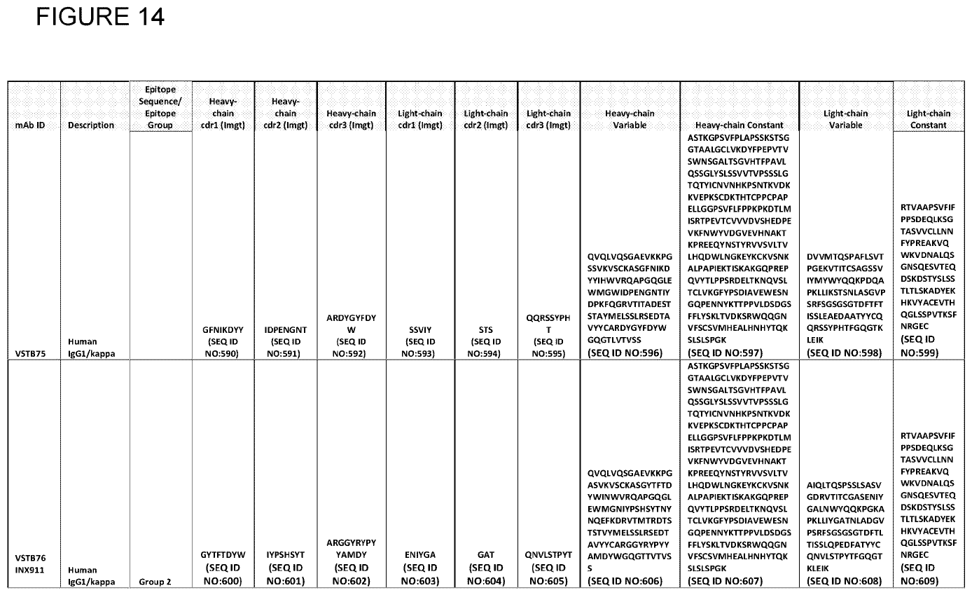

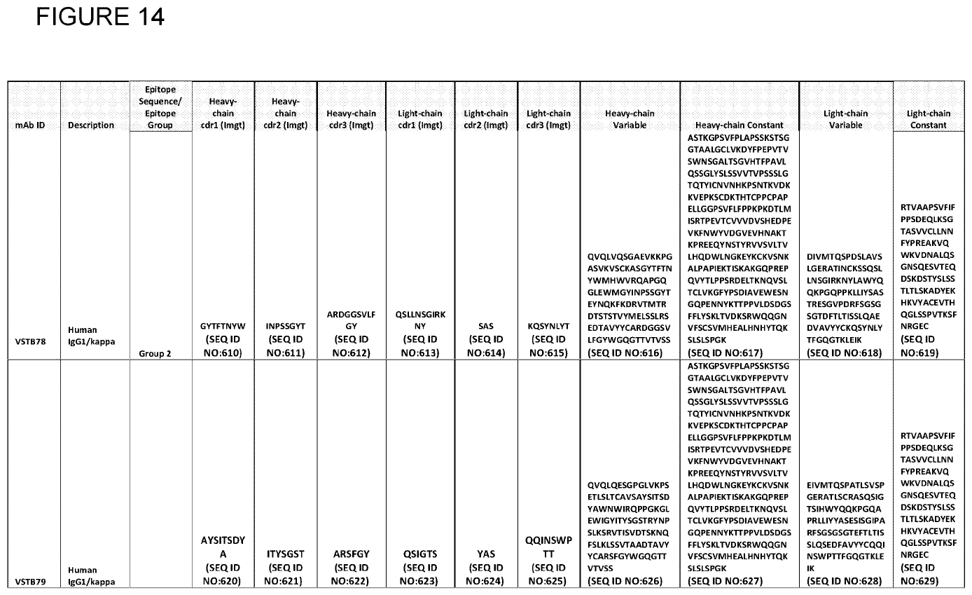

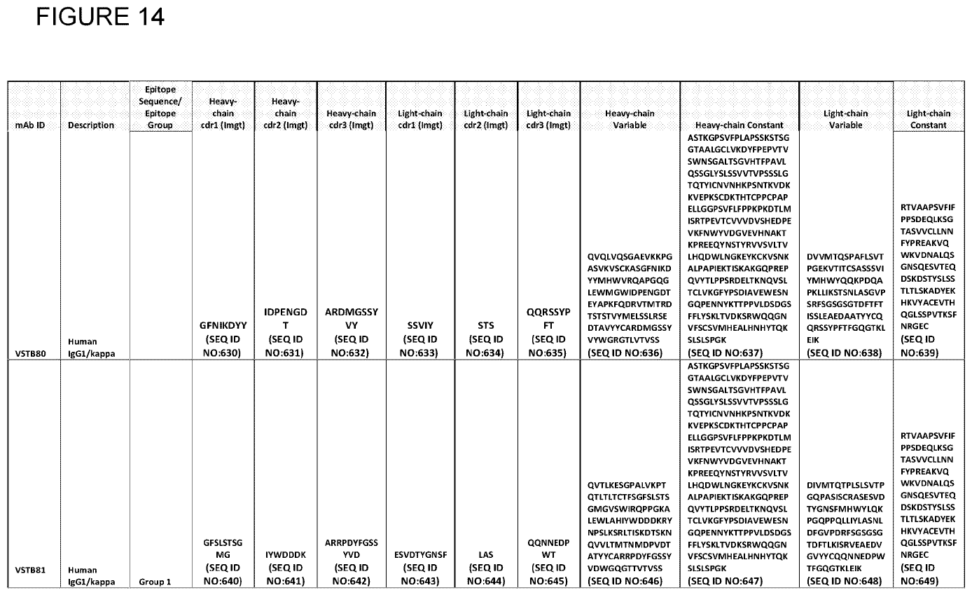

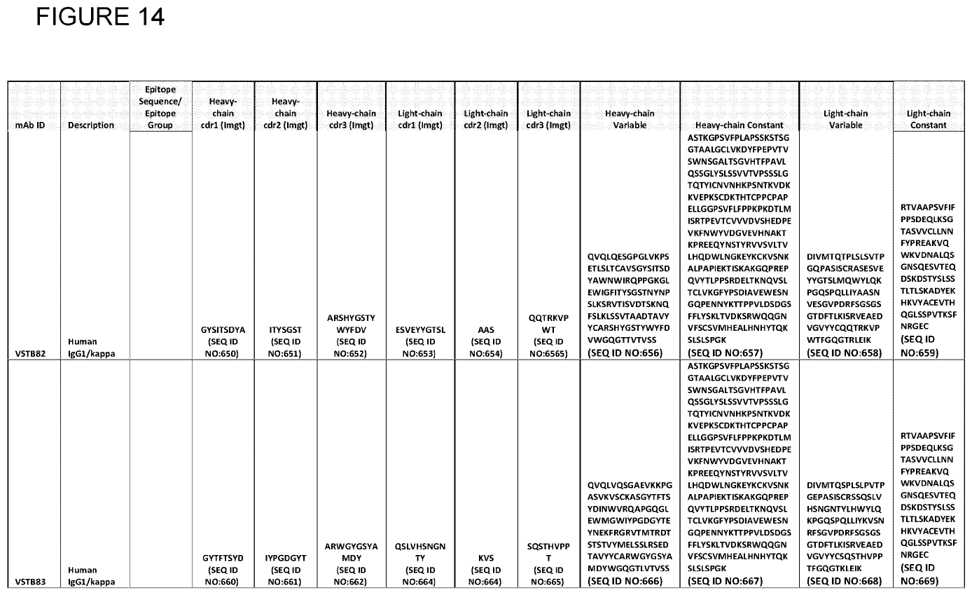

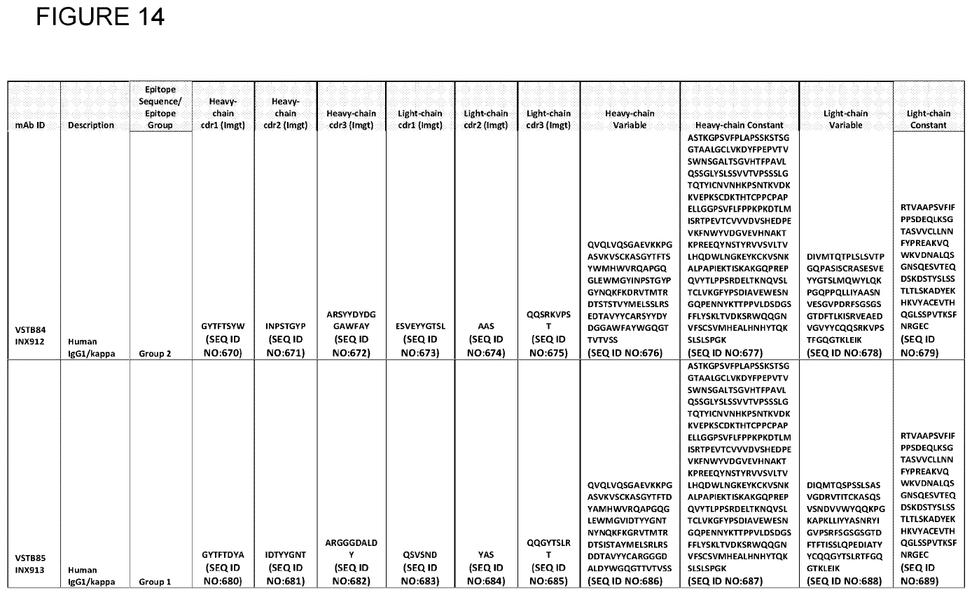

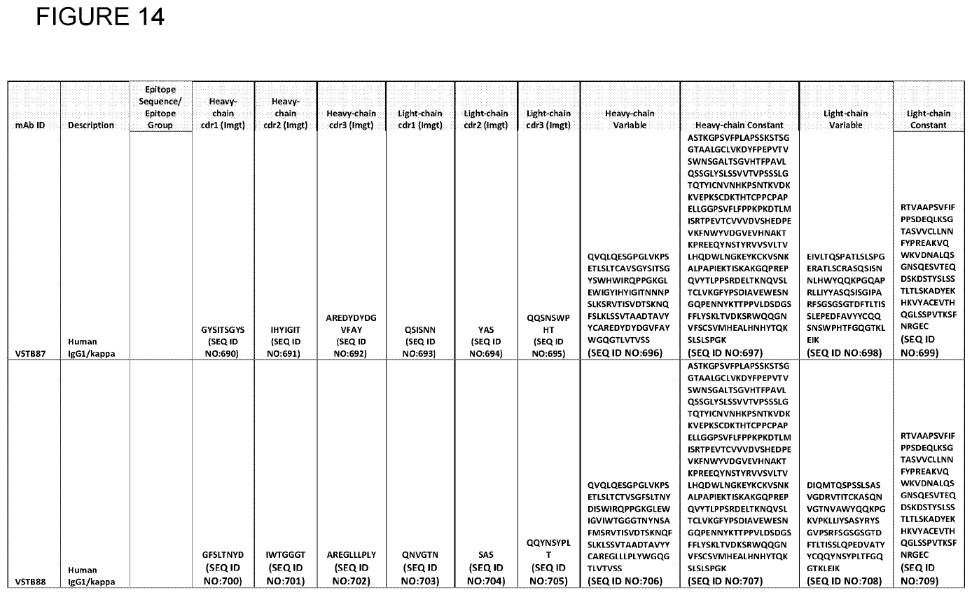

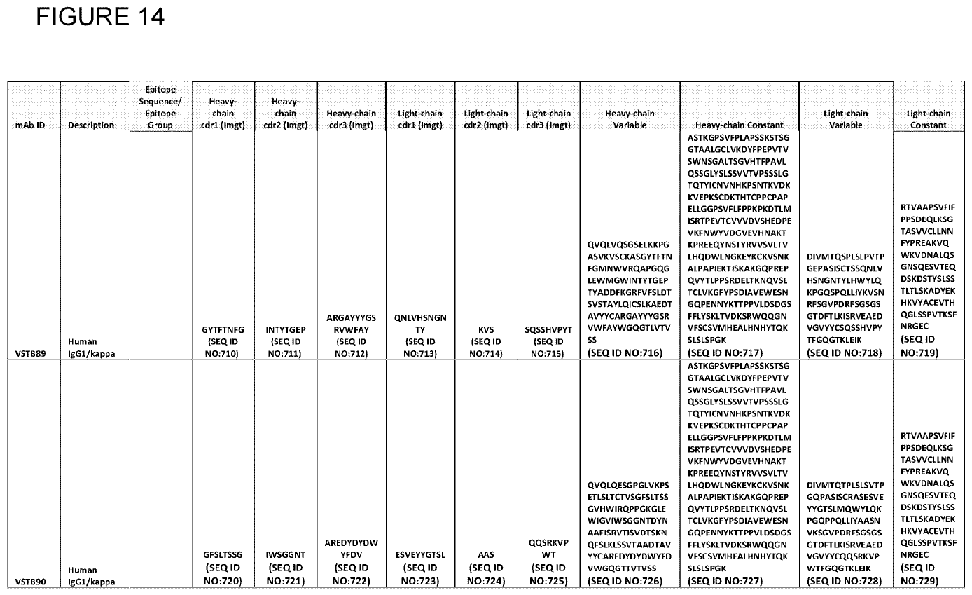

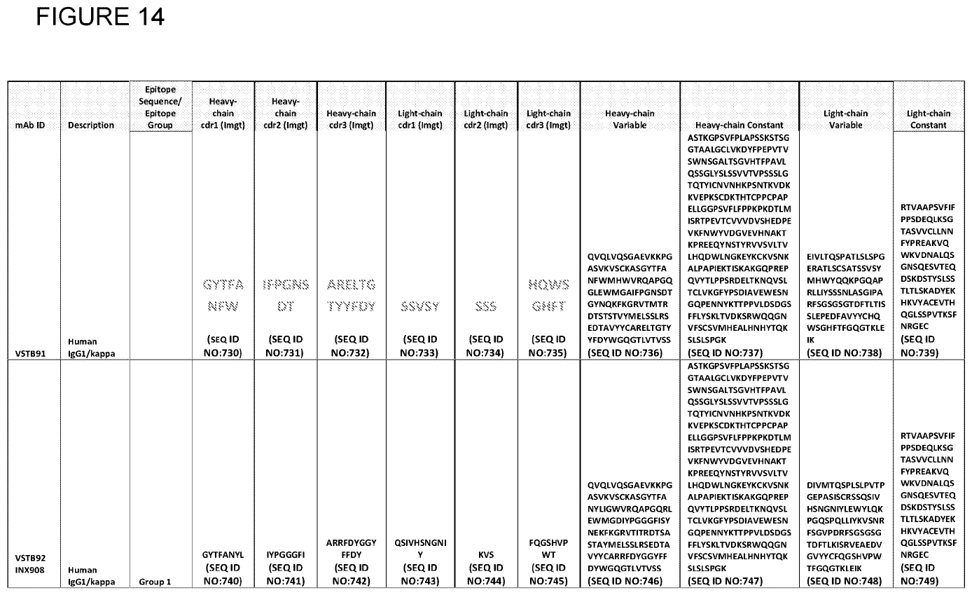

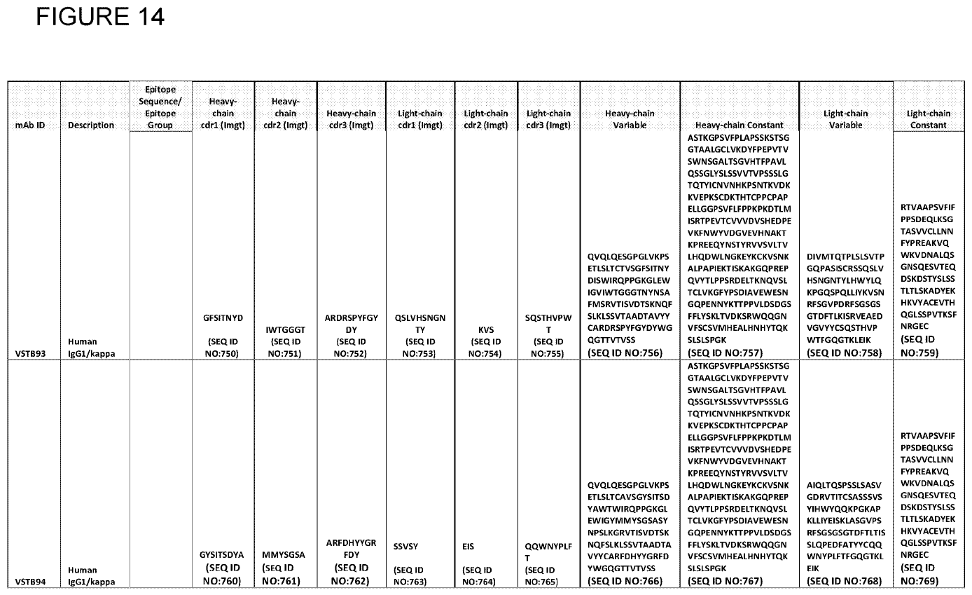

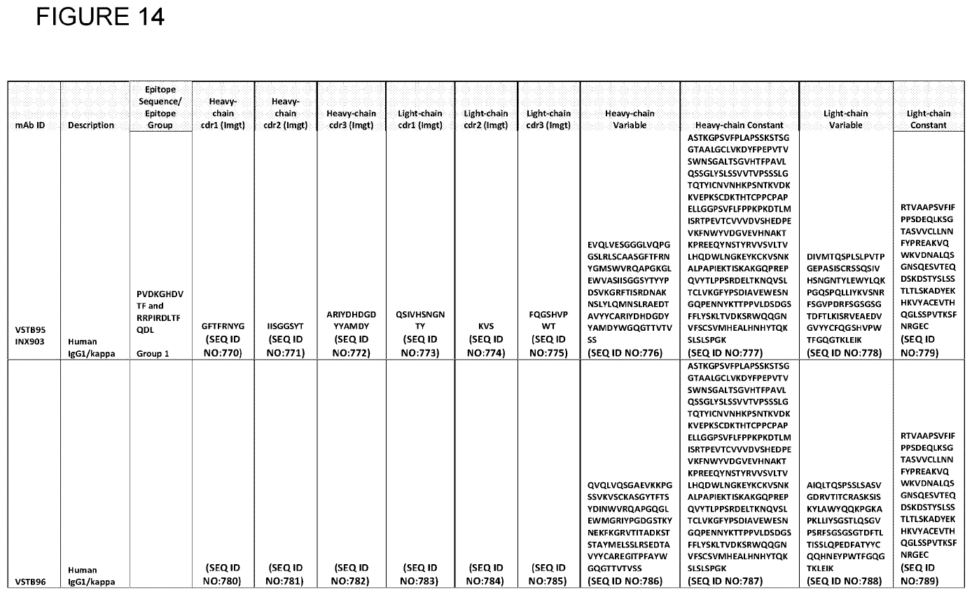

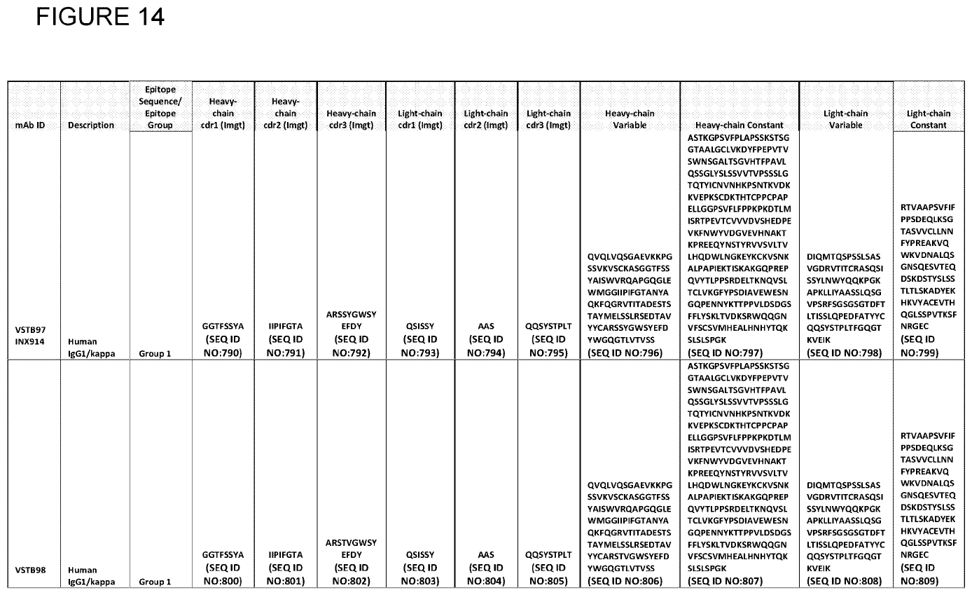

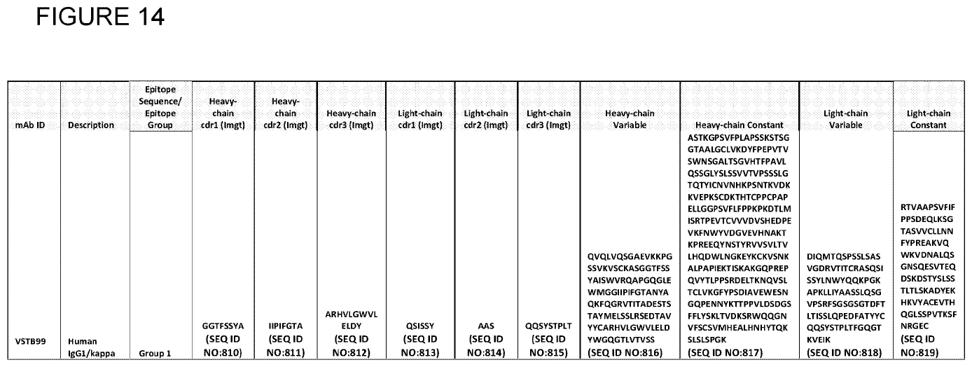

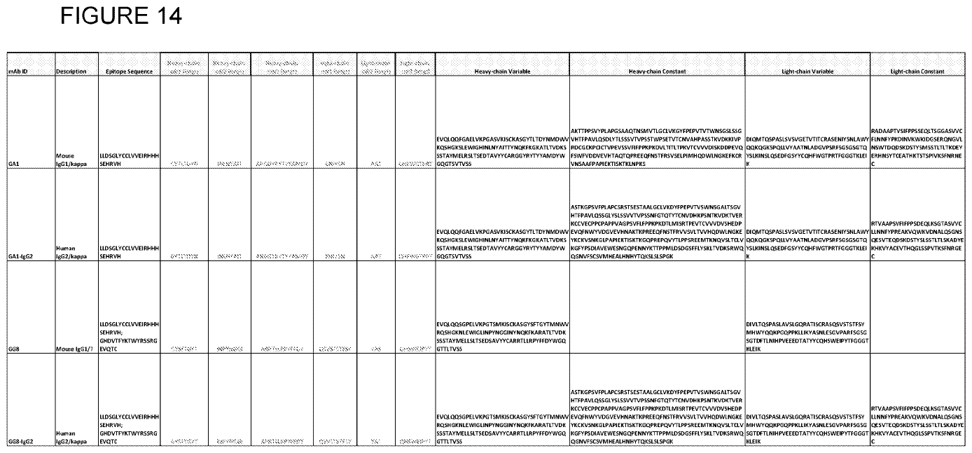

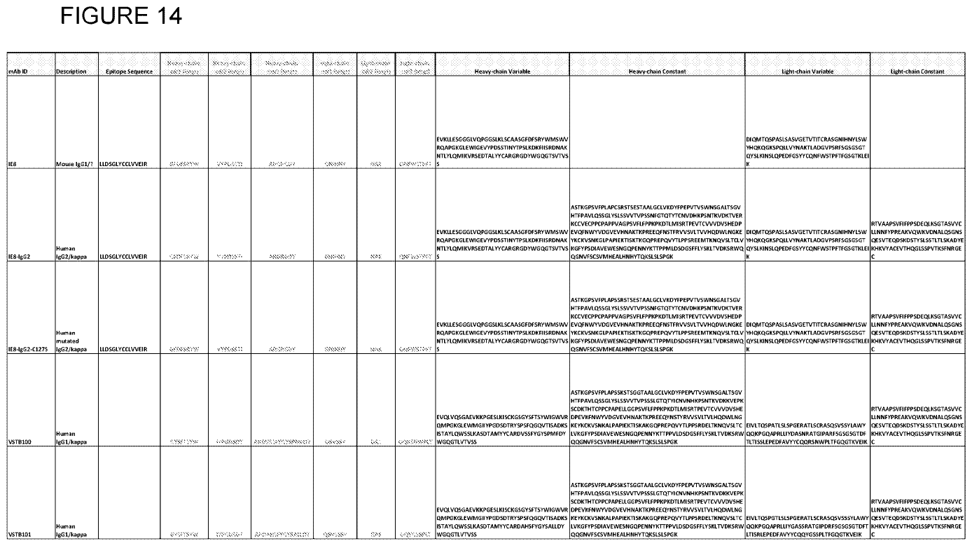

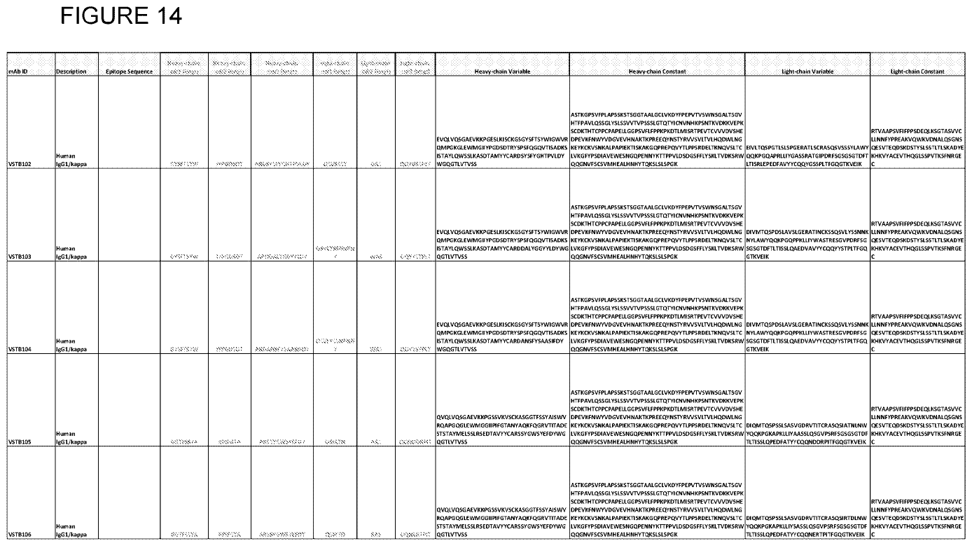

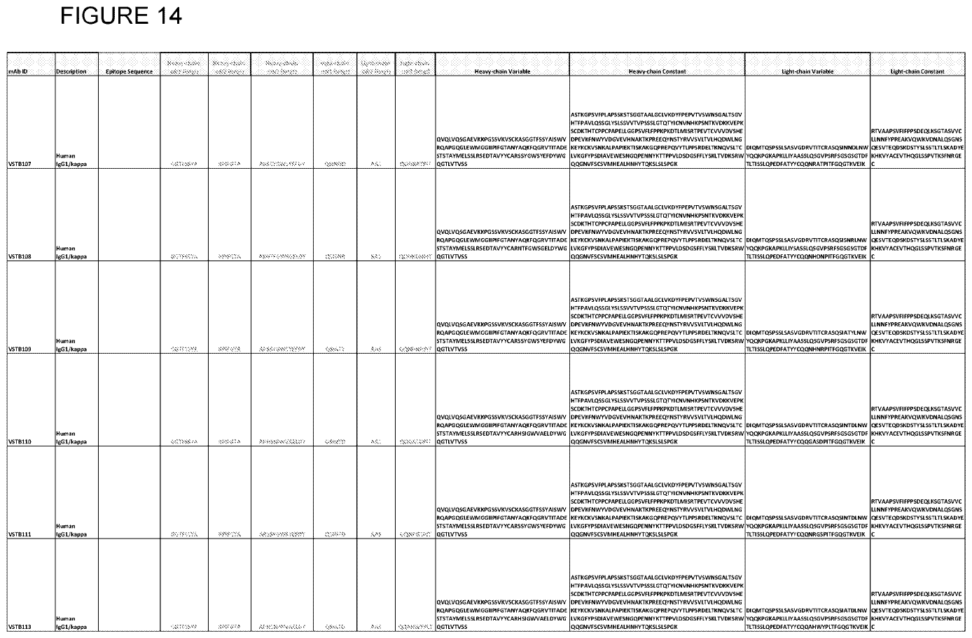

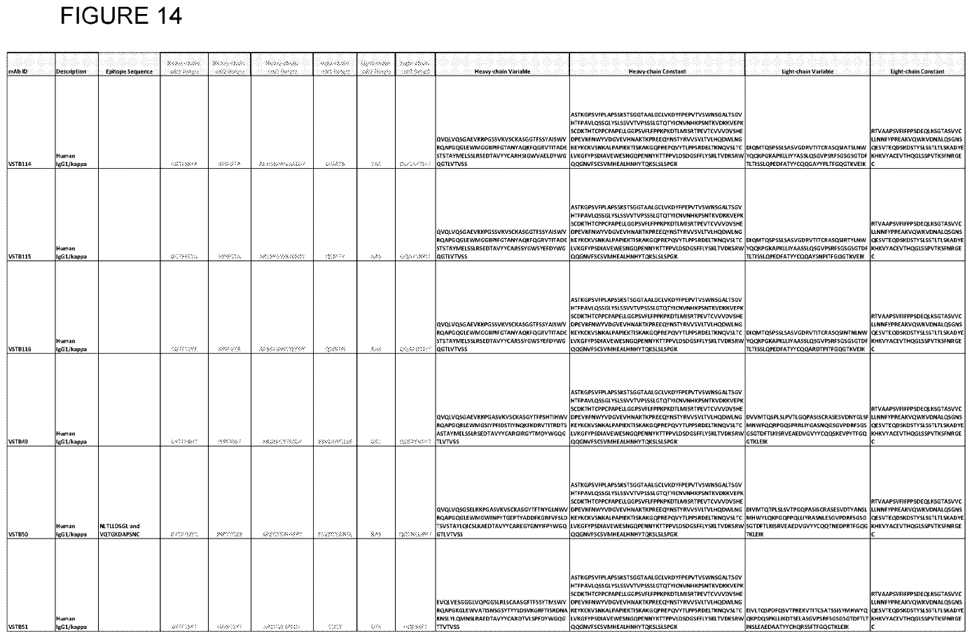

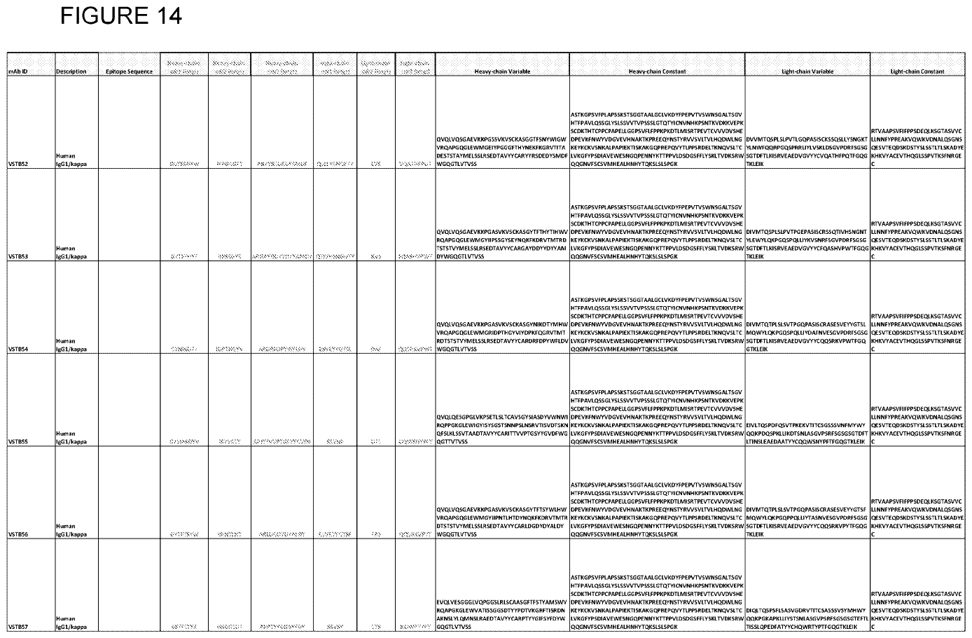

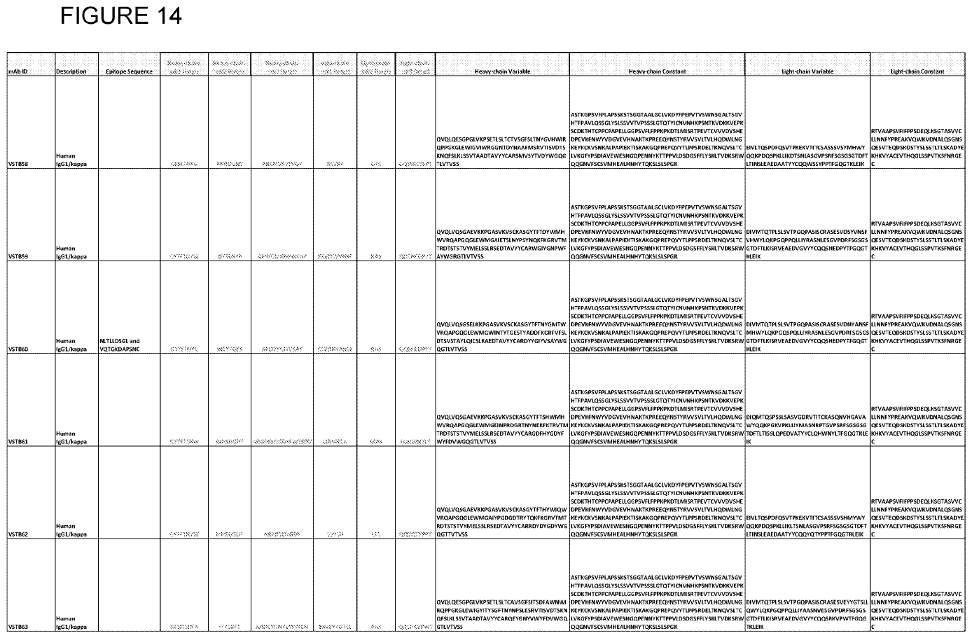

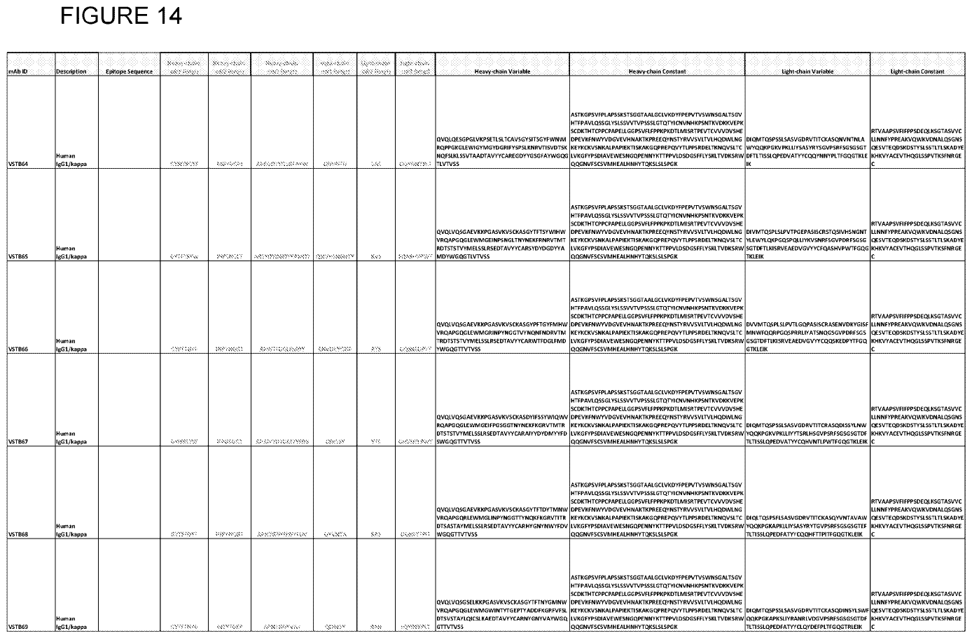

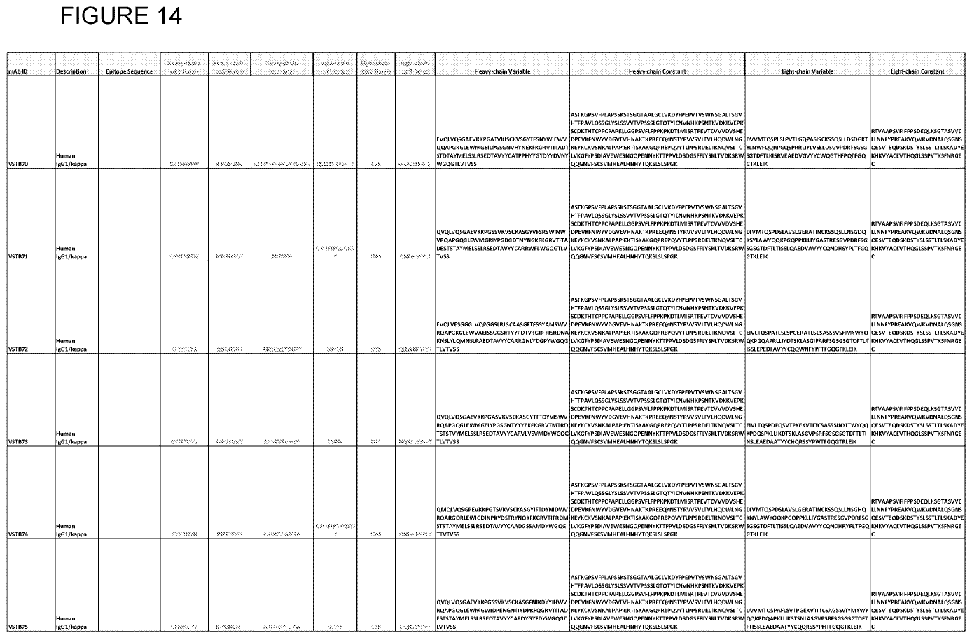

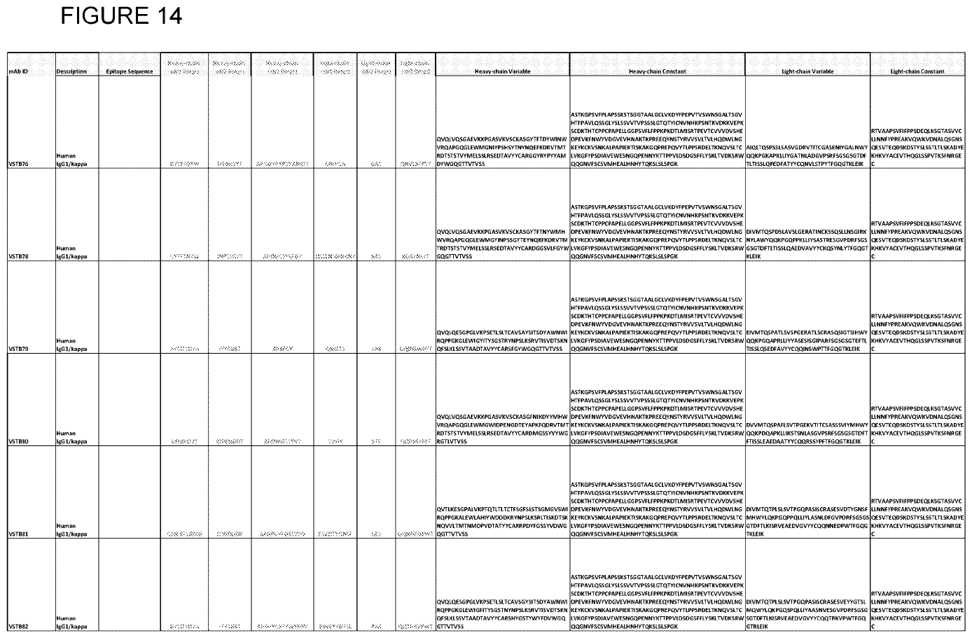

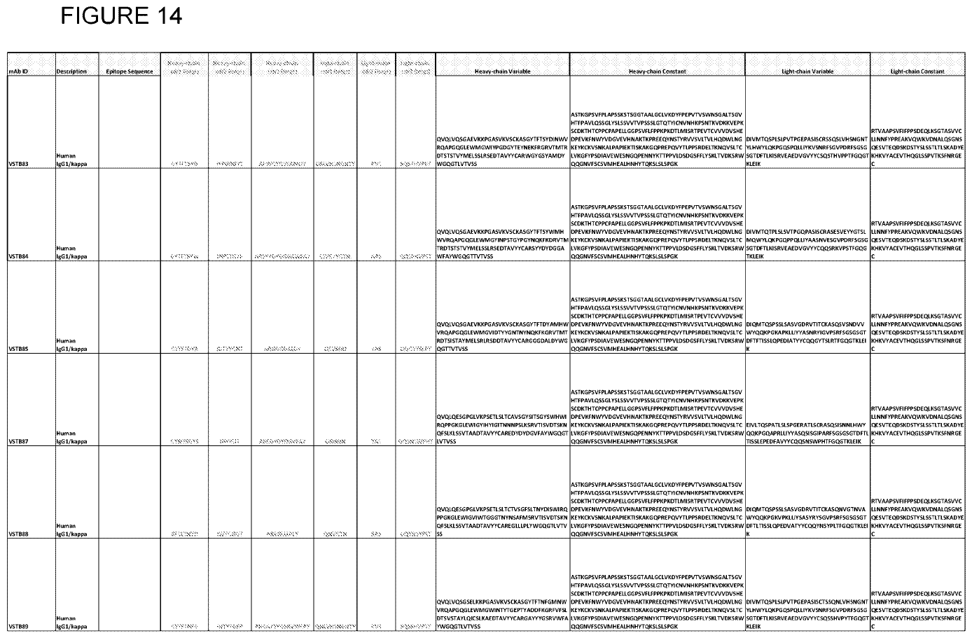

Table A below lists sequences present in FIG. 14 of the subject application and in FIG. 14 of the U.S. provisional priority applications (U.S. Apps. No. 63/506,177 and 63/611,302, identified in the RELATED APPLICATIONS section on page 1 above, all of which are herein incorporated by reference in their entirety), but cannot be included in the 1143260_008613_SL.xml file submitted herewith due to the length of the sequences.

TABLE A

| Item No. | SEQ ID NO: | Length | Type | Organism | Sequence |
|---|---|---|---|---|---|
| 1. | 104 | 3 | PRT | Mus musculus | Ala Ala Thr |
| 2. | 114 | 3 | PRT | Homo sapiens | Ala Ala Thr |
| 3. | 124 | 3 | PRT | Mus musculus | Tyr Ala Ser |
| 4. | 134 | 3 | PRT | Homo sapiens | Tyr Ala Ser |
| 5. | 144 | 3 | PRT | Mus musculus | Asn Ala Lys |
| 6. | 154 | 3 | PRT | Homo sapiens | Asn Ala Lys |
| 7. | 164 | 3 | PRT | Homo sapiens | Asn Ala Lys |
| 8. | 174 | 3 | PRT | Homo sapiens | Asp Ala Ser |
| 9. | 184 | 3 | PRT | Homo sapiens | Gly Ala Ser |
| 10. | 194 | 3 | PRT | Homo sapiens | Gly Ala Ser |
| 11. | 204 | 3 | PRT | Homo sapiens | Trp Ala Ser |
| 12. | 214 | 3 | PRT | Homo sapiens | Trp Ala Ser |
| 13. | 224 | 3 | PRT | Homo sapiens | Ala Ala Ser |
| 14. | 234 | 3 | PRT | Homo sapiens | Ser Ala Ser |
| 15. | 244 | 3 | PRT | Homo sapiens | Ala Ala Ser |
| 16. | 254 | 3 | PRT | Homo sapiens | Ser Ala Ser |
| 17. | 264 | 3 | PRT | Homo sapiens | Ala Ala Ser |
| 18. | 274 | 3 | PRT | Homo sapiens | Ala Ala Ser |
| 19. | 284 | 3 | PRT | Homo sapiens | Ala Ala Ser |
| 20. | 294 | 3 | PRT | Homo sapiens | Ala Ala Ser |
| 21. | 304 | 3 | PRT | Homo sapiens | Tyr Ala Ser |
| 22. | 314 | 3 | PRT | Homo sapiens | Ala Ala Ser |
| 23. | 324 | 3 | PRT | Homo sapiens | Ala Ala Ser |
| 24. | 334 | 3 | PRT | Homo sapiens | Gly Ala Ser |
| 25. | 354 | 3 | PRT | Homo sapiens | Asp Thr Ser |
| 26. | 364 | 3 | PRT | Homo sapiens | Leu Val Ser |
| 27. | 374 | 3 | PRT | Homo sapiens | Lys Val Ser |
| 28. | 384 | 3 | PRT | Homo sapiens | Asp Ala Phe |
| 29. | 394 | 3 | PRT | Homo sapiens | Asp Thr Ser |
| 30. | 404 | 3 | PRT | Homo sapiens | Thr Ala Ser |
| 31. | 414 | 3 | PRT | Homo sapiens | Ser Thr Ser |
| 32. | 424 | 3 | PRT | Homo sapiens | Asp Thr Ser |
| 33. | 434 | 3 | PRT | Homo sapiens | Arg Ala Ser |
| 34. | 444 | 3 | PRT | Homo sapiens | Arg Ala Ser |
| 35. | 454 | 3 | PRT | Homo sapiens | Met Ala Ser |
| 36. | 464 | 3 | PRT | Homo sapiens | Leu Thr Ser |
| 37. | 474 | 3 | PRT | Homo sapiens | Ala Ala Ser |
| 38. | 484 | 3 | PRT | Homo sapiens | Ser Ala Ser |
| 39. | 494 | 3 | PRT | Homo sapiens | Lys Val Ser |
| 40. | 504 | 3 | PRT | Homo sapiens | Ala Thr Ser |
| 41. | 514 | 3 | PRT | Homo sapiens | Tyr Thr Ser |
| 42. | 524 | 3 | PRT | Homo sapiens | Ser Ala Ser |
| 43. | 534 | 3 | PRT | Homo sapiens | Arg Ala Asn |
| 44. | 544 | 3 | PRT | Homo sapiens | Leu Val Ser |
| 45. | 554 | 3 | PRT | Homo sapiens | Gly Ala Ser |
| 46. | 564 | 3 | PRT | Homo sapiens | Asp Thr Ser |
| 47. | 574 | 3 | PRT | Homo sapiens | Asp Thr Ser |
| 48. | 584 | 3 | PRT | Homo sapiens | Gly Ala Ser |
| 49. | 594 | 3 | PRT | Homo sapiens | Ser Thr Ser |
| 50. | 604 | 3 | PRT | Homo sapiens | Gly Ala Thr |
| 51. | 614 | 3 | PRT | Homo sapiens | Ser Ala Ser |
| 52. | 624 | 3 | PRT | Homo sapiens | Tyr Ala Ser |
| 53. | 634 | 3 | PRT | Homo sapiens | Ser Thr Ser |
| 54. | 644 | 3 | PRT | Homo sapiens | Leu Ala Ser |
| 55. | 654 | 3 | PRT | Homo sapiens | Ala Ala Ser |
| 56. | 664 | 3 | PRT | Homo sapiens | Lys Val Ser |
| 57. | 674 | 3 | PRT | Homo sapiens | Ala Ala Ser |
| 58. | 684 | 3 | PRT | Homo sapiens | Tyr Ala Ser |
| 59. | 694 | 3 | PRT | Homo sapiens | Tyr Ala Ser |
| 60. | 704 | 3 | PRT | Homo sapiens | Ser Ala Ser |
| 61. | 714 | 3 | PRT | Homo sapiens | Lys Val Ser |
| 62. | 724 | 3 | PRT | Homo sapiens | Ala Ala Ser |
| 63. | 734 | 3 | PRT | Homo sapiens | Ser Ser Ser |
| 64. | 744 | 3 | PRT | Homo sapiens | Lys Val Ser |
| 65. | 754 | 3 | PRT | Homo sapiens | Lys Val Ser |
| 66. | 764 | 3 | PRT | Homo sapiens | Glu lle Ser |
| 67. | 774 | 3 | PRT | Homo sapiens | Lys Val Ser |
| 68. | 784 | 3 | PRT | Homo sapiens | Ser Gly Ser |
| 69. | 794 | 3 | PRT | Homo sapiens | Ala Ala Ser |
| 70. | 804 | 3 | PRT | Homo sapiens | Ala Ala Ser |
| 71. | 814 | 3 | PRT | Homo sapiens | Ala Ala Ser |

What is claimed is:

1. An antibody-RNA or antibody-nucleic acid conjugate (“ARC” or “ANC”) which comprises (i) an antibody or antibody fragment which binds to an antigen specifically or predominantly expressed by one or more immune cell types; and to which is directly or indirectly conjugated (ii) one or more nucleic acids, preferably RNA or DNA oligonucleotides (“payload” or “payloads”) comprised of wild-type or modified nucleotides, which oligonucleotides specifically bind to a target gene expressed by an immune cell, optionally an immunomodulatory gene or to an RNA encoded thereby; and optionally (iii) a cleavable or non-cleavable linker or adaptor, e.g., a peptide intervening said (i) antibody or antibody fragment and said (ii) one or more nucleic acids; wherein such ARC or ANC when in contact with an immune cell which expresses the antigen bound by the (i) an antibody or antibody fragment is internalized by the immune cell and provides for the release of said (ii) one or more nucleic acids into the immune cell; thereby optionally modulating the expression and/or function of the targeted immune modulator.

2. The ARC or ANC of claim 1, wherein the one or more payloads comprise one or more modified nucleotides, optionally at least one phosphonate and/or ribose modified nucleotide which facilitates direct or indirect attachment of the one or more payloads to the antibody or antibody fragment, optionally via a peptide linker, further optionally a cleavable or non-cleavable linker or adaptor, e.g., a peptide intervening said (i) antibody or antibody fragment and (ii) a payload.

3. The ARC or ANC of any of the previous claims, wherein the payload is directly or indirectly conjugated to the antibody or antibody fragment via a reactive amine, optionally comprised on a lysine residue on the antibody or antibody fragment and/or on a peptide linking the (i) antibody or antibody fragment to (ii) one or more payloads and/or the payload comprises a 3′-sense strand Cy5 label and a 5′-amine on the antisense strand.

4. The ARC or ANC any of the previous claims, wherein the antibody or antibody fragment binds to VISTA, preferably human VISTA.

5. The ARC or ANC of any of the previous claims, wherein the antibody or antibody fragment binds to VISTA, preferably human VISTA, and comprises the same VH and VL CDRs as any one of anti-human VISTA antibodies comprising the sequences in FIG. 14 or in Appendix 1 or Appendix 3.

6. The ARC or ANC any of the previous claims, wherein the antibody or antibody fragment binds to VISTA, preferably human VISTA, and comprises the same VH and/or VL regions and CDRs as any one of anti-human VISTA antibodies comprising the VH and/or VL sequences in FIG. 14 or an antibody or antibody fragment comprising VH and/or VL regions possessing a least 90, 95 or 99% sequence identity to the VH and/or VL regions as any one of anti-human VISTA antibodies comprising the VH and/or VL sequences in FIG. 14; or an antibody or antibody fragment which comprises the VH and/or VL sequences in Appendix 1 or Appendix 3; and which antibody or antibody fragment optionally comprises IgG1, IgG2, IgG3 or IgG4 constant domain polypeptides, further optionally IgG1 constant domain polypeptides, still further optionally IgG1 constant domain polypeptides having the sequences contained in Appendix 1 or 3.

7. The ARC or ANC of any of the previous claims wherein the antibody or antibody fragment comprises a human Fc region, optionally human IgG1, IgG2, IgG3 or IgG4, further optionally modified to impair complement and/or FcR binding and/or to enhance FcRn binding.

8. The ARC or ANC of any of the previous claims which comprises one or more of a short interfering RNA (siRNA), short hairpin RNA (shRNA), microRNA (miRNA), double-stranded RNA (dsRNA), transfer RNA (tRNA), ribosomal RNA (rRNA), or heterogeneous nuclear RNA (hnRNA).

9. The ARC or ANC of any of the previous claims which comprises a polynucleic acid molecule which is from about 10 to about 5000, 10 to about 4000, 10 to about 3000, 10 to about 2000, 10 to about 1000, 10 to about 500, 10 to about 400, 10 to about 300, 10 to about 200, 10 to about 150, 10 to about 100, 10 to about 50, about 10 to about 30, about 15 to about 30, about 18 to about 25, about 18 to about 24, about 19 to about 23, or about 20 to about 22 nucleotides in length; or which comprises a polynucleic acid molecule of about 50 nucleotides, about 45 nucleotides, about 40 nucleotides, about 35 nucleotides, about 30 nucleotides, about 25 nucleotides, about 20 nucleotides, about 19 nucleotides, about 18 nucleotides, about 17 nucleotides, about 16 nucleotides, about 15 nucleotides, about 14 nucleotides, about 13 nucleotides, about 12 nucleotides, about 11 nucleotides, or about 10 nucleotides in length.

10. The ARC or ANC of any of the previous claims which comprises a first polynucleotide and a second polynucleotide, optionally wherein the first polynucleotide is a sense strand or passenger strand and/or the second polynucleotide is an antisense strand or guide strand.

11. The ARC or ANC of any of the previous claims which comprises an siRNA, tRNA, rRNA, or a mRNA.

12. The ARC or ANC of any of the previous claims which comprises, or is encapsulated in or conjugated to a lipid nanoparticle.

13. The ARC or ANC of any of the previous claims wherein at least one payload targets an immunomodulator selected from a cytokine, chemokine, interleukin, interferon, tumor necrosis factor, or receptor of any of the foregoing.

14. The ARC or ANC of any of the previous claims, wherein the payload targets an RNA or DNA sequence encoding an immunomodulator selected from is IL-2, IL-3, IL-4, IL-5, IL-6, IL-7, IL-8, IL-10, IL-11, IL-12, 11-15, IL-17, IL-18, IL-22, IL-37, IL-1β, TGF-β, IFNα, IFNβ, IFNγ, TNF-α, TNF-β, GM-CSF, phosphatidylinositol-4,5-bisphosphate 3-kinase catalytic subunit alpha (PIK3CA), RAR related orphan receptor C (RORC) or any of the molecules having the sequences identified in FIG. 1 or FIG. 2.

15. The ARC or ANC of any of the previous claims, which comprises an siRNA payload which targets an RNA or DNA encoding GLUT3 or PIK3CA, optionally a sequence in Table 11, Appendix 2 or 4.

16. The ARC or ANC of any of the previous claims, wherein the antibody or antibody fragment binds to at least one immune cell selected from PMBCs, a T cell, a T cell progenitor cell, a CD4+ T cell, a helper T cell, a regulatory T cell, a CD8+ T cell, a naïve T cell, an effector T cell, a memory T cell, a stem cell memory T (TSCM) cell, a central memory T (TCM) cell, an effector memory T (TEM) cell, a terminally differentiated effector memory T cell, a tumor-infiltrating lymphocyte (TIL), an immature T cell, a mature T cell, a cytotoxic T cell, a mucosa-associated invariant T (MAIT) cell, a TH1 cell, a TH2 cell, a TH3 cell, a TH17 cell, a TH9 cell, a TH22 cell, a follicular helper T cells, and a/b T cell, a g/d T cell, a Natural Killer T (NKT) cell, a cytokine-induced killer (CIK) cell, a lymphokine-activated killer (LAK) cell, a perforin-deficient cell, a granzyme-deficient cell, a B cell, a myeloid cell, a monocyte, a macrophage, an eosinophil, a neutrophil, and a dendritic cell.

17. The ARC or ANC of any of the previous claims, wherein the antibody or antibody fragment binds to a myeloid cell and/or a T cell.

18. The ARC or ANC of any of the previous claims, wherein the antibody or antibody fragment binds to a T cell or T cell progenitor cell or NK cell.

19. The ARC or ANC of any of the previous claims, which comprises at least one nucleic acid payload, optionally an RNA, further optionally an siRNA or antisense RNA, that binds to a gene or nucleic acid, optionally an RNA or DNA, encoding an antigen selected from the group consisting of: (1) 17-IA, 4-1BB, 4Dc, 6—keto-PGFla, 8-iso-PGF2a, 8-oxo-dG, Al Adenosine Receptor, A33, ACE, ACE-2, Activin, Activin A, Activin AB, Activin B, Activin C, Activin RIA, Activin RIA ALK-2, Activin RIB ALK-4, Activin RIIA, Activin RUB, ADAM, ADAM10, ADAM12, ADAM 15, ADAM 17/T ACE, ADAM8, ADAM9, ADAMTS, ADAMTS4, ADAMTS5, Addressins, aFGF, ALCAM, ALK, ALK-1, ALK-7, alpha-I-antitrypsin, alpha-V/beta-1 antagonist, ANG, Ang, APAF-1, APE, APJ, APP, APRIL, AR, ARC, ART, Artemin, anti-Id, ASPARTIC, Atrial natriuretic factor, av/b3 integrin, Axl, b2M, B7-1, B7-2, B7-H, B-lymphocyte Stimulator (BlyS), BACE, BACE-1, Bad, BAFF, BAFF-R, Bag-1, BAK, Bax, BCA-1, BCAM, Bel, BCMA, BDNF, b-ECGF, bFGF, BID, Bik, BFM, BLC, BL-CAM, BLK, BMP, BMP-2 BMP-2a, BMP-3 Osteogenin, BMP-4 BMP-2b, BMP-5, BMP-6 Vgr-1, BMP-7 (OP-1), BMP-8 (BMP-8a, OP-2), BMPR, BMPR-IA (ALK-3), BMPR-IB (ALK-6), BRK-2, RPK-1, BMPR-II (BRK-3), BMPs, b- NGF, BOK, Bombesin, Bone-derived neurotrophic factor, BPDE, BPDE-DNA, BTC, complement factor 3 (C3), C3a, C4, C5, C5a, CIO, CA125, CAD-8, Calcitonin, cAMP, carcinoembryonic antigen (CEA), carcinoma-associated antigen, Cathepsin A, Cathepsin B, Cathepsin C/DPPI, Cathepsin D, Cathepsin E, Cathepsin H, Cathepsin L, Cathepsin O, Cathepsin S, Cathepsin V, Cathepsin X/Z/P, CBL, CCI, CCK2, CCL, CCL1, CCL11, CCL12, CCL13, CCL 14, CCL15, CCL16, CCL1 7, CCL18, CCL19, CCL2, CCL20, CCL21, CCL22, CCL23, CCL24, CCL25, CCL26, CCL27, CCL28, CCL3, CCL4, CCL5, CCL6, CCL7, CCL8, CCL9/10, CCR, CCR1, CCR10, CCR10, CCR2, CCR3, CCR4, CCR5, CCR6, CCR7, CCR8, CCR9, CD1, CD2, CD4, CD5, CD6, CD7, CD8, CD10, CDIIa, CD11b, CD11c, CD13, CD14, CD15, CD16, CD18, CD19, CD20, CD21, CD22, CD23, CD25, CD27L, CD28, CD29, CD30, CD30L, CD32, CD33 (p67 proteins), CD34, CD38, CD40, CD40L, CD44, CD45, CD46, CD49a, CD52, CD54, CD55, CD56, CD61, CD64, CD66e, CD74, CD80 (B7-1), CD89, CD95, CD123, CD137, CD138, CD140a, CD146, CD147, CD148, CD152, CD164, CEACAM5, CFTR, cGMP, CINC, Clostridium botulinum toxin, Clostridium perfringens toxin, CKb8-1, CLC, CMV, CMV UL, CNTF, CNTN-1, COX, C-Ret, CRG-2, CT-1, CTACK, CTGF, CTLA-4, CX3CL1, CX3CR1, CXCL, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCR, CXCR1, CXCR2, CXCR3, CXCR4, CXCR5, CXCR6, cytokeratin tumor-associated antigen, DAN, DCC, DcR3, DC-SIGN, Decay accelerating factor, des(I-3)-IGF-I (brain IGF-1), Dhh, digoxin, DNAM-1, Dnase, Dpp, DPPIV/CD26, Dtk, ECAD, EDA, EDA-A1, EDA-A2, EDAR, EGF, EGFR (ErbB-1), EMA, EMMPRIN, EN A, endothelin receptor, Enkephalinase, eNOS, Eot, eotaxinl, EpCAM, Ephrin B2/EphB4, EPO, ERCC, E-selectin, ET-1, Factor Ila, Factor VII, Factor VIIIc, Factor IX, fibroblast activation protein (FAP), Fas, FcRI, FEN-1, Ferritin, FGF, FGF-19, FGF-2, FGF3, FGF-8, FGFR, FGFR-3, Fibrin, FL, FLIP, Flt-3, Flt-4, Follicle stimulating hormone, Fractalkine, FZD1, FZD2, FZD3, FZD4, FZD5, FZD6, FZD7, FZD8, FZD9, FZD10, G250, Gas 6, GCP-2, GCSF, GD2, GD3, GDF, GDF-1, GDF-3 (Vgr-2), GDF-5 (BMP-14, CDMP-1), GDF-6 (BMP-13, CDMP-2), GDF-7 (BMP-12, CDMP-3), GDF-8 (Myostatin), GDF-9, GDF-15 (MIC-1), GDNF, GFAP, GFRa-1, GFR-alpha1, GFR-alpha2, GFR-alpha3, GITR, Glucagon, Glut 4, glycoprotein IIb/IIIa (GP IIb/IIIa), GM-CSF, gpl30, gp72, GRO, Growth hormone releasing factor, Hapten (NP-cap or NIP-cap), HB-EGF, HCC, HCMV gB envelope glycoprotein, HCMV) gH envelope glycoprotein, HCMV UL, Hemopoietic growth factor (HGF), Hep B gpl20, heparanase, Her2, Her2/neu (ErbB-2), Her3 (ErbB-3), Her4 (ErbB-4), herpes simplex virus (HSV) gB glycoprotein, HSV gD glycoprotein, HGFA, High molecular weight melanoma-associated antigen (HMW-MAA), HIV gpl20, HIV IIIB gp 120 V3 loop, HLA, HLA-DR, HM1.24, HMFG PEM, HRG, Hrk, human cardiac myosin, human cytomegalovirus (HCMV), human growth hormone (HGH), HVEM, 1-309, IAP, ICAM, ICAM-1, ICAM-3, ICE, ICOS, IFNg, Ig, IgA receptor, IgE, IGF, IGF binding proteins, IGF-1R, IGFBP, IGF-I, IGF-II, IL, IL-1, IL-1R, IL-2, IL-2R, IL-4, IL-4R, IL-5, IL-5R, IL-6, IL-6R, IL-8, IL-9, IL-10, IL-12, IL-13, IL-15, IL-18, IL-18R, IL-23, interferon (INF)-alpha, INF-beta, INF- gamma, Inhibin, iNOS, Insulin A-chain, Insulin B-chain, Insulin-like growth factor 1, integrin alpha2, integrin alpha3, integrin alpha4, integrin alpha4/beta1, integrin, alpha4/beta7, integrin alpha5 (alphaV), integrin alpha5/beta1, integrin alpha5/beta3, integrin alpha6, integrin beta1, integrin beta2, interferon gamma, IP-10, 1-TAC, JE, Kallikrein 2, Kallikrein 5, Kallikrein 6, Kallikrein 11, Kallikrein 12, Kallikrein 14, Kallikrein 15, Kallikrein LI, Kallikrein L2, Kallikrein L3, Kallikrein L4, KC, KDR, Keratinocyte Growth Factor (KGF), laminin 5, LAMP, LAP, LAP (TGF-1), Latent TGF-1, Latent TGF-1 bpl, LBP, LDGF, LECT2, Lefty, Lewis-Y antigen, Lewis-Y related antigen, LFA-1, LFA-3, Lfo, LIF, LIGHT, lipoproteins, LIX, LKN, Lptn, L-Selectin, LT-a, LT-b, LTB4, LTBP-1, Lung surfactant, Luteinizing hormone, Lymphotoxin Beta Receptor, Mac-1, MAdCAM, MAG, MAP2, MARC, MCAM, MCAM, MCK-2, MCP, M-CSF, MDC, Mer, a metalloprotease, MGDF receptor, MGMT, MHC (HLA-DR), MIF, MIG, MIP, MIP-1-alpha, MK, MMAC1, MMP, MMP-1, MMP-10, MMP-11, MMP-12, MMP-13, MMP-14, MMP-15, MMP-2, MMP-24, MMP-3, MMP-7, MMP-8, MMP-9, MPIF, Mpo, MSK, MSP, mucin (Mucl), MUC18, Muellerian- inhibiting substance, Mug, MuSK, NAIP, NAP, NCAD, N-Cadherin, NCA 90, NCAM, Neprilysin, Neurotrophin-3, -4, or -6, Neurturin, Neuronal growth factor (NGF), NGFR, NGF-beta, nNOS, NO, NOS, Npn, NRG-3, NT, NTN, OB, OGG1, OPG, OPN, OSM, OX40L, OX40R, p150, p95, PADPr, Parathyroid hormone, PARC, PARP, PBR, PBSF, PCAD, P-Cadherin, PCNA, PDGF, PDGF, PDK-1, PECAM, PEM, PF4, PGE, PGF, PG12, PGJ2, PIN, PLA2, placental alkaline phosphatase (PLAP), PIGF, PLP, PP14, Proinsulin, Prorelaxin, Protein C, PS, PSA, PSCA, prostate specific membrane antigen (PSMA), PTEN, PTHrp, Ptk, PTN, R51, RANK, RANKL, RANTES, Relaxin A-chain, Relaxin B-chain, renin, respiratory syncytial virus (RSV) F, RSV Fgp, Ret, Rheumatoid factors, RLIP76, RPA2, RSK, S100, SCF/KL, SDF-1, SERINE, Serum albumin, sFRP-3, Shh, SIGIRR, SK-1, SLAM, SLPI, SMAC, SMDF, SMOH, SOD, SPARC, Stat, STEAP, STEAP-II, TACE, TACI, TAG-72 (tumor- associated glycoprotein-72), TARC, TCA-3, T-cell receptors (e.g., T-cell receptor alpha/beta), TdT, TECK, TEM1, TEM5, TEM7, TEM8, TERT, testicular PLAP-like alkaline phosphatase, TfR, TGF, TGF-alpha, TGF-beta, TGF-beta Pan Specific, TGF-beta RI (ALK-5), TGF-beta RII, TGF-beta RIIb, TGF-beta Rill, TGF-beta1, TGF-beta2, TGF-beta3, TGF-beta4, TGF-beta5, Thrombin, Thymus Ck-1, Thyroid stimulating hormone, Tie, TIMP, TIQ, TMEFF2, Tmpo, TMPRSS2, TNF, TNF-alpha, TNF-alpha beta, TNF-beta2, TNFc, TNF-RI, TNF-RII, TNFRSF10A (TRAIL RI Apo-2, DR4), TNFRSFIOB (TRAIL R2 DR5, KILLER, TRICK-2A, TRICK-B), TNFRSF10C (TRAIL R3 DcRI, LIT, TRID), TNFRSF10D (TRAIL R4 DcR2, TRUNDD), TNFRSF11A (RANK ODF R, TRANCE R), TNFRSFIIB (OPG OCIF, TR1), TNFRSF12 (TWEAK R FN14), TNFRSF13B (TACI), TNFRSF13C (BAFF R), TNFRSF14 (HVEM ATAR, HveA, LIGHT R, TR2), TNFRSF16 (NGFR p75NTR), TNFRSF17 (BCMA), TNFRSF 18 (GITR AITR), TNFRSF19 (TROY TAJ, TRADE), TNFRSF19L (RELT), TNFRSFIA (TNF RI CD120a, p55-60), TNFRSFIB (TNF RII CD120b, p75-80), TNFRSF26 (TNFRH3), TNFRSF3 (LTbR TNF Rill, TNFC R), TNFRSF4 (OX40 ACT35, TXGP1 R), TNFRSF 5 (CD40 p50), TNFRSF6 (Fas Apo-1, APT1, CD95), TNFRSF6B (DcR3 M68, TR6), TNFRSF7 (CD27), TNFRSF8 (CD30), TNFRSF9 (4-1BB CD137, ILA), TNFRSF21 (DR6), TNFRSF22 (DcTRAIL R2 TNFRH2), TNFRST23 (DcTRAIL RI TNFRH1), TNFRSF25 (DR3 Apo-3, LARD, TR-3, TRAMP, WSL-1), TNFSF10 (TRAIL Apo-2 Ligand, TL2), TNFSF11 (TRANCE/RANK Ligand ODF, OPG Ligand), TNFSF12 (TWEAK Apo-3 Ligand, DR3 Ligand), TNFSF13 (APRIL TALL2), TNFSF13B (BAFF BLYS, TALL1, THANK, TNFSF20), TNFSF14 (LIGHT HVEM Ligand, LTg), TNFSF15 (TLIA/VEGI), TNFSF18 (GITR Ligand AITR Ligand, TL6), TNFSFIA (TNF-α Conectin, DIF, TNFSF2), TNFSF1B (TNF-b LTa, TNFSF1), TNFSF3 (LTb TNFC, p33), TNFSF4 (OX40 Ligand gp34, TXGP1), TNFSF5 (CD40 Ligand CD154, gp39, HIGM1, IMD3, TRAP), TNFSF6 (Fas Ligand Apo-1 Ligand, APT1 Ligand), TNFSF7 (CD27 Ligand CD70), TNFSF8 (CD30 Ligand CD153), TNFSF9 (4-1BB Ligand CD137 Ligand), TP-1, t-PA, Tpo, TRAIL, TRAIL R, TRAIL-R1, TRAIL-R2, TRANCE, transferring receptor, TRF, Trk, TROP-2, TSG, TSLP, tumor-associated antigen CA 125, tumor-associated antigen expressing Lewis Y related carbohydrate, TWEAK, TXB2, Ung, uPAR, uPAR-1, Urokinase, VCAM, VCAM-1, VECAD, VE-Cadherin, VE-cadherin-2, VEFGR-1 (flt-1), VEGF, VEGFR, VEGFR-3 (flt-4), VEGI, VFM, Viral antigens, VLA, VLA-1, VLA-4, VNR integrin, von Willebrand's factor, WIF-1, WNT1, WNT2, WNT2B/13, WNT3, WNT3A, WNT4, WNT5A, WNT5B, WNT6, WNT7A, WNT7B, WNT8A, WNT8B, WNT9A, WNT9A, WNT9B, WNT10A, WNT10B, WNT11, WNT16, XCL1, XCL2, XCR1, XCR1, XEDAR, XIAP, XPD, CTLA4 (cytotoxic T lymphocyte antigen-4), PD1 (programmed cell death protein 1), PD-L1 (programmed cell death ligand 1), LAG-3 (lymphocyte activation gene-3), TIM-3 (T cell immunoglobulin and mucin protein-3), and hormone receptors; or (2) an antigen selected from the group consisting of: BCMA, CTLA4 (cytotoxic T lymphocyte antigen-4), PD1 (programmed cell death protein 1), PD-L1 (programmed cell death ligand 1), LAG-3 (lymphocyte activation gene-3), TIM-3, CD20, CD2, CD19, Her2, EGFR, EpCAM, FcyRIIIa (CD16), FcyRIIa (CD32a), FcyRIIb (CD32b), FcyRI (CD64), Toll-like receptors (TLRs), TLR4, TLR9, cytokines, IL-2, IL-5, IL-13, IL-6, IL-17, IL-12, IL-23, TNFa, TGFβ, cytokine receptors, IL-2R, chemokines, chemokine receptors, growth factors, VEGF, and HGF; or (3) an antigen selected from CD1a,b,c,d; CD2, CD3, CD4, Cd5,CD6, CD7, CD8, CD9, CD10, CD11a, b, c, d; CDw12, CD13, CD14, oCD15, CD15s, CD15u, CD16, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42a,b,c,d; CD43, CD44, CD45, CD45RO. CD45RA, CD45RB, CD46, CD47, CD48, CD49a, CD49b, CD49c, CD49d, CD49e, CD49f, CD50, CD51, CD52, CD53, CD54, CD55m CD56, CD57, CD58, CD59, CD60a, CD60b, CD61, CD61E, CD62L, CD62P, CD63, CD64, CD65, CD66a, CD66b, CD66c, CD66d,CD66e, CD68, CD69, CD70, CD71, CD72, CD73, CD74, CD75, CD75s, CD77, CD78, CD79α,β, CD80, CD81, CD82, CD83, CDw84, CD85, CD86, CD87, CD88, CD89, CD90, CD91, CD92, Cd92, CD93, CD94, CD95, CD96, CD97, CD98, CD99, CD100, CD101, CD102, CD103, CD104, CD105, CD106, CD107a, CD108, CD109, CD110, CD111, CD112, CD114, CD115, CD116, CD117, CD118, CD119, CD120a, CD120b, CD121a, CDw121b, CD122, CD123, CD124, CD125, CD126, CD127, CDw128, CD129, CD130, CDw131, CD132, CD133, CD134, CD135, CDw136, CDw137, CD138, CD139, CD140a,b, CD141, CD142, CD143, CD144, CD145, CD146, CD147, CD148, CD149, CD150, CD151, CD152, CD153, CD154, CD155, CD156b, CD157, CD158, CD158a, CD159a, CD160, CD161, CD162, CD162R, CD163, CD164, CD165, CD166, CD167a, CD168, CD169, CD170, CD171, CD172a, CD173, CD174, CD175, CD175s, CD176, CD177, CD178, CD179a, CD179b, CD180, CD183, CD184, CD195, CDw197, CD200, CD201, CD202b, CD203c, CD204, CD205, CD206, CD207, CD208, CD209, CDw210, CD212, CD213a1, CD213a2, CDw217, CD220, CD221, CD222, CD223,CD224, CD225, CD226, CD227, CD228, CD229. CD230, CD231, CD232, CD233, CD234, CD235a, CD235b, CD236, CD236R, CD238, CD239, CD240CE, CD240D, CD241, CD242, CD243, CD244, CD245, CD246, CD247 and the like; or (4) an antigen selected from IL4ra, TNFa, BTK, RORgt, PIK3CA, JAK1, JAK3, TYK2 Glut1, Glut3, TAP1, CIITA, cGAS, IRF5, STAT3, STAT6, TAK1 (MAP3K7), HPK1; or any of SOCS1, CD39, Cbl, or PTPN22; or (5) Glut1, PI3K, BTK, TNF, RORC, CD45, or CD39; or (6) any of PD-1, PD-L1, PD-L2, CTLA-4, B7-1, B7-2, LAG-3, HHLA2, TNFRSF12A, HLA-G, NECTIN2, TNFRSF25, TNFSF14, LAIR1, TNFSF15, TNFSF4, KIR2DL4, PDCD1, LGALS9, and PVR.

20. The ARC or ANC of any of the previous claims, which comprises a nucleic acid payload, optionally an RNA, further optionally an siRNA or antisense RNA, having a payload sequence selected from those having the sequences recited in Table 11, Appendix 2 or Appendix 4; or which comprises an INX201 ARC comprising the amino acid sequences and payload sequences recited in in Appendix 2 or which comprises an INX201 ARC having the amino acid sequences and payload sequences recited in Appendix 4.

21. The ARC or ANC of any of the previous claims, which comprises at least 2 different RNA payloads which target the same or different immunomodulatory genes or mRNAs, optionally an immune target recited in any of the previous claims.

22. The ARC or ANC of any of the previous claims, wherein the nucleic acid, optionally RNA payload, is linked to the antibody or antibody fragment via a cleavable or non-cleavable linker.

23. The ARC or ANC of any of the previous claims, which is used to deliver one or more gene editing nucleic acids (e.g., CRISPR guide RNA (gRNA or sgRNA)) and optionally an CRISPR-associated endonuclease or a nucleic acid which encodes for a CRISPR-associated endonuclease.

24. The ARC or ANC of any of the previous claims, which has a PD of at least 1 day, 2 days, 3 days, 4 days, 5 days, a week, 2 weeks, 3 weeks, 4 weeks, 5 weeks, 6 weeks or longer.

25. The ARC or ANC of any of the previous claims, which does not elicit any appreciable toxicity to non-target cells.

26. A composition comprising an ARC or ANC of any of the previous claims, and a pharmaceutically acceptable carrier or excipient, wherein said ARCs or ANCs are optionally comprised in or on a lipid nanoparticle.

27. A method of therapy or prophylaxis, which comprises the administration of an ARC or ANC of any of the previous claims, or a composition containing, to a subject in need thereof.

28. The method of claim 27 wherein the therapy or prophylaxis is for treatment of a neoplastic, proliferative, neurodegenerative, neuroinflammatory, infectious, autoimmune, allergic, or inflammatory condition or a pathologic symptom associated with any of said conditions.

29. The method of claim 27, wherein the therapy or prophylaxis is for the treatment or prevention of an autoimmune disease, e.g., one involving myeloid or T cells.

30. The method of claim 27, wherein the therapy or prophylaxis is for the treatment or prevention of a neoplastic, proliferative, neurodegenerative, neuroinflammatory, infectious, autoimmune or inflammatory disease and/or to prevent or inhibit at least one pathologic symptom associated therewith.

31. The method of claim 27 wherein the therapy or prophylaxis is for the treatment or prevention of an autoimmune disease selected from one or more of Acromegaly, Acquired aplastic anemia, Acquired hemophilia, Agammaglobulinemia, primary, Alopecia areata, Ankylosing spondylitis (AS), Anti-NMDA receptor encephalitis, Antiphospholipid syndrome (APS)|catastrophic antiphospholipid syndrome (CAPS)/Asherson's syndrome, Arteriosclerosis, Autoimmune Addison's disease (AAD), Autoimmune autonomic ganglionopathy (AAG)/autoimmune dysautonomia|autoimmune gastrointestinal dysmotility (AGID), Autoimmune encephalitis|acute disseminated encephalomyelitis (ADEM), Autoimmune gastritis, Autoimmune hemolytic anemia (AIHA), Autoimmune hepatitis (AIH), Autoimmune hyperlipidemia,*Autoimmune hypophysitis, Autoimmune inner ear disease (AIED), Autoimmune lymphoproliferative syndrome (ALPS), Autoimmune myelofibrosis, Autoimmune myocarditis, Autoimmune oophoritis, Autoimmune pancreatitis (AIP), Autoimmune polyglandular syndromes, types I, II, & III (APS type 1, APS type 2, APS type 3, APECED), Autoimmune progesterone dermatitis, Autoimmune retinopathy (AIR), Autoimmune sudden sensorineural hearing loss (SNHL), Balo disease, Behçet's disease, Birdshot chorioretinopathy/birdshot uveitis, Bullous pemphigoid, Castleman disease, Celiac disease, Chagas disease, Chronic inflammatory demyelinating polyneuropathy (CIDP), Chronic urticaria (CU), Churg-Strauss syndrome/eosinophilic granulomatosis with polyangiitis (EGPA),Cogan's syndrome, Cold agglutinin disease, CREST syndrome|limited cutaneous systemic sclerosis, Crohn's disease (CD), Cronkhite-Canada syndrome (CSS), Cryptogenic organizing pneumonia (COP), Dermatitis herpetiformis, Dermatomyositis, Diabetes, type 1, Discoid lupus, Dressler's syndrome/postmyocardial infarction/postpericardiotomy syndrome, Eczema/Atopic Dermatitis, Endometriosis, Eosinophilic esophagitis, Eosinophilic fasciitis, Erythema nodosum, Essential mixed cryoglobulinemia, Evans syndrome, Fibrosing alveolitis/Idiopathic pulmonary fibrosis (IPF), Giant cell arteritis/temporal arteritis/Horton's disease, Giant Cell Myocarditis, Glomerulonephritis, Goodpasture's syndrome/anti-GBM/anti-TBM disease, Granulomatosis with polyangiitis (GPA)/Wegener's granulomatosis, Grave's disease/thyroid eye disease, Guillain-Barre syndrome (GBS), Hashimoto's thyroiditis/chronic lymphocytic thyroiditis/autoimmune thyroiditis, Henoch-Schönlein purpura/IgA vasculitis, Hidradenitis suppurativa, Hurst's disease/acute hemorrhagic leukoencephalitis (AHLE), Hypogammaglobulinemia, IgA nephropathy/Berger's disease, Immune-mediated necrotizing myopathy (IMNM), Immune thrombocytopenia (ITP)/autoimmune thrombocytopenic purpura/autoimmune thrombocytopenia, Inclusion body myositis, IgG4-related sclerosing disease (ISD), Interstitial cystitis, Juvenile idiopathic arthritis/Adult-onset Still's disease. Juvenile polymyositis|Juvenile dermatomyositis|juvenile myositis, Kawasaki disease, Lambert-Eaton myasthenic syndrome (LEMS), Leukocytoclastic vasculitis, Lichen planus, Lichen sclerosus, Ligneous conjunctivitis, Linear IgA disease (LAD)|linear IgA bullous dermatosis (LABD), Lupus nephritis, Lyme disease/chronic Lyme disease/post-treatment Lyme disease syndrome (PTLDS), Lymphocytic colitis/microscopic colitis, Lymphocytic hypophystitis/autoimmune hypophystitis, Meniere's disease, Microscopic polyangiitis (MPA)/ANCA-associated vasculitis, Mixed connective tissue disease (MCTD), Mooren's ulcer, Mucha-Habermann disease, Multifocal motor neuropathy, Multiple sclerosis (MS), Myalgic encephalomyelitis (ME)/Chronic fatigue syndrome (CFS), Myasthenia gravis (MG), Narcolepsy, Neuromyelitis Optica/Devic's disease, Ocular cicatricial pemphigoid, Opsoclonus-myoclonus syndrome (OMS), Palindromic rheumatism, Paraneoplastic cerebellar degeneration, Paraneoplastic pemphigus, Parry-Romberg syndrome (PRS)/Hemifacial atrophy (HFA)/Progressive facial hemiatrophy, Paroxysmal nocturnal hemoglobinuria (PNH), Peripheral uveitis/pars planitis, PANS/PANDAS, Parsonage-Turner syndrome, Pemphigus gestationis/herpes gestationis, Pemphigus foliaceus, Pemphigus vulgaris, Pernicious anemia, POEMS syndrome, Polyarteritis nodosa, Polymyalgia rheumatica, Polymyositis, Postural orthostatic tachycardia syndrome (POTS), Primary biliary cirrhosis (PBC)/primary biliary cholangitis, Primary sclerosing cholangitis (PSC), Psoriasis, Palmoplantar Pustulosis, Psoriatic arthritis, Pulmonary fibrosis, idiopathic (IPF), Pure red cell aplasia (PRCA), Pyoderma gangrenosum, Rasmussen's encephalitis, Raynaud's syndrome/phenomenon, Reactive arthritis/Reiter's syndrome, Reflex sympathetic dystrophy syndrome (RSD)/Complex regional pain syndrome (CRPS), Relapsing polychondritis, Restless leg syndrome (RLS)/Willis-Ekbom disease, Rheumatic fever, Rheumatoid arthritis, Sarcoidosis, Schmidt syndrome/autoimmune polyendocrine syndrome type II, Scleritis, Scleroderma, Sclerosing Mesenteritis/Mesenteric Panniculitis, Serpiginous choroidopathy, Sjogren's syndrome, Stiff person syndrome (SPS), Small fiber sensory neuropathy, Systemic lupus erythematosus (SLE), Subacute bacterial endocarditis (SBE), Subacute cutaneous lupus, Susac syndrome, Sydenham's chorea, Sympathetic ophthalmia, Takayasu's arteritis (vasculitis), Testicular autoimmunity (vasculitis, orchitis), Tolosa-Hunt syndrome, Transverse myelitis (TM), Tubulointerstitial nephritis uveitis syndrome (TINU), Ulcerative colitis (UC), Undifferentiated connective tissue disease (UCTD), Uveitis|anterior/intermediate/posterior, Vasculitis, VEXAS Syndrome, Vitiligo, and Vogt-Koyanagi-Harada syndrome (VKH), and/or to prevent or inhibit at least one pathologic symptom associated therewith.

32. The method of claim 27 wherein the therapy or prophylaxis is for the treatment or prevention of Addison disease, arthritis, celiac disease, lupus, Grave's disease, myasthenia gravis, multiple sclerosis, ITP, rheumatoid arthritis, colitis, inflammatory bowel disease, pernicious anemia, Hashimoto's thyroiditis Sjogren's disease, asthma, type 2 diabetes, and autoimmune type I diabetes and/or to prevent or inhibit at least one pathologic symptom associated therewith.

33. The method of claim 27 wherein the therapy or prophylaxis is for the treatment of an inflammatory disease selected from the group consisting of Fatty liver disease, Endometriosis, Type 2 diabetes, mellitus, Type 1 diabetes mellitus, Inflammatory bowel disease (IBD), Asthma, Rheumatoid arthritis, asthma, Obesity, Fibromyalgia, Lupus SLE, osteoarthritis, Rheumatoid Arthritis, Shingles Herpes Zoster, and Vasculitis and/or to prevent or inhibit at least one pathologic symptom associated therewith.

34. The method of claim 27 wherein the therapy or prophylaxis is for the treatment or prevention of a neurodegenerative or neuroinflammatory disease, e.g., Alzheimer's disease, Amyotrophic lateral sclerosis, Friedreich ataxia, Huntington's disease, Lewy body disease, aphasia, Parkinson's disease or Spinal muscular atrophy and/or to prevent or inhibit at least one pathologic symptom associated therewith.

35. The method of claim 27 wherein the therapy or prophylaxis is for the treatment of cancer or to prevent cancer reoccurrence and/or to inhibit least one pathologic symptom associated with specific immune cell types.

36. The method of claim 27 wherein the therapy or prophylaxis is for the treatment of a solid tumor and/or to prevent or inhibit at least one pathologic symptom associated therewith.

37. The method of claim 27 wherein the therapy or prophylaxis is for the treatment of a hematologic malignancy and/or to prevent or inhibit at least one pathologic symptom associated therewith.

38. The method of claim 27 wherein the therapy or prophylaxis is for a relapsed or refractory cancer, or a metastatic cancer, optionally a relapsed or refractory solid tumor, or a metastatic solid tumor, a relapsed or refractory hematologic malignancy, or a metastatic hematologic malignancy.

39. The method of claim 27 wherein the therapy or prophylaxis is for a solid tumor selected from anal cancer, appendix cancer, bile duct cancer (i.e., cholangiocarcinoma), bladder cancer, brain tumor, breast cancer, cervical cancer, colon cancer, cancer of Unknown Primary (CUP), esophageal cancer, eye cancer, fallopian tube cancer, gastroenterological cancer, kidney cancer, liver cancer, lung cancer, medulloblastoma, melanoma, oral cancer, ovarian cancer, pancreatic cancer, parathyroid disease, penile cancer, pituitary tumor, prostate cancer, rectal cancer, skin cancer, stomach cancer, testicular cancer, throat cancer, thyroid cancer, uterine cancer, vaginal cancer, or vulvar cancer and/or to prevent or inhibit at least one pathologic symptom associated therewith.

40. The method of claim 27 wherein the therapy or prophylaxis is for a hematologic malignancy, optionally a leukemia, a lymphoma, a myeloma, a non-Hodgkin's lymphoma, or a Hodgkin's lymphoma. In some instances, the hematologic malignancy comprises chronic lymphocytic leukemia (CLL), small lymphocytic lymphoma (SLL), high risk CLL, a non-CLL/SLL lymphoma, prolymphocytic leukemia (PLL), follicular lymphoma (FL), diffuse large B-cell lymphoma (DLBCL), mantle cell lymphoma (MCL), Waldenström's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis and/or to prevent or inhibit at least one pathologic symptom associated therewith.

41. The method of claim 27 wherein the therapy or prophylaxis is for a hematologic malignancy selected from chronic lymphocytic leukemia (CLL), small lymphocytic lymphoma (SLL), high risk CLL, a non-CLL/SLL lymphoma, prolymphocytic leukemia (PLL), follicular lymphoma (FL), diffuse large B-cell lymphoma (DLBCL), mantle cell lymphoma (MCL), Waldenström's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some cases, the hematologic malignancy is a relapsed or refractory hematologic malignancy, or a metastatic hematologic malignancy and/or to prevent or inhibit at least one pathologic symptom associated therewith.

42. The method of claim 27 wherein the therapy or prophylaxis is for treatment of an autoimmune disease selected from the group consisting of Addison disease, arthritis, celiac disease, lupus, Grave's disease, myasthenia gravis, multiple sclerosis, ITP, rheumatoid arthritis, colitis, inflammatory bowel disease, pernicious anemia, Hashimoto's thyroiditis Sjogren's disease, asthma, type 2 diabetes, and autoimmune type I diabetes and/or to prevent or inhibit at least one pathologic symptom associated therewith.

43. The method of claim 27 wherein the therapy or prophylaxis is for treatment of cancer, and the ARC or ANC comprises a nucleic acid, optionally an ASO or siRNA which modulates or blocks the expression of any of PD-1, PD-L1, PD-L2, CTLA-4, B7-1, B7-2, LAG-3, HHLA2, TNFRSF12A, HLA-G, NECTIN2, TNFRSF25, TNFSF14, LAIR1, TNFSF15, TNFSF4, KIR2DL4, PDCD1, LGALS9, VSIr (VISTA) and PVR.

44. The method of claim 27 wherein the therapy or prophylaxis is for inhibiting or treating immune senescence associated with disease or aging, and the ARC or ANC optionally comprises a nucleic acid, further optionally an ASO or siRNA which modulates or blocks the expression of any of PD-1, PD-L1, PD-L2, CTLA-4, B7-1, B7-2, LAG-3, HHLA2, TNFRSF12A, HLA-G, NECTIN2, TNFRSF25, TNFSF14, LAIR1, TNFSF15, TNFSF4, KIR2DL4, PDCD1, LGALS9, VSIr (VISTA) and PVR.


SEQUENCE LISTING (870 sequences)

SEQ ID NO: 1-99 - skipped (99 entries)

SEQ ID NO: 100 - protein, 8 aa, Mus musculus
GYTLTDYN

SEQ ID NO: 101 - protein, 8 aa, Mus musculus
INLNYAIT

SEQ ID NO: 102 - protein, 14 aa, Mus musculus
ARGGYRYTYYAMDY

SEQ ID NO: 103 - protein, 6 aa, Mus musculus
ENIYSN

SEQ ID NO: 104 - skipped

SEQ ID NO: 105 - protein, 9 aa, Mus musculus
QHFWGTPRT

SEQ ID NO: 106 - protein, 121 aa, Mus musculus
EVQLQQFGAELVKPGASVKISCKASGYTLTDYNMDWVKQSHGKSLEWIGHINLNYAITTYNQKFKGKATLTVDKSSSTAYMELRSLTSEDTAVYYCARGGYRYTYYAMDYWGQGTSVTVSS

SEQ ID NO: 107 - protein, 222 aa, Mus musculus
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKLNPKS

SEQ ID NO: 108 - protein, 107 aa, Mus musculus
DIQMTQSPASLSVSVGETVTITCRASENIYSNLAWYQQKQGKSPQLLVYAATNLADGVPSRFSGSGSGTQYSLKINSLQSEDFGSYYCQHFWGTPRTFGGGTKLEIK

SEQ ID NO: 109 - protein, 107 aa, Mus musculus
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 110 - protein, 8 aa, Homo sapiens
GYTLTDYN

SEQ ID NO: 111 - protein, 8 aa, Homo sapiens
INLNYAIT

SEQ ID NO: 112 - protein, 14 aa, Homo sapiens
ARGGYRYTYYAMDY

SEQ ID NO: 113 - protein, 6 aa, Homo sapiens
ENIYSN

SEQ ID NO: 114 - skipped

SEQ ID NO: 115 - protein, 9 aa, Homo sapiens
QHFWGTPRT

SEQ ID NO: 116 - protein, 121 aa, Homo sapiens; identical to SEQ ID NO: 106

SEQ ID NO: 117 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 118 - protein, 107 aa, Homo sapiens; identical to SEQ ID NO: 108

SEQ ID NO: 119 - protein, 107 aa, Homo sapiens [sequence omitted]

SEQ ID NO: 120 - protein, 8 aa, Mus musculus
GYSFTGYT

SEQ ID NO: 121 - protein, 8 aa, Mus musculus
INPYNGGI

SEQ ID NO: 122 - protein, 13 aa, Mus musculus
ARRTLLRPYFFDY

SEQ ID NO: 123 - protein, 10 aa, Mus musculus
QSVSTSTFSY

SEQ ID NO: 124 - skipped

SEQ ID NO: 125 - protein, 9 aa, Mus musculus
QHSWEIPYT

SEQ ID NO: 126 - protein, 120 aa, Mus musculus
EVQLQQSGPELVKPGTSMKISCKASGYSFTGYTMNWVRQSHGKNLEWIGLINPYNGGINYNQKFKARATLTVDKSSSTAYMELLSLTSEDSAVYYCARRTLLRPYFFDYWGQGTTLTVSS

SEQ ID NO: 127 - protein, 120 aa, Mus musculus; identical to SEQ ID NO: 126

SEQ ID NO: 128 - protein, 111 aa, Mus musculus
DIVLTQSPASLAVSLGQRATISCRASQSVSTSTFSYMHWYQQKPGQPPKLLIKYASNLESGVPARFSGSGSGTDFTLNIHPVEEEDTATYYCQHSWEIPYTFGGGTKLEIK

SEQ ID NO: 129 - protein, 111 aa, Mus musculus; identical to SEQ ID NO: 128

SEQ ID NO: 130 - protein, 8 aa, Homo sapiens
GYSFTGYT

SEQ ID NO: 131 - protein, 8 aa, Homo sapiens
INPYNGGI

SEQ ID NO: 132 - protein, 13 aa, Homo sapiens
ARRTLLRPYFFDY

SEQ ID NO: 133 - protein, 10 aa, Homo sapiens
QSVSTSTFSY

SEQ ID NO: 134 - skipped

SEQ ID NO: 135 - protein, 9 aa, Homo sapiens
QHSWEIPYT

SEQ ID NO: 136 - protein, 120 aa, Homo sapiens; identical to SEQ ID NO: 126

SEQ ID NO: 137 - protein, 326 aa, Homo sapiens; identical to SEQ ID NO: 117

SEQ ID NO: 138 - protein, 111 aa, Homo sapiens; identical to SEQ ID NO: 128

SEQ ID NO: 139 - protein, 107 aa, Homo sapiens [sequence omitted]

SEQ ID NO: 140 - protein, 8 aa, Mus musculus
GFDFSRYW

SEQ ID NO: 141 - protein, 8 aa, Mus musculus
VYPDSSTI

SEQ ID NO: 142 - protein, 7 aa, Mus musculus
ARGRGDY

SEQ ID NO: 143 - protein, 6 aa, Mus musculus
GNIHNY

SEQ ID NO: 144 - skipped

SEQ ID NO: 145 - protein, 9 aa, Mus musculus
QNFWSTPFT

SEQ ID NO: 146 - protein, 114 aa, Mus musculus
EVKLLESGGGLVQPGGSLKLSCAASGFDFSRYWMSWVRQAPGKGLEWIGEVYPDSSTINYTPSLKDKFIISRDNAKNTLYLQMIKVRSEDTALYYCARGRGDYWGQGTSVTVSS

SEQ ID NO: 147 - protein, 326 aa, Mus musculus; identical to SEQ ID NO: 117

SEQ ID NO: 148 - protein, 107 aa, Mus musculus
DIQMTQSPASLSASVGETVTITCRASGNIHNYLSWYHQKQGKSPQLLVYNAKTLADGVPSRFSGSGSGTQYSLKINSLQPEDFGSYYCQNFWSTPFTFGSGTKLEIK

SEQ ID NO: 149 - protein, 107 aa, Mus musculus [sequence omitted]

SEQ ID NO: 150 - protein, 8 aa, Homo sapiens
GFDFSRYW

SEQ ID NO: 151 - protein, 8 aa, Homo sapiens
VYPDSSTI

SEQ ID NO: 152 - protein, 7 aa, Homo sapiens
ARGRGDY

SEQ ID NO: 153 - protein, 6 aa, Homo sapiens
GNIHNY

SEQ ID NO: 154 - skipped

SEQ ID NO: 155 - protein, 9 aa, Homo sapiens
QNFWSTPFT

SEQ ID NO: 156 - protein, 114 aa, Homo sapiens; identical to SEQ ID NO: 146

SEQ ID NO: 157 - protein, 326 aa, Homo sapiens; identical to SEQ ID NO: 117

SEQ ID NO: 158 - protein, 107 aa, Homo sapiens; identical to SEQ ID NO: 148

SEQ ID NO: 159 - protein, 107 aa, Homo sapiens [sequence omitted]

SEQ ID NO: 160 - protein, 8 aa, Homo sapiens
GFDFSRYW

SEQ ID NO: 161 - protein, 8 aa, Homo sapiens
VYPDSSTI

SEQ ID NO: 162 - protein, 7 aa, Homo sapiens
ARGRGDY

SEQ ID NO: 163 - protein, 6 aa, Homo sapiens
GNIHNY

SEQ ID NO: 164 - skipped

SEQ ID NO: 165 - protein, 9 aa, Homo sapiens
QNFWSTPFT

SEQ ID NO: 166 - protein, 114 aa, Homo sapiens; identical to SEQ ID NO: 146

SEQ ID NO: 167 - protein, 326 aa, Homo sapiens
ASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 168 - protein, 107 aa, Homo sapiens; identical to SEQ ID NO: 148

SEQ ID NO: 169 - protein, 107 aa, Homo sapiens [sequence omitted]

SEQ ID NO: 170 - protein, 8 aa, Homo sapiens
GYSFTSYW

SEQ ID NO: 171 - protein, 8 aa, Homo sapiens
IYPGDSDT

SEQ ID NO: 172 - protein, 16 aa, Homo sapiens
ARDVSSFYGYSPMFDY

SEQ ID NO: 173 - protein, 6 aa, Homo sapiens
QSVSSY

SEQ ID NO: 174 - skipped

SEQ ID NO: 175 - protein, 9 aa, Homo sapiens
QQRSNWPLT

SEQ ID NO: 176 - protein, 123 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARDVSSFYGYSPMFDYWGQGTLVTVSS

SEQ ID NO: 177 - protein, 330 aa, Homo sapiens [sequence omitted]

SEQ ID NO: 178 - protein, 107 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGQGTKVEIK

SEQ ID NO: 179 - protein, 107 aa, Homo sapiens [sequence omitted]

SEQ ID NO: 180 - protein, 8 aa, Homo sapiens
GYSFTSYW

SEQ ID NO: 181 - protein, 8 aa, Homo sapiens
IYPGDSDT

SEQ ID NO: 182 - protein, 16 aa, Homo sapiens
ARDAHSFYGYSALLDY

SEQ ID NO: 183 - protein, 7 aa, Homo sapiens
QSVSSSY

SEQ ID NO: 184 - skipped

SEQ ID NO: 185 - protein, 9 aa, Homo sapiens
QQYGSSPLT

SEQ ID NO: 186 - protein, 123 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARDAHSFYGYSALLDYWGQGTLVTVSS

SEQ ID NO: 187 - protein, 330 aa, Homo sapiens [sequence omitted]

SEQ ID NO: 188 - protein, 108 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGQGTKVEIK

SEQ ID NO: 189 - protein, 107 aa, Homo sapiens [sequence omitted]

SEQ ID NO: 190 - protein, 8 aa, Homo sapiens
GYSFTSYW

SEQ ID NO: 191 - protein, 8 aa, Homo sapiens
IYPGDSDT

SEQ ID NO: 192 - protein, 16 aa, Homo sapiens
ARDSYSFYGHTPVLDY

SEQ ID NO: 193 - protein, 7 aa, Homo sapiens
QSVSSSY

SEQ ID NO: 194 - skipped

SEQ ID NO: 195 - protein, 9 aa, Homo sapiens
QQYGSSPLT

SEQ ID NO: 196 - protein, 123 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARDSYSFYGHTPVLDYWGQGTLVTVSS

SEQ ID NO: 197 - protein, 330 aa, Homo sapiens [sequence omitted]

SEQ ID NO: 198 - protein, 108 aa, Homo sapiens; identical to SEQ ID NO: 188

SEQ ID NO: 199 - protein, 107 aa, Homo sapiens [sequence omitted]

SEQ ID NO: 200 - protein, 8 aa, Homo sapiens
GYSFTSYW

SEQ ID NO: 201 - protein, 8 aa, Homo sapiens
IYPGDSDT

SEQ ID NO: 202 - protein, 14 aa, Homo sapiens
ARDDALYGGYYLDY

SEQ ID NO: 203 - protein, 12 aa, Homo sapiens
QSVLYSSNNKNY

SEQ ID NO: 204 - skipped

SEQ ID NO: 205 - protein, 9 aa, Homo sapiens
QQYYSTPLT

SEQ ID NO: 206 - protein, 121 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARDDALYGGYYLDYWGQGTLVTVSS

SEQ ID NO: 207 - protein, 330 aa, Homo sapiens [sequence omitted]

SEQ ID NO: 208 - protein, 113 aa, Homo sapiens
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPLTFGQGTKVEIK

SEQ ID NO: 209 - protein, 107 aa, Homo sapiens [sequence omitted]

SEQ ID NO: 210 - protein, 8 aa, Homo sapiens
GYSFTSYW

SEQ ID NO: 211 - protein, 8 aa, Homo sapiens
IYPGDSDT

SEQ ID NO: 212 - protein, 16 aa, Homo sapiens
ARDANSFYSAASIFDY

SEQ ID NO: 213 - protein, 12 aa, Homo sapiens
QSVLYSSNNKNY

SEQ ID NO: 214 - skipped

SEQ ID NO: 215 - protein, 9 aa, Homo sapiens
QQYYSTPLT

SEQ ID NO: 216 - protein, 123 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARDANSFYSAASIFDYWGQGTLVTVSS

SEQ ID NO: 217 - protein, 330 aa, Homo sapiens [sequence omitted]

SEQ ID NO: 218 - protein, 113 aa, Homo sapiens; identical to SEQ ID NO: 208

SEQ ID NO: 219 - protein, 107 aa, Homo sapiens [sequence omitted]

SEQ ID NO: 220 - protein, 8 aa, Homo sapiens
GGTFSSYA

SEQ ID NO: 221 - protein, 8 aa, Homo sapiens
IIPIFGTA

SEQ ID NO: 222 - protein, 13 aa, Homo sapiens
ARSSYGWSYEFDY

SEQ ID NO: 223 - protein, 6 aa, Homo sapiens
QSIATN

SEQ ID NO: 224 - skipped

SEQ ID NO: 225 - protein, 9 aa, Homo sapiens
QQNDDRPIT

SEQ ID NO: 226 - protein, 120 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARSSYGWSYEFDYWGQGTLVTVSS

SEQ ID NO: 227 - protein, 330 aa, Homo sapiens [sequence omitted]

SEQ ID NO: 228 - protein, 107 aa, Homo sapiens
DIQMTQSPSSLSASVGDRVTITCRASQSIATNLNWYQQKPGKAPKLLIYAASSLQSGVPSRFSGSGSGTDFTLTISSLQPEDFATYYCQQNDDRPITFGQGTKVEIK

SEQ ID NO: 229 - protein, 107 aa, Homo sapiens [sequence omitted]

SEQ ID NO: 230 - protein, 8 aa, Homo sapiens
GGTFSSYA

SEQ ID NO: 231 - protein, 8 aa, Homo sapiens
IIPIFGTA

SEQ ID NO: 232 - protein, 13 aa, Homo sapiens
ARSSYGWSYEFDY

SEQ ID NO: 233 - protein, 6 aa, Homo sapiens
QSIRTD

SEQ ID NO: 234 - skipped

SEQ ID NO: 235 - protein, 9 aa, Homo sapiens
QQNERTPIT

SEQ ID NO: 236 - protein, 120 aa, Homo sapiens; identical to SEQ ID NO: 226

SEQ ID NO: 237 - protein, 330 aa, Homo sapiens [sequence omitted]

SEQ ID NO: 238 - protein, 107 aa, Homo sapiens
DIQMTQSPSSLSASVGDRVTITCRASQSIRTDLNWYQQKPGKAPKLLIYSASSLQSGVPSRFSGSGSGTDFTLTISSLQPEDFATYYCQQNERTPITFGQGTKVEIK

SEQ ID NO: 239 - protein, 107 aa, Homo sapiens [sequence omitted]

SEQ ID NO: 240 - protein, 8 aa, Homo sapiens
GGTFSSYA

SEQ ID NO: 241 - protein, 8 aa, Homo sapiens
IIPIFGTA

SEQ ID NO: 242 - protein, 13 aa, Homo sapiens
ARSSYGWSYEFDY

SEQ ID NO: 243 - protein, 6 aa, Homo sapiens
QSINND

SEQ ID NO: 244 - skipped

SEQ ID NO: 245 - protein, 9 aa, Homo sapiens
QQNRATPIT

SEQ ID NO: 246 - protein, 120 aa, Homo sapiens; identical to SEQ ID NO: 226

SEQ ID NO: 247 - protein, 330 aa, Homo sapiens [sequence omitted]

SEQ ID NO: 248 - protein, 107 aa, Homo sapiens
DIQMTQSPSSLSASVGDRVTITCRASQSINNDLNWYQQKPGKAPKLLIYAASSLQSGVPSRFSGSGSGTDFTLTISSLQPEDFATYYCQQNRATPITFGQGTKVEIK

SEQ ID NO: 249 - protein, 107 aa, Homo sapiens [sequence omitted]

SEQ ID NO: 250 - protein, 8 aa, Homo sapiens
GGTFSSYA

SEQ ID NO: 251 - protein, 8 aa, Homo sapiens
IIPIFGTA

SEQ ID NO: 252 - protein, 13 aa, Homo sapiens
ARNTFGWSGELDY

SEQ ID NO: 253 - protein, 6 aa, Homo sapiens
QSISNR

SEQ ID NO: 254 - skipped

SEQ ID NO: 255 - protein, 9 aa, Homo sapiens
QQNHDNPIT

SEQ ID NO: 256 - protein, 120 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARNTFGWSGELDYWGQGTLVTVSS

SEQ ID NO: 257 - protein, 330 aa, Homo sapiens [sequence omitted]

SEQ ID NO: 258 - protein, 107 aa, Homo sapiens
DIQMTQSPSSLSASVGDRVTITCRASQSISNRLNWYQQKPGKAPKLLIYSASSLQSGVPSRFSGSGSGTDFTLTISSLQPEDFATYYCQQNHDNPITFGQGTKVEIK

SEQ ID NO: 259 - protein, 107 aa, Homo sapiens [sequence omitted]

SEQ ID NO: 260 - protein, 8 aa, Homo sapiens
GGTFSSYA

SEQ ID NO: 261 - protein, 8 aa, Homo sapiens
IIPIFGTA

SEQ ID NO: 262 - protein, 13 aa, Homo sapiens
ARSSYGWSYEFDY

SEQ ID NO: 263 - protein, 6 aa, Homo sapiens
QSIATY

SEQ ID NO: 264 - skipped

SEQ ID NO: 265 - protein, 9 aa, Homo sapiens
QQNHNRPIT

SEQ ID NO: 266 - protein, 120 aa, Homo sapiens; identical to SEQ ID NO: 226

SEQ ID NO: 267 - protein, 330 aa, Homo sapiens [sequence omitted]

SEQ ID NO: 268 - protein, 107 aa, Homo sapiens
DIQMTQSPSSLSASVGDRVTITCRASQSIATYLNWYQQKPGKAPKLLIYAASSLQSGVPSRFSGSGSGTDFTLTISSLQPEDFATYYCQQNHNRPITFGQGTKVEIK

SEQ ID NO: 269 - protein, 107 aa, Homo sapiens [sequence omitted]

SEQ ID NO: 270 - protein, 8 aa, Homo sapiens
GGTFSSYA

SEQ ID NO: 271 - protein, 8 aa, Homo sapiens
IIPIFGTA

SEQ ID NO: 272 - protein, 13 aa, Homo sapiens
ARHSIGWVAELDY

SEQ ID NO: 273 - protein, 6 aa, Homo sapiens
QSINTD

SEQ ID NO: 274 - skipped

SEQ ID NO: 275 - protein, 9 aa, Homo sapiens
QQGASDPIT

SEQ ID NO: 276 - protein, 120 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARHSIGWVAELDYWGQGTLVTVSS

SEQ ID NO: 277 - protein, 330 aa, Homo sapiens [sequence omitted]

SEQ ID NO: 278 - protein, 107 aa, Homo sapiens
DIQMTQSPSSLSASVGDRVTITCRASQSINTDLNWYQQKPGKAPKLLIYAASSLQSGVPSRFSGSGSGTDFTLTISSLQPEDFATYYCQQGASDPITFGQGTKVEIK

SEQ ID NO: 279 - protein, 107 aa, Homo sapiens [sequence omitted]

SEQ ID NO: 280 - protein, 8 aa, Homo sapiens
GGTFSSYA

SEQ ID NO: 281 - protein, 8 aa, Homo sapiens
IIPIFGTA

SEQ ID NO: 282 - protein, 13 aa, Homo sapiens
ARSSYGWSYEFDY

SEQ ID NO: 283 - protein, 6 aa, Homo sapiens
QSINTD

SEQ ID NO: 284 - skipped

SEQ ID NO: 285 - protein, 9 aa, Homo sapiens
QQNRGSPIT

SEQ ID NO: 286 - protein, 120 aa, Homo sapiens; identical to SEQ ID NO: 226

SEQ ID NO: 287 - protein, 330 aa, Homo sapiens [sequence omitted]

SEQ ID NO: 288 - protein, 107 aa, Homo sapiens
DIQMTQSPSSLSASVGDRVTITCRASQSINTDLNWYQQKPGKAPKLLIYAASSLQSGVPSRFSGSGSGTDFTLTISSLQPEDFATYYCQQNRGSPITFGQGTKVEIK

SEQ ID NO: 289 - protein, 107 aa, Homo sapiens [sequence omitted]

SEQ ID NO: 290 - protein, 8 aa, Homo sapiens
GGTFSSYA

SEQ ID NO: 291 - protein, 8 aa, Homo sapiens
IIPIFGTA

SEQ ID NO: 292 - protein, 13 aa, Homo sapiens
ARHSIGWVAELDY

SEQ ID NO: 293 - protein, 6 aa, Homo sapiens
QSIATD

SEQ ID NO: 294 - skipped

SEQ ID NO: 295 - protein, 9 aa, Homo sapiens
QQAHWYPLT

SEQ ID NO: 296 - protein, 120 aa, Homo sapiens; identical to SEQ ID NO: 276

SEQ ID NO: 297 - protein, 330 aa, Homo sapiens [sequence omitted]

SEQ ID NO: 298 - protein, 107 aa, Homo sapiens
DIQMTQSPSSLSASVGDRVTITCRASQSIATDLNWYQQKPGKAPKLLIYAASSLQSGVPSRFSGSGSGTDFTLTISSLQPEDFATYYCQQAHWYPLTFGQGTKVEIK

SEQ ID NO: 299 - protein, 107 aa, Homo sapiens [sequence omitted]

SEQ ID NO: 300 - protein, 8 aa, Homo sapiens
GGTFSSYA

SEQ ID NO: 301 - protein, 8 aa, Homo sapiens
IIPIFGTA

SEQ ID NO: 302 - protein, 13 aa, Homo sapiens
ARHSIGWVAELDY

SEQ ID NO: 303 - protein, 6 aa, Homo sapiens
QSIATS

SEQ ID NO: 304 - skipped

SEQ ID NO: 305 - protein, 9 aa, Homo sapiens
QQGAYYPLT

SEQ ID NO: 306 - protein, 120 aa, Homo sapiens; identical to SEQ ID NO: 276

SEQ ID NO: 307 - protein, 330 aa, Homo sapiens [sequence omitted]

SEQ ID NO: 308 - protein, 107 aa, Homo sapiens
DIQMTQSPSSLSASVGDRVTITCRASQSIATSLNWYQQKPGKAPKLLIYYASSLQSGVPSRFSGSGSGTDFTLTISSLQPEDFATYYCQQGAYYPLTFGQGTKVEIK

SEQ ID NO: 309 - protein, 107 aa, Homo sapiens [sequence omitted]

SEQ ID NO: 310 - protein, 8 aa, Homo sapiens
GGTFSSYA

SEQ ID NO: 311 - protein, 8 aa, Homo sapiens
IIPIFGTA

SEQ ID NO: 312 - protein, 13 aa, Homo sapiens
ARSSYGWSYEFDY

SEQ ID NO: 313 - protein, 6 aa, Homo sapiens
QSIRTY

SEQ ID NO: 314 - skipped

SEQ ID NO: 315 - protein, 9 aa, Homo sapiens
QQAYSNPIT

SEQ ID NO: 316 - protein, 120 aa, Homo sapiens; identical to SEQ ID NO: 226

SEQ ID NO: 317 - protein, 330 aa, Homo sapiens [sequence omitted]

SEQ ID NO: 318 - protein, 107 aa, Homo sapiens
DIQMTQSPSSLSASVGDRVTITCRASQSIRTYLNWYQQKPGKAPKLLIYAASSLQSGVPSRFSGSGSGTDFTLTISSLQPEDFATYYCQQAYSNPITFGQGTKVEIK

SEQ ID NO: 319 - protein, 107 aa, Homo sapiens [sequence omitted]

SEQ ID NO: 320 - protein, 8 aa, Homo sapiens
GGTFSSYA

SEQ ID NO: 321 - protein, 8 aa, Homo sapiens
IIPIFGTA

SEQ ID NO: 322 - protein, 13 aa, Homo sapiens
ARSSYGWSYEFDY

SEQ ID NO: 323 - protein, 6 aa, Homo sapiens
QSINTN

SEQ ID NO: 324 - skipped

SEQ ID NO: 325 - protein, 9 aa, Homo sapiens
QQARDTPIT

SEQ ID NO: 326 - protein, 120 aa, Homo sapiens; identical to SEQ ID NO: 226

SEQ ID NO: 327 - protein, 330 aa, Homo sapiens [sequence omitted]

SEQ ID NO: 328 - protein, 107 aa, Homo sapiens
DIQMTQSPSSLSASVGDRVTITCRASQSINTNLNWYQQKPGKAPKLLIYAASSLQSGVPSRFSGSGSGTDFTLTISSLQPEDFATYYCQQARDTPITFGQGTKVEIK

SEQ ID NO: 329 - protein, 107 aa, Homo sapiens [sequence omitted]

SEQ ID NO: 330 - protein, 8 aa, Homo sapiens
GYTFPSHT

SEQ ID NO: 331 - protein, 8 aa, Homo sapiens
IYPFIDST

SEQ ID NO: 332 - protein, 11 aa, Homo sapiens
ARGIRGYTMDY

SEQ ID NO: 333 - protein, 10 aa, Homo sapiens
ESVDNYGLSF

SEQ ID NO: 334 - skipped

SEQ ID NO: 335 - protein, 9 aa, Homo sapiens
QQSKEVPYT

SEQ ID NO: 336 - protein, 118 aa, Homo sapiens
QVQLVQSGAEVKKPGASVKVSCKASGYTFPSHTIHWVRQAPGQRLEWMGSIYPFIDSTIYNQKFKDRVTITRDTSASTAYMELSSLRSEDTAVYYCARGIRGYTMDYWGQGTLVTVSS

SEQ ID NO: 337 - protein, 330 aa, Homo sapiens [sequence omitted]

SEQ ID NO: 338 - protein, 111 aa, Homo sapiens
DVVMTQSPLSLPVTLGQPASISCRASESVDNYGLSFMNWFQQRPGQSPRRLIYGASNQGSGVPDRFSGSGSGTDFTLKISRVEAEDVGVYYCQQSKEVPYTFGQGTKLEIK

SEQ ID NO: 339 - protein, 107 aa, Homo sapiens [sequence omitted]

SEQ ID NO: 340 - protein, 8 aa, Homo sapiens
GYTFTNYG

SEQ ID NO: 341 - protein, 8 aa, Homo sapiens
INPYTGEP

SEQ ID NO: 342 - protein, 12 aa, Homo sapiens
AREGYGNYIFPY

SEQ ID NO: 343 - protein, 10 aa, Homo sapiens
ESVDTYANSL

SEQ ID NO: 344 - skipped

SEQ ID NO: 345 - protein, 9 aa, Homo sapiens
QQTNEDPRT

SEQ ID NO: 346 - protein, 119 aa, Homo sapiens
QVQLVQSGSELKKPGASVKVSCKASGYTFTNYGLNWVRQAPGQGLEWMGWINPYTGEPTYADDFKGRFVFSLDTSVSTAYLQICSLKAEDTAVYYCAREGYGNYIFPYWGQGTLVTVSS

SEQ ID NO: 347 - protein, 330 aa, Homo sapiens [sequence omitted]

SEQ ID NO: 348 - protein, 111 aa, Homo sapiens
DIVMTQTPLSLSVTPGQPASISCRASESVDTYANSLMHWYLQKPGQPPQLLIYRASNLESGVPDRFSGSGSGTDFTLKISRVEAEDVGVYYCQQTNEDPRTFGQGTKLEIK

SEQ ID NO: 349 - protein, 107 aa, Homo sapiens [sequence omitted]

SEQ ID NO: 350 - protein, 8 aa, Homo sapiens
GFTFSSYT

SEQ ID NO: 351 - protein, 8 aa, Homo sapiens
ISNSGSYT

SEQ ID NO: 352 - protein, 11 aa, Homo sapiens
ARDTVLSPFDY

SEQ ID NO: 353 - protein, 5 aa, Homo sapiens
SSISY

SEQ ID NO: 354 - skipped

SEQ ID NO: 355 - protein, 7 aa, Homo sapiens
HQRSSFT

SEQ ID NO: 356 - protein, 118 aa, Homo sapiens
EVQLVESGGGLVQPGGSLRLSCAASGFTFSSYTMSWVRQAPGKGLEWVATISNSGSYTYYLDSVKGRFTISRDNAKNSLYLQMNSLRAEDTAVYYCARDTVLSPFDYWGQGTTVTVSS

SEQ ID NO: 357 - protein, 330 aa, Homo sapiens [sequence omitted]

SEQ ID NO: 358 - protein, 104 aa, Homo sapiens
EIVLTQSPDFQSVTPKEKVTITCSATSSISYMHWYQQKPDQSPKLLIKDTSELASGVPSRFSGSGSGTDFTLTINSLEAEDAATYYCHQRSSFTFGQGTKLEIK

SEQ ID NO: 359 - protein, 107 aa, Homo sapiens [sequence omitted]

SEQ ID NO: 360 - protein, 8 aa, Homo sapiens
GGTFSNYW

SEQ ID NO: 361 - protein, 8 aa, Homo sapiens
IYPGGGFT

SEQ ID NO: 362 - protein, 14 aa, Homo sapiens
ARYYRSDEDYSMDF

SEQ ID NO: 363 - protein, 11 aa, Homo sapiens
QSLLYSNGKTY

SEQ ID NO: 364 - skipped

SEQ ID NO: 365 - protein, 9 aa, Homo sapiens
VQATHFPQT

SEQ ID NO: 366 - protein, 121 aa, Homo sapiens
QVQLVQSGAEVKKPGSSVKVSCKASGGTFSNYWIGWVRQAPGQGLEWMGEIYPGGGFTHYNEKFKGRVTITADESTSTAYMELSSLRSEDTAVYYCARYYRSDEDYSMDFWGQGTLVTVSS

SEQ ID NO: 367 - protein, 330 aa, Homo sapiens [sequence omitted]

SEQ ID NO: 368 - protein, 112 aa, Homo sapiens
DVVMTQSPLSLPVTLGQPASISCKSSQSLLYSNGKTYLNWFQQRPGQSPRRLIYLVSKLDSGVPDRFSGSGSGTDFTLKISRVEAEDVGVYYCVQATHFPQTFGQGTKLEIK

SEQ ID NO: 369 - protein, 107 aa, Homo sapiens [sequence omitted]

SEQ ID NO: 370 - protein, 8 aa, Homo sapiens
GYTFTHYT

SEQ ID NO: 371 - protein, 8 aa, Homo sapiens
IIPSSGYS

SEQ ID NO: 372 - protein, 16 aa, Homo sapiens
ARGAYDDYYDYYAMDY

SEQ ID NO: 373 - protein, 11 aa, Homo sapiens
QTIVHSNGNTY

SEQ ID NO: 374 - skipped

SEQ ID NO: 375 - protein, 9 aa, Homo sapiens
FQASHVPWT

SEQ ID NO: 376 - protein, 123 aa, Homo sapiens
QVQLVQSGAEVKKPGASVKVSCKASGYTFTHYTIHWVRQAPGQGLEWMGYIIPSSGYSEYNQKFKDRVTMTRDTSTSTVYMELSSLRSEDTAVYYCARGAYDDYYDYYAMDYWGQGTLVTVSS

SEQ ID NO: 377 - protein, 330 aa, Homo sapiens [sequence omitted]

SEQ ID NO: 378 - protein, 112 aa, Homo sapiens
DIVMTQSPLSLPVTPGEPASISCRSSQTIVHSNGNTYLEWYLQKPGQSPQLLIYKVSNRFSGVPDRFSGSGSGTDFTLKISRVEAEDVGVYYCFQASHVPWTFGQGTKLEIK

SEQ ID NO: 379 - protein, 107 aa, Homo sapiens [sequence omitted]

SEQ ID NO: 380 - protein, 8 aa, Homo sapiens
GYNIKDTY

SEQ ID NO: 381 - protein, 8 aa, Homo sapiens
IDPTHGYV

SEQ ID NO: 382 - protein, 13 aa, Homo sapiens
ARDRFDPYWFLDV

SEQ ID NO: 383 - protein, 10 aa, Homo sapiens
ESVEYYGTSL

SEQ ID NO: 384 - skipped

SEQ ID NO: 385 - protein, 9 aa, Homo sapiens
QQSRKVPWT

SEQ ID NO: 386 - protein, 120 aa, Homo sapiens
QVQLVQSGAEVKKPGASVKVSCKASGYNIKDTYMHWVRQAPGQGLEWMGRIDPTHGYVIYDPKFQGRVTMTRDTSTSTVYMELSSLRSEDTAVYYCARDRFDPYWFLDVWGQGTLVTVSS

SEQ ID NO: 387 - protein, 330 aa, Homo sapiens [sequence omitted]

SEQ ID NO: 388 - protein, 111 aa, Homo sapiens
DIVMTQTPLSLSVTPGQPASISCRASESVEYYGTSLMQWYLQKPGQSPQLLIYDAFNVESGVPDRFSGSGSGTDFTLKISRVEAEDVGVYYCQQSRKVPWTFGQGTKLEIK

SEQ ID NO: 389 - protein, 107 aa, Homo sapiens [sequence omitted]

SEQ ID NO: 390 - protein, 9 aa, Homo sapiens
GYSIASDYV

SEQ ID NO: 391 - protein, 7 aa, Homo sapiens
ISYSGST

SEQ ID NO: 392 - protein, 17 aa, Homo sapiens
ARITTVVPTGSYYGVDF

SEQ ID NO: 393 - protein, 5 aa, Homo sapiens
SSVNF

SEQ ID NO: 394 - skipped

SEQ ID NO: 395 - protein, 9 aa, Homo sapiens
QQWSNYPFT

SEQ ID NO: 396 - protein, 124 aa, Homo sapiens
QVQLQESGPGLVKPSETLSLTCAVSGYSIASDYVWNWIRQPPGKGLEWIGYISYSGSTSNNPSLNSRVTISVDTSKNQFSLKLSSVTAADTAVYYCARITTVVPTGSYYGVDFWGQGTTVTVSS

SEQ ID NO: 397 - protein, 330 aa, Homo sapiens [sequence omitted]

SEQ ID NO: 398 - protein, 106 aa, Homo sapiens
EIVLTQSPDFQSVTPKEKVTITCSGSSSVNFMYWYQQKPDQSPKLLIKDTSNLASGVPSRFSGSGSGTDFTLTINSLEAEDAATYYCQQWSNYPFTFGQGTKLEIK

SEQ ID NO: 399 - protein, 107 aa, Homo sapiens [sequence omitted]

SEQ ID NO: 400 - protein, 8 aa, Homo sapiens
GYTFTSYW

SEQ ID NO: 401 - protein, 8 aa, Homo sapiens
IIPNTLHT

SEQ ID NO: 402 - protein, 13 aa, Homo sapiens
ARLDGDYDYALDY

SEQ ID NO: 403 - protein, 10 aa, Homo sapiens
ESVEYYGTSF

SEQ ID NO: 404 - skipped

SEQ ID NO: 405 - protein, 9 aa, Homo sapiens
QQSRKVPYT

SEQ ID NO: 406 - protein, 120 aa, Homo sapiens
QVQLVQSGAEVKKPGASVKVSCKASGYTFTSYWLHWVRQAPGQGLEWMGYIIPNTLHTDYNQKFKDRVTMTRDTSTSTVYMELSSLRSEDTAVYYCARLDGDYDYALDYWGQGTLVTVSS

SEQ ID NO: 407 - protein, 330 aa, Homo sapiens [sequence omitted]

SEQ ID NO: 408 - protein, 111 aa, Homo sapiens
DIVMTQTPLSLSVTPGQPASISCRASESVEYYGTSFMQWYLQKPGQPPQLLIYTASNVESGVPDRFSGSGSGTDFTLKISRVEAEDVGVYYCQQSRKVPYTFGQGTKLEIK

SEQ ID NO: 409 - protein, 107 aa, Homo sapiens [sequence omitted]

SEQ ID NO: 410 - protein, 8 aa, Homo sapiens
GFTFSTYA

SEQ ID NO: 411 - protein, 8 aa, Homo sapiens
ISSGGSDT

SEQ ID NO: 412 - protein, 14 aa, Homo sapiens
ARPTYYGIFSYFDY

SEQ ID NO: 413 - protein, 5 aa, Homo sapiens
SSVSY

SEQ ID NO: 414 - skipped

SEQ ID NO: 415 - protein, 8 aa, Homo sapiens
HQWRTYPT

SEQ ID NO: 416 - protein, 121 aa, Homo sapiens
EVQLVESGGGLVQPGGSLRLSCAASGFTFSTYAMSWVRQAPGKGLEWVATISSGGSDTYYPDTVKGRFTISRDNAKNSLYLQMNSLRAEDTAVYYCARPTYYGIFSYFDYWGQGTLVTVSS

SEQ ID NO: 417 - protein, 330 aa, Homo sapiens [sequence omitted]

SEQ ID NO: 418 - protein, 105 aa, Homo sapiens
DIQLTQSPSFLSASVGDRVTITCSASSSVSYMHWYQQKPGKAPKLLIYSTSNLASGVPSRFSGSGSGTEFTLTISSLQPEDFATYYCHQWRTYPTFGQGTKLEIK

SEQ ID NO: 419 - protein, 107 aa, Homo sapiens [sequence omitted]

SEQ ID NO: 420 - protein, 8 aa, Homo sapiens
GFSLTNYG

SEQ ID NO: 421 - protein, 7 aa, Homo sapiens
IWRGGNT

SEQ ID NO: 422 - protein, 11 aa, Homo sapiens
ARSMVSYTVDY

SEQ ID NO: 423 - protein, 5 aa, Homo sapiens
SSVSY

SEQ ID NO: 424 - skipped

SEQ ID NO: 425 - protein, 9 aa, Homo sapiens
QQWSSYPPT

SEQ ID NO: 426 - protein, 117 aa, Homo sapiens
QVQLQESGPGLVKPSETLSLTCTVSGFSLTNYGVHWIRQPPGKGLEWIGVIWRGGNTDYNAAFMSRVTISVDTSKNQFSLKLSSVTAADTAVYYCARSMVSYTVDYWGQGTLVTVSS

SEQ ID NO: 427 - protein, 330 aa, Homo sapiens [sequence omitted]

SEQ ID NO: 428 - protein, 106 aa, Homo sapiens
EIVLTQSPDFQSVTPKEKVTITCSASSSVSYMHWYQQKPDQSPKLLIKDTSNLASGVPSRFSGSGSGTDFTLTINSLEAEDAATYYCQQWSSYPPTFGQGTKLEIK

SEQ ID NO: 429 - protein, 107 aa, Homo sapiens [sequence omitted]

SEQ ID NO: 430 - protein, 8 aa, Homo sapiens
GYTFTDYW

SEQ ID NO: 431 - protein, 8 aa, Homo sapiens
IETSLNYP

SEQ ID NO: 432 - protein, 13 aa, Homo sapiens
ARWGIYGNPWFAY

SEQ ID NO: 433 - protein, 10 aa, Homo sapiens
ESVDSYVNSF

SEQ ID NO: 434 - skipped

SEQ ID NO: 435 - protein, 9 aa, Homo sapiens
QQSNEDPYT

SEQ ID NO: 436 - protein, 120 aa, Homo sapiens
QVQLVQSGAEVKKPGASVKVSCKASGYTFTDYWMHWVRQAPGQGLEWMGAIETSLNYPSYNQKFKGRVTMTRDTSTSTVYMELSSLRSEDTAVYYCARWGIYGNPWFAYWGRGTLVTVSS

SEQ ID NO: 437 - protein, 330 aa, Homo sapiens [sequence omitted]

SEQ ID NO: 438 - protein, 111 aa, Homo sapiens
DIVMTQTPLSLSVTPGQPASISCRASESVDSYVNSFVHWYLQKPGQPPQLLIYRASNLESGVPDRFSGSGSGTDFTLKISRVEAEDVGVYYCQQSNEDPYTFGQGTKLEIK

SEQ ID NO: 439 - protein, 107 aa, Homo sapiens [sequence omitted]

SEQ ID NO: 440 - protein, 8 aa, Homo sapiens
GYTFTNYG

SEQ ID NO: 441 - protein, 8 aa, Homo sapiens
INTYTGES

SEQ ID NO: 442 - protein, 12 aa, Homo sapiens
ARDYYGIYVSAY

SEQ ID NO: 443 - protein, 10 aa, Homo sapiens
ESVDNYANSF

SEQ ID NO: 444 - skipped

SEQ ID NO: 445 - protein, 9 aa, Homo sapiens
QQSHEDPYT

SEQ ID NO: 446 - protein, 119 aa, Homo sapiens
QVQLVQSGSELKKPGASVKVSCKASGYTFTNYGMTWVRQAPGQGLEWMGWINTYTGESTYADDFKGRFVFSLDTSVSTAYLQICSLKAEDTAVYYCARDYYGIYVSAYWGQGTLVTVSS

SEQ ID NO: 447 - protein, 330 aa, Homo sapiens [sequence omitted]

SEQ ID NO: 448 - protein, 111 aa, Homo sapiens
DIVMTQTPLSLSVTPGQPASISCRASESVDNYANSFMHWYLQKPGQSPQLLIYRASNLESGVPDRFSGSGSGTDFTLKISRVEAEDVGVYYCQQSHEDPYTFGQGTKLEIK

SEQ ID NO: 449 - protein, 107 aa, Homo sapiens [sequence omitted]

SEQ ID NO: 450 - protein, 8 aa, Homo sapiens
GYTFTSHW

SEQ ID NO: 451 - protein, 8 aa, Homo sapiens
INPRDGRT

SEQ ID NO: 452 - protein, 16 aa, Homo sapiens
ARGDFHYGDYFWYFDV

SEQ ID NO: 453 - protein, 6 aa, Homo sapiens
QNVHGA

SEQ ID NO: 454 - skipped

SEQ ID NO: 455 - protein, 8 aa, Homo sapiens
LQHWNYLT

SEQ ID NO: 456 - protein, 123 aa, Homo sapiens
QVQLVQSGAEVKKPGASVKVSCKASGYTFTSHWMHWVRQAPGQGLEWMGEINPRDGRTNYNEKFKTRVTMTRDTSTSTVYMELSSLRSEDTAVYYCARGDFHYGDYFWYFDVWGQGTLVTVSS

SEQ ID NO: 457 - protein, 330 aa, Homo sapiens [sequence omitted]

SEQ ID NO: 458 - protein, 106 aa, Homo sapiens
DIQMTQSPSSLSASVGDRVTITCKASQNVHGAVAWYQQKPGKVPKLLIYMASNRPTGVPSRFSGSGSGTDFTLTISSLQPEDVATYYCLQHWNYLTFGQGTKLEIK

SEQ ID NO: 459 - protein, 107 aa, Homo sapiens [sequence omitted]

SEQ ID NO: 460 - protein, 8 aa, Homo sapiens
GYTFTHYW

SEQ ID NO: 461 - protein, 8 aa, Homo sapiens
IYPGDGDT

SEQ ID NO: 462 - protein, 10 aa, Homo sapiens
ARRDYDYGDY

SEQ ID NO: 463 - protein, 5 aa, Homo sapiens
SSVSH

SEQ ID NO: 464 - skipped

SEQ ID NO: 465 - protein, 9 aa, Homo sapiens
QQYQTYPPT

SEQ ID NO: 466 - protein, 117 aa, Homo sapiens
QVQLVQSGAEVKKPGASVKVSCKASGYTFTHYWIQWVRQAPGQGLEWMGAIYPGDGDTRYTQKFKGRVTMTRDTSTSTVYMELSSLRSEDTAVYYCARRDYDYGDYWGQGTTVTVSS

SEQ ID NO: 467 - protein, 330 aa, Homo sapiens [sequence omitted]

SEQ ID NO: 468 - protein, 106 aa, Homo sapiens
EIVLTQSPDFQSVTPKEKVTITCSASSSVSHMYWYQQKPDQSPKLLIKLTSNLASGVPSRFSGSGSGTDFTLTINSLEAEDAATYYCQQYQTYPPTFGQGTRLEIK

SEQ ID NO: 469 - protein, 107 aa, Homo sapiens [sequence omitted]

SEQ ID NO: 470 - protein, 9 aa, Homo sapiens
GFSITSDFA

SEQ ID NO: 471 - protein, 7 aa, Homo sapiens
ITYSGFT

SEQ ID NO: 472 - protein, 14 aa, Homo sapiens
ARQEYGNYVWYFDV

SEQ ID NO: 473 - protein, 10 aa, Homo sapiens
ESVEYYGTSL

SEQ ID NO: 474 - skipped

SEQ ID NO: 475 - protein, 9 aa, Homo sapiens
QQSRKVPWT

SEQ ID NO: 476 - protein, 121 aa, Homo sapiens
QVQLQESGPGLVKPSETLSLTCAVSGFSITSDFAWNWIRQPPGKGLEWIGYITYSGFTNYNPSLESRVTISVDTSKNQFSLKLSSVTAADTAVYYCARQEYGNYVWYFDVWGQGTLVTVSS

SEQ ID NO: 477 - protein, 330 aa, Homo sapiens [sequence omitted]

SEQ ID NO: 478 - protein, 111 aa, Homo sapiens
DIVMTQTPLSLSVTPGQPASISCRASESVEYYGTSLLQWYLQKPGQPPQLLIYAASNVESGVPDRFSGSGSGTDFTLKISRVEAEDVGVYYCQQSRKVPWTFGQGTKLEIK

SEQ ID NO: 479 - protein, 107 aa, Homo sapiens [sequence omitted]

SEQ ID NO: 480 - protein, 9 aa, Homo sapiens
GYSITSGYF

SEQ ID NO: 481 - protein, 7 aa, Homo sapiens
MGYDGRI

SEQ ID NO: 482 - protein, 14 aa, Homo sapiens
AREGDYYGSGFAYW

SEQ ID NO: 483 - protein, 6 aa, Homo sapiens
QNVNTN

SEQ ID NO: 484 - skipped

SEQ ID NO: 485 - protein, 120 aa, Homo sapiens
QVQLQESGPGLVKPSETLSLTCAVSGYSITSGYFWNWIRQPPGKGLEWIGYMGYDGRIFYSPSLKNRVTISVDTSKNQFSLKLSSVTAADTAVYYCAREGDYYGSGFAYWGQGTLVTVSS

SEQ ID NO: 486 - protein, 120 aa, Homo sapiens; identical to SEQ ID NO: 485

SEQ ID NO: 487 - protein, 330 aa, Homo sapiens [sequence omitted]

SEQ ID NO: 488 - protein, 107 aa, Homo sapiens
DIQMTQSPSSLSASVGDRVTITCKASQNVNTNLAWYQQKPGKVPKLLIYSASYRYSGVPSRFSGSGSGTDFTLTISSLQPEDVATYYCQQYNNYPLTFGQGTKLEIK

SEQ ID NO: 489 - protein, 107 aa, Homo sapiens [sequence omitted]

SEQ ID NO: 490 - protein, 8 aa, Homo sapiens
GYTFTSYW

SEQ ID NO: 491 - protein, 8 aa, Homo sapiens
INPSNGLT

SEQ ID NO: 492 - protein, 15 aa, Homo sapiens
ARSYDYDGDYYAMDY

SEQ ID NO: 493 - protein, 11 aa, Homo sapiens
QSIVHSNGNTY

SEQ ID NO: 494 - skipped

SEQ ID NO: 495 - protein, 9 aa, Homo sapiens
FQASHVPWT

SEQ ID NO: 496 - protein, 122 aa, Homo sapiens
QVQLVQSGAEVKKPGASVKVSCKASGYTFTSYWIHWVRQAPGQGLEWMGEINPSNGLTNYNEKFRNRVTMTRDTSTSTVYMELSSLRSEDTAVYYCARSYDYDGDYYAMDYWGQGTLVTVSS

SEQ ID NO: 497 - protein, 330 aa, Homo sapiens [sequence omitted]

SEQ ID NO: 498 - protein, 112 aa, Homo sapiens
DIVMTQSPLSLPVTPGEPASISCRSTQSIVHSNGNTYLEWYLQKPGQSPQLLIYKVSNRFSGVPDRFSGSGSGTDFTLKISRVEAEDVGVYYCFQASHVPWTFGQGTKLEIK

SEQ ID NO: 499 - protein, 107 aa, Homo sapiens [sequence omitted]

SEQ ID NO: 500 - protein, 8 aa, Homo sapiens
GYPFTGYF

SEQ ID NO: 501 - protein, 8 aa, Homo sapiens
INPYNGGT

SEQ ID NO: 502 - protein, 12 aa, Homo sapiens
ARWTFDGLFMDY

SEQ ID NO: 503 - protein, 10 aa, Homo sapiens
ENVDKYGISF

SEQ ID NO: 504 - skipped

SEQ ID NO: 505 - protein, 9 aa, Homo sapiens
QQSKEDPYT

SEQ ID NO: 506 - protein, 119 aa, Homo sapiens
QVQLVQSGAEVKKPGASVKVSCKASGYPFTGYFMHWVRQAPGQGLEWMGRINPYNGGTVYNQNFNDRVTMTRDTSTSTVYMELSSLRSEDTAVYYCARWTFDGLFMDYWGQGTTVTVSS

SEQ ID NO: 507 - protein, 330 aa, Homo sapiens [sequence omitted]

SEQ ID NO: 508 - protein, 111 aa, Homo sapiens
DVVMTQSPLSLPVTLGQPASISCRASENVDKYGISFMNWFQQRPGQSPRRLIYATSNQGSGVPDRFSGSGSGTDFTLKISRVEAEDVGVYYCQQSKEDPYTFGQGTKLEIK

SEQ ID NO: 509 - protein, 107 aa, Homo sapiens [sequence omitted]

SEQ ID NO: 510 - protein, 8 aa, Homo sapiens
DYIFSSYW

SEQ ID NO: 511 - protein, 8 aa, Homo sapiens
IFPGSGGT

SEQ ID NO: 512 - protein, 15 aa, Homo sapiens
ARAIYYDYDMYYFDS

SEQ ID NO: 513 - protein, 6 aa, Homo sapiens
QDISSY

SEQ ID NO: 514 - skipped

SEQ ID NO: 515 - protein, 9 aa, Homo sapiens
QHVNTLPWT

SEQ ID NO: 516 - protein, 122 aa, Homo sapiens
QVQLVQSGAEVKKPGASVKVSCKASDYIFSSYWIQWVRQAPGQGLEWMGEIFPGSGGTNYNEKFKGRVTMTRDTSTSTVYMELSSLRSEDTAVYYCARAIYYDYDMYYFDSWGQGTTVTVSS

SEQ ID NO: 517 - protein, 330 aa, Homo sapiens [sequence omitted]

SEQ ID NO: 518 - protein, 107 aa, Homo sapiens
DIQMTQSPSSLSASVGDRVTITCRASQDISSYLNWYQQKPGKVPKLLIYYTSRLHSGVPSRFSGSGSGTDFTLTISSLQPEDVATYYCQHVNTLPWTFGQGTKLEIK

SEQ ID NO: 519 - protein, 107 aa, Homo sapiens [sequence omitted]

SEQ ID NO: 520 - protein, 8 aa, Homo sapiens
GYTFTDYT

SEQ ID NO: 521 - protein, 8 aa, Homo sapiens
INPYNGGT

SEQ ID NO: 522 - protein, 13 aa, Homo sapiens
ARHYGNYNWYFDV

SEQ ID NO: 523 - protein, 6 aa, Homo sapiens
QYVNTA

SEQ ID NO: 524 - skipped

SEQ ID NO: 525 - protein, 9 aa, Homo sapiens
QQHFTTPIT

SEQ ID NO: 526 - protein, 120 aa, Homo sapiens
QVQLVQSGAEVKKPGASVKVSCKASGYTFTDYTMNWVRQAPGQRLEWMGLINPYNGGTTYNQKFKGRVTITRDTSASTAYMELSSLRSEDTAVYYCARHYGNYNWYFDVWGQGTTVTVSS

SEQ ID NO: 527 - protein, 330 aa, Homo sapiens [sequence omitted]

SEQ ID NO: 528 - protein, 107 aa, Homo sapiens
DIQLTQSPSFLSASVGDRVTITCKASQYVNTAVAWYQQKPGKAPKLLIYSASYRYTGVPSRFSGSGSGTEFTLTISSLQPEDFATYYCQQHFTTPITFGQGTKLEIK

SEQ ID NO: 529 - protein, 107 aa, Homo sapiens [sequence omitted]

SEQ ID NO: 530 - protein, 8 aa, Homo sapiens
GYTFTNYG

SEQ ID NO: 531 - protein, 8 aa, Homo sapiens
INTYTGEP

SEQ ID NO: 532 - protein, 10 aa, Homo sapiens
ARNYGNYVAY

SEQ ID NO: 533 - protein, 6 aa, Homo sapiens
QDINSY

SEQ ID NO: 534 - skipped

SEQ ID NO: 535 - protein, 9 aa, Homo sapiens
LQYDEFPLT

SEQ ID NO: 536 - protein, 117 aa, Homo sapiens
QVQLVQSGSELKKPGASVKVSCKASGYTFTNYGMNWVRQAPGQGLEWMGWINTYTGEPTYADDFKGRFVFSLDTSVSTAYLQICSLKAEDTAVYYCARNYGNYVAYWGQGTTVTVSS

SEQ ID NO: 537 - protein, 330 aa, Homo sapiens [sequence omitted]

SEQ ID NO: 538 - protein, 107 aa, Homo sapiens
DIQMTQSPSSLSASVGDRVTITCKASQDINSYLSWFQQKPGKAPKSLIYRANRLVDGVPSRFSGSGSGTDFTLTISSLQPEDFATYYCLQYDEFPLTFGQGTRLEIK

SEQ ID NO: 539 - protein, 107 aa, Homo sapiens [sequence omitted]

SEQ ID NO: 540 - protein, 8 aa, Homo sapiens
GYTFSNYW

SEQ ID NO: 541 - protein, 8 aa, Homo sapiens
ILPGSGNV

SEQ ID NO: 542 - protein, 16 aa, Homo sapiens
ATPPHYYGYDYYDVNY

SEQ ID NO: 543 - protein, 11 aa, Homo sapiens
QSLLDSDGKTY

SEQ ID NO: 544 - skipped

SEQ ID NO: 545 - protein, 9 aa, Homo sapiens
WQGTHFPQT

SEQ ID NO: 546 - protein, 123 aa, Homo sapiens
EVQLVQSGAEVKKPGATVKISCKVSGYTFSNYWIEWVQQAPGKGLEWMGEILPGSGNVHYNEKFKGRVTITADTSTDTAYMELSSLRSEDTAVYYCATPPHYYGYDYYDVNYWGQGTLVTVSS

SEQ ID NO: 547 - protein, 330 aa, Homo sapiens [sequence omitted]

SEQ ID NO: 548 - protein, 112 aa, Homo sapiens
DVVMTQSPLSLPVTLGQPASISCKSSQSLLDSDGKTYLNWFQQRPGQSPRRLIYLVSELDSGVPDRFSGSGSGTDFTLKISRVEAEDVGVYYCWQGTHFPQTFGQGTKLEIK

SEQ ID NO: 549 - protein, 107 aa, Homo sapiens [sequence omitted]

SEQ ID NO: 550 - protein, 8 aa, Homo sapiens
GYVFSRSW

SEQ ID NO: 551 - protein, 8 aa, Homo sapiens
IYPGDGDT

SEQ ID NO: 552 - protein, 6 aa, Homo sapiens
AARWFL

SEQ ID NO: 553 - protein, 12 aa, Homo sapiens
QSLLNSGDQKSY

SEQ ID NO: 554 - skipped

SEQ ID NO: 555 - protein, 9 aa, Homo sapiens
QNDHSYPLT

SEQ ID NO: 556 - protein, 113 aa, Homo sapiens
QVQLVQSGAEVKKPGSSVKVSCKASGYVFSRSWINWVRQAPGQGLEWMGRIYPGDGDTNYNGKFKGRVTITADESTSTAYMELSSLRSEDTAVYYCARRWFLWGQGTLVTVSS

SEQ ID NO: 557 - protein, 330 aa, Homo sapiens [sequence omitted]

SEQ ID NO: 558 - protein, 113 aa, Homo sapiens
DIVMTQSPDSLAVSLGERATINCKSSQSLLNSGDQKSYLAWYQQKPGQPPKLLIYGASTRESGVPDRFSGSGSGTDFTLTISSLQAEDVAVYYCQNDHSYPLTFGQGTKLEIK

SEQ ID NO: 559 - protein, 107 aa, Homo sapiens [sequence omitted]

SEQ ID NO: 560 - protein, 8 aa, Homo sapiens
GFTFSSYA

SEQ ID NO: 561 - protein, 8 aa, Homo sapiens
ISSGGSHT

SEQ ID NO: 562 - protein, 11 aa, Homo sapiens
ARRGNLYDGPY

SEQ ID NO: 563 - protein, 5 aa, Homo sapiens
SSVSH

SEQ ID NO: 564 - skipped

SEQ ID NO: 565 - protein, 9 aa, Homo sapiens
QQWNFYPFT

SEQ ID NO: 566 - protein, 118 aa, Homo sapiens
EVQLVESGGGLVQPGGSLRLSCAASGFTFSSYAMSWVRQAPGKGLEWVAEISSGGSHTYYPDTVTGRFTISRDNAKNSLYLQMNSLRAEDTAVYYCARRGNLYDGPYWGQGTLVTVSS

SEQ ID NO: 567 - protein, 330 aa, Homo sapiens [sequence omitted]

SEQ ID NO: 568 - protein, 106 aa, Homo sapiens
EIVLTQSPATLSLSPGERATLSCSASSSVSHMYWYQQKPGQAPRLLIYDTSKLASGIPARFSGSGSGTDFTLTISSLEPEDFAVYYCQQWNFYPFTFGQGTKLEIK

SEQ ID NO: 569 - protein, 107 aa, Homo sapiens [sequence omitted]

SEQ ID NO: 570 - protein, 8 aa, Homo sapiens
GYTFTDYV

SEQ ID NO: 571 - protein, 8 aa, Homo sapiens
IYPGSGNT

SEQ ID NO: 572 - protein, 10 aa, Homo sapiens
ARVLVSVMDY

SEQ ID NO: 573 - protein, 5 aa, Homo sapiens
SSINY

SEQ ID NO: 574 - skipped

SEQ ID NO: 575 - protein, 9 aa, Homo sapiens
HQRSSYPWT

SEQ ID NO: 576 - protein, 117 aa, Homo sapiens
QVQLVQSGAEVKKPGASVKVSCKASGYTFTDYVISWVRQAPGQGLEWMGEIYPGSGNTYYYEKFKGRVTMTRDTSTSTVYMELSSLRSEDTAVYYCARVLVSVMDYWGQGTLVTVSS

SEQ ID NO: 577 - protein, 330 aa, Homo sapiens [sequence omitted]

SEQ ID NO: 578 - protein, 106 aa, Homo sapiens
EIVLTQSPDFQSVTPKEKVTITCSASSSINYITWYQQKPDQSPKLLIKDTSKLASGVPSRFSGSGSGTDFTLTINSLEAEDAATYYCHQRSSYPWTFGQGTRLEIK

SEQ ID NO: 579 - protein, 107 aa, Homo sapiens [sequence omitted]

SEQ ID NO: 580 - protein, 8 aa, Homo sapiens
GYIFTDYN

SEQ ID NO: 581 - protein, 8 aa, Homo sapiens
INPKYDST

SEQ ID NO: 582 - protein, 10 aa, Homo sapiens
AADGSSAMDY

SEQ ID NO: 583 - protein, 12 aa, Homo sapiens
QSLLNSGHQKNY

SEQ ID NO: 584 - skipped

SEQ ID NO: 585 - protein, 9 aa, Homo sapiens
QNDHRYPLT

SEQ ID NO: 586 - protein, 117 aa, Homo sapiens
QMQLVQSGPEVKKPGTSVKVSCKASGYIFTDYNIDWVRQARGQRLEWIGDINPKYDSTRYNQKFKGRVTITRDMSTSTAYMELSSLRSEDTAVYYCAADGSSAMDYWGQGTTVTVSS

SEQ ID NO: 587 - protein, 330 aa, Homo sapiens [sequence omitted]

SEQ ID NO: 588 - protein, 113 aa, Homo sapiens
DIVMTQSPDSLAVSLGERATINCKSSQSLLNSGHQKNYLAWYQQKPGQPPKLLIYGASTRESGVPDRFSGSGSGTDFTLTISSLQAEDVAVYYCQNDHRYPLTFGQGTKLEIK

SEQ ID NO: 589 - protein, 107 aa, Homo sapiens [sequence omitted]

SEQ ID NO: 590 - protein, 8 aa, Homo sapiens
GFNIKDYY

SEQ ID NO: 591 - protein, 8 aa, Homo sapiens
IDPENGNT

SEQ ID NO: 592 - protein, 10 aa, Homo sapiens
ARDYGYFDYW

SEQ ID NO: 593 - protein, 5 aa, Homo sapiens
SSVIY

SEQ ID NO: 594 - skipped

SEQ ID NO: 595 - protein, 9 aa, Homo sapiens
QQRSSYPHT

SEQ ID NO: 596 - protein, 116 aa, Homo sapiens
QVQLVQSGAEVKKPGSSVKVSCKASGFNIKDYYIHWVRQAPGQGLEWMGWIDPENGNTIYDPKFQGRVTITADESTSTAYMELSSLRSEDTAVYYCARDYGYFDYWGQGTLVTVSS

SEQ ID NO: 597 - protein, 330 aa, Homo sapiens [sequence omitted]

SEQ ID NO: 598 - protein, 106 aa, Homo sapiens
DVVMTQSPAFLSVTPGEKVTITCSAGSSVIYMYWYQQKPDQAPKLLIKSTSNLASGVPSRFSGSGSGTDFTFTISSLEAEDAATYYCQQRSSYPHTFGQGTKLEIK

SEQ ID NO: 599 - protein, 107 aa, Homo sapiens [sequence omitted]

SEQ ID NO: 600 - protein, 8 aa, Homo sapiens
GYTFTDYW

SEQ ID NO: 601 - protein, 8 aa, Homo sapiens
IYPSHSYT

SEQ ID NO: 602 - protein, 14 aa, Homo sapiens
ARGGYRYPYYAMDY

SEQ ID NO: 603 - protein, 6 aa, Homo sapiens
ENIYGA

SEQ ID NO: 604 - skipped

SEQ ID NO: 605 - protein, 9 aa, Homo sapiens
QNVLSTPYT

SEQ ID NO: 606 - protein, 121 aa, Homo sapiens
QVQLVQSGAEVKKPGASVKVSCKASGYTFTDYWINWVRQAPGQGLEWMGNIYPSHSYTNYNQEFKDRVTMTRDTSTSTVYMELSSLRSEDTAVYYCARGGYRYPYYAMDYWGQGTTVTVSS

SEQ ID NO: 607 - protein, 330 aa, Homo sapiens [sequence omitted]

SEQ ID NO: 608 - protein, 107 aa, Homo sapiens
AIQLTQSPSSLSASVGDRVTITCGASENIYGALNWYQQKPGKAPKLLIYGATNLADGVPSRFSGSGSGTDFTLTISSLQPEDFATYYCQNVLSTPYTFGQGTKLEIK

SEQ ID NO: 609 - protein, 107 aa, Homo sapiens [sequence omitted]

SEQ ID NO: 610 - protein, 8 aa, Homo sapiens
GYTFTNYW

SEQ ID NO: 611 - protein, 8 aa, Homo sapiens
INPSSGYT

SEQ ID NO: 612 - protein, 11 aa, Homo sapiens
ARDGGSVLFGY

SEQ ID NO: 613 - protein, 12 aa, Homo sapiens
QSLLNSGIRKNY

SEQ ID NO: 614 - skipped

SEQ ID NO: 615 - protein, 8 aa, Homo sapiens
KQSYNLYT

SEQ ID NO: 616 - protein, 118 aa, Homo sapiens
QVQLVQSGAEVKKPGASVKVSCKASGYTFTNYWMHWVRQAPGQGLEWMGYINPSSGYTEYNQKFKDRVTMTRDTSTSTVYMELSSLRSEDTAVYYCARDGGSVLFGYWGQGTTVTVSS

SEQ ID NO: 617 - protein, 330 aa, Homo sapiens [sequence omitted]

SEQ ID NO: 618 - protein, 112 aa, Homo sapiens
DIVMTQSPDSLAVSLGERATINCKSSQSLLNSGIRKNYLAWYQQKPGQPPKLLIYSASTRESGVPDRFSGSGSGTDFTLTISSLQAEDVAVYYCKQSYNLYTFGQGTKLEIK

SEQ ID NO: 619 - protein, 107 aa, Homo sapiens [sequence omitted]

SEQ ID NO: 620 - protein, 9 aa, Homo sapiens
AYSITSDYA

SEQ ID NO: 621 - protein, 7 aa, Homo sapiens
ITYSGST

SEQ ID NO: 622 - protein, 6 aa, Homo sapiens
ARSFGY

SEQ ID NO: 623 - protein, 6 aa, Homo sapiens
QSIGTS

SEQ ID NO: 624 - skipped

SEQ ID NO: 625 - protein, 7 aa, Homo sapiens
QQINSWP

SEQ ID NO: 626 - protein, 113 aa, Homo sapiens
QVQLQESGPGLVKPSETLSLTCAVSAYSITSDYAWNWIRQPPGKGLEWIGYITYSGSTRYNPSLKSRVTISVDTSKNQFSLKLSSVTAADTAVYYCARSFGYWGQGTTVTVSS

SEQ ID NO: 627 - protein, 330 aa, Homo sapiens [sequence omitted]

SEQ ID NO: 628 - protein, 107 aa, Homo sapiens
EIVMTQSPATLSVSPGERATLSCRASQSIGTSIHWYQQKPGQAPRLLIYYASESISGIPARFSGSGSGTEFTLTISSLQSEDFAVYYCQQINSWPTTFGQGTKLEIK

SEQ ID NO: 629 - protein, 107 aa, Homo sapiens [sequence omitted]

SEQ ID NO: 630 - protein, 8 aa, Homo sapiens
GFNIKDYY

SEQ ID NO: 631 - protein, 8 aa, Homo sapiens
IDPENGDT

SEQ ID NO: 632 - protein, 10 aa, Homo sapiens
ARDMGSSYVY

SEQ ID NO: 633 - protein, 5 aa, Homo sapiens
SSVIY

SEQ ID NO: 634 - skipped

SEQ ID NO: 635 - protein, 9 aa, Homo sapiens
QQRSSYPFT

SEQ ID NO: 636 - protein, 117 aa, Homo sapiens
QVQLVQSGAEVKKPGASVKVSCKASGFNIKDYYMHWVRQAPGQGLEWMGWIDPENGDTEYAPKFQDRVTMTRDTSTSTVYMELSSLRSEDTAVYYCARDMGSSYVYWGRGTLVTVSS

SEQ ID NO: 637 - protein, 330 aa, Homo sapiens [sequence omitted]

SEQ ID NO: 638 - protein, 106 aa, Homo sapiens
DVVMTQSPAFLSVTPGEKVTITCSASSSVIYMHWYQQKPDQAPKLLIKSTSNLASGVPSRFSGSGSGTDFTFTISSLEAEDAATYYCQQRSSYPFTFGQGTKLEIK

SEQ ID NO: 639 - protein, 107 aa, Homo sapiens [sequence omitted]

SEQ ID NO: 640 - protein, 10 aa, Homo sapiens
GFSLSTSGMG

SEQ ID NO: 641 - protein, 7 aa, Homo sapiens
IYWDDDK

SEQ ID NO: 642 - protein, 13 aa, Homo sapiens
ARRPDYFGSSYVD

SEQ ID NO: 643 - protein, 10 aa, Homo sapiens
ESVDTYGNSF

SEQ ID NO: 644 - skipped

SEQ ID NO: 645 - protein, 9 aa, Homo sapiens
QQNNEDPWT

SEQ ID NO: 646 - protein, 121 aa, Homo sapiens
QVTLKESGPALVKPTQTLTLTCTFSGFSLSTSGMGVSWIRQPPGKALEWLAHIYWDDDKRYNPSLKSRLTISKDTSKNQVVLTMTNMDPVDTATYYCARRPDYFGSSYVDWGQGTTVTVSS

SEQ ID NO: 647 - protein, 330 aa, Homo sapiens [sequence omitted]

SEQ ID NO: 648 - protein, 111 aa, Homo sapiens
DIVMTQTPLSLSVTPGQPASISCRASESVDTYGNSFMHWYLQKPGQPPQLLIYLASNLDFGVPDRFSGSGSGTDFTLKISRVEAEDVGVYYCQQNNEDPWTFGQGTKLEIK

SEQ ID NO: 649 - protein, 107 aa, Homo sapiens [sequence omitted]

SEQ ID NO: 650 - protein, 9 aa, Homo sapiens
GYSITSDYA

SEQ ID NO: 651 - protein, 7 aa, Homo sapiens
ITYSGST

SEQ ID NO: 652 - protein, 14 aa, Homo sapiens
ARSHYGSTYWYFDV

SEQ ID NO: 653 - protein, 10 aa, Homo sapiens
ESVEYYGTSL

SEQ ID NO: 654 - skipped

SEQ ID NO: 655 - protein, 9 aa, Homo sapiens
QQTRKVPWT

SEQ ID NO: 656 - protein, 121 aa, Homo sapiens
QVQLQESGPGLVKPSETLSLTCAVSGYSITSDYAWNWIRQPPGKGLEWIGFITYSGSTNYNPSLKSRVTISVDTSKNQFSLKLSSVTAADTAVYYCARSHYGSTYWYFDVWGQGTTVTVSS

SEQ ID NO: 657 - protein, 330 aa, Homo sapiens [sequence omitted]

SEQ ID NO: 658 - protein, 111 aa, Homo sapiens
DIVMTQTPLSLSVTPGQPASISCRASESVEYYGTSLMQWYLQKPGQSPQLLIYAASNVESGVPDRFSGSGSGTDFTLKISRVEAEDVGVYYCQQTRKVPWTFGQGTRLEIK

SEQ ID NO: 659 - protein, 107 aa, Homo sapiens [sequence omitted]

SEQ ID NO: 660 - protein, 8 aa, Homo sapiens
GYTFTSYD

SEQ ID NO: 661 - protein, 8 aa, Homo sapiens
IYPGDGYT

SEQ ID NO: 662 - protein, 12 aa, Homo sapiens
ARWGYGSYAMDY

SEQ ID NO: 663 - protein, 11 aa, Homo sapiens
QSLVHSNGNTY

SEQ ID NO: 664 - skipped

SEQ ID NO: 665 - protein, 9 aa, Homo sapiens
SQSTHVPPT

SEQ ID NO: 666 - protein, 119 aa, Homo sapiens
QVQLVQSGAEVKKPGASVKVSCKASGYTFTSYDINWVRQAPGQGLEWMGWIYPGDGYTEYNEKFRGRVTMTRDTSTSTVYMELSSLRSEDTAVYYCARWGYGSYAMDYWGQGTLVTVSS

SEQ ID NO: 667 - protein, 330 aa, Homo sapiens [sequence omitted]

SEQ ID NO: 668 - protein, 112 aa, Homo sapiens
DIVMTQSPLSLPVTPGEPASISCRSSQSLVHSNGNTYLHWYLQKPGQSPQLLIYKVSNRFSGVPDRFSGSGSGTDFTLKISRVEAEDVGVYYCSQSTHVPPTFGQGTKLEIK

SEQ ID NO: 669 - protein, 107 aa, Homo sapiens [sequence omitted]

SEQ ID NO: 670 - protein, 8 aa, Homo sapiens
GYTFTSYW

SEQ ID NO: 671 - protein, 8 aa, Homo sapiens
INPSTGYP

SEQ ID NO: 672 - protein, 15 aa, Homo sapiens
ARSYYDYDGGAWFAY

SEQ ID NO: 673 - protein, 10 aa, Homo sapiens
ESVEYYGTSL

SEQ ID NO: 674 - skipped

SEQ ID NO: 675 - protein, 9 aa, Homo sapiens
QQSRKVPST

SEQ ID NO: 676 - protein, 122 aa, Homo sapiens
QVQLVQSGAEVKKPGASVKVSCKASGYTFTSYWMHWVRQAPGQGLEWMGYINPSTGYPGYNQKFKDRVTMTRDTSTSTVYMELSSLRSEDTAVYYCARSYYDYDGGAWFAYWGQGTTVTVSS

SEQ ID NO: 677 - protein, 330 aa, Homo sapiens [sequence omitted]

SEQ ID NO: 678 - protein, 111 aa, Homo sapiens
DIVMTQTPLSLSVTPGQPASISCRASESVEYYGTSLMQWYLQKPGQPPQLLIYAASNVESGVPDRFSGSGSGTDFTLKISRVEAEDVGVYYCQQSRKVPSTFGQGTKLEIK

SEQ ID NO: 679 - protein, 107 aa, Homo sapiens [sequence omitted]

SEQ ID NO: 680 - protein, 8 aa, Homo sapiens
GYTFTDYA

SEQ ID NO: 681 - protein, 8 aa, Homo sapiens
IDTYYGNT

SEQ ID NO: 682 - protein, 10 aa, Homo sapiens
ARGGGDALDY

SEQ ID NO: 683 - protein, 6 aa, Homo sapiens
QSVSND

SEQ ID NO: 684 - skipped

SEQ ID NO: 685 - protein, 9 aa, Homo sapiens
QQGYTSLRT

SEQ ID NO: 686 - protein, 117 aa, Homo sapiens
QVQLVQSGAEVKKPGASVKVSCKASGYTFTDYAMHWVRQAPGQGLEWMGVIDTYYGNTNYNQKFKGRVTMTRDTSISTAYMELSRLRSDDTAVYYCARGGGDALDYWGQGTTVTVSS

SEQ ID NO: 687 - protein, 330 aa, Homo sapiens [sequence omitted]

SEQ ID NO: 688 - protein, 107 aa, Homo sapiens
DIQMTQSPSSLSASVGDRVTITCKASQSVSNDVVWYQQKPGKAPKLLIYYASNRYIGVPSRFSGSGSGTDFTFTISSLQPEDIATYYCQQGYTSLRTFGQGTKLEIK

SEQ ID NO: 689 - protein, 107 aa, Homo sapiens [sequence omitted]

SEQ ID NO: 690 - protein, 9 aa, Homo sapiens
GYSITSGYS

SEQ ID NO: 691 - protein, 7 aa, Homo sapiens
IHYIGIT

SEQ ID NO: 692 - protein, 13 aa, Homo sapiens
AREDYDYDGVFAY

SEQ ID NO: 693 - protein, 6 aa, Homo sapiens
QSISNN

SEQ ID NO: 694 - skipped

SEQ ID NO: 695 - protein, 9 aa, Homo sapiens
QQSNSWPHT

SEQ ID NO: 696 - protein, 120 aa, Homo sapiens
QVQLQESGPGLVKPSETLSLTCAVSGYSITSGYSWHWIRQPPGKGLEWIGYIHYIGITNNNPSLKSRVTISVDTSKNQFSLKLSSVTAADTAVYYCAREDYDYDGVFAYWGQGTLVTVSS

SEQ ID NO: 697 - protein, 330 aa, Homo sapiens [sequence omitted]

SEQ ID NO: 698 - protein, 107 aa, Homo sapiens
EIVLTQSPATLSLSPGERATLSCRASQSISNNLHWYQQKPGQAPRLLIYYASQSISGIPARFSGSGSGTDFTLTISSLEPEDFAVYYCQQSNSWPHTFGQGTKLEIK

SEQ ID NO: 699 - protein, 107 aa, Homo sapiens [sequence omitted]

SEQ ID NO: 700 - protein, 8 aa, Homo sapiens
GFSLTNYD

SEQ ID NO: 701 - protein, 7 aa, Homo sapiens
IWTGGGT

SEQ ID NO: 702 - protein, 10 aa, Homo sapiens
AREGLLLPLY

SEQ ID NO: 703 - protein, 6 aa, Homo sapiens
QNVGTN

SEQ ID NO: 704 - skipped

SEQ ID NO: 705 - protein, 9 aa, Homo sapiens
QQYNSYPLT

SEQ ID NO: 706 - protein, 116 aa, Homo sapiens
QVQLQESGPGLVKPSETLSLTCTVSGFSLTNYDISWIRQPPGKGLEWIGVIWTGGGTNYNSAFMSRVTISVDTSKNQFSLKLSSVTAADTAVYYCAREGLLLPLYWGQGTLVTVSS

SEQ ID NO: 707 - protein, 330 aa, Homo sapiens [sequence omitted]

SEQ ID NO: 708 - protein, 107 aa, Homo sapiens
DIQMTQSPSSLSASVGDRVTITCKASQNVGTNVAWYQQKPGKVPKLLIYSASYRYSGVPSRFSGSGSGTDFTLTISSLQPEDVATYYCQQYNSYPLTFGQGTKLEIK

SEQ ID NO: 709 - protein, 107 aa, Homo sapiens [sequence omitted]

SEQ ID NO: 710 - protein, 8 aa, Homo sapiens
GYTFTNFG

SEQ ID NO: 711 - protein, 8 aa, Homo sapiens
INTYTGEP

SEQ ID NO: 712 - protein, 15 aa, Homo sapiens
ARGAYYYGSRVWFAY

SEQ ID NO: 713 - protein, 11 aa, Homo sapiens
QNLVHSNGNTY

SEQ ID NO: 714 - skipped

SEQ ID NO: 715 - protein, 9 aa, Homo sapiens
SQSSHVPYT

SEQ ID NO: 716 - protein, 122 aa, Homo sapiens
QVQLVQSGSELKKPGASVKVSCKASGYTFTNFGMNWVRQAPGQGLEWMGWINTYTGEPTYADDFKGRFVFSLDTSVSTAYLQICSLKAEDTAVYYCARGAYYYGSRVWFAYWGQGTLVTVSS

SEQ ID NO: 717 - protein, 330 aa, Homo sapiens [sequence omitted]

SEQ ID NO: 718 - protein, 112 aa, Homo sapiens
DIVMTQSPLSLPVTPGEPASISCTSSQNLVHSNGNTYLHWYLQKPGQSPQLLIYKVSNRFSGVPDRFSGSGSGTDFTLKISRVEAEDVGVYYCSQSSHVPYTFGQGTKLEIK

SEQ ID NO: 719 - protein, 107 aa, Homo sapiens [sequence omitted]

SEQ ID NO: 720 - protein, 8 aa, Homo sapiens
GFSLTSSG

SEQ ID NO: 721 - protein, 7 aa, Homo sapiens
IWSGGNT

SEQ ID NO: 722 - protein, 13 aa, Homo sapiens
AREDYDYDWYFDV

SEQ ID NO: 723 - protein, 10 aa, Homo sapiens
ESVEYYGTSL

SEQ ID NO: 724 - skipped

SEQ ID NO: 725 - protein, 9 aa, Homo sapiens
QQSRKVPWT

SEQ ID NO: 726 - protein, 119 aa, Homo sapiens
QVQLQESGPGLVKPSETLSLTCTVSGFSLTSSGVHWIRQPPGKGLEWIGVIWSGGNTDYNAAFISRVTISVDTSKNQFSLKLSSVTAADTAVYYCAREDYDYDWYFDVWGQGTTVTVSS

SEQ ID NO: 727 - protein, 330 aa, Homo sapiens [sequence omitted]

SEQ ID NO: 728 - protein, 111 aa, Homo sapiens
DIVMTQTPLSLSVTPGQPASISCRASESVEYYGTSLMQWYLQKPGQPPQLLIYAASNVKSGVPDRFSGSGSGTDFTLKISRVEAEDVGVYYCQQSRKVPWTFGQGTKLEIK

SEQ ID NO: 729 - protein, 107 aa, Homo sapiens [sequence omitted]

SEQ ID NO: 730 - protein, 8 aa, Homo sapiens
GYTFANFW

SEQ ID NO: 731 - protein, 8 aa, Homo sapiens
IFPGNSDT

SEQ ID NO: 732 - protein, 12 aa, Homo sapiens
ARELTGTYYFDY

SEQ ID NO: 733 - protein, 5 aa, Homo sapiens
SSVSY

SEQ ID NO: 734 - skipped

SEQ ID NO: 735 - protein, 8 aa, Homo sapiens
HQWSGHFT

SEQ ID NO: 736 - protein, 119 aa, Homo sapiens
QVQLVQSGAEVKKPGASVKVSCKASGYTFANFWMHWVRQAPGQGLEWMGAIFPGNSDTGYNQKFKGRVTMTRDTSTSTVYMELSSLRSEDTAVYYCARELTGTYYFDYWGQGTLVTVSS

SEQ ID NO: 737 - protein, 330 aa, Homo sapiens [sequence omitted]

SEQ ID NO: 738 - protein, 105 aa, Homo sapiens
EIVLTQSPATLSLSPGERATLSCSATSSVSYMHWYQQKPGQAPRLLIYSSSNLASGIPARFSGSGSGTDFTLTISSLEPEDFAVYYCHQWSGHFTFGQGTKLEIK

SEQ ID NO: 739 - protein, 107 aa, Homo sapiens [sequence omitted]

SEQ ID NO: 740 - protein, 8 aa, Homo sapiens
GYTFANYL

SEQ ID NO: 741 - protein, 8 aa, Homo sapiens
IYPGGGFI

SEQ ID NO: 742 - protein, 13 aa, Homo sapiens
ARRFDYGGYFFDY

SEQ ID NO: 743 - protein, 11 aa, Homo sapiens
QSIVHSNGNIY

SEQ ID NO: 744 - skipped

SEQ ID NO: 745 - protein, 9 aa, Homo sapiens
FQGSHVPWT

SEQ ID NO: 746 - protein, 120 aa, Homo sapiens
QVQLVQSGAEVKKPGASVKVSCKASGYTFANYLIGWVRQAPGQRLEWMGDIYPGGGFISYNEKFKGRVTITRDTSASTAYMELSSLRSEDTAVYYCARRFDYGGYFFDYWGQGTLVTVSS

SEQ ID NO: 747 - protein, 330 aa, Homo sapiens [sequence omitted]

SEQ ID NO: 748 - protein, 112 aa, Homo sapiens
DIVMTQSPLSLPVTPGEPASISCRSSQSIVHSNGNIYLEWYLQKPGQSPQLLIYKVSNRFSGVPDRFSGSGSGTDFTLKISRVEAEDVGVYYCFQGSHVPWTFGQGTKLEIK

SEQ ID NO: 749 - protein, 107 aa, Homo sapiens [sequence omitted]

SEQ ID NO: 750 - protein, 8 aa, Homo sapiens
GFSITNYD

SEQ ID NO: 751 - protein, 7 aa, Homo sapiens
IWTGGGT

SEQ ID NO: 752 - protein, 12 aa, Homo sapiens
ARDRSPYFGYDY

SEQ ID NO: 753 - protein, 11 aa, Homo sapiens
QSLVHSNGNTY

SEQ ID NO: 754 - skipped

SEQ ID NO: 755 - protein, 9 aa, Homo sapiens
SQSTHVPWT

SEQ ID NO: 756 - protein, 118 aa, Homo sapiens
QVQLQESGPGLVKPSETLSLTCTVSGFSITNYDISWIRQPPGKGLEWIGVIWTGGGTNYNSAFMSRVTISVDTSKNQFSLKLSSVTAADTAVYYCARDRSPYFGYDYWGQGTTVTVSS

SEQ ID NO: 757 - protein, 330 aa, Homo sapiens [sequence omitted]

SEQ ID NO: 758 - protein, 112 aa, Homo sapiens
DIVMTQTPLSLSVTPGQPASISCRSSQSLVHSNGNTYLHWYLQKPGQSPQLLIYKVSNRFSGVPDRFSGSGSGTDFTLKISRVEAEDVGVYYCSQSTHVPWTFGQGTKLEIK

SEQ ID NO: 759 - protein, 107 aa, Homo sapiens [sequence omitted]

SEQ ID NO: 760 - protein, 9 aa, Homo sapiens
GYSITSDYA

SEQ ID NO: 761 - protein, 7 aa, Homo sapiens
MMYSGSA

SEQ ID NO: 762 - protein, 12 aa, Homo sapiens
ARFDHYYGRFDY

SEQ ID NO: 763 - protein, 5 aa, Homo sapiens
SSVSY

SEQ ID NO: 764 - skipped

SEQ ID NO: 765 - protein, 9 aa, Homo sapiens
QQWNYPLFT

SEQ ID NO: 766 - protein, 119 aa, Homo sapiens
QVQLQESGPGLVKPSETLSLTCAVSGYSITSDYAWTWIRQPPGKGLEWIGYMMYSGSASYNPSLKGRVTISVDTSKNQFSLKLSSVTAADTAVYYCARFDHYYGRFDYWGQGTTVTVSS

SEQ ID NO: 767 - protein, 330 aa, Homo sapiens [sequence omitted]

SEQ ID NO: 768 - protein, 106 aa, Homo sapiens
AIQLTQSPSSLSASVGDRVTITCSASSSVSYIHWYQQKPGKAPKLLIYEISKLASGVPSRFSGSGSGTDFTLTISSLQPEDFATYYCQQWNYPLFTFGQGTKLEIK

SEQ ID NO: 769 - protein, 107 aa, Homo sapiens [sequence omitted]

SEQ ID NO: 770 - protein, 8 aa, Homo sapiens
GFTFRNYG

SEQ ID NO: 771 - protein, 8 aa, Homo sapiens
IISGGSYT

SEQ ID NO: 772 - protein, 15 aa, Homo sapiens
ARIYDHDGDYYAMDY

SEQ ID NO: 773 - protein, 11 aa, Homo sapiens
QSIVHSNGNTY

SEQ ID NO: 774 - skipped

SEQ ID NO: 775 - protein, 9 aa, Homo sapiens
FQGSHVPWT

SEQ ID NO: 776 - protein, 122 aa, Homo sapiens
EVQLVESGGGLVQPGGSLRLSCAASGFTFRNYGMSWVRQAPGKGLEWVASIISGGSYTYYPDSVKGRFTISRDNAKNSLYLQMNSLRAEDTAVYYCARIYDHDGDYYAMDYWGQGTTVTVSS

SEQ ID NO: 777 - protein, 330 aa, Homo sapiens [sequence omitted]

SEQ ID NO: 778 - protein, 112 aa, Homo sapiens
DIVMTQSPLSLPVTPGEPASISCRSSQSIVHSNGNTYLEWYLQKPGQSPQLLIYKVSNRFSGVPDRFSGSGSGTDFTLKISRVEAEDVGVYYCFQGSHVPWTFGQGTKLEIK

SEQ ID NO: 779 - protein, 107 aa, Homo sapiens [sequence omitted]

SEQ ID NO: 780 - protein, 8 aa, Homo sapiens
GYTFTSYD

SEQ ID NO: 781 - protein, 8 aa, Homo sapiens
IYPGDGST

SEQ ID NO: 782 - protein, 10 aa, Homo sapiens
AREGITPFAY

SEQ ID NO: 783 - protein, 6 aa, Homo sapiens
KSISKY

SEQ ID NO: 784 - skipped

SEQ ID NO: 785 - protein, 9 aa, Homo sapiens
QQHNEYPWT

SEQ ID NO: 786 - protein, 117 aa, Homo sapiens
QVQLVQSGAEVKKPGSSVKVSCKASGYTFTSYDINWVRQAPGQGLEWMGRIYPGDGSTKYNEKFKGRVTITADKSTSTAYMELSSLRSEDTAVYYCAREGITPFAYWGQGTTVTVSS

SEQ ID NO: 787 - protein, 330 aa, Homo sapiens [sequence omitted]

SEQ ID NO: 788 - protein, 107 aa, Homo sapiens
AIQLTQSPSSLSASVGDRVTITCRASKSISKYLAWYQQKPGKAPKLLIYSGSTLQSGVPSRFSGSGSGTDFTLTISSLQPEDFATYYCQQHNEYPWTFGQGTKLEIK

SEQ ID NO: 789 - protein, 107 aa, Homo sapiens [sequence omitted]

SEQ ID NO: 790 - protein, 8 aa, Homo sapiens
GGTFSSYA

SEQ ID NO: 791 - protein, 8 aa, Homo sapiens
IIPIFGTA

SEQ ID NO: 792 - protein, 13 aa, Homo sapiens
ARSSYGWSYEFDY

SEQ ID NO: 793 - protein, 6 aa, Homo sapiens
QSISSY

SEQ ID NO: 794 - skipped

SEQ ID NO: 795 - protein, 9 aa, Homo sapiens
QQSYSTPLT

SEQ ID NO: 796 - protein, 120 aa, Homo sapiens; identical to SEQ ID NO: 226

SEQ ID NO: 797 - protein, 330 aa, Homo sapiens [sequence omitted]

SEQ ID NO: 798 - protein, 107 aa, Homo sapiens [sequence omitted]

SEQ ID NO: 799 - protein, 107 aa, Homo sapiens [sequence omitted]

SEQ ID NO: 800 - protein, 8 aa, Homo sapiens
GGTFSSYA

SEQ ID NO: 801 - protein, 8 aa, Homo sapiens
IIPIFGTA

SEQ ID NO: 802 - protein, 13 aa, Homo sapiens
ARSTVGWSYEFDY

SEQ ID NO: 803 - protein, 6 aa, Homo sapiens
QSISSY

SEQ ID NO: 804 - skipped

SEQ ID NO: 805 - protein, 9 aa, Homo sapiens
QQSYSTPLT

SEQ ID NO: 806 - protein, 120 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARSTVGWSYEFDYWGQGTLVTVSS

SEQ ID NO: 807 - protein, 330 aa, Homo sapiens [sequence omitted]

SEQ ID NO: 808 - protein, 107 aa, Homo sapiens [sequence omitted]

SEQ ID NO: 809 - protein, 107 aa, Homo sapiens [sequence omitted]

SEQ ID NO: 810 - protein, 8 aa, Homo sapiens
GGTFSSYA

SEQ ID NO: 811 - protein, 8 aa, Homo sapiens
IIPIFGTA

SEQ ID NO: 812 - protein, 13 aa, Homo sapiens
ARHVLGWVLELDY

SEQ ID NO: 813 - protein, 6 aa, Homo sapiens
QSISSY

SEQ ID NO: 814 - skipped

SEQ ID NO: 815 - protein, 9 aa, Homo sapiens
QQSYSTPLT

SEQ ID NO: 816 - protein, 120 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARHVLGWVLELDYWGQGTLVTVSS

SEQ ID NO: 817 - protein, 330 aa, Homo sapiens [sequence omitted]

SEQ ID NO: 818 - protein, 107 aa, Homo sapiens [sequence omitted]

SEQ ID NO: 819 - protein, 107 aa, Homo sapiens [sequence omitted]

SEQ ID NO: 820 - protein, 219 aa, synthetic construct
DIVMTQSPLSLPVTPGEPASISCRSSQSIVHSNGNIYLEWYLQKPGQSPQLLIYKVSNRFSGVPDRFSGSGSGTDFTLKISRVEAEDVGVYYCFQGSHVPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 821 - protein, 450 aa, synthetic construct
QVQLVQSGAEVKKPGASVKVSCKASGYTFANYLIGWVRQAPGQRLEWMGDIYPGGGFISYNEKFKGRVTITRDTSASTAYMELSSLRSEDTAVYYCARRFDYGGYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHRDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 822 - protein, 223 aa, synthetic construct
QVQLVQSGAEVKKPGASVKVSCKASGYTFANYLIGWVRQAPGQRLEWMGDIYPGGGFISYNEKFKGRVTITRDTSASTAYMELSSLRSEDTAVYYCARRFDYGGYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 823 - RNA, 25 nt, synthetic construct
  source 1..25: mol_type = other RNA
accctgaagttcatctgcaccaccg

SEQ ID NO: 824 - RNA, 27 nt, synthetic construct
  source 1..27: mol_type = other RNA
cggtggtgcagatgaacttcagggtca

SEQ ID NO: 825 - DNA, 25 bp, synthetic construct
  source 1..25: mol_type = other DNA
  misc_feature 1..23: note = RNA
  misc_feature 24..25: note = DNA
gagaaaggacgcatgctgtttctta

SEQ ID NO: 826 - RNA, 27 nt, synthetic construct
  source 1..27: mol_type = other RNA
taagaaacagcatgcgtcctttctcca

SEQ ID NO: 827 - DNA, 16 bp, synthetic construct
  source 1..16: mol_type = other DNA
  modified_base 1: mod_base = OTHER; note = Locked nucleic acid with phosphorothioate linkage
  modified_base 2: mod_base = OTHER; note = Locked nucleic acid with phosphorothioate linkage
  modified_base 3: mod_base = OTHER; note = Locked nucleic acid with phosphorothioate linkage
  modified_base 4: mod_base = OTHER; note = Nucleotide with phosphorothioate linkage
  modified_base 5: mod_base = OTHER; note = Nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Locked nucleic acid with phosphorothioate linkage
  modified_base 15: mod_base = OTHER; note = Locked nucleic acid with phosphorothioate linkage
  modified_base 16: mod_base = OTHER; note = Locked nucleic acid
ggttgtgtgctaccat

SEQ ID NO: 828 - RNA, 25 nt, synthetic construct
  source 1..25: mol_type = other RNA
cttgggaaatcgatgagaaattgaa

SEQ ID NO: 829 - RNA, 27 nt, synthetic construct
  source 1..27: mol_type = other RNA
ttcaatttctcatcgatttcccaaggc

SEQ ID NO: 830 - DNA, 25 bp, synthetic construct
  source 1..25: mol_type = other DNA
  misc_feature 1..23: note = RNA
  misc_feature 24..25: note = DNA
ggtctactttgggatcattgccctg

SEQ ID NO: 831 - RNA, 27 nt, synthetic construct
  source 1..27: mol_type = other RNA
cagggcaatgatcccaaagtagacctg

SEQ ID NO: 832 - RNA, 25 nt, synthetic construct
  source 1..25: mol_type = other RNA
cggtcttttttgaaggcaaatacgg

SEQ ID NO: 833 - RNA, 27 nt, synthetic construct
  source 1..27: mol_type = other RNA
ccgtatttgccttcaaaaaagaccgtg

SEQ ID NO: 834 - RNA, 25 nt, synthetic construct
  source 1..25: mol_type = other RNA
ccgggccaagagtgtgctaaagaag

SEQ ID NO: 835 - RNA, 27 nt, synthetic construct
  source 1..27: mol_type = other RNA
cttctttagcacactcttggcccggtt

SEQ ID NO: 836 - DNA, 25 bp, synthetic construct
  source 1..25: mol_type = other DNA
  misc_feature 1..23: note = RNA
  misc_feature 24..25: note = DNA
cagactgaatttgcgatgaaaatat

SEQ ID NO: 837 - RNA, 27 nt, synthetic construct
  source 1..27: mol_type = other RNA
atattttcatcgcaaattcagtctgtc

SEQ ID NO: 838 - DNA, 25 bp, synthetic construct
  source 1..25: mol_type = other DNA
  misc_feature 1..23: note = RNA
  misc_feature 24..25: note = DNA
gtccaaaaaggtaaactttgaagat

SEQ ID NO: 839 - RNA, 27 nt, synthetic construct
  source 1..27: mol_type = other RNA
atcttcaaagtttacctttttggactt

SEQ ID NO: 840 - DNA, 16 bp, synthetic construct
  source 1..16: mol_type = other DNA
  modified_base 1: mod_base = OTHER; note = Locked nucleic acid with phosphorothioate linkage
  modified_base 2: mod_base = OTHER; note = Locked nucleic acid with phosphorothioate linkage
  modified_base 3: mod_base = OTHER; note = Locked nucleic acid with phosphorothioate linkage
  modified_base 4: mod_base = OTHER; note = Nucleotide with phosphorothioate linkage
  modified_base 5: mod_base = OTHER; note = Nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Locked nucleic acid with phosphorothioate linkage
  modified_base 16: mod_base = OTHER; note = Locked nucleic acid
gtttgtgtgagagctt

SEQ ID NO: 841 - DNA, 25 bp, synthetic construct
  source 1..25: mol_type = other DNA
  misc_feature 1..23: note = RNA
  misc_feature 24..25: note = DNA
tacggaaagatgatcagctattttc

SEQ ID NO: 842 - RNA, 27 nt, synthetic construct
  source 1..27: mol_type = other RNA
gaaaatagctgatcatctttccgtaca

SEQ ID NO: 843 - DNA, 25 bp, synthetic construct
  source 1..25: mol_type = other DNA
  misc_feature 1..23: note = RNA
  misc_feature 24..25: note = DNA
aatctgagaggaaagcgtttattgt

SEQ ID NO: 844 - RNA, 27 nt, synthetic construct
  source 1..27: mol_type = other RNA
acaataaacgctttcctctcagattca

SEQ ID NO: 845 - RNA, 25 nt, synthetic construct
  source 1..25: mol_type = other RNA
ggatggacatgatattgagaacgag

SEQ ID NO: 846 - RNA, 27 nt, synthetic construct
  source 1..27: mol_type = other RNA
ctcgttctcaatatcatgtccatcctg

SEQ ID NO: 847 - RNA, 25 nt, synthetic construct
  source 1..25: mol_type = other RNA
aggtcatggatgccaagcacaagaa

SEQ ID NO: 848 - RNA, 27 nt, synthetic construct
  source 1..27: mol_type = other RNA
ttcttgtgcttggcatccatgaccttc

SEQ ID NO: 849 - DNA, 25 bp, synthetic construct
  source 1..25: mol_type = other DNA
  misc_feature 1..23: note = RNA
  misc_feature 24..25: note = DNA
aaggtttaaggagaatctaagcatt

SEQ ID NO: 850 - RNA, 27 nt, synthetic construct
  source 1..27: mol_type = other RNA
aatgcttagattctccttaaaccttcc

SEQ ID NO: 851 - RNA, 25 nt, synthetic construct
  source 1..25: mol_type = other RNA
ggaggcttctgatacgtgtatgaga

SEQ ID NO: 852 - RNA, 27 nt, synthetic construct
  source 1..27: mol_type = other RNA
tctcatacacgtatcagaagcctccac

SEQ ID NO: 853 - DNA, 25 bp, synthetic construct
  source 1..25: mol_type = other DNA
  misc_feature 1..23: note = RNA
  misc_feature 24..25: note = DNA
caccatctggtaataaactcatgtt

SEQ ID NO: 854 - RNA, 27 nt, synthetic construct
  source 1..27: mol_type = other RNA
aacatgagtttattaccagatggtgga

SEQ ID NO: 855 - DNA, 25 bp, synthetic construct
  source 1..25: mol_type = other DNA
  misc_feature 1..23: note = RNA
  misc_feature 24..25: note = DNA
gattttctattgttactctggttct

SEQ ID NO: 856 - RNA, 27 nt, synthetic construct
  source 1..27: mol_type = other RNA
agaaccagagtaacaatagaaaatcca

SEQ ID NO: 857 - RNA, 27 nt, synthetic construct
  source 1..27: mol_type = other RNA
atcttcaaagtttacctttttggactt

SEQ ID NO: 858 - protein, 24 aa, Mus musculus
LLDSGLYCCLVVEIRHHHSEHRVH

SEQ ID NO: 859 - protein, 21 aa, Mus musculus
GHDVTFYKTWYRSSRGEVQTC

SEQ ID NO: 860 - protein, 15 aa, Mus musculus
LLDSGLYCCLVVEIR

SEQ ID NO: 861 - protein, 9 aa, Homo sapiens
NLTLLDSGL

SEQ ID NO: 862 - protein, 11 aa, Homo sapiens
VQTGKDAPSNC

SEQ ID NO: 863 - protein, 10 aa, Homo sapiens
PVDKGHDVTF

SEQ ID NO: 864 - protein, 12 aa, Homo sapiens
RRPIRDLTFQDL

SEQ ID NO: 865 - protein, 6 aa, Homo sapiens
ARRWFL

SEQ ID NO: 866 - protein, 9 aa, Homo sapiens
QQYNNYPLT

SEQ ID NO: 867 - protein, 9 aa, Homo sapiens
QQINSWPTT

SEQ ID NO: 868 - protein, 24 aa, Homo sapiens
LLDSGLYCCLVVEIRHHHSEHRVH

SEQ ID NO: 869 - protein, 21 aa, Homo sapiens
GHDVTFYKTWYRSSRGEVQTC

SEQ ID NO: 870 - protein, 15 aa, Homo sapiens
LLDSGLYCCLVVEIR